SEQUENCE LISTING (269 sequences)

SEQ ID NO: 1 - DNA, 813 bp, Sus scrofa
  source 1..813: mol_type = genomic DNA
atgccccgagacaacatggcctccctgatccaacggatcgcccgccaggcatgcctcaccttccggggcagcgggggcggccgcagctcttccgatcgcggcgcggcgccaggccctgaggcgcctgtgccgcagggcttcccggagaacctgagcaagctgaagagcctgctgacacaggtccgcgcagaggacctgaacatctccccgcgcaaggccacgctgcagccgttgccacccaacctgccgcccgtcacctatatgcacatctacgagactgacggcttcagcctcggcgtgttcttgcttaagagcggcacatccatcccgctccacgaccaccctggcatgcatggcatgctcaaggtgctctatggcaccgtgcgcatcagctgcatggacaagctggaggcaggcagcgggcaacggccgcgggccccgccaccagagcagcagttcgaaccgccgctgctggcccgggagcgggacgcggtgcggccgggagtgctgcgctcgcgggccgagtacactgaggccagcggtccctgcgtcctcacgccgcaccgggacaacctgcaccagatcgacgctgtggatgggcctgccgccttcttggatatcctggccccgccctacgacccggacgacggccgggactgtcactattaccgggtgctggagcctgtcagggccaaagaggcctccgactcggcctgtgacctgccccgagaggtgtggcttctggagaccccgcaggccgatgacttttggtgcgagggggagccctatccaggtcccagggtcttcccttga

SEQ ID NO: 2 - protein, 270 aa, Sus scrofa
MPRDNMASLIQRIARQACLTFRGSGGGRSSSDRGAAPGPEAPVPQGFPENLSKLKSLLTQVRAEDLNISPRKATLQPLPPNLPPVTYMHIYETDGFSLGVFLLKSGTSIPLHDHPGMHGMLKVLYGTVRISCMDKLEAGSGQRPRAPPPEQQFEPPLLARERDAVRPGVLRSRAEYTEASGPCVLTPHRDNLHQIDAVDGPAAFLDILAPPYDPDDGRDCHYYRVLEPVRAKEASDSACDLPREVWLLETPQADDFWCEGEPYPGPRVFP

SEQ ID NO: 3 - DNA, 1582 bp, Sus scrofa
  source 1..1582: mol_type = genomic DNA
atgatcacatctccgttgctggcttatgtggcgattctgtttttgtgtgtcttaaaagccagctccctggatacctttatcgccgctgtttatgaacatgcagtgatattgcctgatgcccccctcacaccagtatcccatgaagaggctttgatgttaatgaaccgcaatctggatcttttggagggcgcaatcatgtcagcagcaaagcagggtgcacatattattgtgactccagaagatggtgtttacggctttttcttcagcagggaatctatttactcatacttagaggacatcccagagcctcacgtgaactggatcccttgtaccaatcccaacagatttggccacactccagtacaaaaaagactcagctgcctggccagggacaactccatctacattgtggcaaatattggggacaagaagccatgcaatgccagtgaccccgactgtcctcatgacggccgttaccagtacaacactgatgtggtgtttgattctgagggcagactggtggcacgctatcataagcaaaatcttttcttgggtgaagatcaattcgatgcacccaaggaacccgagatcgtgactttcgacaccacctttggaagatttggcattttcacgtgctttgatattctcttccatgatcctgctgtgactctggtgaaagatttccaggtggacaccatactcttcccgacagcttggatgaatgttttgccacatttgacagctattgaattccactcagcttgggctatgggcatgagagtcaacttccttgcagccaatatacatttccccttaagaaaaatgacaggaagtggcatctatgcacctgattctccaagagcatttcattacgatatgaagacaaaggagggaaaactcctcctctcacaattggactcccacccacaccgcccagcagtgaattggacttcttatgccagtggtttaccagcccccttagtgggaaaccaggaatttaaaagcattgtcttttttgacgagttcaccttcttcgagctcaaaggagttgcaggaaattacacagtttgtcagaaagatctctgctgtcagctaagttacaggatgcttgagatgagagaagacgaagtttatgcactaggggcattcgatggactacatactgtggaagggagctattatttacagatttgtactttgttgaagtgtaaaacaatggacttacactcctgtggtgactcggtggaaactgcttccaccaggtttgaaatgttctccctcagtggcacttttggaacccagtatgtgttccctgaggtgttgctgagtgacatacagcttgcacctggagaatttcaggtgtcaagtgatggacgcttgttcagcctgaagccaccgtctggacctgtcttgactgtcactctgtttgggaggttatatgagagggatagtgcatcaggtgcttcagcagacctcgtggcacaaggactgagagtcatgctcggagttataataactattatgtactcattaagttggtagaatatttatacttttctctctttcacttcggatcctttaa

SEQ ID NO: 4 - protein, 513 aa, Sus scrofa
MITSPLLAYVAILFFCVLKASSLDTFIAAVYEHAAILPDAPLTPVSHEEALMLMNRNLDLLEGAVTSAAKQGAHIIVTPEDGVYGFFFSRESIYSYLEDIPDPHVNWIPCTNPSRFGHTPVQKRLSCLARDNSIYIVANIGDKKPCNASDPDCPHDGRYQYNTDVVFDSEGRLVARYHKQNLFLGEDQFDAPKEPEIVTFDTTFGRFGIFTCFGILFHDPAVTLVKDFQVDTILFPTAWMNVLPHLTAIEFHSAWAMGMRVNFLAANIHFPLRKMTGSGIYAPDSPRAFHYDMKTKEGKLLLSQLDSHPHRPAVNWTSYASGLPTPLVGNQEFKSTVFFDEFTFLELKGVAGNYTVCQKDLCCQLSYRMLEKREDEVYALGAFDGLHTVEGSYYLQICTLLKCKTMDLHSCGDSVETASTRFEMFSLSGTFGTQYVFPEVLLSDIQLAPGEFQVSSDGRLFSLKPPSGPVLTVTLFGRLYERDSASGASADLVAQGLRVMLGVIITIMYSLSW

SEQ ID NO: 5 - DNA, 1599 bp, Sus scrofa
  source 1..1599: mol_type = genomic DNA
atggccaagcgagttgcaattgtgggagctggggtcagtggcctggcctccatcaagtgctgcctggaggaagggctggagcccacctgctttgagaggagcgatgaccttggggggctgtggagattcactgaacatgttgaagaaggcagagccagcctctacaagtctgtggtttccaacagctgcaaggagatgtcttgttacccagactttccattcccagaagattacccaaactatgtgccaaattctcacttcctggaatatctcagaatgtatgcaaaccagttcaaccttctgaagtgcattcaattcaagactaaagtctgcagtgtaacaaaacatgaagattttaataccaccggtcaatgggatgtagtcactctgtgtgaaggaaagcaagagtcagccgtctttgatgctgtcatggtctgcactggttttctcactaacccatatttgccattggactccttcccaggcataaatacttttaaaggccagtatttccatagccgacagtataaacatccagatatatttaaggacaagagtgttcttgtggttggaatgggaaattcgggcacagacattgctgtggaggccagccacctggcaaaaaaggtgttcctcagcaccactggaggagcatgggtgatcagccgtgtctttgactcagggtacccatgggacatggtgttcatgacacgatttcagaacatgttcagaaattctctcccaactccaattgtgaattggttgatagcaaaaaagatgaacagctggttcaatcatgcaaattatggcttaatacccgaagacaggatacaactaagagagcctgtgctgaatgatgagctcccaggccgtatcatcactgggaaagtgctcatcaagccaagtataaaggaggtgaaggaaaactccgtggtatttaacagctccccagaggaagagcccattgatatcattgtctttgccactggatacacctttgctttccccttccttgatgagagtgtagtgaaggttgaagatggccaggcatcactatacaagtacatcttccctgcacatctgcagaagccaactctggctgttattggcctcatcaaacccttgggctccttactacccacaggggatacacaagctcggtgggctgttcgggtcctgaaaggtgtgaataagttaccaccatcaagtgtcatgatacaggaagttaatacaagaaaagaaaacaagcccagtgggtttggcttgtgttactgcaaagctttacaatccgattatattgcatacatagatgaactgctgacctatatcgatgcaaagcccaacatgttttctctgctcctgacggatccacacctggctttgaccatcttctttggcccatgcacaccataccagttccgcctgactggtccagggaaatgggaaggagccaggaatgctatcatgacccagtgggaccgaacattcaaggtcaccaaaactcgaattgtaaaagaatccccatctccctttgcaagcttacttaaactctttagctttctagccttgcttgtggccattttccagattttcctctaa

SEQ ID NO: 6 - protein, 532 aa, Sus scrofa
MAKRVAIVGAGVSGLASIKCCLEEGLEPTCFERSDDLGGLWRFTEHVEEGRASLYKSVVSNSCKEMSCYPDFPFPEDYPNYVPNSHFLEYLRMYANQFNLLKCIQFKTKVCSVTKHEDFNTTGQWDVVTLCEGKQESAVFDAVMVCTGFLTNPYLPLDSFPGINTFKGQYFHSRQYKHPDIFKDKSVLVVGMGNSGTDIAVEASHLAKKVFLSTTGGAWVISRVFDSGYPWDMVFMTRFQNMFRNSLPTPIVNWLIAKKMNSWFNHANYGLIPEDRIQLREPVLNDELPGRIITGKVLIKPSIKEVKENSVVFNSSPEEEPIDIIVFATGYTFAFPFLDESVVKVEDGQASLYKYIFPAHLQKPTLAVIGLIKPLGSLLPTGDTQARWAVRVLKGVNKLPPSSVMIQEVNTRKENKPSGFGLCYCKALQSDYIAYIDELLTYIDAKPNMFSLLLTDPHLALTIFFGPCTPYQFRLTGPGKWEGARNAIMTQWDRTFKVTKTRIVKESPSPFASLLKLFSFLALLVAIFQIFL

SEQ ID NO: 7 - DNA, 251 bp, Corynebacterium glutamicum
  source 1..251: mol_type = genomic DNA
cttgcagcgttttgctctgccatgaatccattgtgcaccttagctactccattagtgtgatcggggttattttttcacttcaatgggtggctaaaagacgtgggcacgtgagtaaactcatgcgcgcgaaacgatggaagtgaacccatacttttatatatgggtatcggcggtctatgcttgtgggcgtacctgtcccgcgagtgaggtcttacgcgcgggattcgtcttgtgaaaggttagctgacctg

SEQ ID NO: 8 - DNA, 250 bp, Corynebacterium glutamicum
  source 1..250: mol_type = genomic DNA
gccataacaggaatgttcctttcaaaaattgaggaagccttatgacctacaaccctactcagctgccgattattccgggtttgtgaccagctacccgataaataggtcggctgaaaaatttcgttgcaatatcaacaaaaaggcctatcattgggaagtgtcgcaccaagtacttttgcgaagcgccatctgacggattttcaaaagatgtatatgctcggtgcggaaacctacgaaaggatttttaccc

SEQ ID NO: 9 - DNA, 250 bp, Corynebacterium glutamicum
  source 1..250: mol_type = genomic DNA
gcgtctgaccgagctcgtagcttccaagctctaattagttcacatcgctaacgtgggcgatcgatgctcacggtgatgtgtcatcccaatagcccggggtgtgcctcggcgcaccccgggctattttgtgtctttaatcaatacaattgaataccggtgccagcgccacacaatgtgtggcaatctgggacagtgcatcacattgcaccagaagaattttttaaacaatcaaatctccaaggagtacggc

SEQ ID NO: 10 - DNA, 250 bp, Corynebacterium glutamicum
  source 1..250: mol_type = genomic DNA
tagatatcttgaaatcggctttcaacagcattgatttcgatgtatttagctggccgttaccctgcgaatgtccacagggtagctggtagtttgaaaatcaacgccgttgcccttaggattcagtaactggcacattttgtaatgcgctagatctgtgtgctcagtcttccaggctgctgatcacagtgaaagcaaaaccaattcgtggctgcgaaagtcgtagccaccacgaagtccaggaggacataca

SEQ ID NO: 11 - DNA, 250 bp, Corynebacterium glutamicum
  source 1..250: mol_type = genomic DNA
tagccccgatagtgtatgtgctgacctgcgtttatgcattttccttaggttgaaggaaatatcacacgacaaaagttgagtgatgcaggcataattggctatgggcaactgaagatgccacaatcaatgtttgatcaaacagaatcagacattgtgaatgtgggattatcggttcgcgcttcaccatgtttctgcatgatgaaattacatacatagttcagtgacagtcaccttttggaggagacacctt

SEQ ID NO: 12 - DNA, 250 bp, Corynebacterium glutamicum
  source 1..250: mol_type = genomic DNA
gcacttgattcagggtagttgactaaagagttgctcgcgaagtagcacctgtcacttttgccacaaataccaaattgcttatcagcatatgggctgtttgcttgccccagtcccagcagctgagacgcatccgctaaagccccaggaaccctgtacagaaagaaaacactcctctggctaggtagacacagtttataaaggtagagttgagcgggtaactgtcagcacgtagatcgaaaggtgcacaaag

SEQ ID NO: 13 - DNA, 250 bp, Corynebacterium glutamicum
  source 1..250: mol_type = genomic DNA
cacgtcacagctcccagacttaagattggtcacacctttgacacatttggaccacagttggttataaaatgggttcaacatcactatggttagaggtgttgacgggtcagattaagcaaagactactttcggggtagatcacctttgccaaatttgaaccaattaacctaagtcgtagatctgatcatcggatctaacgaaaacgaaccaaaactttggtcccggtttaacccaggaaggattgaccacc

SEQ ID NO: 14 - DNA, 250 bp, Corynebacterium glutamicum
  source 1..250: mol_type = genomic DNA
gcggatactgcagtcataggcgttgcttttcgacgtcgcaaagcgcaatttcctacctttaagatcctaatctgttgaggtcagccacaatttttcagaaaagttttgatagatcgacaggtaatgctttatactgacaacgtcgcaaggactacatttgcagccaagtctactacttgatcttcaaaggtcagcaattgtgaacaaagctacaaataaaccgttccgcccatgtcaatgaggagtcacc

SEQ ID NO: 15 - DNA, 250 bp, Corynebacterium glutamicum; identical to SEQ ID NO: 14
  source 1..250: mol_type = genomic DNA

SEQ ID NO: 16 - DNA, 250 bp, Corynebacterium glutamicum
  source 1..250: mol_type = genomic DNA
gagcgagcgaagtttcaaaatgccttcctgggggtgaaattagatcctgttaatgtactaaatttttcaaaagttttctcgttgaaaattaagaagctgggaaggtgaattgaattttgaggctttaaagcaaaaatgaacagcttggtctatagtggctaggtaccctttttgttttggacacctgtagggtggccgaagcaatgaaataggacaacaacgctcgaccgcgattattttttggagaatc

SEQ ID NO: 17 - DNA, 251 bp, Corynebacterium glutamicum
  source 1..251: mol_type = genomic DNA
ccgtaattggcgttgttgagtgcttcaagttcgtcagttgttaaagctctggtggcggcaagttctgcaagcgaaagcagatcttggggttgatcatcgcgggaagtcatattttattactctagtcggcctaaaatggttggattttcacctgctgtgacctggtaaaatggccactacccccaaatggtcacaccttttaggccgattttgctgacaccgggctatgccgtcaagtacgatcaataaca

SEQ ID NO: 18 - DNA, 250 bp, Corynebacterium glutamicum
  source 1..250: mol_type = genomic DNA
gttttcttgcgctgctgcagtgggcattgataccaaaaaggggctaagcgcagtcgaggcggcaagaactgctactaccctttttattgtcgaacggggcattacggctccaaggacgtttgttttctgggttagttaccccaaaaagcatatacagagaccaatgatttttcattaaaaaggcagggatttgttataagtatgggtcgtattctgtgcgacgggtgtacctcggctagaatttctcccc

SEQ ID NO: 19 - DNA, 250 bp, Corynebacterium glutamicum
  source 1..250: mol_type = genomic DNA
tcgtttagaaactcaaatccgctcgcagttggcgttttctggggcggttcagctagagttatgcgaaggatcccgtgcggcgtttatcttgtgaactcccccagggcaggaatgcagcaagggtcagcgagctctgacgggtgcgcgggatcccctaaaacgtctagagtagtggcttgaggtcactgctctttttttgtgcccttttttggtccgtctattttgccaccacatgcggaggtacgcagtt

SEQ ID NO: 20 - DNA, 250 bp, Corynebacterium glutamicum
  source 1..250: mol_type = genomic DNA
catccggataaaccaccatgaacggcagtggatcaaaagtcctgttggtgaagctgcgccccacagatcctgactgctgggagccatgaaaatagatcagcgcatccgtggtggaaccaaaaggctcaacaatacgaaacgttcgctttcggtcctgatgaaagagatgtccctgaatcatcatctaagtatgcatctcggtaagctcgaccaggacagtgccaccacaattttggaggattacaagaac

SEQ ID NO: 21 - DNA, 250 bp, Corynebacterium glutamicum
  source 1..250: mol_type = genomic DNA
aagaggcggaaaccacaagcgccaaggaactacctgcggaacgggctgtgaagggcaacttaagtctcatatttcaaacatagttccacctgtgtgattaatccctagaacggaacaaactgatgaacaatcgttaacaacacagaccaaaacggtcagttaggtatggatatcagcaccttctgaacgggtacgtctagactggtgggcgtttgaaaaactcttcgccccacgaaaatgaaggagcata

SEQ ID NO: 22 - DNA, 201 bp, Corynebacterium glutamicum
  source 1..201: mol_type = genomic DNA
cgcaaagctcacacccacgagctaaaaattcatatagttaagacaatatttttggctgtaaaagacagccgtaaaaacctcttgctcatgtcaattgttcttatcggaatgtggcttgggcgattgttatgcaaaagttgttaggttttttgcggggttgtttaacccccaaatgagggaagaaggtaaccttgaactcta

SEQ ID NO: 23 - DNA, 29 bp, Corynebacterium glutamicum
  source 1..29: mol_type = other DNA
ttgacaattaatcatcggctcgtataatg

SEQ ID NO: 24 - DNA, 812 bp, Balaenoptera musculus
  source 1..812: mol_type = genomic DNA
atgccccgagacaacatggcctccctgatccaacggatagcccgccaggcgtgcctcaccttccggggcagcgggggcagccgcagctcttccgaccgcggcgaggcgccaggccccgaggcgccgatgtcgcagggcttcccggagaacctgagcaagctgaagagcctgctgaccctggtccgcgcggaggacctgaacatctccccgcgcaaggccacgctgcagccgctgaggcccaacctgccgccagtcacctatatgcacatctgcgagacagacggcttcagcctcggcgtgttcctgctgaagagcggcacctccatcccgctgcacgaccacccgggcatgcacggcatcctcaaggtgctctacggcaccgtgcgcatcagctgcatggacaagctggaggtgggcagcgggcaacggccgcgggccccgccacccgagcagcagttcgagccggcgttgcagcccggggagcgggacgcggtaaggccgggtgtgctgcgctcgcgggcggagtacacggcggccagcggcccctgcgtcctcacgccgcaccgggacaacctgcaccagatcgacgctgtggaggggcccgccgctttcctggacatcctggccccgccctacgaccccgacgacggccgggactgccactattaccgggtgctggagcctgtcagggccagggaggcctccggctcggcctgtgacctgccccgagaggtgtggctcctggagaccccgcaggccgatgacttctggtgcgagggggagccctatccaggtcccaaggtcttcccttg

SEQ ID NO: 25 - DNA, 798 bp, Molossus molossus
  source 1..798: mol_type = genomic DNA
atggcctccctgatccaacggatcgcccgccaggcgtgcctcaccttccgaggcagcgggggcggccgcagtgcttccgaccgcggctcggtgccaggccccgaggcgccggggccgcagggcttcccggagaacctgagcaagctgaagagtctgctgacccaggtccgtgctgaggacctgaatatcgccccgcgcaaagcgacgctgcagccgctgccacccaacctgccacccgttacctacatgcacatctacgagacggacggcttcagcctcggcgtgttcctgctcaagagcggcacgtccatccctctgcacgaccacccgggcatgcacggcatgctcaaggtgctctacggcaccgtgcgcatcagctgcatggacaagctggaggcaggcggcgggcagcggcagcgagccccaccgccagaacagcagttcgagccgccgctgcagccccgggagcgggacgctgtccggcctggcgtgctccgctcgcgggccgagtacaccgaggccagcggtccctgcgtccttacgccgcaccgggacaacctgcaccaaatcgacgctgtggacgggcccgctgccttcctggacatcctggccccgccctacgaccccgacgacggccgggactgccactattaccgggtgctggaaccggtcagggccaaggaggcctccggatcgacctgtgacctgcctcgagaggtgtggcttctcgagactccgcaggccgatgacttctggtgcgagggggagccctatccaggtcccaaggtcgtcccttga

SEQ ID NO: 26 - DNA, 798 bp, Felis catus
  source 1..798: mol_type = genomic DNA
atggcctccctgatccaacggatcgcccgccaggcgtgtctcaccttccggggcagcgggggctgccgcagcgcttccgaccacggcgcggcgccgggctccgaggcccctatgcctcagggcttcccggagaacctgagcaagctgaagaacctgctgacccaagtccgcgcggaggacctgaacatcgccccgcgcaaggccacggtgcagccgttgccgcccaacctaccaccggtcacctatatgcacatttacgaaaccgacggcttcagcctcggcgtgttcctgctcaagagcggcacgtccattccgcttcatgaccacccgggcatgcacggcatgctcaaggtgctctacggcaccgtgcgcatcagctgcatggacaagctggaggcgggcggcgggcagcggccacgggccccgccgccagagcagcagttcgagccgccgctgaaagcccgggagcgggacgccgtgcggctgggcgtgctgcgctcgagggccgagtacaccgaggccagcggcccctgcgtcctcaccccacaccaggacaacctgcaccagatcgacgctgtggacgggcccgccgccttcctggacattctggccccgccctatgacccggacgatggccgggactgccactattaccgggtgctggagcctgtcagggacaaggagctttccggctcggcttgcgacctgcccagagaggtgtggctcctggagacccctcaagccgatgacttctggtgcgagggggagccctatccaggtcccaaggtcttcccttga

SEQ ID NO: 27 - DNA, 813 bp, Heterocephalus glaber
  source 1..813: mol_type = genomic DNA
atgccccgagacaacatggcttcccttatccaacggatagcccgccaggcttgcctcaccttccggggcaccgggggcgtccacggcgcttccgaccgcggcgcgacggctggccccgaggcgccgatgccccccggcttccccgagaacctgagcaagctgaagggcctgctgacccaggtccgcgccgaggacctcaacatcgccccgcgtaaggccacgctgcagccgctgcctcccaatctgccgccggtcacctacatgcacatctacgagacgcccggctttagcctcggcgtgttcctactcaagagcggcacgtctatcccgctgcacgaccaccctggcatgcatggtatcctcaaggtgctctacggcaccgtccgcatcagctgcatggacaagctggaagcgggcggtgggcagcgtccgagggcccagtcgctcgagcagcagttcgacccgccgctgcaggcccgggagcgggaggccatgcggccaggcatcctgcgctctcgggccgagtacaccgaagccagtggcccctgtgttctcacgccgcaccgagacaacctgcaccagatcgacgccgtggacggacccgctgccttcctggacatcttagccccgccctacgacccggatgacggccgggactgccactattaccgggtgctggagcctgtcaggcccaaagaggcctccagctcggcctgtgacctgccccgagaggtgtggctgctggagacgccgcaggccgatgacttctggtgcgaaggagaaccctatccaggacccaaggtcttcccttga

SEQ ID NO: 28 - DNA, 813 bp, Elephas maximus
  source 1..813: mol_type = genomic DNA
atgccccgagacaacatggcctccctgatccaacggatcgcccgccaggcgtgtctcaccttccggggtagcgggggcggccgcagcgcttccgaccgcggcgcggcgccaggccccgaggcgccgatgccgccgggcttcctagagaacctgagcaagctgaagagcctgctgacccaggtccgcgctgaggacctgaacatcgccccgcgcaaggccacgctgcagccactgccgcccagcctgccgcccgtcacctacatgcacatctacgagacggacggcttcagcctcggggtgtttctgctcaaaagcggcacgtccatcccactgcacgaccacccgggcatgcacggcatgctcaaggtgctctacggcaccgtgcgcatcagctgcatggacaagctggaggcgggcgacgggcaacggccgcgggcccctccgtccgagcagcagttcgagccgccgctgcaggcccgggagcgggacgccgtgcagctgggcgtgctgcgctcgcgggccgagtacaccgaggccagcggcctctgcatcctcacgccgcaccgggacaacctgcaccagatcgacgcagtggacgggccagccgccttcctagacatcctggccccgccctacgaccccgacgacggccgggactgccactattaccgggtgctggagcccgccgccaggcccaaggaggcctccggctcgacctgtgacctgccccgagaggtgcgtcagtcttggcatctcaggaaggactctgtcgtcactcatctgttgcagatcattctgaatcttgtactctag

SEQ ID NO: 29 - DNA, 903 bp, Mus caroli
  source 1..903: mol_type = genomic DNA
atggcctccctgatccagcgcatcgctcgccaggcgtgtctcaccttccgcggcggctcgacgggctccgaagggccggcgccgggcttcccggagaacctgagcctgctcaagagcctgctgacccaggtgcgcgccgaggacctcaacatcgcgccgcgcaaggcgctgccgcagccgctgccccgcaacctcccgccggtcacctacatgcacatctacgagacggagggcttcagcctgggcgtgttcctgctcaagagcggcacgtgcatcccgctgcacgaccacccgggcatgcacggtatgctcaaggtgctgtacggcacggtccgcatcagctgcatggacaagctggacacgggggccgggcatcggcggccgccgccagagcagcagttcgagcccccgctgcagcccttggagcgggaggccgtgcgaccgggcgtgctgcgttcccgggccgagtacaccgaggccagtgggccctgcgtgctcactccacaccgggacaacctgcaccaaattgatgctgtggacgggccagctgccttcctggacatcctggccccaccctacgacccggacgacggccgggactgccactattaccgtgtggtggagcccatcagacccaaggaggcttccggctctgcctgcgaccttccccgagaagtgtggctcctggagacaccacaggccaacaacttctggtgtgagggagagccctatccccaaggtcctaccttgaagctcctgccgcccggggaactgtgggccaaagatgtaccctgccctgctcaagaaaggcctggctgccctctaccttccataagggctcttctctctctgccccctcacctggaccatggatttactggagtgaccagcgccacatcttga

SEQ ID NO: 30 - protein, 270 aa, Capra hircus
MPRDNMASLIQRIARQACLTFRGSGGGRSSSDFGEAPGPEAPIPQGFPENLSKLKSLLTQVRAEDLNISPRKATLQPLRPNLPPVTYMHICETDGFSLGVFLLKSGTSIPLHDHPGMHGILKVLYGTVRISCMDKVEVGSGQRPRAPPPEQQFEPPLQPRERDAVQPGVLRSRAEYTEASGPCFLTPHRDNLHQIDAVDGPAAFLDILAPPYDPDDGRDCHYYRVLEPVRAKEASGSACDLPREVWLLETPQADDFWCEGEPYPGPKVFP

SEQ ID NO: 31 - protein, 270 aa, Homo sapiens
MPRDNMASLIQRIARQACLTFRGSWGGRGASDRDAASGAEAPMQPGFPENLSKLKSLLTQLRAEDLNIAPRKATLQPLPPNLPPVTYMHIYETDGFSLGVFLLKSGTSIPLHDHPGMHGMLKVLYGTVRISCMDKLDAGGGQRPRALPPEQQFEPPLQPREREAVRPGVLRSRAEYTEASGPCILTPHRDNLHQIDAVEGPAAFLDILAPPYDPDDGRDCHYYRVLEPVRPKEASSSACDLPREVWLLETPQADDFWCEGEPYPGPKVFP

SEQ ID NO: 32 - protein, 273 aa, Ursus maritimus
MPRDNMASLIQRIARQACLTFRGSGGCRSASDLGAAPGPEAPMPQGFPENLSKLKNLLTQVRAEDLNIAPRKATLQPLSPNLPPVTYMHIYETDGFSLGVFLLKSGTSIPLHDHPGMHGMLKVLYGTVRISCMDKLEAGGGPQPRAPPPEQQFEPPLQARERDAVRPGVLRSRAEYTEASGPCVLTPHQDNLHQIDAVDGPAAFLDILAPPYDPDDGRDGRDCHYYRVLEPVRDKEAPGSACDLPREVWLLETPQADDFWCEGEPYPGPRVFP

SEQ ID NO: 33 - protein, 270 aa, Nycticebus coucang
MPRDNMASLIQLIARQACLTFRGGGGGRRASDRGAASGPEAPMPPGFPENLSKLKSLLTQIRAEDLNIARRKATLQPLPPNLPPVTYMHIYETEGFSLGVFLLKSGMSIPLHDHPGMHGMLKVLYGTVRISCMDKLEAGGEQRPRAPPLEQQFEPPLQPRERDAMRPGVLRSRAEYTEASGPCVLTPHRDNLHQIDAVDGPAAFLDILAPPYDPDDGRDCHYYRVLEPVRSKEASGLACDLPREVWLLETPQADDFWCEGEPYPGPKVFP

SEQ ID NO: 34 - protein, 256 aa, Mus musculus
MPRDNMASLIQRIARQACLTFRGSSTGSEGPAPGFPENLSLLKSLLTQVRAEDLNIAPRKALPQPLPRNLPPVTYMHIYETEGFSLGVFLLKSGTCIPLHDHPGMHGMLKVLYGTVRISCMDKLDTGAGHRRPPPEQQFEPPLQPLEREAVRPGVLRSRAEYTEASGPCVLTPHRDNLHQIDAVDGPAAFLDILAPPYDPEDGRDCHYYRVVEPIRPKEASGSACDLPREVWLLETPQADDFWCEGEPYPGPKVLP

SEQ ID NO: 35 - protein, 250 aa, Salvelinus alpinus
MMPRDNMTSLVQKIGRQALVTFRNPSLVGDKAFLDNQGKLQTLMTEIRAADLKIAPRKVDCASTPLPHNPPVTYMHICETDQFSMGVFLLKSGASIPLHDHPGMYGMLKVMYGKVRITCFDRLDKSTNVASDTQFKPPLLPFQRDALRMSILRSVGEFTEESGPCILTPDRDNLHQIDAVDGPTAFLDILAPPYDPNDGRDCHYYKVLESDSEDKKAEAQGQKEVWLMEVPQPSDFWCGGEPYPGPEVNI

SEQ ID NO: 36 - protein, 278 aa, Phrynosoma platyrhinos
MPRDNMAASLIQRVARQARLTFRPPGTTTTSGGGGGGDGPRSAEALHRLRDLLGEVRAEDLRLSPRGPSAVPGRAQPPVSYMHICETESFSMGVFVLRAGACIPLHDHPGMNGLLKVLYGTLRIACLDTLPEDHDDDDDGGGGGGGAGASGPPLPHGRHHRHRHRGRHRALLRSHRLYTPSSAPCLLSPETDNLHQIDAVGGGPAAFLDILAPPYDPEHGRDCHYYRLLDGPGLLPPPPPSPEEDPPGLPKELWLQETPQAPDFWCGGEPYPGPQVSP

SEQ ID NO: 37 - protein, 284 aa, Vombatus ursinus
MPRDNMATLIQRIARQARLTFRRGGGGGGAPEAGFPENLQQLQQLLSQLRAEDLNISPRRAALPSPPPARPPHLPPVTYMHICETAEFSLGVFLLKSGTSIPLHDHPGMHGVLKVLYGTLRISCLDPLPGEGPGAPREGPQPQPHPPPPQQAPAAPPRFEPPLRPGQRAVVRRALLRSRAEYTEASAPCLLSPHRDNLHQIDAVDGPAAFLDILAPPYDPDDGRDCHYYRLLEAAAPAGPPDAGLGCGPAAPLPVPREVWLLETPQAADFWCEGEPYPGPKVTP

SEQ ID NO: 38 - protein, 247 aa, Notechis scutatus
MPRDEMAASLIQRVARQARATFGAGAGEAPAELQRLRELLGQVSAEDLGLLQPRGPSAVPGPSQPPVSYMHICETARFSMGVFVLRAGACIPLHDHPGMQGLLKVLYGTLRIACFDAADEEPEEQPDAASGAASSSPRPRRLRALLRSHRLYTAAAPPCLLSPQRDNLHQIAAEEAAGPAAFLDILAPPYDPARGRDCHYYRLADGQSLPPPPAQPVPPSALWLLETPQAPDFWCGGEPYPGPRVWP

SEQ ID NO: 39 - protein, 251 aa, Salmo salar
MPRDNKKSLIQKIANQANITFKDFNASAIGDNKVLLDNKGDLITLLTEIRAADLKMKPNFSHSEHNPPVTYMHICETESFSMGVFLLNNGASIPLHDHPGMNGMLKVLYGKVSISCFDLLDKPSDQVQSEIQFDPPLLPFQKDSLRRAVLRSTMWFTHDSSPCILTPQRENLHQIDTVDGPAAFLDILAPPYDPEDGRDCHYYKVLQTVPDADSKVEAQQQWEEETWLLEIGQPDGFWCGGEPYPGPKVSF

SEQ ID NO: 40 - protein, 242 aa, Adelges cooleyi
MASKIECVLKQSLITFSGSHTSKTFHDNLIKLKRYANELDANSITLDNRLLRCDTYELNSSFDKLSGSVLIRRVRAPVTYIEVYEDQNVSIGIFVLRNGAKIPLHDHPCMYGVLKVIHGKVRIQSYTPKKKDYSQLNECIISAVKMAPIIIGESDEPCVLCPVEGNIHQVDTVDGPAAFVDILAPPYRTDIPDVGKRYCRYFKELNMSKDIKLEVVPDPKDYWSDTAPYTGPQFNLNIDENS

SEQ ID NO: 41 - protein, 194 aa, Lipotes vexillifer
MPRDNMASLIQRIARQACLTFRGGSLLKSGTSIPLHDHPGMHGILKVLYGTVRISCMDKLEVGSGQRPRAPPPEQQFEPPLQPRERDALRPGVLRSRAEYTAASGPCVLTPHRDNLHQIDAVEGPAAFLDILAPPYDPDDGRDCHYYRVLEPVRAREASGSACDLPREVWLLETPQADDFWCEGEPYPGPKVFP

SEQ ID NO: 42 - protein, 245 aa, Mytilus edulis
MVSNIQKVAQTAYRLFSRICGNSDVLLSSQNVGLLTSMMNTIKDKDVNFDMSKISLESETPVSYIDIYEDPVMTMAIFIIRDGCKIPLHDHPGMYGFCKVIHGSARMKSFSDPQNFVGNPPENIASKIRPYLRQSIKPVVLEEESIFSSDDNCCVVTPNHGTYHELVAVNGPVAFLDILAPSYDHASGARICQYYEEIKGQNEEIKHLGKPLIWFTPISQPSDFWCDPEEYEGPSVVDALNEIVK

SEQ ID NO: 43 - protein, 228 aa, Pan troglodytes
MQPGFPENLSKLKSLLTQLRAEDLNIAPRKATLQPLPPNLPPVTYMHIYETDGFSLGVFLLKSGTSIPLHDHPGMHGMLKVLYGTVRISCMDKLDAGGGQRPRALPPEQQFEPPLQPREREAVRPGVLRSRAEYTEASGPCILTPHRDNLHQIDAVEGPAAFLDILAPPYDPDDGRDCHYYRVLEPVRPKEASSSACDLPREVWLLETPQADDFWCEGEPYPGPKVFP

SEQ ID NO: 44 - protein, 254 aa, Manis javanica
MPRDNMASLIQRIARQACLTFRGSGGGLSASDRCTAPGPEAPMPQGFPENLNKLKSLLTQVRAEDLNIAPRKATLQPLPPNLPPVTYMHICETDGFSLGVFLLKGGTCIPLHDHPGMHGMLKVLYGTVRISCIDKLEAGGGQRPRAPPPEKQFDPPLQARERDAVRPGVLRSRAEYTEASGPCVLTPHRDNLHQIDAVDGPAAFLDILAPPYDPDNGRDCHYYRVLEPIRAKEASGSACDLPREGSAHYYSTLT

SEQ ID NO: 45 - DNA, 1857 bp, Propithecus coquereli
  source 1..1857: mol_type = genomic DNA
atggtaaaatgctttcccttgaggaaaacagaacagcactctgaagtttgtccacacggagcctcctccggcaattcaccaacttcaatccaagcaatccttaagatacggtactggcagcacaggactcagccccagggaactttggacagagacacacacacgggacaacgctatctgaagagacacagggagaagacagccatttgcaagtcaaagagagaagcctggaacgtagccttccctcgccgccacaggaggaaacgaccctgctggggactcctaacctccagaatttcactggacttcagcatgatcacttctccgtcgctggcttatgtggcgattttggttttctgtgtctcaaaaaccagctctctggacacttttattgcagctgtttatgagcatgcggtgctattgcctaataccagcctcacaccagtgtctcgtgaggaggcgctggcgttaatgaatcggaatctggatcttttggaaggagcgatcgcgtcagcagcggagcagggcgcacatattatagtgaccccagaagatggtatttatggctggaacttcaacagggaatctctctacccgtacttggaggatatcccacaccctcaagtaaactggatcccctgtaatgatcctaacagatttggccacacccccgtacaagaaagactcagctgcctggccaaggagaactctatctatgttgtggcaaatattggggacaagaagccatgtaatgccagtgaccctcagtgtccccctgatggccgttaccagtacaacactgatgttgtgtttgattctcagggaaggctggtggcacgctaccataagcaaaatcttttcttgggtgaagatcaattcaatgcacccaaggagcctgaggttgtgactttcaacaccacctttggaaggtttggcatttttacgtgctttgatatactcttccaccatcctgctgttaccctggtgaaagatttccacgtggacaccatacttttcccaacagcttggatgaatgttttgccacatttgtcagctattgaattccactcagcgtgggctatgggcatgggggtcaatttccttgcatctaatatacattacccctcaatgaggatgacaggaagtggcatttatgcacccaattctgcaagagtatttcattatgatatgaagacaaaggagggaaaactcctcctctcacaactggattcccacccacaccaccctgtagtggtgaattggccatcttatgccagcagtgtggaagcactctcgactggaaaccaggaatttaagggcattatcttttttgatgaatacaccttcgtggagctcaaaggagtcggggggaattacacagtatgtcagaaagatctctgttgtcatctaagctacaagatgtcagagaagagatcagatgaagtttatgccctaggggcatttgatggactgcacaccgtggaaggacgctattatctacagatttgtactctggtgaagtgtaaaacgaccgacttaaacacttgtggtgactcagttgaaaccgcttctaccaggtttgaaatgttctccctcagtggcacttttggaacccagtatgtcttccctgaggtgttgttgagtgagattcagcttgcacctggagaatttcaggtgtcaagtgatggacgcttgtatagcctgaagccaacatctggacctatcttaacagtgactctgtttgggaggttgtatgagaaggacggagcatcaaacgcttcatcagacctcatagcgcaagcactaagaataatgctagcagttataatacctattgtatactcattaagttggtag

SEQ ID NO: 46 - DNA, 1545 bp, Equus asinus
  source 1..1545: mol_type = genomic DNA
atgatttcttctcagctgctcgcttatgtggcagttttggttttctgcaccttccaagcccgctccctggagacctttattgcagctgtttacgagcacgcagtgatattgcccaatgccaccctaacgccagtgtcccgggaagaggcattgctattaatgaaccgcaatctggatcttctggagggagcaatcgcctcagcagccaagcagggtgcgcacatcatcgtgactccagaagatggtgtttacggctggaacttcagcagggacacgctctatccgtacttggaggacatcccagaccccgaagtgaactggatcccttgcaacaatccccacagatttggccacactccagtccaagagaggctcagctgcctggccaaggacaactctatctacgtcgtggcaaatttcggggacaagaagccatgcaatgccagtgaccctgagtgcccccctgatggccgttaccagtacaacactgatgtggtgtttgatgctcagggaaaacttgtggcacgctaccataagcaaaaccttttcatgggtgaagatcaattcaatgcacccaaggagcctgaggtcgtgactttcaacaccacctttggaaagtttggcattttcacgtgcttcgatattctcttctacgaccctgctgttaccctggtgacagatttccaagtggacaccatcctcttcccgacagcctggatgaatgttttgccacatttgtcagctattgaatttcactcggcttgggctatgggcatgggggtcaatttccttgcatccaatatacattacccctcaatgaaaatgacaggaagtggcatctatgctcctgattctccaagagcatttcattatgatatgaagacggaggagggaaaactactcctctcacagctggactcccacccgcaccacccagcagtggtgaactggacttcttttgccagtggtgtgaaaacactctcaacgggaaaccaggaatttaacagcactgtcttttttgatgaattcaccttcctggagctcaccgaagtcacaggaaattacacagtttgtcagaaagctctctgctgtcacctaagctacaagatgtctgagaagagatcagatgaagtgtacgccctaggggcatttgatggcttacatactgttgaagggagttattatttacaggtttgcactctgttgaagtgtagaaccacggacttacacacttgtggtgactcggtggaaactgcttccactaggttcgatacgttctccctcagtggcacttttgggacccagcatgtcttccctgaggtgctgctgagtgaaattcagctcgcacctggagaattccaggtgtcaagtgatggacgcttgttcagtctgcagcccccctcgggacctgtcctgacagtgactctttatgggaggctgtacgagaaggaccaggtatccgacgcctcatcagacctcacagcacaagcactgagagtaatgctcgtagttctaatacctgttgggttctcaataagttggtag

SEQ ID NO: 47 - DNA, 1383 bp, Neomonachus schauinslandi
  source 1..1383: mol_type = genomic DNA
atgaaccgaaatctggatcttttggaggcagcaatcacttcagcagcaaaccagggtgcacatattattgtgactccagaagatggtatttatggctggaactttaccagggaaactatctacccatacctggaggatatcccagaccctggagtgaactggatcccttgtaataatcccaaaaggtttggctacaccccagtgcaagaaagactcagctgcctggccaaggacaactctatctacgttgtggcaaatattggggacaagaagccatgcaatgccagtgaccctcagtgtccccccgatggtcgttaccagtacaacaccgatgtggtgtttgattctcagggaaaactggtggcacgctatcataagcacaaccttttcatgggtgaaaatcaattcaatgtacccaagaagcctgaggttgtgacttttgacaccatctttggaagatttggcattttcacgtgcttcgatatactcttctatgatcctgctgttactctggtgaaagatttccatgtcgataccatagtcttcccaacagcatggatgaatgttttgccacatttgtcagctattcagttccactcagcttgggcaatgggcatgggggtcaacttccttgcatccaatatacatcacccctcaaagagaatgacaggaagtggcatctatgcacctgattctccaagagcatttcattatgatatgaagacaaaggagggaaaactccttctctcacaactggattctcactcacacagccctgtagtgaattgggcttcttatgccagtagtataaaggcattctcaacaggaaatcaggaatttaagggtactgccttttttgatgaattcaccttcttggagctcacaagagtcacaggaaattacacagtttgtcagaaaaaactctgctgtcatctaagctacaagatgtctgaaaagagaagagatgaagtttacgccctgggggcatttgatggactacatattgtcgaagggacgtattatttacagatttgtaccctgctgaaatgtaaaacaacaaacttaaacatttgtggtggtgcagttgaaacagcttctaccaggtttgaaatgttttccctcagtggcacttttggaactcagtatgtcttccctgaggtgttgctaagtgaaagtcagcttgcacctggagaatttcaggtgtcgagtgatggacgcttgttcagcgtgaagccgccatctggacctctcttgaccgtgactctatttgggagaatgtatgagaaggaccagacattaaaggcttcatcagacccccaggcacaaacactgggaataatgctcttagtcataatgccagttgtgtgctcattaagttggtag

SEQ ID NO: 48 - DNA, 1545 bp, Carlito syrichta
  source 1..1545: mol_type = genomic DNA
atggttgcttctcagttgctggcttatatggcaatcttggtgttctgtgtcttaaaagccagctccctggacacgtttcttgcagctgtttatgagcatgcagtgatactgcctgatgccaccctgacaccagtgtctcgtgaagaagcattggtgctaatgaataggaatttggatcttctggaaagagcggtcatatcagctgcacagaagggtgcacatattattgtgactccagaagatgccatttacggctggaacttcagcagggaatctgtctacccctacttggaggatatcccacaccctcaagtgaactggatcccttgtaataatccgaacagatttggccacactccagtccaagaaagactcagctgcctggccaagaacaactccatctatgtcgtggcaaatattggagacaagaagccatgcaatgccagtgaccctcagtgtccccctgatggccgttaccaatacaacactaatgtggtatttgattctcagggtacgctggtggcacgctaccataagcagcaccttttcataggtgaagatcacttcaatgcacccaagagccctgaggtcgtgactttcaacactacatttggaaggtttggcattttcacatgctttgatatactcttccatgatcctgcagttaccctggtgaaagatctccacgtgaacaccatactatttcccacagcttggatgaatgttctgccacatttgtcagcgattgaattccactcagcttgggctatgggcatgggagtcaacttccttgcctccaatatccattaccctttgatgaaaatgacaggaagtggcatctatgcacccaattctccaagggcatttcattatgatatgaagacagaagaaggaaaactcctcttctcgcaactggatgcccgcccataccaccccagagtggtgaattggacttcttacgccagtagtatagaggcagtctcatcagaaaaacagaaatttaagggagtcgtcttctttgatgaattcaccttcgtggagctcacagatgttatgggaaattacacagtttgtcagaaagatctctgctgtcatctgagctacaagatgtctgagaagagatcagatgaagtttacgcccttggggcatttgatgggctgcacacggtggaagggtgctattatctacaggtttgtactctgctgaagtgtaaaacaactgacttacacacgtgtggtgactcagtggaaacggcttctaccacgtttgagatgttctccctcagcggcacctttggaacccaatatgtcttccctgaggtgctgctgagtgaaatgcgacttgcacccggagaatttcaggtgtcagatgatggatgcttgtctagcctgaagccaacatctagacctgtcttaacagtgactctttttgggaggttgtatgagaaggattgtgcatcaaatgcttcatcagacctcacagcacaagcacgaagaaccacgctgatagtaatgatatgtattgtccatttattaagttggtag

SEQ ID NO: 49 - DNA, 1632 bp, Dipodomys ordii
  source 1..1632: mol_type = genomic DNA
atggtgattgctcggttgctggcttatttggcattttcagttttctgtgtcttaccagccaactgcctggacacttttactgcagctgtttatgaacatgccgtaatactgcctaacttcaccacaacaccagtgtctcgtgaagaggcactggtgttaatgaatcagaatctagacattttggaagaagcaatcacctcagcagcaaaacagggtgcacaaattattgtgactccagaagatggtgtttatggtgtgaatttcagcagggacaccatttacccatatttagaggacattccagacccggaagtcaattggatcccctgtaatcatcccaaaaggtttggtgatactcaaatacaagaaagactcagctgtctggccaagagcaactctatctatgtggtggcaaatataggagacaagaaaccatgcaatgccagtgaccctcgatgtccccctgatggtcgttaccaatacaacactaatgtggtgttcgattcacagggaaaactggtagcccgctaccacaagcaacatcttttcatgagtgaatcttttcacttcaatgcacccaaagatcctgaggtcgtgactttcaacactccctttggaaaatttggcatttttacatgctttgacatcctcttctacgaccctgccattaccctggtgaaagatttccatgtggacactatcctcttcccaacagcttggatggatgttctaccacacttgtcagccattgaattccactcagcttgggctatgggcatgggagtcaatttacttgcatccaatttacatcgcccccagaacagaatgacaggaagtggcatctatgcacctgattctccaagagcttttcattatgacaggaagacagaaaagggaaagctcgtcctctctcagctggattcccacccctaccgccccgaagtgaactggagttcttatgccagcaacataggagcactctcaactggaaaccaagaatttaagggcagtgccttttatgatgagtacactttcgtggagctcacaggaatcacaggaaattacactgtttgccagaaagatctctgctgtcatctaaactaccagatgtctgagaagagatccaatgaaatttatgctctaggggcatttgatggactgcacacagttgaagggtgttaccatctacaggtctgcactatgttaaagtgtaaaaccactaacagacatacctgtggtgcttcagttgacacggcttctactaggtttgaaatgttctccctcagtggcacttttgggacccagtatgtctttcctgaggtgttgttgagtgaagttcggctcgcacctggagaatttaaggtaacaacctttgcttcatttccccagttaattgcatgtgaaaggaaatactggtttatttcaaaagctactgaaatcatacatatttccattgggaaaagggaaagattatctttgaggatcaggaaaggcaccgcaagcagcctctgccataaagtcttaccaccagcatgttctgccttactatggacttaccaacacacccaaggatcactggccataggaatgaaaaatctgagtgacacagagaaagcagctttatttatatga

SEQ ID NO: 50 - DNA, 1257 bp, Ictidomys tridecemlineatus
  source 1..1257: mol_type = genomic DNA
atgttttggaaggagcgatcacgttagctgcaaatcagatttggctacaacccagtacaagaaagactcagctgcctggccaagaataactctatctatgtggtggcaaatataggggacaagaagtcatgcaaagccagtgagcctcagtgtcctcctgatggccgttaccaatacaatactaacgtggtgtttgattctcagggaaaactagtggcccgctaccataagcaaaaccttttcatgggggaagaccaattcaatgcacctaaagagcctgagatggtgacatttaacacaacctttgggaagtttggcattttcacttgctttgatattctcttccatgatcctgctgttgccctggtgaaagacctccacgtggacaccgtactcttcccaacagcttggatgaatgtcctgccacatttgtcagcggttgagttccactcagcttgggctatgggcatgggagtcaatttccttgcatctaatttacataacccctcaaatagaatgacaggaagtggtatttatgcacctgattttccaagagcatttcattatgatatgaagacaccagagggaaaacttatcatctcaacactggattctcacccatcccattccatagtgaattggtcttcttacgccagcagtatacatgcattcccaacaggaaaccaagaattcaagggcactgtcttttttgatgaattcacctttgtggagctcacagaaattgcaggaaattacacagtttgtcagaaagagctctgctgtcatttaagctatgaaatgtctgaaaagagatcagatgaagtttatgccttaggagcatttgatggactgcacgttgttgaagggcgctattacctacagatttgtactctgctgaaatgtgaaaccactgacttgcacacctgtggtggttcagttgacacagcttccactaggtttgaatcgttctccctcagtggcactttcggaacccaatatgtcttccctgaagtactgctgagtgacgttcagcttgcacctaaagaatttcaggtgtcaagtgatggacgtttgtttagcttgaagccaacatctggacctctgttaacggtcactctctttgggagggtgtacgagaaggatatggcatcaaatgggtcacctgacctctcaacatattcagtaaagataatgttagtagttatgatacctattgtgttctcgttatgttggtagaatatttacattttctctctt

SEQ ID NO: 51 - DNA, 1539 bp, Rattus norvegicus
  source 1..1539: mol_type = genomic DNA
atgagcctgtctcggtggctggcgtgggctgcagcgttttccgtcttctgtgtcttaaaagtcagctctctggacactttcctcgcagctgtttacgagcatgctgtgatcctgcctaaagtcaccctgttacctgtgtctcacagtgaggcactggcattaatgaaccagaatctggaccttctggaaggagcgatcttatcagcagctaagcagggtgcgcacattattgtgactccagaagatggtatatacggtgtgcagttcaccagggataccatctacccatacctggaggatatcccagaccctcaagtaaactggataccctgtgataatccggaaagatttggctctaccccggtgcaggagagactcagctgcttggccaagaacaactcaatctatgtcgtggcgaacatgggagacaagaagccatgcaacaccagcgactctcactgtccacctgacggccgattccaatacaacactgatgtggtgtttgattcccggggtaaattggttgccagataccataagcaaaaccttttcatgggagaagagcaattcaatgcccccccggagcctgaggtcgtgacttttgacaccccctttggaaagtttggcatcttcacctgtttcgatattctcttccatgatcctgctgtcaccctagtgacagagttccaggtggacaccatactgttcccaactgcctggatggacgtgcttcctcatttggcagccattgaattccactcagcttgggctataggcatgggagtcaatttcctagcagctaatctacatattcccttgaggagaatgacaggaagtggtatctatgcacccgattctccacgggcctttcattatgacaggaagacacaggaaggaaagctcctccttgcacagctggactctcacccgagtcactccccagtgaactggacttcctatgccagcagtgtagaagcacccccaactgaaaaggaggaatttcggagttttgttttctttgatgagtttacctttgtggagctcaaagggatcacaggaaattacactgtttgccagaacgacctctgctgtcatctaagctaccagatgtctgagaagcgagctgatgaagtttatgctcttggggcttttgacgggttgcacaccgtggaagggcagtatcacctgcagatttgcattctgctaaagtgtaaaactaccaatttacgcacctgtggtagttcagtggacacagcttctaccaggtttgaaatgttctccctcagtggcacttttggaacgcggtatgtcttccctgaggtgttgctgagtgaagtcaagcttgctcctggggagtttcaggtgttgagtgacgggcgcctggttagcctgaagccaacgtctggacctgtgttaacagtcggtctctttgggaggttgtatgagaaggactgggcatcaaatgcttcctcagacctcatagcacactcactgataataatgctgatcctagctcctattatacattacttgtgttaa

SEQ ID NO: 52 - DNA, 1542 bp, Homo sapiens
  source 1..1542: mol_type = genomic DNA
atgactactcagttgccagcttacgtggcaattttgcttttctatgtctcaagagccagctgccaggacactttcactgcagctgtttatgagcatgcagcgatattgcccaatgccaccctaacaccagtgtctcgtgaggaggctttggcattaatgaatcggaatctggacattttggaaggagcgatcacatcagcagcagatcagggtgcgcatattattgtgactccagaagatgctatttatggctggaacttcaacagggactctctctacccatatttggaggacatcccagaccctgaagtaaactggatcccctgtaataatcgtaacagatttggccagaccccagtacaagaaagactcagctgcctggccaagaacagctctatctatgttgtggcaaatattggggacaagaagccatgcgataccagtgatcctcagtgtccccctgatggccgttaccaatacaacactgatgtggtatttgattctcaaggaaaactggtggcacgctaccataagcaaaaccttttcatgggtgaaaatcaattcaatgtacccaaggagcctgagattgtgactttcaataccacctttggaagttttggcattttcacatgctttgatatactcttccatgatcctgctgttaccttggtgaaagatttccacgtggacaccatagtattcccaacagcttggatgaatgttttgccacatttgtcagctgttgaattccactcagcttgggctatgggcatgagggtcaatttccttgcatccaacatacattacccctcaaagaaaatgacaggaagtggcatctatgcacccaattcttcaagagcatttcattatgatatgaagacagaagagggaaaactcctcctctcgcaactggattcccacccatcccattctgcagtggtgaactggacttcctatgccagcagtatagaagcgctctcatcaggaaacaaggaatttaaaggcactgtctttttcgatgaattcacttttgtgaagctcacaggagttgcaggaaattatacagtttgtcagaaagatctctgctgtcatttaagctacaaaatgtctgagaacataccaaatgaagtgtacgctctaggggcatttgacggactgcacactgtggaagggcgctattatctacagatttgtaccctgttgaaatgtaaaacgactaatttaaacacttgcggtgactcagctgaaacagcttctaccaggtttgaaatgttctccctcagtggcactttcggaacccagtatgtctttcctgaggtgttgctgagtgaaaatcagcttgcacctggagaatttcaggtgtcaactgacggacgcttgtttagtctgaagccaacatccggacctgtcttaacagtaactctgtttgggaggttgtatgagaaggactgggcatcaaatgcttcatcaggcctcacagcacaagcaagaataataatgctaatagttatagcacctattgtatgctcattaagttggtag

SEQ ID NO: 53 - DNA, 1539 bp, Mus musculus
  source 1..1539: mol_type = genomic DNA
atgggcatgtcttggtggctggcgtgtgctgcagcgttttctgccctctgtgtcttaaaagccagttcgctggatactttcctcgcggctgtttacgagcatgctgtgatcctgcctaaggacaccctgttgccagtgtctcacagtgaggctctggcattaatgaaccagaatctggaccttctggaaggagcgatcgtatctgcagcgaacgagggtgcgcacattattgtgactccagaagatggcatatacggtgtgcgtttcaccagggatacgatctacccatacctggaggagatcccagaccctcaagtaaactggataccctgtgataaccctaaaagatttggctctaccccggtgcaggagagactcagctgcttggccaagaacaactccatctatgttgtggcgaacatgggagacaagaagccgtgtaacaccagcgactctcactgtccacctgacggcagattccagtacaacactgatgtggtgtttgattcccagggtaaactggttgcgagataccataagcaaaacattttcatgggagaagatcagttcaatgtccccatggagcctgagtttgtgactttcgacaccccctttggaaagtttggcgtcttcacctgtttcgatattctcttccatgatcccgctgtcaccctggtgacagaattccaggtggacaccatactgttcccaaccgcctggatggacgtccttcctcatttggcagccattgaattccactcagcttgggctatgggcatgggggtcaatttcctagcagctaatctacataatccctcgaggagaatgacaggaagtggtatctatgcacccgattctccaagggtctttcactacgacaggaagacccaagaaggaaaactcctcttcgctcagctgaaatcccacccaattcactccccggtgaactggacttcctatgctagcagtgtagaatcaaccccaaccaaaacccaggaatttcagagtattgtcttttttgatgagtttacctttgtggagctcaaagggatcaaaggaaattacactgtttgccagaatgacctctgctgtcacctaagctaccagatgtctgagaagcgagcagatgaggtttatgcctttggagcctttgatgggctgcacaccgtggaagggcagtactacctacagatctgcatcctgctaaaatgtaaaactaccaatttacgcacctgtggtagttcagtggacacggcttttaccaggtttgaaatgttctcgctcagcggcacttttggaacccggtatgtcttccctgaagtgttgctgagtgaggtcaagctcgcacctggggagtttcaggtgtcaagtgatgggcgcctggttagcctgaagccaacctcgggacctgtgttaaccatcgggctctttgggaggttgtatgggaaggactgggcatccaatgcttcctcagacttcatagcacactcgctgataataatgctgattgtgacgcctattatacattacttgtgctga

SEQ ID NO: 54 - DNA, 1311 bp, Mesocricetus auratus
  source 1..1311: mol_type = genomic DNA
atgctgtgatcctgccaaatgacaccctgacacccgtgtctcacagtgaggcgctggcactaatgaatcagaatctgaatcttctggaaggagcgatcgtgtctgcagcaaagctgatttggctacaacccagtacaggagagactcagctgcttggccaagaataattcaatctatgttgtggcgaacatgggagacaagaaaccatgcaacactagtgaccctcagtgtcctcctgatgggcgtttccagtacaacactaatgtggtgtttgattccctgggcaaactggttgcacgataccataagcaaaaccttttcatgggtgaagaacaattcaacgcacccatggagcccgagttggtgactttcaacaccccgtttggaaggtttggcatcttcacatgcttcgatatcctcttccacgatcctgctgtcaccctggtgaccaagtttcaggtggacaccatactgttcccaaccgcttggatggacgttctgcctcatttggcagccattgaattccactcagcttgggctatgggcatgggggtcaatttacttgcagccaatatacatagtccctcgaggagaatgacaggaagtggtatctatgccccagactctccaaaggcctttcactatgacaggaaaaccaatgagggaaaacttgttctagcgcagctggattcccacccaagtcgctccactgtgaactggacttcttatgccagcagtgtagaagcacccgcaacaggaaaccaggaatttcagagcatcattttctttgatgagtttacctttgtggagctcaaaggaatcacaggaaattacactgtttgccagaatgatctctgctgtcatctaagctaccagatgtctgagaagcgagctgatgaagtttatgcccttggggcatttgatggcttgcacactgtggaagggcagtatcatctgcagatctgtactctgctgaaatgtaaaaccaccagtttacacacctgtggtgattcagtggacacagcttctaccaggtttgaaatgttctccctcagtggcacatttggaactcagtatgtcttccccgaggtgttgctgagtgaagtcaagcttgcacctggggaatttcaggtgtcaagtgacggacgtttgtttagcctgaagccaacatctggacctgtgttaacagtcactctgtttgggaggttgtatgagaaggactcggcatctagtgcttcctcagacctcataacacactcactaatgataataacggtgattgtcctacctattgtacattacttatgttaa

SEQ ID NO: 55 - protein, 512 aa, Physeter catodon
MSQLLDYVAIFFFCVLKASSLDTFTAAVYEHAVILPNATLIPVSHEEALLLMNQNLDLLEAAITSASKQGAHIIVTPEDGIYGFNFNRESIYPYLEDIPDPQVNWIPCNNPDRFGHTPVQERLSCLAKDNSIYIVANIGDKKPCNTSDPHCPPDGRYQYNTDVVFDSEGKLVARYHKQNLFLGEDQFNAPKKPEVVTFNTTFGKFGIFTCFDILFHDPAMTLVKDFHVDTILFPTAWMNVLPHLSAIEFHSAWAMGMGVNFLASNLHYPSMKMTGSGIYAPDSPRAFHYDMKTEKGKLLLSQLASHPHPTAVVNWTSYASGIEAPSMGNQEFKGIIFFDEFTFLELKEVTGNYTVCQKDLCCHLSYKMSEKRSDEVYVLGAFDGLHTVEGSYYLQICTLLKCKTMDFHTCGDSVETASTRFETFSLSGTFETQYVFPEVLLSEIQLAPGEFQVSSDGRLFSQKPLSGPILTVTLFGRLYKKDSAPNAFSDLMAQALRVMLIVVIPIVYSLGW

SEQ ID NO: 56 - protein, 514 aa, Myotis myotis
MITSQLLTCVAISIFCALKASSLDTFIAAVYEHAVILPNATLAPVSRELALGLMNRNLDLLEGAITSAAKQGAHIIVTPEDGLYGWNFNRESIYPYLEDIPDPQVNWIPCNHPNRFGHTPVQERLSCLAKENSIYVVANIGDKKPCNASDPQCPSDGRYQYNTDVVFDSQGKLVARYHKQNLFLGEDQFNVPKEPEVVTFDTTFGRFGIFTCFDILFHDPAVALVKDFHVDTILFPTAWMNVLPHLSAIEFHSAWAMGMKVNFLASNIHHPSKKMTGSGIYAPDSPRAFHYDMKTEKGKLLLSRLDSHPKHPVMVNWTSYASGIEAFSMRNKEFKGTVFFDEYTFLELRGVTGNYTVCQKDLCCHLSYKMSEKRADEVYALGAFDGLHTVEGNYHLQICTLLKCKTTSLHTCGHAVETASTRFEMFSLSGTFGTQYVFPEVLLSEIQLAPGEFQVLSDGRLLSLKPPPGPVLTVTLFGRVYEKDHTSNASSGFMAYTLTIMLIVITSILYSLSR

SEQ ID NO: 57 - protein, 512 aa, Mus musculus
MGTSWWLACAAAFSALCVLKASSLDTFLAAVYEHAVILPKDTLLPVSHSEALALMNQNLDLLEGAIVSAAKQGAHIIVTPEDGIYGVRFTRDTIYPYLEEIPDPQVNWIPCDNPKRFGSTPVQERLSCLAKNNSIYVVANMGDKKPCNTSDSHCPPDGRFQYNTDVVFDSQGKLVARYHKQNIFMGEDQFNVPMEPEFVTFDTPFGKFGVFTCFDILFHDPAVTLVTEFQVDTILFPTAWMDVLPHLAAIEFHSAWAMGMGVNFLAANLHNPSRRMTGSGIYAPDSPRVFHYDRKTQEGKLLFAQLKSHPIHSPVNWTSYASSVESTPTKTQEFQSIVFFDEFTFVELKGIKGNYTVCQNDLCCHLSYQMSEKRADEVYAFGAFDGLHTVEGQYYLQICILLKCKTTNLRTCGSSVDTAFTRFEMFSLSGTFGTRYVFPEVLLSEVKLAPGEFQVSSDGRLVSLKPTSGPVLTIGLFGRLYGKDWASNASSDFIAHSLIIMLIVTPIIHYLC

SEQ ID NO: 58 - protein, 513 aa, Homo sapiens [sequence omitted]

SEQ ID NO: 59 - protein, 512 aa, Monodelphis domestica
MIPFQLQSYIAFFALWAFKTNCLDTFIAAVYEHAVILPPDNIKLDSREEALALMNENMDILEEAVISAAKQGAHIIVTPEHGIYGFIFNRVNLYPYLEDIPHPKVNWTPCTDDSRFGSTPVQKRLSCMARNNSIYMVANIGDRKSCDPTESKCPSNGYYQYNTNVVYDSEGKLVARYYKKYMLMNEAQFDTPTEPQLVTFDTTFGKFGILTGFDILFHDPVVPLVEKLKVDTILLPVAWMNTLPHLYPTAFHAAWAMGMRVNLLVANIHSISNQMTGSGIYAPDSVKAYYFDKKSDDGKLLVSELESHPIVRPPVNWTLYASSIKPFSSEQEEFKALVLFDMFTFKELINDTGNYTVCQNTLCCHLSYKMSEKRTDEVYVVGAFDGLHIAEGEYYLQICTLLKCLTTLIETCGQPVENASTWFDMFSLSGTFDSPHVFPELLLSGLQIAPTEFEILSDGRIINQNGTSAPVLMLTLFGRQYEKTKPPNASVDLETTSVMMITMFFVLYSTIW

SEQ ID NO: 60 - protein, 512 aa, Mus pahari
MNMSWWLACTAAFSVLCVLKASSLDTFLAAVYEHAVILPKDTLLPVSHSEALALMNQNLDLLEGAIVSAAKQGANIIVTPEDGIYGVRFTRDTIYPYLEDIPDPQVNWIPCDNPKRFGSTPVQERLSCLAKNNSIYVVANMGDKKPCNTSDSHCPPDGRFQYNTDVVFDSQGKLVARYHKQNIFMGEDQFNVPMEPEFVTFDTPFGKFGIFTCFDILFHDPAVTLVTEFQVDTILFPTAWMDVLPHLAAIEFHSAWAMGMGVNFLAANLHNPSRRMTGSGIYAPDSPRVFHYDRKTQEGKLLLAQLDSHPSHSPVNWTSYASSVESTPTKNQEFQSIVFFDEFTFVELKGIKGNHTVCQNDLCCHLSYQMSEKRADEVYAFGAFDGLHTVEGQYYLQICILLKCKTTNLRTCGSSVDTASTRFEMFSLSGTFGTRYVFPEVLLSEVKLAPGEFQVSSDGRLVSLKPTSGPVLTVGLFGRLYGKDWSSNASSDFIAHSLIIMLIMTPIIHYLC

SEQ ID NO: 61 - protein, 517 aa, Nematostella vectensis
MFRWFVAKFSLLALVIISHIKSVPESEFIAAVYEHVPPLNRPKEITRANALGIMMRNIDTYEEQMVIARDKNSSIIVFPEYGLTGWNQTRSVFKHFLENIPDPKISSNPCLDPGINKTTPILYRLSCLARKYAMYLVVNMGDIKPCQKASDPHCPGDGRYQYNTNVVFSDNGTLVARYHKQHPFMNEMKVVNRPRVPELVTFQTPFGKFGTFVCFDVLFQAPAVQLVTSVGIDHVVFPTAWFDVLPLFPAIGFHSSWARGIGVNFLAANTHVPSFANTGSGIYSSSGAREYYRSFSASGKLLIASLPKSPKKSPSNMTYVKTPGNTNDDVFYSDLFGDRFLFKELDKAEAIIRVCYPGSKSCCTLHYKMAAKLPDELYALGVFDGLHTLEGQYYLRVCTLIKCFGMSRSSCGSQVSSASTIFDSFALRAELNASHVFPAVVAHGVTLLRDEWDHDVPVKQVESKRRLSKGLLSATLFGRVYSLDGGKTSSTGRATCGPTFILTLRIVCIAMISVNLT

SEQ ID NO: 62 - protein, 510 aa, Bos taurus
MIMSQLLNYVAVLFFCVSRASSLDTFIAAVYEHAVILPNATLVPVSPEEALAVMNRNLDLLEGAVTSASKQGAHIIVTPEDGIYGFNFTRESIYPYLEDIPDPQVNWIPCNNPDRFGHTPVQQRLSCLAKDNSIYIVANIGDKKSCNASDPQCPPDGRYQYNTDVVFDTKGKLVARYHKQNLFLNEDQFNAPKEPEVVTFNTTFGKFGIFTCFDILFHDPAVTLVRDSRVDTILFPTAWMNVLPHLSAIEFHSAWAMGMRVNFLASNLHYPLKKMTGSGIYAPDSPRAFHYDMKTEEGKLLLAQLDSHPHPTPVVNWTSYASGVEAHSVGNQEFTGIIFFDEFTFLELKEIGGNYTVCQRDLCCHLSYKMSEKRSDEVYALGAFDGLHTVEGSYYLQICTLLKCKTTDLHTCGDSVETASTRFEMFSLSGTFGTQYVFPEVLLSEIQLAPGEFQVSNDGRLFSLKPTSGPVLTVTLFGRLYEKDSAPNTLSDLTTQALRLNPKTDAWKSK

SEQ ID NO: 63 - protein, 586 aa, Bulinus truncatus
MSLVLWVPGFSTVEEEKKSSRDIMTSSIVAVLHVTALLLMCVGGVFTSRSKFKAAVYGHAVILPEDPTKVVTRAEAVLNMNKNLDVYREQARKAREQDAEIIVFPEDGLYGFRFKRESIYPYLEYIPDPRVVSWSPCDDPRRHHDTEVQMSLSCMAKENSIYVVANMGDKQACDVLTDPKCPSDGRYQFNTNVAFGPDGTLLARYHKYHLFYELQFDTPELELIHFDTPFGRFGLMVCFDVLFYDPGIPLILNLNISNIVFPTAWMDALPLLSALGFHSSFARGLGVNFLSANIHLPDYRFHGSGIYGPDGAEISYYGAKSTSKPKLLVAEVNSLGNPKPNLNLSASFPFSNRLGELRDSTETCDSESDEFNLKRYFKMPNINDTFESVLFGDVFTFVSLDKPSGHLAVCQKNVCCDLQYSIDEQSFGRELFAFGAFDGLHTYEGQYYFQICTLVRCAAADNRTTCGSVTSTSETNFLHLSMSAEMQTDFIYPQIVLSNNDGELQLAEPQSWDFSDGYLSTKKGFNKTLLSAALFGRYYSRDGCISQEQTDSVEIKVKLGNDGSSSILSFPWILCISILECVLIFK

SEQ ID NO: 64 - protein, 547 aa, Caerostris darwini
MARSRALLFLILFVHSSCLFAKKDFYRAAVLELSQFTNISYPAADILRINLNIYDLAASTAAKHEADIIVFPESGLLPFEKPNREWLLDFIEDIPDPKKMLTNPCAEPDLFSDRPIITKLSCLARHNNIYVVANTADFKKCETDESCDKDNSNCTVCPKDGHFFYNTNVVFNREGTLIAKYYKRHLYFEPEMNTPHKPEDAYFDTDFGNFSTFICFDLMFKEPVKAIGNLSVLNVAYPTYWFDHTPLIFFATPYQQAWAMANKVNLLAANGHHPQTGSLGSGIYSSSKGALIYTHNPDGRSKLLISNVPISPRDDTIDTNNLNPMRFFIENGTIVAAKGEEKPIFKKECLNTVLGDGKPGDYRCGPTNVDQYEFKKLQGNKGKITTCSNKLCCWLDYDAHSMNEDYYFGVSGKDLNFYGEFSFGTEACFIARCESYEGKACTNFKLLSHTIFRKVEIRGNFTTKYILPFAVDSEMRLTNRDNWSLDESRIMYDNVNKKSSLLFFGVYGRLFEKDKDLYNQPDGAVRISCTGNWLIFGLLVSLFLFYF

SEQ ID NO: 65 - protein, 496 aa, Chrysochloris asiatica
MIASQLLAFVAVPVFCILKVSAMDTFIAAVYEHAVVLGNTSLTPVSREEALALMNQNLDLLEGAITSAAMQGAHIIVTPEDGIYGWHFTRDALYPYLEDIPDPQVNWIPCNDPNRFGHTPVQERLSCLAKNNSIYVVANIGDKKLCHTSDPQCPSDGRYQYNTNVVFDSEGKLVARYHKQNLFMGEVQFNSPKMAEVVTFNTTFGRFGIFTCFDILFHDPAVTLVKDFHVDTILFPTAWMNVLPHLSAVEFHSAWAMGMGVNFLAANIHHPMMKMTGSGIYAPDSPPAFHYDMKTKEGKLLLSSLNSHPYSSRVNWSSHASRMEAHSKGNQEFKNTVFFDEFTFVELMGIKGNYTVCQKDLCCHLSYKMTEKRSDEVYALGAFDGLHTVEGTYYLQICTMLKCKTTNIKTCGNSVETASTRFEMFSLSGTFGTQYVFPEVLLSEVQLAPGDFQVSSDGQLSSLKPTSGPVLTVTLFGRWYEKDQMANASSTTEHNH

SEQ ID NO: 66 - protein, 488 aa, Ptilonorhynchus violaceus
MLPFQTLLPTVVFAVTALQALASDTFIAAVYEHAVILPHPTGEPVSPDQALALMNQNMNVLEGAIKEAARQGAHIIVTPEDGIYGWRFTRESIYPYLEDIPDPVVNWIPCTDPSRFGPAPVQERLSCMARNNSIYVVANIGDKKPCNSSDPDCPRDGRYQYNTDVVFDPEGKLVARYHKYNLFMVETQFNYPKEPEAVTFETPFGKFGIFTCFDILFYEPAVVLVNKMQVDTVLFPTAWMNVLPFLTAVEFHSAWAMGMRVNLLAANTHKTILAMTGSGIYAPAGARTYSYNMETEDGHLLIAELDAHPRLSPASPPAVSWNSYALSVERFSQNDHEFTGIIFHDRFTFTELTKPEGNLTVCQKDLCCHLSYKMAGKRDDEAYVLGAFDGLHVVEGQYYLQICTLLKCQSTDLSTCGQPVETAQTKFEMFSLSGTFGTSYVFPEVLYSGVQLAPGEFEVLNDGRLISKAKPTKPVVTVTLFGRWYEKD

SEQ ID NO: 67 - protein, 544 aa, Nephila pilipes
MERDKVTVGNETSLLTGDEDDCLSDNVPQHPNENLETMIAALLSLTAFLAGELSSVKSDGDFYTAAVYEHARYDSATDSAQVIIQRNLANYTIATEIAKSKGADCIVFPEYGVFSPGERSQLKRFLEDIPDPKASRVNPCEESETYSDRPILYALSCLAKNNSIFVVANTGDIKKCEGEPDCPSDGDFHFNTNVVFDRTGTLLVRYHKEHLFYEFGMDLARETQQDTTFETDFGTFATYICFDIDYADMSRVARQPDVDGIMFPTMWVEQPPQMKSIQIWEAWAIANNSTILASNIQLPGYNALGSGIFHGSGPLTYTFNPDGISKLLVAKVPKSGSEPVSPDASITAITETRTWDWKDDGKDVGYECSSRVLGQATDIRTEYRCVEENVTDYSYTELEGPSGHFEICNNGLCCSLDYTTTGLFNRFFLGAFNGLYNHFGRYSWYEEDCILVRCDARGDRPCATFPMKSETVFTQVLLKGNFSSEIVFPSVLENDMRLTPKSDWRFERPNEQEAFIQFNSETGRKVITVGMKGRSYDKDPPYQR

SEQ ID NO: 68 - protein, 467 aa, Sylvietta virens
MLPSQALLPAVLLALAALQALASDTFIAAVYEHAVILPDATEEPVPPSQALALMNKNMDVLEGAVEEAARQGAHIIVTPEDGIYGWRFTRESIYPYLEDIPDPAVNWIPCTDPSRFGPAPVQERLSCMARNHSIYVVANIGDKKPCDSSDPDCPEDGHYQYNTDVVFDTQGKLVARYHKYNLFFESQFNYPKEPEAVTFETPFGKFGIFTCFDILFYEPAVVLVSKMQVDTVLFPTAWMNFLPFLTAVQLHSAWAMGMRVNVLAANTHNTSMEMTGSGIYAPAGAKTYFYNTKTEDGHLLIAELDAHPRLSPASPPAVSWNSYALSVERFSQNDHEFTGVIFEDPFTFMELTKPGGNVTVCQKDFCCHLRYKMAEKRDDEVYVLGAFDGLHVVEGQYYMQICTLLKCPSSNLSTCGGEHVETAQTKFEMFSLSGTFGTSYVFPEVLYSGVQLAPGEFEVTGHPSLLS

SEQ ID NO: 69 - protein, 444 aa, Poecile atricapillus
MLPSQVLLPAVLFALAALQALASDTFMAAVYEHAVILPGPTEEPVSPDSALTLMNKNMDVLEGAIKEAAQQGAHIIVTPEDGIYGWRFTRESIYPYLEDIPDPVVNWIPCTDPSRFGPAPVQERLSCMARNNSIYVVANIGDKKPCDSSDPDCPRDGRYQYNTDVVFDTQGKLVARYHKYNLFQGESQFNYPKEPEAVIFETPFGKFGIFTCFDILFYEPAVVLVSKMQVDTVLFPSAWMNVLPFLTAIEFHSAWAMGMRVNVLAANTHNTSMAMTGSGIYAPAGARTYSYNMKTEDGHLLIAELDAHPRLSPASPPAVSWNSYALNVGRFSQNDHEFTGIIFEDPFTFTELTKPGGNLTVCQKDLCCHLTYKMAEKRDDEVYVLGAFDGLHVIEGQYYLQICSLLKCLSTNLSTCGQPVEMAQTKFEMFSLSGTFGTSYVFPE

SEQ ID NO: 70 - protein, 441 aa, Araneus ventricosus
MSYAEYIPDPKVQKFNPCVENTKDRTILYTVSCLARENNIYIVANLVGSEPCETTCDAAEVENCDAKCPDDGVFFYNTDVAFDREGNLIARYRKVHPYYREIVNVPVETEVGIFETEFGKFGMIVCFDLVFKEAVLLKDEYDIDTLLFPTYWYDDIVPLNAVEFQQAWAIGNGVNVLSANTHAPGTGSMGSGIFSKDDGALVYTYEPDGKSKLLLANVGIKGDSEIPNISSLIEITPDSAVPITETGEKFPVHCYDKVAGPALNGEIEYRCLKLKLENYSLTKLEKPSGEVQACNNELCCTLSYSTQNLQDEYYLAVYNGLNDAFDYFHWWEEICVLTRCDPFGRNPCALQPSISKTVFQKVELKGNFKSEKVYPTVSKTNNRLAPKSEWNFEQGETKAVLSFQSTLNLPLLKVGLLGRSYDRDPEFIPYFPKEFKKQQIN

SEQ ID NO: 71 - DNA, 1599 bp, Phacochoerus africanus
  source 1..1599: mol_type = genomic DNA
atggccaagcgagttgcaattgtgggagctggggtcagtggcctggcctccatcaagtgctgcctggaggaagggctggagcccacctgctttgagaggagcgatgaccttggggggctgtggagattcactgaacatgttgaagaaggcagggcaagcctctacaagtctgtggtttccaacagctgcaaggagatgtcttgttacccagactttccattcccagaagattatccaaactatgtgccaaattctcagttcctggaatatctcagaatgtatgcaaaccagttcaaccttctgaagtgcattcaattcaagactaaagtctgcagtgtaacaaaacgtgaagattttaataccactggccaatgggatgtagtcactctgtgtgaaggaaagcaagagtcagccgtctttgatgctgtcatggtctgcactggttttcttactaacccatatttgccattggactccttcccaggtataaatacttttaaaggccagtacttccatagccgacagtataaacatccagatatatttaaggacaagagtgttcttgtggttggaatgggaaattctggcacagacattgctgtggaggccagccacctggcaaaaaaggtgttcctcagcaccactggaggagcatgggtgatcagccgtgtctttgactcagggtacccatgggacatggtgttcacgacacgatttcagaacttgttcagaaattctctcccaactccaattgtgaattggttgatagcaaaaaagatgaacagctggttcaatcatgcaaattatggcttaatacccgaagacaggatacaactaagagagcctgtgctgaatgatgagctcccaggccgtatcatcactgggaaagtgctcatcaagccaagtataaaggaggtgaaggaaaactctgtggtatttaacagctccccagaggaagagcccattgatatcattgtctttgccactggatacacctttgctttccccttccttgatgagagtgtagtgaaggttgaagatggccaggcatcactatacaagtacatcttccctgcacatctgcagaagccaactctggctgttattggcctcatcaaaccctttggctccatactacccacaggggaaacacaagctcggtgggctgttcgggtcctgaaaggtgtgaataagttaccaccatcaagtgtcatgatacaggaagttaatacaagaaaagaaaacaagcccagtgggtttggcttgtgttactgcaaagctttacaatctgattatatcacatacatagatgaactgctgacctatatcgatgcaaagcccaacatgttttctctgctcctgaaggatccacacctggctttgaccatcttctttggcccatgcacaccataccagttccgcctgactggtccagggaaatgggaaggagccaggaacgctatcatgacccagtgggaccgaacattcaaggccaccaaaactcgaattgtaaaagaatccccatctccctttgcaagcttacttaaactctttagctttctagtcttgcttgtggccattttccagattttcctctaa

SEQ ID NO: 72 - DNA, 1611 bp, Lagenorhynchus obliquidens
  source 1..1611: mol_type = genomic DNA
atggccaagagagttgcaactgtgggagctggggtcagtggcctggcctccatcaagcgctgtccggaagaaggactagagcccacctgcttcgagaggagcgacgaccttggggggctgtggagattcaccgaacatgttgaagaaggcagagccagcctctacaagtctgtggtttccaacagctgcaaggagatgtcttgttacccagactttccattcccagaagattatccaaactatgtgccaaattctgtattcctggaatatctcaaaatgtatgcaaaccagttcaaccttctgaaatgcattcaattcaagactaaagtcttcagtgtaacaaaacgcccagatttttctgtgaccggccaatgggaggtagtcactctgcatgaagagaagcaagagtcagccatctttgacgctgtcatggtctgcactagttttcttactaacccatatttgccactggactcctttccaggtataaatactgttaaaggccagtattttcatagctgagagtataaacatccagatatatttagagacaagagagttcttgtgattggaatggggaattctggcacagacattgctatggaggccagccacgtggcaaagaaggtatcattcttgatagttaccgaacacccctgggcgtgagtgatcagccaggtctttgactcaggctacccatgggacatggtgctcgtgatgcgatttcagaccatgttcagaaattctctcccaactccagttgtgaattggttgatagcaagaaggatgaagagctggttcaatcgtgaaaattatggcttaataccagaagacaggacacaactaagagagtctgtgctaagtaatgagctcccaggccgtatcatcactgggaaagtgctcatcaagccaagcgtaaaggaggtgaaggaaaactctgttgtatttaacaacaccccaaaggaaaagcccattgatatcattgtctttgccactggatacacctttgctttccccttcctccatgagactgtggtgaaagttgaagatggccaagcatcactgtacaagtacatcttccctgcacatctgcaaaagccaaccctggctgttattggcctcatcaaacccttgggctccatgataaccacaggggaaatgcaagcttgatgggctgttcaggtcctgaaaggtgttaataaattaccaccatcaagtgtcatgatagaggaagttaatgcaggaaaaaaaaaaaggccagtgggtaagtttggcttgtgttactgcaaagctttacaatcccattatatcccatgcatagatgaactcctgacctatattgatgcaaagcccaacctgttctctctgctcctgacagatccacgcctggctttgaccatcttcttcggcccatgcacaccataccagttccgtttgacccgcccagggaaatgggaaggagccagaaatgctatcatgaccctgtgggaccaaacattccaggtcacaaaaactagagttgtacaagaatccccttctccctttgcaagcttacttaaactcttcagctttctagctttgcttgtggccattttcctgattttcctataa

SEQ ID NO: 73 - DNA, 1609 bp, Meriones unguiculatus
  source 1..1609: mol_type = genomic DNA
atggtgaagcgagtcgcgattgtgggagccggggtcagcggcctggcctccatcaagtgctgcctggaggagggactggagcccgtctgcttcgagaggagcggggacctggggggactttggagattcactgaagatgttgaagaaggaagagccagtctttacaagtctgtggtttctaacagcagcaaggagatgtcctgttacccagattttccttttccagaagattacccaaactttgtgccaaattctttattcctggaatatctcaaactgtatgcaaacaagttcaaccttctacgatgcattcatttcaataccaaagtgtgcagtataagcaaatgcccagattttgctgtctccggacagtgggaagtggtcactgcccgtggacaagaacagacgcagagctcagccatctttgatgccgtcatggtctgcactggttttctaactaaccctcacttgcccctggattcctttccaggtatacaaacttttaaagggcagtacttccacagccgacagtataagcatccggatgtatttaaggataagcgagtccttgtggttggaatgggaaattctggcacggatattgccgtggaggccagccacttagcaaaaaaggtgttcctgagcaccaccggaggggcatgggtgatcagcagagtctttgattcggggtacccgtgggacatggtattcatgacacggtttcagaacatgttcagaaactctctcccaacgcccattgtgagttggttgatatcaaataagatgaacagctggttcaaccatgtgaattacggtgtggctccagaagacaggattcagctgagagaacctgtcctaaatgatgagctcccaggccgcatcatcactgggaaagtgtttatcaagccaagcattaaggaggtgaaggaaaactctgtcatattcaacagcagcccaaaggaagagcctattgatgtcatcgtttttgccactggatacaccttcgctttccccttcctcgatgaatctattgtcaaagttgaggatggccaggcatcactatacaagtacatcttccctgcgcatctgccaaaaccaactctggctgtgattggcctcatcaaacccctgggctccatgttacccacaggcgacacacaagcccgatgggttgttcaggtcttgaaaggtgcggttacattaccaccaccgagtgtcatgatgcaagaagttgatgaaaggaagaaaaacaagcacagcgggtttggcttgtgctactgcaaggctctgcaaacagattacataacatacatagatgacctcctgacctctatcaacgcaaaaccgaatctgtggtccatgctcctgactgacccgcgcctggctctgagcatcttcttcggcccctgctcaccttaccatttccgcctgactggtccagggaaatgggaaggagccagaaaggccatcttgacccagtgggaccgaaccctgaaagtcaccagaactcgaattgtacaagaatccccatattcctttgaaactttgtttaaactctttagtgttctggctttgcttgtagctattttcttgattttcctgtaagtga

SEQ ID NO: 74 - DNA, 1599 bp, Dipodomys ordii
  source 1..1599: mol_type = genomic DNA
atggtgaagcgggtggccatcgtgggagccggggtcagcggcctggcctccaccaagtgctgcctggaggaaggtctggagcccacctgcttcgagaggagcgaggacctgggcggcctctggaggtacaccgaatatgtggaagagggcagagccagtctctacaactctgtggtctccaacagcagcaaggaaatgtcctgttattcagattttccattcccagaagattacccaaactttgtgccaaattctctcttcctggaatatctcaaaatgtatgcaaaccgatttgatcttctgaaatgcattcaattcaagaccaaagtgtgcagtgttaccaagcgcccagatttcgccacttctggccagtgggaagtggtcactctgcacgaagggaagcagagctcagccaccttcgatgcagtcatggtctgcaccgggtttcttactaacccttctgtgccgctggattccttcccaggcatcaaaacctttaaaggccagtacttccatagccggcactataaacatccaaatatatttaaagacaagcgagtccttgtggttggaatggggaattccggcaccgacattgctgtggaggccagccacctggccaaaaaggtgttcctcagcaccactggaggggcgtgggtgatcagccgtgtctttgactcggggtacccctgggacatggtgttcatgacacgattccagaatatgttcagaaattttcttccaactccaattgtgtcttggttgatagccaaaaagatgaacagctggttcaatcatgaaaattacggtgtaataccagaagacagggttcagatgagagagcctgtgctgaatgatgagctgcctggccgcatcatcaccgggaaagtgtcgatcaagcctagcattaaggaagtgaaggagaactcggtcatattcaacaacaccccgaaggaagagcccattgatatcatcatctttgccacgggctacaccttcgatttccccttcctcgaggaatcggtagtgaaagttgaagatggccaggcatcactgtacaagtatatctttccagtgcatctgccgaagccaacgctggccgtgattaacctcatcaaacctctgggctccatgctgcccacaggagaaacacaagctcgatgggtggtccaagtattcaaaggtaccactctactaccaccagcgagcgtcatgatgaaggaagtcaatgagagaaaagagaacaaacccagtgggtttggcttgtgctactgcaaggctctacagtcggattacatcccatacatcgatgatctcttgacggccatcaacgcgaagcccagcttgtggtccatgttcctgaccgacccgcggctggcctgcgccgtcttctttggcccctgcacaccataccagttccgcctggcgggcacaggaaagtgggaaggagccagacaagccatcctgacgcagtgggaccgagccctgaaagtcaccaaaactcgcactgtgcaaaaacccccctctgcctttgacatctcgcttaaactctttggcttgctggctttgctggtagctgtgttcttggttttcctctga

SEQ ID NO: 75 - DNA, 1410 bp, Bos mutus
  source 1..1410: mol_type = genomic DNA
atggccaaacgagttgcgattgtgggcgctggggccactggcctggcttccatcaagtgctgtctggaggaagggctaaagcccacctgcttcgagaggagtgatgaccttggggggctgtggagattcaccacaaaagtctgcagtgtaagaaaacgcccagattttactgccacaggccagtgggaggtagtcactctgtgtgaagggaagcaagagtcagccatctttgatgctgtcatggtctgtactggtttttttactaatccgaatctaccactggactcctttccaggtataaataattttaaaggccagtatttccacagccgagagtataaacatccagatatatttagggacaagagcgttcttgttattggaatgggaaattctggcacagacattgctgtggaggccagccacctggcaaagaaggtgttcctcagcaccaccggaggggcatgggtgatcagccgtgtctttgactcagggtacccatgggacatggttttcatgacacgatttcagaacacattcagaaattctcttccaactccaattgtgaactggttgatagcgaaaaggatgaacagctggttcaatcatgcaaattatggcttaataccagaagacagggtacagctaagagagcctgtgctaaatgacgagctcccaggccgtatcatcactgggaaagtactcatcaagccaagagttaaggaagtgaaggaaaactctgtcgtatttagcaacaccccaaaggaagagcccatcgatatcattgtttttgccactggatacacttttgctttccccttccttgatgagactgtagtgaaagtcgaagatggccaggcatcgctatacaagtacatctttcctgcacatctgccaaagccaaccctggctgtcattggtctcatcaaacccttgggctccttactacccacaggagatacgcaagctcgatgggctgttcgggtcctgaaaggtgtgattaagttaccaccatcaagcacaatgattgaggaagttaatgaaaggaaaaaaaacaagcccagtgggtttggcttgtattactgcaaagctttggaaaccgattatatcacatatatagatgaactcctgaccgctatcaacgcaaagcccaacctgttctctctgctcctgacggatccacgcctggctttggccatcttctttggcccatgcacaccataccagttccgtttgactggtccagggaaatgggaaggagcccgaaatgctatcatgacccagtgggaccgaacattcaaggccaccaaaactcgaattgtacaagaatccccgtctctctttgcaagcttacttaaagtcttcagctttctagctttgcttgtggccattttcctgattttcctataa

SEQ ID NO: 76 - DNA, 1170 bp, Moschus berezoyskii
  source 1..1170: mol_type = genomic DNA
atggtctgcactggttttcttactgatccatatttaccactggactgctttccaggtataaatacttttaaaggccagtatttccacagccgagagtataaacatccagatatatttagggacaagagagttcttgttattggaatgggaaattccggcacagacattgctgtggaggccagccacctggcaaaaaaggtgttcctcagcaccaccggaggggcatgggtgatcagccgtgtctttgactcagggtacccatgggacatggttttcatgacacgatttcagaacacattcagaaattctcttccaactccaattgtgaattggttgatagcaagaaggatgaacagctggttcaatcatgcaaattacggcttaataccagaagacagggtacagctaagagagcctgtgctaaatgacgagctcccaggccgtatcatcactgggaaagtactcatcaagccaagcgttaaggaagtgaaggaaaactctgttgtatttagcaacaccccaaaggaagagcccatcgatatcattgtttttgccactggatacacttttgctttccccttccttgacgagactgtagtgaaagtcgaagatggccaggcatcgctatacaagtacatctttcctgcacatctaccaaagccaacgctggctatcattggtatcatcaaacccttgggctccttactacccacaggggatatacaagctcgatgggctattcaggtcctgaaaggtatgattaagctaccaccatcaagcataatgatcgaggaagttaatgaaaggaaaaaaaacaagcccagtgggtttggcttgccttactgcagagctttgcaaaccgattacatcacatatgtagatgaacttctgaccgctatcaatgcaaagcccaacctgttctctctgcttctgacggatccacgcctggctctggccatcttctttggtccatgcacaccataccagttccgtctgaccggtccagggaaatgggaaggagcccgaaatgccatcatgacccagtgggaccgaacattcaaggccaccaaaacacgaattgtacaagaacccccgtctctctttgcaagcttacttaaactcttcagctttctagctttgcttgtggccattttcctgattttcctataa

SEQ ID NO: 77 - DNA, 1410 bp, Chrysochloris asiatica
  source 1..1410: mol_type = genomic DNA
atggccaagagagttgcaatagtgggagccggggtcagtggcctggcctgcatcaagtgctgtctggaggaaggattggagcccatctgctttgagaggagtgacgacttgggagggctgtggagattcactaccaaagtctgcagtataacaaagcgcccagatttttctgtctctggccaatgggatgtgatcactctacatgaagggaagcaagagtccgctgtctttgatgctgtcatggtctgcactggttttcttactaatccaagtttgcccctggactcctttccaggtataaaagcttttaaaggccaatactttcatagccgagaatataagcatccagatatatttaaggacaagtgtgtccttgtgattggaatgggtaactctggtacagacattgctgtggaagccagccacctggcaaaaaaggtgttcctgagcacaactgcaggggcatgggtgataagccgagtgtatgactcagggtacccatgggacatggtgttcatgacacgatttcagaacatgttcagaaatgctctcccaactccaattgtgtcttggttgcttgcaagaaagatgaacagttggttcaatcacacaaattacggcgtagttccagaagacagggtacagctgagagagcctgtgctaaatgatgagcttccaggtcgtatcatcacgggcaaagtgctcatcaagccgaatgtaaaggaggtgaaggaacattcagtcatatttaaaaactccccaaaagaagaacccattgatatcattgtctttgccactggatacacctttgcttttcccttccttgatgagtctgtagtgaaagttgaagatggccaggcatcactgtacaagtatatcttccccacgcacctgcaaaagtcaactctggctttcatcggtctcatcaaacccttgggctcgatgatacccacaggagaaatgcaagctcgatgggctgttcgagtactgaaaggtgtgaatacattaccaccatcaagtgttatggcaaaggaggttaacgaaaggaaagaaaacaagcctagtgggtttggcttgtgttactgcaaggctttacaatcagattatatcccatacatggatgaacttctaacctatattgatgcaaaacccaacctgttctccatgctcctgacggatccacgcctggctttgaccatcttctttggtccatgttcaccataccagttccgcttgactggaccagggaaatgggaaggagccagaaacgccatcctgacccagtgggaccgaacattcaaggtcaccaaaactcgagttttactagactcctcatctgtgtttgaaaacttacttaaactctttagctttctggctttgcttatggccattttcctgattttcttgtaa

SEQ ID NO: 78 - DNA, 1599 bp, Erinaceus europaeus
  source 1..1599: mol_type = genomic DNA
atggctagacaagtggcaatcgtgggtgctggggtcagtggcttggcctctatcaagtgctgcctggaggagggattggaacccacctgctttgagaggagcgatgatctcgggggactatggagattcaccgaacatgtggaagaaggcagagccagcctctacaagtctgtggtttccaacagctgcaaggagatgtcctgttactcagactttccattcccagaacattacccaaactacgtgccaaattcccagttcctggaatatctcaaaatgtatgcacatcgatttgatcttctgaaatgcattcaatttaagactaaagtctgcagtgtaacaaaatgcccaaactttgatgtcactggccaatgggaagtggtcactctgcatgaagggaaacaagagtcaagtgtctttgatgcagtcatggtctgcactggttttcttactaacccccacttgccactggactcctttccaggtataaaaacctttaaaggccagtacttccacagccgacagtataaacatccagatatatttaaggacaagagagttctcgtggttggcatgggaaattctggcactgacatcgctgtggaggctagtcatgtggcggagaaggtatacctcagcaccacaggaggagcctgggtaatgagccgagtctttgattcagggtacccatgggacatggtattcatgacacgcttccagaacatgtttagaaatttgctccccactccagttgtgtccaggttgatagcaagaaagatgaacagctggttcgatcacacaaattatggtcttgtaccagaagacagaattcagctgaaggagcttgtgctgaatgatgagcttccaggccggatcatcaccgggaaagtgttcatcaggccaagcataaaggaggtgaaggcgcactcagtcatgttcaacaacagtccaaaagaagagcccattgacatcatagtctttgccacaggctacacttttgccttccccttccttgatgagagtgtaataaaagttgaagctggccaggcatcactctataagtatatcttccctgcacatctgaagaagtcaaccctggcagttattggcctcatcaaacccttgggctccatgatacccactggagaaacacaagctcgctgggctgtccgagtcctgaaaggtgtgaataagttaccacctccaagtgtcatgatagaagaagttaatgcaagaaaagaaaacaaacccactgggtttggcttgtgctactgcaaagctttacaatcagattacattctatacatcgatgaactcctgacttccatcaatgcaaaacccaacctgttctccttgctcctgacagacccacacctggcttgggccatcttctttggcccctgctcaccataccagtttcgcctaactggaccagggaagtgggagggggctcgaaatgccatcctgacccagtgggaccgaacattcaaggccaccagaactcgaattgtccaagaaccgccatctccttttgtaagcttccttaagttcttcagttttctggctttgctggtggctgttttcctgatccttctttaa

SEQ ID NO: 79 - DNA, 1407 bp, Artibeus jamaicensis
  source 1..1407: mol_type = genomic DNA
atgtcctgttactcagactttccattcccggaagattatccaaactatgtgcccaatgcccagttcctggaatatctcaaaatgtatgcaaaccacttcaaccttctgaaatgcattcaattcaagactaaagtgtgcagtgtaactaaatgcccaaatttcactgtcactggccaatgggacgtggtcactctgcacgaagggaagcaagagtccactgtctttgatgctgtcatggtctgcactggtttgcttacttacccaaatttgccactggactcctttccaggtataaatacattcaaaggccaatactttcatagccgacagtataaacatccagacatatttaaggacaagagagttcttgtgatcggagcaggaaattctggtgcagacatagctgtggaggccagccacgtggcaaaaaaggtgttcctcagcaccacaggaggcgcgtgggtgatcagccgcctctgcaactcagggttgccctgggacatggtgttcacgactcgatttcagagcatgttcagaaattctctcccaaccccaattgtgacttggtggatggcaagaaagataaacagctggttcaatcatgcaaattacggcttagtcccaaaagacaggacacagctgaaagagtatgtgataaatgatgagctcccaggccgcatcatcactggaagagtgttcatcaagccaagcgtaaaggaagtgaaggaagactcagtcatatttaacgaccccccagaggaacagcccattgacatcattgtcttcgccactggatacacctttgctttccccttccttgatgaaaccgtggtgaaagtcgaaaatgaccaggcatcgctgtacaagtacatcttcccggcacatctggaaaagccaaccctggctgtcattggcctcatcaactccttaggctccatgatacccacaggagaaacacaagctcgatgggctgttcgagtcctgaaaggtgtgaataacttaccaccttgcagtgtcatgagagaagaagttaataggaggaaagagaacaagcccagtgggtttggcttccgctactgcaaagttttacaatcagattatatcccatacatagatgagcttctgacttatatcaatgctaaacccaacctgttctctctgctcctgacggacccacgcctggcgttgaccatcttcttcggcccatgcacaccataccagttccgcctgaccggcccaggaaaatggaaaggagccagaaaagccatcttgacgcagtgggaccgaaccatcaaggtcaccaaaacccgcactgtacatgaatccccatttccctttgcaagcttactcactgcctttctggttttgcttgtggccattttcctgacttgcctaaagatgagctaa

SEQ ID NO: 80 - DNA, 1407 bp, Gracilinanus agilis
  source 1..1407: mol_type = genomic DNA
atggtcaaacgagttgcaattattggagctggtgtgagtggcctcacctccatcaaatcctgcttagaagaggggctggagcccacctgctttgaaaggagtggtgacattggagggttatggcaatttacaactattgtctgcaatgtgaaaaaaagaccagatttcgctacttctggccagtgggaggtaaccactaagcatgaagggaagcaagaatcagctatatttgatgctgtcatggtctgcactggttatctcactaacccatttttgccactagactcttttccaggtatcaaaacctttaagggtcagtacttccatagcagagagtacaagcatccagacttgttcaaggataagagagtccttgtggttggcatgggcaactctggaacagacattgctgtggaggccagtcatgtggctaaaaaggttttgctcagcaccactggaggtgcatggttgataagtcgagtttttgaccatgggtatccttgggacatggtgttcactactcgatttcaaagtgtattaagaaattgcctcccaactccagttgtaaattggctaattggcaaaaggatgaatggctggttcgatcatgcaaactatggtttggttccagaaaacaggactttcttaagagaactaatattgaatgatgagctcccaggacgcataattaccgggaaggtatcgattaaacccagtgtggaggcagtgaaggaaaattctgttttgtttaagaataccccaaaagaagagccaattgatataattgtctttgccacaggatatacttttgattttcctttccttgatgaatctatagtgaaagttgaaaataatcaggcttctctatataagtacatattccctgttcatctggaaaagccaactttggctgtcattggcctcatcaaacctttggtatctttagtctgcacaacagaagtacaagcccgatgggtcacgcgtgtcctaaagggcataaataagttaccaccaccagaggtcatgagagaagatgttaacaaattaaaattaaatagagccactgggtttggcttacacttcaatatggttttgcaaacagatactatcagatacttagatgagctcttgtcctccatcgatgcaaaacccaatatcttgtctctgttcctggaggacccgctcctggctttcaagattttctttggcccacttacctcataccagtaccgtctgactggcccaggaaaatgggatggagccaggaatgccattctaacccagtgggaccggtcattcaagccaatgaaaacgcgagttgtgcaagaggttccacatccctttttcaacctgctcaaaatctttggttttttagttctccttgtagctatatttcttacattctaa

SEQ ID NO: 81 - DNA, 1494 bp, Cercocebus atys
  source 1..1494: mol_type = genomic DNA
atggccaagcgagttgccattgtgggagctggggtcagcggcctggcctccatcaagtgctgtctggaagaaggactggagcccacctgctttgagaggagcgacgaccttggagggctgtggagattcaccgaacatgttgaagaaggcagagccagtctctacaagtctgtggtttccaacagctgcaaggagatgtcttgttactccgactttccattcccagaagattatccaaactatgtgccaaattctcaattcctggaatatctcaaaatgtatgcaaaccacttcaaccttctgaaacacattcaattcaagaccaaagtctgcagtgtaacaaaatgttcagattttactgtctctggccagtgggaggtggtcactatgcgtgatgagaagcaagagtcagccatctttgatgctgtcatggtctgcactggctttcttactaacccttatttgccactggactcctttccaggtattaatgcctttaaaggccagtactttcatagccggcaatataagcatccagatatatttaaggacaagagagtccttgtgattggaatgggaaattctggcacagacattgctgtggaggccagccacctggcggaacaggtgttcctcagcaccaccggagggggatgggtgatcagccgaatctttgactcaggctacccatgggacatggtgttcatgacacgatttcagaacatgttgagaaattctctcccaactccaattgtgacttggttgatgatgcgaaagataaacaactggctcaatcatgcaaattacggcttaataccagaagagagaactcagctgaaagagtttgtactaaatgatgagctcccaggccgcatcatcactgggaaagtgttcatcaggccaagcataaaagagtctgtagtgaaagttgaagatggccaggcctcactgtacaagtatatcttccctgcacatctgcaaaagccaaccctggccattattggcctcatcaaacccttgggctccatgatacctacaggagaaacacaagctcgttgggctgttcgagtcctgaaaggtgtaaataagttaccaccaccgagtgtcatgatagaggaaattaatgcaaggagagaaaacaagcccagttggtttggcttgtgctactgcaaggctttacaatcagattatatcacatacatagatgaactcctgacctatatcaatgcaaaacccaagctgttctctatgctcctaacggatccacacctggctctgaccgtcttctttggcccatgctcaccataccagttccgcttgactggcccaggaaaatgggaaggagccagaaatgccatcatgacccagtgggaccgaacattcaaggtcaccaaaggtcgagttgtacaagagtctccatctccctttgaaagttttcttaagctctttagctttctggctttgcttgtggctatttttctgattttgctataa

SEQ ID NO: 82 - protein, 532 aa, Phacochoerus africanus
MAKRVAIVGAGVSGLASIKCCLEEGLEPTCFERSDDLGGLWRFTEHVEEGRASLYKSVVSNSCKEMSCYPDFPFPEDYPNYVPNSQFLEYLRMYANQFNLLKCIQFKTKVCSVTKREDFNTTGQWDVVTLCEGKQESAVFDAVMVCTGFLTNPYLPLDSFPGINTFKGQYFHSRQYKHPDIFKDKSVLVVGMGNSGTDIAVEASHLAKKVFLSTTGGAWVISRVFDSGYPWDMVFTTRFQNLFRNSLPTPIVNWLIAKKMNSWFNHANYGLIPEDRIQLREPVLNDELPGRIITGKVLIKPSIKEVKENSVVFNSSPEEEPIDIIVFATGYTFAFPFLDESVVKVEDGQASLYKYIFPAHLQKPTLAVIGLIKPFGSILPTGETQARWAVRVLKGVNKLPPSSVMIQEVNTRKENKPSGFGLCYCKALQSDYITYIDELLTYIDAKPNMFSLLLKDPHLALTIFFGPCTPYQFRLTGPGKWEGARNAIMTQWDRTFKATKTRIVKESPSPFASLLKLFSFLVLLVAIFQIFL

SEQ ID NO: 83 - protein, 532 aa, Bos taurus
MAKRVAIVGAGATGLASIKCCLEEGLKPTCFERSDDLGGLWRFTEHVEEGRASLYKSVVSNSCKEMSCYPDFPFPEDYPNYVPNSQFLDYLKMYANRFNLLECIQFKTKVCSVRKRPDFTATGQWEVVTLCEGKQESAIFDAVMVCTGFLTNPNLPLDSFPGINNFKGQYFHSREYKHPDIFRDKSVLVIGMGNSGTDIAVEASHLAKKVFLSTTGGAWVISRVFDSGYPWDMVFMTRFQNTFRNSLPTPIVNWLIAKRMNSWFNHANYGLIPEDRVQLREPVLNDELPGRIITGKVLIKPRVKEVKENSVVFSNTPKEEPIDIIVFATGYTFAFPFLDETVVKVEDGQASLYKYIFPAHLPKPTLAVIGLIKPLGSLLPTGDTQARWAVRVLKGVIKLPPSSIMIEEVNERKKNKPSGFGLCYCKALETDYITYIDELLTAINAKPNLFSLLLTDPRLALAIFFGPCTPYQFRLTGPGKWEGARNAIMTQWDRTFKATKTRIVQESPSLFASLLKVFSFLALLVAIFLIFL

SEQ ID NO: 84 - protein, 532 aa, Bos taurus
MGKRVAIVGAGVSGLASIKCCLEEGLEPTCFERSDDLGGLWRFTEHVEEGRASLYESVVSNSCKEMSCYSDFPFPEDYPNYVPNSQFLEYLKMYANHFNLLKHIQFKTKVCSVTKCSDFTVSGQWEVVTLHKEKQESAIFDAVMVCTGFLTNPYLPLDSFPGINAFKGQYFHSRQYKHPDIFKDKRVLVIGMGNSGTDIAVEASHLAKKVFLSTTGGAWVISRIFDSGYPWDMVFMTRFQNMLRNSLPTPIVTWLMARKINNWLNHANYGLMPDDRTQLKEFVLNDELPGRIITGKVFIRPSIKEVKENSVIFNNTSKEEPIDIIVFATGYTFAFPFLDESVVKVEDGQASLYKYIFPANLQKSTLAIIGLIKPLGSMIPTGETQARWAVRVLKGVNKLPPPNVMIEEVNSRKENKPGWFGLCYCKALQADYITYIDELLTYINAKPNLFSMLLMDPHLALTVFFGPCSPYQFRLTGPGKWEGARNAIMTQWDRTFKVTKVRVVQKSPSPFESFLKLFSFLALLVAIFLIFL

SEQ ID NO: 85 - protein, 532 aa, Suncus etruscus
MARAVAIVGAGVAGLASIKCCLEEGLEPTCFERSDDLGGLWRFTEHVEEGRASLYKSVVSNSCKEMSCYSDFPFPEHYPNYVPNSYFLDYLRMYADHFDLLRCIRFKTKVCSVTKSPDFSVTGKWEVITQQEGKQESATFDAVMVCTGFLTIPHLPLDSFPGIKNFKGEYFHSRRYKHPDIFKDKRVLVIGMGNSGTDIAVEASHVAKKVYLSTTGGAWVMSRVFDSGYPWDMVFTTRFQNLLRNSLPTSLVTRLIARKMNSWFNHANYGVIPKDRTQLREPVLNDELPGRIITGKVLIRPSIREVRAHSVLFCDMPEEEPVDIIVLATGYTFAFPFLDETVVKVEEGQASLYKYIFPAHLPKPTLAVIGLIKPLGSMVPTMETQARWAVRVLKGVNKLPPSAVMMEEVRVRKQNKHTGFGLHFCQALQSDYIPYIDELLDAIGAKPNLLALLLTDPRLAWALFFGPFTPYQFRLMGPGKWAGARHAILSQWDRTVKVTKTRVVQESTSSVISFLKLIGFLVVLMAVFLIFL

SEQ ID NO: 86 - protein, 469 aa, Pan paniscus
MAKRVAIVGAGVSGLASIKCCLEEGLEPTCFERSDDLGGLWRFTSKVCSVTKCSDFAVSGQWEVVTMHEEKQESAIFDAVMVCTGFLTNPYLPLDSFPGINAFKGQYFHSRQYKHPDIFKDKRVLVIGMGNSGTDIAVEASHLAEKVFLSTTGGGWVISRIFDSGYPWDMVFMTRFQNMLRNSLPTPIVTWLMERKINNWLNHANYGLIPEDRTQLKEFVLNDELPGRIITGKVFIRPSIKEVKENSVIFNNTSKEEPIDIIVFATGYTFAFPFLDESVVKVEDGQASLYKYIFPAHLQKPTLAIIGLIKPLGSMIPTGETQARWAVQVLKGVNKLPPPSVMIEEINARKENKPSWFGLCYCKALQSDYITYIDELLTYINAKPNLFSMLLTDPHLALTVFFGPCSPYQFRLTGPGKWEGARNAIMTQWDRTFKVIKARVVQESPSPFESFLKVFSFLALLVAIFLIFL

SEQ ID NO: 87 - protein, 469 aa, Homo sapiens
MAKRVAIVGAGVSGLASIKCCLEEGLEPTCFERSDDLGGLWRFTTKVCSVTKCSDSAVSGQWEVVTMHEEKQESAIFDAVMVCTGFLTNPYLPLDSFPGINAFKGQYFHSRQYKHPDIFKDKRVLVIGMGNSGTDIAVEASHLAEKVFLSTTGGGWVISRIFDSGYPWDMVFMTRFQNMLRNSLPTPIVTWLMERKINNWLNHANYGLIPEDRTQLKEFVLNDELPGRIITGKVFIRPSIKEVKENSVIFNNTSKEEPIDIIVFATGYTFAFPFLDESVVKVEDGQASLYKYIFPAHLQKPTLAIIGLIKPLGSMIPTGETQARWAVRVLKGVNKLPPPSVMIEEINARKENKPSWFGLCYCKALQSDYITYIDELLTYINAKPNLFSMLLTDPHLALTVFFGPCSPYQFRLTGPGKWEGARNAIMTQWDRTFKVIKARVVQESPSPFESFLKVFSFLALLVAIFLIFL

SEQ ID NO: 88 - protein, 530 aa, Desmodus rotundus
MAKRVAIVGAGVSGLASIKCCLEEGLEPTCFERSDDLGGLWRFTEHVEEGRASLYKSVVSNSCKEMSCYSDFPFPEDYPNYVPNAQFLEYLKMYANHFNFLKCIQFKTKVCSVTKRPDFTVTGQWDVVTLREGKQESTVFDAVMVCTGLLTDPNLPLDSFPGINTFKGQYFHSRQYKYPDIFKDKRVLVIGAGNSGADIAVEASHVAKKVFLSTTGGAWVISRVCDSGLPWDMLFTTRFQNVFRNSLPTPVVTWWMARKINSWFNHANYGLVPEDRTQLKEYVINDELPGRIITGRVLIKPSVKEVKEDSVIFNGSPEEQPIDIIVFATGYTFAFPFLDETVVKVENGQASLYKYIFPAHLEKPTLAVIGLINSLGSMIPTGETQARWAVRVLKGVNNLPPQSVMREEVNARKENKPSGFGLCYCKALQSDYIPYIDELLTYIDAKPNLFSLLLTDPRLALTIFFGPCTPYQFRLTGPGKWKGARNAILTQWDRTIKVTKTRTVHESPFPFASFLTVFLVLLVAIFLTCL

SEQ ID NO: 89 - protein, 532 aa, Gopherus flavomarginatus
MVKRVAIIGAGVSGLTSIKCCLDEGLEPSCFERSDDIGGLWRFTEDVEDGRPSLYKSMVSNTCKEMSAFTDFPYPEDFPVFVSNSQVLEYLRMYAKHFGLLKHIQFKTTVISVRKCPDFSATGQWAIVTESNGKQESSVFDAVMVCIGYLSDPSLPLESFPGIETFQGEYFHSRYYKHPDVFEGKRVLVIGMGNSGVDIAVEASHVAKKVMISTGRGAWVISRVFDNGYPWDMVFLTRFMNVIRNSLPGPITTWLVAHRTSQWFDHANYGLIPKDRDVIREPVLNDDLPGCIITGKITIKPSLKEIKENSVIFQNSPTEEPVDIIVFATGYKFSFPFLEESVVKVEKKQASLYKYIFPPHLEKPTLAVIGLIKPFGAVMPVVEIQARWVTRVFKGLCQLPPVSTMVEEINETKKNKIRWFGLSYDEILKTDCLVYIDELASLIGTKPNVLALLLKDPVLAMKIFFGPCTAYQYCLTGPGKWNGARHAILTQWDRTVKPTRTRVVEEPPNFCFNLLKLISLFALFAAIFLGFK

SEQ ID NO: 90 - protein, 519 aa, Dromaius novaehollandiae
MRVAVVGAGVSGLTALKCCLAEGLAPTCFEQSRDVGGLWRYTEHIEDGRPSLYTSVVSNTSKEMSAFSDFPFPEDFPVFLPHARLLEYLQRYAERFALRQHIHFGTTVVSVRKRPDFAATGQWDVVTEADGTRESHVFDAVMVCSGNFSDPSLPLHCFPGIERFQGQYFHSRQYKHPDVFQGKRVLVVGMGNSGVDIAVEASRVAAKVTVCTGRGAWVFSRVFDHGYPWDMVFNTRFMNLIRNSLPGALTRGLISYRVNQWFNHENYGLQPEKSCLVREPVLNDDLPSYIITGKITIKPGVKEFKENSAIFYNCPEEEPIDIVVFCTGYNISFPFLEEADVKVENNHASLYKYVFPAHLQRPTLAVLGLIKPLGAIMPVTEMQARWVTRVFKGRRSLPHLLFSDSLRFGLSFDEVLKIDCLVYSDEIASFIGVKPNVLALLFRDPKLALSVFFGPCTPYQFRLQGPGCWDGARQAILTQWDRTLKPTRTRVTSSSSGIWPSFLTLFGFLLLMAAVFFGF

SEQ ID NO: 91 - protein, 535 aa, Agelaius phoeniceus
MRVAVVGAGVSGLAATKCCLDEGLEPTCFEQSQDIGGLWRYTEHIEAGRPSLYPSVISNTSKEMSAFSDFPFPEHFPVFLPNALLLDYLRRYTEHFSLRQHIRFGSTVVSIRKHPDFATTGQWDVVTEAGGKQTSHVFDAVMVCSGNFSEPSLPLQCFPGIERFRGQHFHSRQYKHPDVFQGKRVLVVGMGNSGVDIAVEASRVAAKVTISTTRGAWLLSRVFEHGYPWDMILNTRLMSLIKTNLPGPLSWWLINYKVNQWFNHENYGLQPENRYSWCWLVREPVLNDELPSYILTGRITIKPGVKEFKDNSVLFHNCPEEEPIDIVVFCTGYTISFPFLEESIVRVENKHASLYKYVFPPQLQKPTLAVLGLIKPFGAIMPVAEMQARWVSRVFKGRRANVTLLCPKMQLRQNWTWFVPSPREIPELRILKTEWLVYLDTLASFIGAKPSVLRLLCTDPWLALTIFFGPCSPYQYRLGGPGRWQGARQAILTQWDRVLKPTRTRVPAGSSSSSLSLLTVVGFLLLLAAVIFGFL

SEQ ID NO: 92 - protein, 521 aa, Crypturellus undulatus
MRVAVVGAGVSGLTAIKCCLDEGLKVTCFERSQDIGGLWRYTENIEDGRPSLYTSVVSNTSKEMSAFSDFPYPEDFPVFLPHARLLEYLRRYAERFALRQRIQLETTVLSVRKRPDFATTGQWDVVTEAGGKRTSHVFDAVMVCSGNFWEPSLPLHAFPGIEKFQGKYFHSRQYKHPDVFQGKRVLVVGMGNSGVDIAVEASRVAAKVTLSTGQGAWVISRVFDHGYPWDMVFNTRFMSLLRNNLPGPLARGLISHRVNQWFNHENYGLQPKKRYFSEPVLNDDLPSYIITGKITIKPGVKEFKENSVVFYSCPEEEPIDIVVFCTGYNVSFPFLEEADVKVENKHASLYKYVFPTHLQRPTLAVLGLIKPLGAIMPVIEMQARWVTRVFKGRRLMPVSLHPIFSNSLRFGLLFDEVLKTDCLVYSDELASFIGAKPNVLALLFSDPKLAFTIFFGPCTPYQFRLRGPGRWDGARQAIFTQWDRMLKPMRTRVTSRSSSVWPSLLTVFGFLLLVAAVVFGF

SEQ ID NO: 93 - protein, 524 aa, Regulus satrapa
MRVAVVGAGVSGLTATKCCLDEGLEPTCFEQSQDLGGLWRYTEHIEAGRPSLYPSVISNTSKEMSAFSDFPFPEHFPVFLPNALLLDYLRRYAERFSLREHIKFGTTVVSIHKHPDFATTGQWNVVTEAGGKQTLDVFDAVMVCSGNFSEPSLPLQCFPGTRFQGQYFHSRQYKHPDVFQGKRVLVVGMGNSGVDIAVEASRVAAKVTISTTRGAWLLSRVFEHGYPWDMILNTRLMSLIKTNLPGPLSWWLINYKVNQWFNHENYGLQPEKRYLPVLNDDLPSYILTGRITIKPGVKEFKDNSVLFHNCSEEEPIDIVVFCTGYIVSFPFLEESIVRVENKHASLYKYVFPTHLQKPTLAVLGLIKPFGAIMPVAEMQARWVSRVFKGRETNVSLPVPQNAAQTGQVLSLQFGLTFDEVLKTEWLVYLDTLASFIGAKPSVLGLLCTDPRLALTIFFGPCSPYQYRLEGPGRWEGARQAILTQWDRVLKPTRTRVPAGSSSSFPSLLTVVGLLLLLAAVIFGF

SEQ ID NO: 94 - protein, 495 aa, Mirounga angustirostris
MPCESGKEHVEEGRASLCKSVVSNSCKEMSCYSDFPFPEDYPNYVPNSQFLEYLKMYANWFSLLECIQFKTKVCKVTKCPDFTVTSQWEVVTQHEGKQESAIFDAVMVCSGFLTNPYLPLDSFPGINTFKGQYFHSQQYKHPDIFKDKRVLVIGMGNSGTDIAVEASHLAKQVFLSTTGGAWVMSRVFDSGYPWDMVFMTXFQNMFRNSLPTPFVTWLMARKMNSWFNHANYGLVPEDRTQLREPVLNDELPGCIITGKLLIRPSIKEVNENSVVFNNTPKEEPIDIIVFATGYTFAFPFLDETVVKVENGQASLYKYIFPAHLPQPTLAVINLIKPLGSMIPIGETQARWVVRVLKGINKLPPRSVMIDEVNARKEDTPSGFGLCYCKALQSDYITYIDELLTSINAKPNLFSLLLMDPRLALTIFFGPCTPYQFRLTGPGKWKGARNAILNQWDRTFKVTKTRIVQESPSPFASLLKLLSLLALLMAIFLIFL

SEQ ID NO: 95 - protein, 468 aa, Artibeus jamaicensis
MSCYSDFPFPEDYPNYVPNAQFLEYLKMYANHFNLLKCIQFKTKVCSVTKCPNFTVTGQWDVVTLHEGKQESTVFDAVMVCTGLLTYPNLPLDSFPGINTFKGQYFHSRQYKHPDIFKDKRVLVIGAGNSGADIAVEASHVAKKVFLSTTGGAWVISRLCNSGLPWDMVFTTRFQSMFRNSLPTPIVTWWMARKINSWFNHANYGLVPKDRTQLKEYVINDELPGRIITGRVFIKPSVKEVKEDSVIFNDPPEEQPIDIIVFATGYTFAFPFLDETVVKVENDQASLYKYIFPAHLEKPTLAVIGLINSLGSMIPTGETQARWAVRVLKGVNNLPPCSVMREEVNRRKENKPSGFGFRYCKVLQSDYIPYIDELLTYINAKPNLFSLLLTDPRLALTIFFGPCTPYQFRLTGPGKWKGARKAILTQWDRTIKVTKTRTVHESPFPFASLLTAFLVLLVAIFLTCLKMS

SEQ ID NO: 96 - protein, 483 aa, Bubalus carabanensis
MNKWFCFIVPTQDNMAKRVAIVGAGVTGLASIKCCLEEGLKPTCFERSDDLGGLWRFTTKVCSVRKRPDFTATGQWEVVTLCEGKQESAIFDAVMVCTGFLTNPYLPLDSFPGINTFKGQYFHSREYKHPDIFRDKSVLVIGMGNSGTDIAVEASHLAKKVFLSTTRGAWVISRVFDSGYPWDMIFMTRFQNKFRNSLPTPIVNWLIARRMNSWFNHANYGLVPEDRVQLREPVLNDELPGRIITGKVLIKPSVKEVKENSVIFSNTPKEEPIDIIVFATGYTFAFPFLDETVVKVEDGQASLYKYIFPAHLPKPTLAVIGLIKPLGSLLPTGDTQARWAVRVLKGVIKLPPSSIMIEEVNERKKNKPSGFGLCYCKALETDYITYIDELLTAINAKPNLFSLLLTDPRLALAIFFGPCTPYQFRLTGPGKWEGARNAIMTQWDRTFKATKTRIVQESPSLFASLLKVFSFLALLVAIFLIFL

SEQ ID NO: 97 - protein, 389 aa, Moschus berezovskii
MVCTGFLTDPYLPLDCFPGINTFKGQYFHSREYKHPDIFRDKRVLVIGMGNSGTDIAVEASHLAKKVFLSTTGGAWVISRVFDSGYPWDMVFMTRFQNTFRNSLPTPIVNWLIARRMNSWFNHANYGLIPEDRVQLREPVLNDELPGRIITGKVLIKPSVKEVKENSVVFSNTPKEEPIDIIVFATGYTFAFPFLDETVVKVEDGQASLYKYIFPAHLPKPTLAIIGIIKPLGSLLPTGDIQARWAIQVLKGMIKLPPSSIMIEEVNERKKNKPSGFGLPYCRALQTDYITYVDELLTAINAKPNLFSLLLTDPRLALAIFFGPCTPYQFRLTGPGKWEGARNAIMTQWDRTFKATKTRIVQEPPSLFASLLKLFSFLALLVAIFLIFL

SEQ ID NO: 98 - DNA, 1542 bp, Synthetic construct
  source 1..1542: mol_type = other DNA
atgatcacctctccgttgctggcttatgttgcgattctgttcttgtgtgtcctgaaagccagctccctggatacctttatcgccgctgtttatgaacacgcggtgattcttcctgatgcccccttgacaccagtatcccacgaagaggctctcatgctgatgaaccgcaacctggatcttttggaaggcgcaatcatgtcagcagcaaagcaaggtgcgcacatcattgtgactccagaagatggtgtttacggcttcttcttcagccgcgaatctatctactcctacctcgaggacatcccagagccgcacgtgaactggatcccttgcaccaatcccaaccgctttggccacaccccagtacaaaagcgactcagctgcctggcccgggacaactccatctacattgtggccaatatcggagacaagaagccatgcaacgcctcggaccccgactgtcctcatgacggccgttaccagtacaacaccgatgtggtctttgattccgagggccgactggtcgcacgctaccacaagcaaaatcttttcttgggtgaagatcaattcgatgcacccaaggagcccgagatcgtgacgttcgacaccacctttggccgttttggcatcttcacgtgctttgatattctcttccatgatcctgctgtgaccctggtcaaagatttccaggtagacaccatcctcttcccgaccgcgtggatgaacgttctcccacatttgaccgctattgaattccactcggcttgggctatgggcatgcgtgtcaacttccttgcggccaatatccacttcccgctgcgaaaaatgacaggatcgggcatctatgcacctgattctccacgcgcctttcattacgatatgaagaccaaggaaggaaaactcctcttgtcacaattggattcccacccgcaccgcccagcagtgaattggacgtcttatgcctcaggtttgccagcccccttggtgggtaaccaggaattcaaatccattgtcttttttgacgagttcaccttcttcgagcttaaaggtgttgcgggaaactacacagtttgccagaaggatctctgctgccagctttcttaccggatgcttgagatgcgtgaagacgaagtttatgcgctcggcgcattcgatggacttcatactgtggaaggtagctattacttgcagatttgcacattgctgaagtgtaaaaccatggacctgcactcctgtggtgactcggtcgaaactgcttccacccgctttgaaatgttctccctcagcggcacgtttggcacccagtatgtgttccctgaggtgttgctgtcggacattcagcttgcgcctggagaatttcaggtttcatccgatggccgcttgttcagcctgaagccaccgtctggaccggtccttactgtcactctgtttggccgcctctacgagcgtgattctgcatcaggtgcttcagcagacctcgtggcacaaggactgcgagtcatgctcggtgttatcattactatcatgtactccttgtcctggtaa

SEQ ID NO: 99 - DNA, 1599 bp, Synthetic construct
  source 1..1599: mol_type = other DNA
atggccaagcgcgttgcaattgttggagctggtgtctcaggcctggcctcgatcaagtgctgcctggaagagggcctggagcccacctgcttcgagcgttccgatgaccttggcggcctgtggcgattcaccgaacacgttgaagaaggtcgcgcctctctctacaagtctgtggtttccaactcctgcaaggagatgtcttgttacccagacttcccattcccagaagattaccctaactacgtgccaaattctcacttcctggaatatctccgaatgtatgcaaaccagttcaaccttctgaagtgcattcaattcaagaccaaagtctgctcggtaaccaaacatgaggactttaataccaccggtcaatgggatgtagtcactctgtgtgagggaaagcaagagtcagccgtcttcgatgcggtcatggtctgtactggttttctcacgaacccatatttgcctttggactccttcccgggcatcaatacttttaaaggccagtatttccacagccgtcagtacaagcacccggatatttttaaggacaagtctgttcttgtggttggtatgggtaactcgggcacagacattgctgtggaagcgagccacctggcaaaaaaggtgttcctcagcaccacgggcggagcatgggtgatcagccgtgtctttgactcaggctacccgtgggacatggtgttcatgacacgctttcagaacatgttccgcaattcgctccctaccccaattgtgaattggttgattgcaaaaaagatgaacagctggttcaatcatgcgaattatggcctgatccccgaagaccgcatccaactgcgtgagcctgtgctgaacgatgagctcccaggtcgtatcatcactggaaaagtgcttatcaagccatccatcaaggaagtgaaggaaaactccgtggttttcaacagctccccagaggaagagcccattgatatcattgtctttgccactggatacacctttgctttcccgttccttgatgagtccgtagtgaaggttgaagatggccaggcatcactttacaagtacatcttccctgcacatctgcagaagccaactctggctgttattggcctcatcaaacccttgggctccttgttgcccaccggcgacacccaagctcggtgggcggttcgggtccttaaaggtgtgaacaagttgccgccttcctcggtcatgatccaggaagttaatacacgcaaagaaaacaagccctcaggatttggtttgtgttactgcaaagctctccaatccgattatatcgcatacatcgatgaactgctgacctacatcgatgcgaagcctaacatgttttctctgctcctgacggatccacacctggcgttgaccatcttctttggcccgtgcacaccttaccagttccgcctgaccggtccgggaaaatgggaaggtgcccgaaacgctatcatgacccagtgggaccgcaccttcaaggtcaccaaaacgcgcattgtaaaagagtccccatctccctttgcatccctcttgaaactctttagctttcttgcgttgcttgtggccattttccagattttcctctaa

SEQ ID NO: 100 - DNA, 1563 bp, Bos taurus
  source 1..1563: mol_type = genomic DNA
atgatcacttcctctttacaaacctctgtggcagtttttgccctagtaattctacatgtcagcactctggatacttttaaagctgcagtgtatgaacatgctgtcatactgccaaatgacacagaaacacctgtttctccggatgaggccttgttcctcatgaacaagaacatagatattctggagaaggcaattaagcaggcagctgagcagggtgctcagattatcgtgaccccagaagatgcactttatggatggaaatttaccagggaaaccattttcccttatctggagaatatcccagatcctcaggtgaactggattccatgtcaagaaccccacagatttggtcatacaccagtacaagcaagactcagctgcctggccaagaacaactctatctatgtcgtggcaaatatgggtgacaaaaagccatgtaattcccataataccacttgtccttctaatggccattatcaatacaacaccaacgtggtgtatgatgcaaagggaaagctggtggcgcgttaccataagtaccacctctaccgtgagattcagtttgatgtccccgaaaagccagagttggtgactttcaacaccacctttggaaagtttggcattttcacgtgctttgatattctcttccatgatcctgctgtgaccctggtgaaagattcccacgtggacaccatattgtttcccacagcttggatgaacgttttgccctttatgacagctattgaatttcattcggcttgggcaatgggaatgagagttaatcttcttgcagcaaacacacataatgtcagcctaaaaatgacaggcagtggtatctatgcaccacactctcccaaagtctaccatcatgatatggaaacagagtcaggaaagatcctctttgcagaggtggattcccatccccgaaactcctcgacctacccaccagccatgaattggagtgcctacgccacctccatcacaccctttccaggacctaaaaattccttcaggggatttatttcccgtgatgagttcaactttacaaaactctctgaaagtgcaggaaaccttacagtctgtcaaaaagagctctgctgtcatttaagctacagaatgttagcaaaggagggggatgaagtgtatgttctaggagcttttgcaggacttcatgggagaaggagaagagaatattggcaggtatgcacaatgctgaaatgcagatctactgatgtgacaacttgtggacagccagtagaaactgctttgacgagatttgaaatgttctctctaagtggcacatttggaacagagtatgtttttcctgaagtgctacttactaaaatcaagctggcacctgaaaaatttgaggtactagaagatgggcgtttggtaaacaagaatggattatctgagcctgtcctaacagtgtcgctctttgggagatggtacaccaaggacgcacattccatctcaggcgggaccagcaactcagcaacagcgtacctgatgatacccacattattaatgatcataaccttgcgggatgctgtgatggtgtag

SEQ ID NO: 101 - DNA, 1563 bp, Ovis aries
  source 1..1563: mol_type = genomic DNA
atgatcacttcctctttacaaacctctgtggcagtttttgccctaataattctacatgtcagtgctctggatacctttaaagctgcagtgtatgaacatgctgtcatactgccaaacgacacaaaaacacctgtttctccggatgaggctttgttcctcatgaacaagaacatagatatcctggagaaggcaattaagcaggcagctgagcagggtgctcagatcattgtgaccccagaagatgcactttatggatggaaatttaccagggaaaccattttcccttatctggagaatatcccagatcctcaggtgaactggattccatgtcaagacccccacagatttggtcatacaccagtacaagcaagactcagctgcctggccaagaacaactctatctatgtcgtggcaaatatgggtgacaaaaagccatgtagttcccgtaataccacgtgtccttctaatggccattatcagtacaacaccaacgtggtatatgatgcaaagggaaagctggtggcgcgttaccataagtaccacctctaccatgagcttcaatttgatgtccccgaaaagccagagctggtgactttcaacaccgcctttggaaagtttggcatcttcacgtgctttgatattctcttccatgatcctgctgtgaccctggtgaaagatttccacgtggacaccatattgtttcccacagcttggatgaacgttttgccctttttgacagctattgaatttcattcggcatgggcaatgggaatgagagttaatcttcttgcagcaaacacacataatgtcagcctgaaaatgacaggcagtggtatctatgcaccacactctcccaaagtctaccatcatgatatggaaacagagtcaggaaagatcctctttgcagaggtggattcccatccccgaaactcctcgacctccccaccagccgtgaactggagtgcctacgccacctccatcacaccctttccaggacctaaaaattccttcaggggatttatttcccgcgatgagttcaacttcacaaagctctctgaaagtgcaggaaaccttacagtctgtcaaaaagagctctgctgtcatttaagctacagaatgttaggaaaggaggaggatgaagtgtatgttctaggagcttttgcaggacttcatgggagaaggagaagagaatattggcaggtatgcacaatgctgaaatgcagatctactaatttgacaacttgtggacagtcagtagaaactgctctgacgagatttgaaatgttctctctaagcggcacatttggaacagagtatgtttttcctgaagtgctacttactaaaatcaagctggcacctgaaaaatttgaggtactagaagatgggcgtttggtaaacaagaatggattatctgaacctgtcctaacagtgtcgctctttgggaggtggtacaccaaggacatacattccatctcaggcgggaccagcaactcagcaacaacgtacctgatgatacccacattattaatgatcatagtcttgcagtatgctgtgatggtgtag

SEQ ID NO: 102 - DNA, 1563 bp, Balaenoptera sp.
  source 1..1563: mol_type = genomic DNA
atgatcacttcctcttttcaaacttccgtggcagttttggccctaattattctgcatgtcagcgctttggatactttcaaagctgcagtatatgaacatgcggtcatattgccgaatcacacagaaacacgtgtttctcaggatgatgccttgctcctcatgaacaagaatatagatattctggagatagcaattaagcaggccgctgaacagggtgctcagatcattgtgactccagaagatgcaatttatggatggaaatttaccagggaagccattttcccttatctggaggatattccagatcctctggtgaattggattccatgtcaagacccacacagatttggtcgtacaccagtacaagcaagactcagctgcctggccaagaacaactctatctatgtcttggcaaatattggggacaaaaagccatgtaattcctgtgactccacgtgtcctcctaatggccattatcaatacaacaccaacgtggtgtatgatgcaaagggaaagctggtggcacgttaccataagtacaacctctactctgagcttcagtttgatgtccccaaaaagccggagttggtgactttcaacacctcctttggaaagtttggcattttcacatgctttgatattctcttccatgatcctgctgtgaccctggtgaaagatttccacgtggacaccatattgtttcccacagcttggatgaatgttttgccctttttgacagctattgaatttcattcagcttgggcaatgggaatgagagttaatcttcttgtggccaacacacataatgtcagcctaaagatgacaggcagtggtatctatgcaccacactcgcccaaagtatatcattatgatatggaaacagagctgggaaagatcctctttgcagaggtggattcgcatccccgaaactctctgacctacccaccagctgtgaattggagtgcctacgccaccaccatcaaaccattcccaggacagaaaaactcctttaggggctttatttcccgggatgagttcaacttcacagaactctttgaaaatgcaggaaaccttacagtctgtcaaaaagagctgtgctgtcatttaagctacagaatgttaggaaaagaggagaatgaagtgtatgttctaggagcttttgctggacttcatggccgaaggagaagagagtattggcaggtatgcacaatggtgagatgtaaatctactaatctgacaacttgtggacagccagtagacactgctctgacaagatttgaaatgttctccctaagtggcacatttagaacggagtatgttttccctgaagtgctacttactaaaatcaatctggcacctggaaaatttgaggtactaaaagatgggcgtttggtaaacaagaatggatcatctgagcctatcctaacagtgtcactgtttgggagatggtacactgaagacgtacgttccatctcaggcaggaccagcaactcagcgacaacttacctgataatatccacattattaatgatcatagctttgcaaaatattgtgatggtgtag

SEQ ID NO: 103 - DNA, 1566 bp, Sus scrofa
  source 1..1566: mol_type = genomic DNA
atgatcgcttcctcttttcaaactacgttagcagtttttgccctaattatcctgcatgtccgtgcactggatacttttaaggctgctgtatatgaacatgctgtcatcctgccaaatgacacaaaaacacctgtttcttgggatgatgctttgctcctcatgaacaagaatatggatattctggagagagcaattaagcaggcagctgagcagggttctcaaatcattgtgactccagaagatgcactttatggatggaaatttaccagggaaaccatttttccctatctggaggatgtcccagatcctcagaagaactggattccatgtcaagacccccgcagatttggtcacacaccagtgcaagtaagactcagctgcctggccaagaataactctatctatgtcttggcgaatattggagacaaaaagccctgtaattcccatgattccaaatgtcctcctaatggctattatcaatacaacaccaacgtggtatatgattcagagggaaagctggtggcacgttaccataagtaccacctctaccatgagcttcagtttgatgtccctgaaaagccggaggtggtgactttcaacaccccttttggaaagttcggcattttcacgtgcttcgatattctcttccgtgatcctgctgtgacgctggtgcaagatttccacgtggacaccatattgtttccggcagcttggatgaatgtgttgccccttttgacagctattgaattccattcggcttgggcaatgggcatgagagttaatcttcttgcagccaacgtacatcatgtccgcctaaatatgacaggcagtggtatctatgctccacattctcccaaagtataccattatgacatggaaacggactcgggaaaaatcctccttgcggaggtggatgcgcatccccgcacctccccgacctacccaacagccgtgaactggggtgcctacaccctaagcatccagccgttcccaatactgcaaaactccttcaggggattcatttcgcgggatgagttccacttcacagaactctctgaaagtgcaggaaaccttacggtctgtcataaagagctctgctgtcatttaagctacagaatgctagcgaaggaggagaatgaagtgtatgttctaggagcttttacaggacttcatggccgaaggaganaggagtattggcaggtatgcacaatgttgaaatgcaaatctactaatctgacaacttgcggacagcctgtagaaactgctctgacgagatttgagttgttctctctaagtggtacatttggaacagagtatgtttttcccgaagtgctccttactaaaatcaatctggcacctggaaaatttgaggtactgaaagatgggcgtttggtaaacaagaacggaacatctgagcctgtcctaggagtgtcactgtttgggagatggtacattagggatgtaccactttccatctcgggaggaccagcacctcagcaaaaaatgtacctgataatttccgcattattaatgatcatagctttgcaaaatattgtcatggtatag

SEQ ID NO: 104 - DNA, 1563 bp, Echinops telfairi
  source 1..1563: mol_type = genomic DNA
atgactgctgcttctttccaaacctccgcagcagtgtttgccctacttaccctgcatgtgggtgccctgaatactttcacagccgcggtgtacgaacatgccgtcatactgccaaataaaacactgacaccggtttctcagcaagatgccttggccctgatgaacaagaatatagatatcctggaggaagttattaagcaggcagctgagcagggcgctcaaatcattgtgactcctgaagatgctctctatggatgggaatttggatgggaaactgtcttcccttttttggaggatgtcccagcccctgaggtggactggattccatgccgagaccctcacagatttggtcacacaccagtacaaacaagactcagttgcatggccaagaacaactctatctatgtcgtggcaaatattggggacaaaaagaagtgtaactcccaggactccacctgcccttccactggctattttcagtacaataccaatgtggtgtacaacgcggaggggaaactggtggctcgttatcataagcaccacctctacggtgagtttcaatttaatgccactagaaagccagaagtggtgacattcgacaccacctttggaaagtttggtattttcacctgctttgatatactcttccgtgagcctgctgtgcgactggtggaagagtcccaagtcgacaccatattgttcccaacagcttggatgaatagcttaccctttctgacagctattgaatttcattcagcttgggcaatgcaaatgagagtgaatcttcttgctgctaacctacatcataccagtaaaaggatgacagggagtggcatctatggcccgaagggccccagcgtgtatcactatgatatggaaacggcatcgggacagctcctcctctcccaggtggactcccacccccggcgctcccctgactaccccgcagctgtcgactgggacgcctatgccaggagcatcaaaccattcccaacacagaagcatgcctttgggggcaacgttcacgctgaccagttcaacttcaccgagctgtatgaacgtgcaggaaaccttactgtctgccagaaagagctctgctgtcatttaagctacagaatgttaggaaaagaagagagtgaagtctatgttctaggagctttcacgggactccacgggaaaagggcgaaagagtactggcaggtctgcacgttgctgaaatgcaaaacgactatcttgacatcttgtggagaggtggtagaaactgcttctacaagatttgaaatgttctctctaagtggcaccttcgaaaccgagtatgtttatcccgaagtgttacttagtaaaattcagctgtcacctggaaaatttgaggtactaaaagatggacgtttggtagacaagaatgggacatctggacctattgtaaccgtgacactgtttggaaggtggtacgaaagggacagacttcagaattcagccaagaccagcaattcagcaatacacttcctactgatcgccacatcattcatgatcacagctttgcaaaatattgccatgacatag

SEQ ID NO: 105 - DNA, 1563 bp, Oryctolagus cuniculus
  source 1..1563: mol_type = genomic DNA
atgatcacttcctcttttccaacccgcgtggcagtttttgccctgatcagcctgcgtgtcggtgctctggacacttttgtagctgcagtgtatgaacatgccgtcgttttgccaaagaaaacagaaacgcctgtttctcaagacgatgccttgcttctcatgaacaagaatatagatattctggagagtgcgattaagcaggcagcagggaagaatgctcgaatcattgtgactccagaagatgcactttatgggtggaaatttaccagagaaacaattttcccttatctggaggatatcccagatcctcaggtgaactggatcccatgtcaagacccccacagatttggtcacacaccagtacaagcaagactcagctgcctggccaaaaacaattctatctatgtcttggcaaatattggagacaagaagccttgtaattcccatgactccacatgtcctcataatggctattatcaatacaataccaatgtggtgtacgattcagagggaaaactggtggcacgttatcataagtaccacctatactctgaacctcagtttgatgttcctgagaaaccagagctggtaactttcaacaccacatttggaaagtttggcattttcacatgttttgatatactcttccatgatcctgctgttaccttggtgaaagatttccatgtggacaccatattgctccctacagcttggatgaatgttctgcccctcttgacagctattgaattccattcagcttgggccatgggaatgagagttaatcttcttgtggccaatacacatcatgtcagcttaaatatgacagggagtggcatctatgtaccacattctcccaaagtgtaccactatgacatggaaacagacttgggaaagctcctcctttcagaggtggattcacatccccaaacctcttcttcctacccaacagctgtgaattggagtgcctatgccatggccatcagaccattctcagtacagaaagacactttcagaggatttatttctcgggacgagttcaattttacagaactttttgaaaatgcaggaaatattacaatttgtcaaaaagagctctgctgtcatttaagctacagagtgctagaaaaagaagcagatgaaatttatgtcctaggagcctttacaggatttcatggccgaaggaggagagaatactggcaggtatgcacaatgttgaaatgtaaaactactaatctgaccacatgtggacggccagtagagactgcttctacacgatttgaaatgttctccctcagtggcacatttggaacagagtatgtttttcctgaagttctactcactggaattcatctatcacctggaaaatttgaagtgctaaaagatgggcgtttggtaaataagaatggattatctgggcctatcctaacagtgtcactgtttgggaggtggtacacaaaggacacactttacaaatcatctgggaccaacaattcagcaataccttacctgctaatcttcattgtcctaatgactgcagattttcaaaatattgtattgctgtaa

SEQ ID NO: 106 - DNA, 1563 bp, Lepus europaeus
  source 1..1563: mol_type = genomic DNA
atgatcacttcctcttttccaacccatgtggcagtttttgccctgatcagcctgcgcgtcggtgctctggacacttttgtagctgcagtgtatgaacatgccgtcattttgccaaagaaaacagaaacgcctgtttctcaagacgatgccttgctcctcatgaacaagaatatagatattctggagagtgcgattaagcaggcagcagggaagaatgctcgaatcattgtgactccagaagatgcactttatgggtggaaatttaccagagaaacgattttcccatatctggaggatatcccagaccctcaggtgaactggatcccatgtcaagacccccacagatttggtcacacaccagtacaagcaagactcagctgcctggccaagaacaactctatctatgtcttggcaaatattggagacaagaagccttgtaattcccatgactccacatgtcctcataatggctattatcaatacaataccaatgtggtgtacgattcagagggaaaactggtggcacgttatcataagtaccacctatactctgagcctcagtttgatgttcctgagaaaccggagttggtaactttcaacaccacatttggaaagtttggcattttcacatgttttgatatactcttccatgatcctgctgttaccttggtgaaagatttccatgtggacaccatattgttccccacagcatggatgaatgttctgcccctcttgacagctattgaattccattcagcttgggccatgggaatgagagttaatcttcttgtggccaatacacatcatgtcagcctaaatatgacagggagtggcatctatgcaccacattctcccaaagtgtaccactatgacatggaaacagacttgggaaagctcctcctttcagaggtggattcacatcccaaaacctcttcttcctacccaacggctgtgaattggagtgcctatgccatggccatcagaccattctcagtacagaaagacactttcagaggatttatttctcgggacgagttcaattttacagaactttttgaaaatgcaggaaatattacaatttgtcaaaaagagctctgctgtcatctaagctacagaatactagaaaaagaagcagatgaaatttatgtcctaggagcctttacaggatttcatggccgaaggaggagagaatactggcaggtatgtactatgctgaagtgtaaaactactaatctgaccacatgtggacagccggtcgagactgcttctacacgatttgaaatgttctccctcagtggcacatttggaacagagtatgtttttcctgaaattctactcactgaaattcatctatcacctggaaaatttgaagtgctaaaagatgggcgtttggtaaataagaatggattatctgggcctatcctaacagtgtcactgttcggaaggtggtacacaaaggacacactttacaaatcatctgggaccagcaattcagcaataccttacctgctaatcttcattgtcctaatgattgcagatttgcaaaatattgtattgctgtaa

SEQ ID NO: 107 - DNA, 1548 bp, Dasypus novemcinctus
  source 1..1548: mol_type = genomic DNA
atgatcacgtcaccttttctaccttcggcagtagttttggccctaatcattctgaatgtcggtgcactgggcacttttaaagctgccgtgtacgagcatgctgtgatattgccaaaccaaacactcagagttgtttctcaggaagatgcgctgctcttgatgaacaagaatctagatatcctggaggcagagataaagcaggcagctcagcagggtgctcaaatcattgtgactccagaagatgccctttatggttggactttcaccagggagactgttttcccttatctggaggacatcccagacccccaggagaactggattccatgtcagaacccccagagatttggtcacacaccaatacaaaaaagactcagctgcctggccaaggacaactctatatatatcctggcaaatattggggacaaaaagcagtgcgattcccggaactccgcctgtcctcgcaatggttattatcaatacaataccaacgttgtgtatgataaagagggaaaactggtggcgcgttaccataagtaccatctctacaatgagcctcagtttaacgcccctaaaaggccggaattggtgaccttcaacactgattttggaaagtttggcattttcacgtgctttgatatactctttaaagagcctgcagtgaccctggtggaagaattacaagtggacaccatactgtttgccacagcttggatgaatgtcttgccctttttgacagctattgaattccactcggcttgggcaatgggaatgagagtcaatgttcttgcagccaacatacacaatcccagcctaaatatgacagggagtggtatctactcaccacattcccccaaagcataccattatgatatgaaaacaaagtttggaaggctccttctttcagagttggattcacatccccgaggttctgtatcttacccaccagctgtgagctggacaacctacgccaagtccatcaaaccattcccagtacagaaacccgccttcctgagaagtatttccagagacgagttcaacttcacggaactttttgaaagtgcaggaaatcttacagtctgtcaaaaagagctctgctgtcatttaagctacagaatgttagaaaaagacacaaaagaatcctacgttctcggagcatttacaggacttcacggcagaaggcaacaagagtactgggaggtatgtacaatactgaaatgtgaagctaataatttcatgacttgtggacggacaggggaaactgcttctacaagatttgaaatgttctccctaagtggcacatttaaaacagagtatgtctttcctgaagtgttacttgataaaattcacctgtcacccggaaaatttgaggtactaaaggatggccgtttggtaaacaagaatgggacatctgggcagatcctaacagtgtcactgtttggcaggtggtatgccaaggatgagctttccaactcaggcaagacccacagttcagcagtgccaacgctggcattagtaatgatagtagctttgatgtatgatgtgtga

SEQ ID NO: 108 - DNA, 1404 bp, Camelus dromedarius
  source 1..1404: mol_type = genomic DNA
atgaacaagaatatggatattctggagaaagcaattaagcaggcagctgagcagggtgctcaaattattgtgactcctgaagatgccatttatggctggaaatttacccgggaaaccattttcccttatcttgagtatatcccagaccctcaggtgaactggattccatgtcaacaaccccacagatttggtcacacaccagtacaagtaagactcagctgcctggccaagaacaactctatctatgtcttggcgaatattggggacaaaaagccatgtaattccaggggctccacgtgccctcctagtggctattaccaatacaacaccaacgtagtgtatgatacacagggaaagctggtggcacgttaccataagtaccacctctactctgagcctcagtttgatgtccctgaaaagccagaggtggtgactttcaacaccaccttcggaaggtttggcattttcacgtgcttcgatatactcttccatgatcctgctgtgaccctggtgaaagatttccaggtggacaccgtattgtttcccacagcgtggatgaatgttttgccccttttgacagctattgaattccattcagcctgggctatgggcatgggagttaatcttttggtggccaacacacatcacgtcagcctaagaatgacaggcagcggtatctatgcaccacactctcccaaggtctattattatgataaggaaacagagttgggaaagcttctccttgcagaggtggactcgcatccccgaatctctccgacctacccaacagccgtgaattggagcgcctacgccacaaccatcaaaccattcccagtacagaagcacactttcgggggatttatttcccgggatgagttcaacttcacagagctctttgaaaatgcaggaaacctgacagtctgtcaaaaagagctctgctgtcatttaagctacagaatgctgggaaaagaggagaatgaagtgtatgtgctaggagcttttacaggacttcatggccgaaggagaagtgaatattggcaggtatgcaccctggtgaaatgcaaatctactaacttgacgacttgtggacagccagtagaaactgctctgacaagatttgaaatgttctccctaagtggcacatttggaacagagtatgtttttcctgaagtgctacttactaatatccatctggcacctggaaaatttgaggttctaaatgatgggcgtttggtaaacaagaatggattatctgagcctatcctaacagtgtcactgtttgggaggtggtacactaaagacgcactctccatctcaggtgggaccagcaacgcagcaacaacttacctgttaatattcacatcattaatgatcatagctttacaaaatactgtgatggtataa

SEQ ID NO: 109 - DNA, 1380 bp, Homo sapiens
  source 1..1380: mol_type = genomic DNA
atggtcacttcctcttttccaatctctgtggcagtttttgccctaataaccctgcaggttggtactcaggacagttttatagctgcagtgtatgaacatgctgtcattttgccaaataaaacagaaacaccagtttctcaggaggatgccttgaatctcatgaacgagaatatagacattctggagacagcgatcaagcaggcagctgagcagggtgctcgaatcattgtgactccagaagatgcactttatggatggaaatttaccagggaaactgttttcccttatctggaggatatcccagaccctcaggtgaactggattccgtgtcaagacccccacagatttggtcacacaccagtacaagcaagactcagctgcctggccaaggacaactctatctatgtcttggcaaatttgggggacaaaaagccatgtaattcccgtgactccacatgtcctcctaatggctactttcaatacaataccaatgtggtgtataatacagaaggaaaactcgtggcacgttaccataagtaccacctgtactctgagcctcagtttaatgtccctgaaaagccggagttggtgactttcaacaccgcatttggaaggtttggcattttcacgtgctttgatatattcttctatgatcctggtgttaccctggtgaaagatttccatgtggacaccatactgtttcccacagcttggatgaacgttttgccccttttgacagctattgaattccattcagcttgggcaatgggaatgggagttaatcttcttgtggccaacacacatcatgtcagcctaaatatgacaggaagtggtatttatgcaccaaatggtcccaaagtgtatcattatgacatgaagacagagttgggaaaacttctcctttcagaggtggattcacatcccctatcctcgcttgcctacccaacagctgttaattggaatgcctacgccaccaccatcaaaccatttccagtacagaaaaacactttcaggggatttatttccagggatgggttcaacttcacagaactttttgaaaatgcaggaaaccttacagtctgtcaaaaggagctttgctgtcatttaagctacagaatgttacaaaaagaagagaatgaagtatacgttctaggagcttttacaggattacatggccgaaggagaagagagtactggcaggtctgcacaatgctgaagtgcaaaactactaatttgacaacttgtggacggccagtagaaactgcttctacaagatttgaaatgttctccctcagtggcacatttggaacagagtatgtttttcctgaagtgctacttaccgaaattcatctgtcacctggaaaatttgagcaaacctga

SEQ ID NO: 110 - DNA, 1560 bp, Globicephala melas
  source 1..1560: mol_type = genomic DNA
atgatcatttcctcttttcaaacctccgtggcagtttttgcgctaattattctgcatgtcagtgctttggctactttcaaagctgcagtatatgaacatgctgtcatattgccaaatgacacagaaacacccgtttctcaggattatgccttgctcctcatgaacaggaatatagatattctggagagagcaattaagcaggctgctgaacagggtgctcagatcattgtgactccggaagatgcaatttacggatggaaatttaccagtgaagccattttcccttatctggaggatattccagatcctctggtgaattggattccatgtcaagacccacacagatttggtcatacaccagtacaagcaagactcagctgcctggccaaggacaactctatctatgtcgtggcaaatatcggggacaaaaagccatgtcattcccgtgactccgcgtgtcctcctaatggccattatcaatacaacaccaacgtggtgtatgatgcaaatggaaagctggtggcacgttaccataagtacaacctctactctgagcttcagtttgatgtccccgaaaagccagagctggtgactttcaacacctcctttgggaagtttggcattttcacatgctttgatatcctcttccatgatcctgctgtgaccctggtgaaagatttccacgtggacactatattgtttcccacagcttggatgaatgttttgccctttttgacagctattgagtttcattcagcttgggcaatgggaatgagagttaatcttcttgtggccaacacacataatgtccgcctaaaaatgacaggcagtggtatctatgcaccacactctcccaaagcgtaccattatgatatggaaacagagttgggaaagatcctctttgcagaggtggattcgtatccccgaaactctccaacctacccaccagctgtgaattggagtgcctacgccaccaccatcaaaccattcccaggacagaaaaattcctttaggggctttatttcctgggatgagttcaacttcacagaactctttgaaaatgcaggaaaccttacagtctgtcaaaaagagctgtgctgtcatttaagctacagaatattaggaaaagagaatgaagtgtatgttctaggagcttttgctggacttcatggcagaaggagaagagagtattggcaggtatgcacaatggtgaaatgtaaatctactaatctggcaacttgtggacagccagtagacactgctctgacaagatttgaaatgttctccctaagtggcacatttagaacggagtatgtttttcctgaagtgctacttactaaaatcaatctggcacctggaaaatttgaggtactaaaagatgggcgtttggtaaacaagaatggatcatctgagcctatcctaacagtgtcactgtttggcagatggtacactaaagacgtacgttccatctcaggcgggaccagcaactcagcaacgacttacctgataatatccacattattaatgatcatagctttgcaaaatattgtgatggtgtag

SEQ ID NO: 111 - DNA, 1542 bp, Pteronotus mesoamericanus
  source 1..1542: mol_type = genomic DNA
atgcaatttcatttctgggagagaggccaggctaggacgtccttcaggctgtggcagagcccaaggctcctcctcaccatcctgagttcctcggtgtctttgtcatgtctccattgcgtggttgacgtgaatctgctgtgtccttctcggcaagatccaggagttggtgcccaggagaaccttgtgaaggatataaagcagggtgctcaaattattgtgactccagaagacgcaatttatggatggaattttaccagggaaaccattttcccttacctggaggatatcccagaccctcaggtggactggattccatgtcaagacccccacagatttggtcacacgccagtacaagaaagactcagctgcctggccaagaacaactctatctatgttttggcaaatattggagacaaaaagccatgtaattctaatgacttcacatgtcctcctaatggctattatcaatacaatactaatgtggtatatgataaaaatggacacctggtggcacgttaccacaagtaccacctctattctgaacctcagtttgatgtgcctgaaaagccggagctggtgactttcaacaccacctttggaaggttgggcattttcacctgcttcgatatactcttccacgatcctgctgtgaccctggtgaaggatctccacgtggacaccatactgtttcccacagcttggatgaatgtcttgccccttttaacagctattgaattccattcagcttgggcaatgggaatgagagtcaatcttcttgtggccaatacacatcatgtccgcctaaatatgacaggcagtggcatttatgcaccacagtctcccaaagtataccactacgatatggacacggagctgggaaagatccttctttcagaagtggattcgcatcctcgaagctcacctgcctacccagcggctgtgaactggagtgactatgccacgaccatcaaaccattcccagtacagaaaaacactttcaggggatttatttcccgggatgcgttcaacttcacagaactttctgcaattgcaggaaaccttacagtctgtcaaaaagatctctgctgtcatttaagctatggaatgttaagaaaagaagagaatgaagtatacgctctaggagctttctcaggacttcatggccgaaggagaagagagtattggcaggtatgcacaatgctgaaatgcaaaacaactaatttgacaacttgtggacaaccagtagaaactgctgtgacaagatttaatatattctccctaagtggcacatttggaacagagtatgtttttcctgaagtgctacttactaaaattcatctgtcacctggaaaatttgaggtactaaaagatgggcgcttgataaacaagaatggaacatctgagcctatcctaacagtgacactctttgggaggtggtacgctaaggactcacattccgtctcaggagggatcagcaatttggaaacaacttacctgttaatatccactttatcaatgatcatagctttacaaatattgtga

SEQ ID NO: 112 - DNA, 1554 bp, Equus caballus
  source 1..1554: mol_type = genomic DNA
atgattgcttcctcttttcgaacctctgcggctgtttttgccgtaattatcctgcatgttggggccctggatactttcatagctgcagtttatgaacacgccgtcatactgccaaaaacaacagtgacacctgtctctcaggacgatgccttgctcctcatgaacaagaatatagatattctggagagagcgattaagcaggcagcaaagcagggtgctcaaatcattgtgactccagaagatgccctttatggttggacatttaccagggaaaccattttcccttatctggaggacgtcccggaccctcaggcgaactggattccgtgtcaggactccaacagatttggttacacaccagtacaagtaagactcagctgcctggccaagaacaactcaatctatgtcttggcaaatatgggggacaagaagccatgtaatcgccataactccatgtgtcctcccaatggctattatcaatacaacaccaatgtggtatatgatgcggaggggaaactggtagcacgctaccataagtaccacctctactctgagcctcagtttgatgtccctgaaaagcccgagttggtgactttcaacaccacctttggaaggtttggcattttcacgtgctttgatattttcttccacgatcctgctgtcaccctggtgaaagatttccatgtggataccatcctgtttcccacagcttggatgaatgtcttgccccttctgacagctattgaattccactcggcttgggctgtgggcatgagagttaatcttcttgcggccaacacccacaatatccacatgaatatgacaggcagcggtatctatgcaccacagtctcccaaagtataccaccatgacatggagacagagtcgggaaagctcctccttgcggaagtggattcgcatccccgagactctcctgcctacccaacagctgtgaactggagtgcccatgccacaaccatcagaccattcccaggaaagaaaaacaccttccgggcatttatttcccgggacgctttcaacttcacagaactttttgaaaatgcaggaaaccttacagtctgtcaaaaagatctctgctgtcatttaagctacaggatgttaggaaaggaagaggatgaagtgtatgctctgggagctttttcaggacttcacggccgaaggagaaaagagtactggcaggtgtgcacaatgctgaaatgcaaaactactaatttgacaacttgtgggcggccggtagaaactgcttctacaagatttgaaatgttctccttaagtggcacatttggaacagagtatgtcattccagaagtgctacttactaaaattcacctgtcacctggaaaatttgaggtactacaagatgggcgtttggtgaacaagaacggaccatccgagcctatcctgacagtgtctctctttgggagatggtatgctaaggattcatcttccatcccaggcggaaccagcaattcagcaacaacttacctgctgatacccattttattgatgatcatagcttggcagatactgtga

SEQ ID NO: 113 - DNA, 1332 bp, Jaculus jaculus
  source 1..1332: mol_type = genomic DNA
atgggcgctcagatcattgtgactccagaggatgcactttatggatggaagtttactagggaaagcattttcccttacttggaagatgtccccgaccctcaggtggactggattccatgtcgagatccccacagatttggtcacacacccatacaagaaagactcagctgcctggccaagaacaactctatctatgtcgtagcaaatattggagacagaaagccatgcaagtcccatggatgccctcataatggctactatcagtacaatactgatgtggtatacgataaagagggaaaactcgtggcacgctatcataagtaccacctctactcagagattcagtttgatatccccgaaaaaccagagttggtgactttcaacaccacatttggaaggtttggcattttcacgtgctttgatatcctcttccatgaccccgccgtgaccctagtgagagatttccacgtggacaccatactgtttcccacagcatggatgaatgttttgccccttttgacagctattgaattccattcagcatgggcaatgggcatgggagttaatcttcttgcagctaacctacatcatgtcaggctaaatatgacagggagtggtatctatgcaccacactctccaaaagtgtaccactatgacatggaaacagacttgggaaagatgctcttggcagaactcgattcccatccccggtcttctcctagctacccagtagcagtgaattggagtacctacgccgtggctcaagagccattcccgctacagagaaaagcttttggcggatttatttcccgggatgagttcaatttcacagaactttttgaagatgcaggaaatcttacagtctgccaaaaagaattatgttgtcacttaagctacagaatgttaggaaaagaagagcatgaagtgtatgttgtgggagctttcacgggacttcatggccgaaggagaagagagtactggcaggtatgcacaatgctgaagtgtaaatcggctagtctcaagacttgtggacaaccggtagaaaccgcttccacgagatttgatatgttttccctcagtggtacattcggaacagagtatgtttttcctgaagtgctcctttctaaagtgcatctgtcccctggaaatttcgaggtgctggaagatgggcgtttgacaaacaagaatgggacacctggaccgatcttaaccgtgtctctgtttgggaggtggtacacgaaggattccctttacagctcatgtatgaccaacaatcccatgttaatttacctgctcatttccacactattaatgatgtttaaaatattgtaa

SEQ ID NO: 114 - protein, 520 aa, Bos taurus
MITSSLQTSVAVFALVILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLSCLAKNNSIYVVANMGDKKPCNSHNTTCPSNGHYQYNTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAGNLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYTKDAHSISGGTSNSATAYLMIPTLLMIITLRDAVMV

SEQ ID NO: 115 - protein, 520 aa, Bos javanicus
MITSSLQTSGAVFALIILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLSCLAKNNSIYVVANMGDKKPCNSRNTTCPSNGHYQYNTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAANLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYTKDAHSISGGTSNSATAYLMIPTLLMIITLRDAVMV

SEQ ID NO: 116 - protein, 520 aa, Moschus berezovskii
MITSSLQTSVAVFALIILHVSTLDTFKAAVYEHAVILPNDTKTPVSPDEALFLMNKNIDILEKAIKQAAEQGAQIIVTPEDALYGWKFTRETIFPYLENIPDPQVNWIPCQDPHRFGQTPVQARLSCLAKNNSIYVVANMGDKKPCSSRNTTCPSNGHYQYNTNVVYDAKGKLVARYHKYHLYSELQFDVPEKPELVTFNTTFGKFGIFTCFDILFHDPAVTLVKDFHVDTILFPTAWMNVLPLLTAIEFHSAWAMGMRVNLLAANTHNVSLKMTGSGIYAPHSPKVYHHDMETESGKILFAEVDSHPRNSSTYPPAVNWSAYATSITPFPGAKNSFRGFISRDEFNFTKLSESAGNLTVCQKEICCHLSYRMLGKEGNEVYVLGAFAGLHGRRRREYWQVCTMLKCRSTNLTTCGQPVETALTRFEMFSLSGTFGTEYVFPEVLLTEIKLAPEKFEVLEDGRLVNKNGSSEPVLTVSLFGRWYTKDTHSISGGTSNSATMHLMIPTLLMIITLQYTVMV

SEQ ID NO: 117 - protein, 538 aa, Orcinus orca
MIISSFQTSVAVFALIILHVSALATFKAAVYEHAVILPNDTETPVSQDYALLLMNRNIDILERAIKQAAEQGAQIIVTPEDAIYGWKFTREAIFPYLEDIPDPLVNWIPCQDPHRFGHTPVQARLSCLAKDNSIYVVANIGDKKPCHSRDSTCPPNGHYQYNTNVVYDANGKLVARYHKYNLYSELQFDVPEKPELVTFNTSFGKFGIFTCFDILFHDPAVTLVKDFHVDTVLFPTAWMNVLPFLTAIEFHSAWAMGMRVNLLVANTHNVCLKMTGSGIYAPHSPKAYHYDMETELGKILFAEVDSYPRNSPTYPPAVNWSAYATTIKPFPGQKNSFRGFISRDEFNFTELFENAGNLTVCQKELCCHLSYRMLGKENEVYVLGAFAGLHGRRRREYWQVCTMVKCKSTNLATCGQPVDTALTRFEMFSLSGTFRTEYVFPEVLLTKINLAPGKFEVLKDGRLVNKNGSSEPILTVSLFGRWYTKDVRSISGSANRGRWRETEDWTRSGETSVCSPISVVPSSVPYLTAVVCSSSTWF

SEQ ID NO: 118 - protein, 520 aa, Mesoplodon densirostris
MIISSFQTSVAVFALIILHVSALDTFKAAVYEHAVILPNHTETHVSQDDALHLMNKNIDILERAIKQAAEQGAQIIVTPEDAIYGWKFTRETIFPYLEDIPDPLVNWIPCQDPHRFGHTPVQRRLSCLAKNNSIYVLANIGDKKPCNSYDSACPPNGHYQYNTNVVYDAKGKLVARYHKYHLYSELQFDVPEKPELVTFNTSFGKLGIFTCFDILFHDPAVTLVKDFHVDTILFPTAWMNVLPFLTAIEFHSAWAMGMRVNLLVANTHNVSLKMTGSGIYAPHSPKAYHYDMETELGKILFAEVDSYPRNSPTYPPAVNWSAYATTVKPFPGQKNSFRGFISRDEFNFTELFENAGNLTVCRNELCCHLSYRMLGKEENEVYVLGAFAGLHGRRRREYWQVCTMVKCKSTNLTTCGQPVDTALTRYEMFSLSGTFRTEYVFPEVLLTKINLAPGKFEVLKDGRLVNKNGSSEPILTVSLFGRWYTKDVRSISGGTSNSATTYLIIYTLLMIIALQNTVMV

SEQ ID NO: 119 - protein, 519 aa, Globicephala melas
MIISSFQTSVAVFALIILHVSALATFKAAVYEHAVILPNDTETPVSQDYALLLMNRNIDILERAIKQAAEQGAQIIVTPEDAIYGWKFTSEAIFPYLEDIPDPLVNWIPCQDPHRFGHTPVQARLSCLAKDNSIYVVANIGDKKPCHSRDSACPPNGHYQYNTNVVYDANGKLVARYHKYNLYSELQFDVPEKPELVTFNTSFGKFGIFTCFDILFHDPAVTLVKDFHVDTILFPTAWMNVLPFLTAIEFHSAWAMGMRVNLLVANTHNVRLKMTGSGIYAPHSPKAYHYDMETELGKILFAEVDSYPRNSPTYPPAVNWSAYATTIKPFPGQKNSFRGFISWDEFNFTELFENAGNLTVCQKELCCHLSYRILGKENEVYVLGAFAGLHGRRRREYWQVCTMVKCKSTNLATCGQPVDTALTRFEMFSLSGTFRTEYVFPEVLLTKINLAPGKFEVLKDGRLVNKNGSSEPILTVSLFGRWYTKDVRSISGGTSNSATTYLIISTLLMIIALQNIVMV

SEQ ID NO: 120 - protein, 520 aa, Sus scrofa
MIASSFQTTLAVFALIILHVRALDTFKAAVYEHAVILPNDTKTPVSWDDALLLMNKNMDILERAIKQAAEQGSQIIVTPEDALYGWKFTRETIFPYLEDVPDPQKNWIPCQDPRRFGHTPVQVRLSCLAKNNSIYVLANIGDKKPCNSHDSKCPPNGYYQYNTNVVYDSEGKLVARYHKYHLYHELQFDVPEKPEVVTFNTPFGKFGIFTCFDILFRDPAVTLVQDFHVDTILFPAAWMNVLPLLTAIEFHSAWAMGMRVNLLAANVHHVRLNMTGSGIYAPHSPKVYHYDMETDSGKILLAEVDAHPRTSPTYPTAVNWGAYTLSIQPFPILQNSFRGFISRDEFHFTELSESAGNLTVCHKELCCHLSYRMLAKEENEVYVLGAFTGLHGRRRXEYWQVCTMLKCKSTNLTTCGQPVETALTRFELFSLSGTFGTEYVFPEVLLTKINLAPGKFEVLKDGRLVNKNGTSEPVLGVSLFGRWYIRDVPLISGGPAPQQKMYLIISALLMIIALQNIVMV

SEQ ID NO: 121 - protein, 504 aa, Phyllostomus discolor
MPFHFQEGGQTRTSFRLWRSQRLLLTILSSSVSLSCRHHVADVHLLCPAWQDPGVGAQGAEIIVTPEDALYGWSFTRETIFPYLEDIPDPRVNWIPCQDPHKFGHTPVQVRLSCLAKDNSIYVLANIGDKKPCKSNDPTCPSNGYYQYNTNVVYDKKGQLVARYHKYHLYSEPQFDVPEKPELVTFNTNFGKFGIFTCFDILFHDPAVKLVKDFRVDTILLPTAWMNVLPLLTAIEFHSAWAMGMRVNLLVANTHHVRLNMTGSGIYAPHSPKAYHYDMDTELGKILFSKVDSHPRSSPDFPAAVNWADYATTIKPFPVQKGTFKGFISRDAFNFTELSASAGDLTVCQKDLCCHLSYEMSRKEENEVYALGAFSGLHGRRGREYWQVCTMLKCRTTDVTTCGQPVETALTRFNVFSLSGTFGTEYVFPEVLLTKIRLSPGKFEVLKDGRLVNKNGMSEPILTVSLFGRWYAKDAHSASGGIGNLETTNLLISTLLMITILQIL

SEQ ID NO: 122 - protein, 520 aa, Homo sapiens
MVTSSFPISVAVFALITLQVGTQDSFIAAVYEHAVILPNKTETPVSQEDALNLMNENIDILETAIKQAAEQGARIIVTPEDALYGWKFTRETVFPYLEDIPDPQVNWIPCQDPHRFGHTPVQARLSCLAKDNSIYVLANLGDKKPCNSRDSTCPPNGYFQYNTNVVYNTEGKLVARYHKYHLYSEPQFNVPEKPELVTFNTAFGRFGIFTCFDIFFYDPGVTLVKDFHVDTILFPTAWMNVLPLLTAIEFHSAWAMGMGVNLLVANTHHVSLNMTGSGIYAPNGPKVYHYDMKTELGKLLLSEVDSHPLSSLAYPTAVNWNAYATTIKPFPVQKNTFRGFISRDGFNFTELFENAGNLTVCQKELCCHLSYRMLQKEENEVYVLGAFTGLHGRRRREYWQVCTMLKCKTTNLTTCGRPVETASTRFEMFSLSGTFGTEYVFPEVLLTEIHLSPGKFEVLKDGRLVNKNGSSGPILTVSLFGRWYTKDSLYSSCGTSNSAITYLLIFILLMIIALQNIVML

SEQ ID NO: 123 - protein, 520 aa, Ochotona princeps
MITSSFLTCVAIFALITLHVGALDTFTAAVYEHAVILPKETETPISQDDALLLMNKNMNVLESAVKEAAKMNAQIIVTPEDALYGWRFTREIIFPYLEEIPDPQVNWIPCQDPHRFGHTPVQTRLSCLAKNNSIYVLANIGDKKPCNSQSSTCPRNGYYQYNTNVVYDSEGKLVARYHKYHLYSEPQFDVLEKPELITFNTTFGRFSIFTCFDILSHDPAVTLVKEFHVDTVLFPSAWMNVLPPLTAVGFHSAWAMGMRVNLLVANIHHVSQNMTGSGIYTPDSPKAYYYDTKRESGHLLVSELDSHPRNSPSYPTAVNWSAYAVTIAPFPVQKNTFIGFISQDGFNFTELLENDGNITVCHKELCCHLSYRMLEKEKDXVYVLGAFTGLHGRRRREYCQVCTMLKCQTTNLTTCGKPVETASTRFEMFSLSGTFAAEYVFPEVLYSEIRLSPGKFEVLKDGRLVNKNGVSEPILTGSLFGRWYAKDLPHKSCGNNNSTIFYMPVFTILMIAYLQNIVMV

SEQ ID NO: 124 - protein, 497 aa, Alligator mississippiensis
MMSLQPYLYAMISTLSVLQVFASDTYTAAVYEHAVILPDVTDKPVSLDEALVLMNRNIDVLEVAIKEAAQQGAHIIVTPEDSVYGWNFTREALYPYLEDIPDPQVNWIPCTDPQRFAPAPVQERLSCLARNNSIYVVANMGDKKPCNCSDPKCPRDGHYQYNTNVVFDSQGKLVARYHKYNLFRSETQFDYPKEPEMVTFNTSFGNFGIFTCFDILFHDPAVVLVKKLHVDTVLFPTAWMNVLPFLSAIEIHSAWAMGIGVNLLASNTHNTLMAMTGSGIYAPDGPRAYHYDMETENGHLLTTQLSSHPHLSPTYPAAVNWSSYATSIKQFHVDKNTFMGAVRSDNFTFTELITIAGNHTVCQKDLCCHLIYRMVEKREDEVYVLGAFDGLHGSVIKYYWQICTLLKCKTMDLKTCGQPVTTTQTRFEAFSLSGTFGTKYVFPEVLLSGVRLAPGEFQVLNDGCLKNNNGPAKLILTVTLFGRYYEKDPPHPLRNSP

SEQ ID NO: 125 - protein, 497 aa, Ceuthmochares aereus
MLPSQNLLHTAVLALTVLQALASDTFIAAVYEHSVILPNATDKPVSPNDALMLMNKNMDVLERAIKEAALQGAHIIVTPEDGIYGWRFTRETIYPYLEDIPDPAVNWIPCINPTRFSPAPVQERLSCIARNNSIYVVANTGDKKPCNSSNHDCPSDGRYQYNTDVVFDPEGKLVARYHKYNLFRGETQFNYPKEPEIVTVETPFGKFGIITCFDILFREPATVLVSKLQVDTVLFPTAWMNVLPFLTAVEFHSAWAMGMGVNLLSANTHNINASMTGDGLFTPEGPAAYRYDSKTEEGRLLLAELSSRPRLSPTYPPSVNWSLYATSIKKFPEEKDTFAKAVQRDIFTFSELGYTAGNRTVCQGDLCCHLVYQMSGKSKDEVYVLGAFDGLGGSLIKYHWQICTLLKCKSTDLNTCGQPVEMAQTKFEMFSLSGTFGTNYVFPEVLYSEVQLAPGEFEVLHDGRLKSKHGTSKPLLTATLFGRLYEKDPPHPLRTSV

SEQ ID NO: 126 - protein, 518 aa, Felis catus
MITPRLLAYVAISVSCVLKVSSLDTFIAAVYEHAVILPNTTLIPVSREEALALMNRNLDLLEGAITSAAKQGAHIIVTPEDGIYGWNFNRETIYPYLEDIPDPRVNWIPCNYPNRFGHTPVQERLSCLAKHNSIYIVANIGDKKPCNASDPQCPPDGRYQYNTDVVFDSQGKLVARYHKYNLFMGEDQFNVPKKPEVVTFDTIFGRFGIFTCFDILFHDPAVTLVKDFHVDTIVFPTAWMNVLPHLSAIEFHSAWAVGMGVNFLAANIHHPHKRMTGSGIYAPDSPRAFHYDMKTKEGKLLISRLNSSSYDPGVVNWASYASSIKAFSTGNQEFKSTAFFDEFTFLELTRVTGNYTVCQKELCCHLSYKMSEKKPDEVYALGAFDGLHIAEGSYYLQICTLLKCKTPNLRTCGDAVESASTRFEMFSLSGTFGTQYVFPEVLLSDSQLAPGEFQVSSDGRLFSVKPPSGPVLTVTLFGRLYEKDWILKASSDLRAQAPGVMLVVIALSVCSFSWSPIG

SEQ ID NO: 127 - protein, 513 aa, Phodopus roborovskii
MSVSRWLACAVLSVFYVLQVSSLDTFVAAVYEHAVIMPSDTPSPLSHSEALAIMNRNLDLMEGAIVSAAKQGAHIIVTPEDGIYGVHFTRDTIYPYLEDIPDPQVNWIPCDNANRFGYTPVQERLSCLAKNNSIYVVANMGDKKPCNTSDPQCPPDGRFQYNTNVVFDSLGKLVARYHKQNLFMGEEQFNAPTEPELVTFNTPFGRFGIFTCFDILFHDPAVTLVTKFQVDTILFPTAWMDVLPHLAAIEFHSAWAMGMGVNFLAANIHSPLRRMTGSGIYAPDSPRAFHYDRKTNEGKLLLAQLDSHPSSSSVNWTSYASSVETPSTGDQEFQSVVFFDEFTFVELKGITGNYTVCQNDLCCHLSYQMSEKRADEVYALGAFDGLHTVEGQYHLQICTLLKCKTTNLRTCGSSVDTAATRFEMFSLSGTFGTQYVFPEVLLSQVKLAPGEFQVSSDGRLFSLKPTSGPVLTVTLFGRLYEKDWVSSTSSDLLTHSLMIIIMIIITPIIHYLC

SEQ ID NO: 128 - protein, 553 aa, Moschus berezovskii
MIMSQLLNYVAFLFFCVSRADSLDTFIAAVYEHPVILPNATLVPVSPEEALAVMNRNLDLLEGAVTSASKQGAHIIVTPEDGIYGFNFTRESIYPYLEDIPDPQENWIPCNNPNRFGHTPVQQRLSCLAKDNSIYIVANIGDKKSCNASDPQCPPDGRYQYNTDVVFDSKGKLVARYHKQNLFLSEDQFNAPKEPEVVTFNTTFGKFGIFTCFDILFHDPAVTLVRDSHVDTILFPTAWMNVLPHLSAIEFHSAWAMGMRVNFLAANLHYPLKKMTGSGIYAPDSPRAFHYDMKTEEGKILLAQLDSHPHPTPVVNWTSYASGVEVHSMGNQEFTDTIFFDEFTFLELKGIGGNYTVCQRDLCCHLSYKMSEKRADEVYALGAFDGLHTVEGSYYLQICTLLKCKTTDLHTCGDSVETASTRFEMFSLSGTFGTQYVFPEVLLSEIQLAPGEFQVSSDGRLFSLKPTSGPVLTVTLFGRLYEKDSAPNALSDLTTQALRIRRLVSIHNYRSECCIIPWRRKWQPTPVFLPGEFPWTEEPGGLQYMGSRRVGHD

SEQ ID NO: 129 - protein, 491 aa, Amazona guildingii
MLPSQPLLHTAVFALTVLQALASNTFIAAVYEHAVILPKAAIKPVSPDDALALMNSNMDILEGAIKEAAQQGAHIIVTPEDGIYGWRFTRETIYPYLEDIPDPVVNWIPCTDPTSIYLELKEVPFRNPAAYTVCCITQGWSEWCQQRTSRGLKFVHSPPLPXVVYDPEGKLVARYHKYNLFKGETQFNYPKEPEATTFETPFGKFGIFTCFDILFHEPAVVLVSELQVDTVLFPTAWMNVLPFLTAVEFHSAWAIGMGVNLLSANTHNINFAMTGSGLFTPEGPVAYHYDSETDEGRLLVAELSAQPRLSPNYPPAINWSLYATSIKKFPEEDTFLGAVRKDIFTFSELRHKAGNYTVCQRDLCCHLIYQMSNKSKDEVYVLGAFNGLHGSFIKYYWQICTLLKCKSTDPETCGHPVETAQTKFEMFSLSGTFGTNYVFPEVLYSGVQLAPGEFEVLSDGRLKSKHGTSKPLITATLFGRLYEKDLPHPLR

SEQ ID NO: 130 - protein, 520 aa, Patella sp.
MKKNLDIYAEQTRIASSQNVDIIVFPEDGIYGLNFNRDTIKPYLEDIPDPTEITWNPCNQSEYTNGVEVQITLSCMARNNGIYIVANMGDKKVCDADSICPDDGQRQYNTDVVFDPQGNLVARYHKQHLYFEAQFDTPSTVERVTFDTPFGKFGIITCFDILFKSPAVDLVEDLGVKNVIFPTAWMDSLPLLSAIQFHSAFAKLFKVNLLAANLHFPLFNFHGSGIYSPRRTEAYYYNDGLFSPGKLLIADVKSSPTHYRRHQVVPDTHGFRGGNSLGASFVHGQDTAEVNTQSRRNGGHYNTASRNGIISHQLADQASGVHLNNDLGPLTHRSIRNRIHNHAHLHRVNRHMVENGWATIMADNDNYQYLPLTEPKAKVKICQNEVCCYLDYQKQNTSNDELYALGVFDGLHQIEGLSWYTQVCVLLKCEFTDIASCGQPTKIAAGHFEYLKLWGTFTTEYVFPEVLVSDDGKLRLEHEHWKYLHNRIEFVNRGKESPLLSATLYGRHYDRDNSTVSNSV

SEQ ID NO: 131 - protein, 657 aa, Dreissena polymorpha
MFLELRLNLSIDRKNNIMEQNCLTKYFKTLLVCSLFCLTQCLKTDIKKFFTAAVYEHAVYQPPVTPLPLPRAQALEIMMTNIRVYQEQTKIATSKGAHIIVFPEYGVYGLSYKTRELIFPFMENIPEPERNWNPCEHPDVYPETEIQFALSCMARENNIYVVANIGDKQPCNVSLDPSCPRTGYYQYNTNVAYDAMGTLVAKYHKYNLFYENWFDKPSKVDISVFNTPFGRFGLIICFDILFEHPPIDLICRNNISNIAFPTAWMDAHPFYTSIQFHSAFAVGLGVNFLAANIHYPSLRFHGSGIYTQQGAAGYCFNTSNESGGQLIVQKIPVVDQSRLDACRGLNISLSDLNDQMLELDKTVNNGRETLQHTKRKGWGKGREKGRGNGQGQGKMHGHGQRNGQGQGKRKETRQEKGRGKRKGQGNGQGKGKGPRKLDLLSQPKFHKDMFPYDLYKNPFNLVPLRPEGRKTIVCHTDLCCHLSFSFEPTDRRSAEKPTRKSHAPDDFQASQHSTDLRNFTENRKDQDDIRAHLADNQRSSVEDFYAIGAYDGRYKNRSTGYYIQVCIVLRCRKQQNVVNCSASMAEDFRIRLSHFRLVGNFSATFQYPSILLQGESDFRLTEHSRRWSFSNGQLVANERFSSPLAVATIFARDYGRD

SEQ ID NO: 132 - protein, 533 aa, Patella vulgata
MLPVILVVAGILLNSMSTNAYNRKTFTAAVYEHSLIFPPNRSTVASRHNALADVMVNLRIYDQQAKFAASQDVDILVFPEDGLYGYTFNRDSIKHYLEEIPDPKSEVWNPCTQPNRYSKTEIQVYLSCMALNNSMYIVANMGNKQRCTRRTDSNCPSDGQYQYNTNVVYAPNGRLIARYHKQNLFYEYQFDRPRSVNYAFFETPFGRFGTFTCFDILFEEPAITLIERYNVSNIVFPTAWMDALPLLSAIQFHSAFAQGMGVNFLAANIHVPNKRFHGSGIYTPDGPKKFYYNDVGYGGGKLIISQLNVIENRHQHVAYNKVQRPSSEQKTFHSYVFYDKFTFVELQRSSGVLSVCNNRLCCHLEYAINKDNSDMYAFGAFDGLHTHEGQYYLQICTLLRCANKTRSSCGQPTKQAYSEFNDIKIYGTFQTNYVFPEVLKSRKRDGQLNLATNGWKYNNGVLRATRKTTHPVLSVSLFGRIYSRDKLPPGYHFTGSGSKSLSSSLYVSLLTPALLLAVYGSSIACGLFTRMNN

SEQ ID NO: 133 - protein, 517 aa, Mizuhopecten yessoensis
MIIKFIYVAVLTSSVGGFLFQNETRTTFRAAVYEHAVHLFPSSSSTTRQTALANMMMNLEEYKRQADIAGGQDVDILVFPEMGIYGLGLSREGIAPYLENIIDPEKEVWSPCDYPHRYPNSEVQHFLSCLAKNNSLYIVANFGDRQPCSQSLDPNCQAQYQYNTDLVYDPRGILVAKYHKVNLYFEKSFDFPPVNKAVTFDTPFGVFALAICFDVLFYDPIIDLIINHGVRNVAFPTAWMDRLPFLSSIEYHSAFAAGMGVNMLAANINYPRMRFHGSGIYTPNGASRFIYNDQTASGKLLVSDLSVINKHGSVTLPPRTPTLKTPHVTGSIFKSKLHYDSYNLTLISGISGHATVCHNTICCQLNYSRQNTNETFAFGAFDGLHGRNGSYFMYLQVCTLLKCTRTGNTSCDDRNMQSQTMFNNVHITGYNFTSYIYPEVLLSQNGSLGLSIGRWTYSYGVLHAPTGYEAPLVSTSLYGRLYSTDDSNGRESCSIRLINRKSLITDLVVTLTCRISE

SEQ ID NO: 134 - protein, 520 aa, Octopus sp.
MNFVYTIIFFLQLPVLLSAAEKTFTAAVFEPTIPRRSSMGLVSRDNAIEQMKHVLKGYAEVAARASKKGVDILVFPENGLIAHLDGVTRLGIRNFLDLIPEPNKEKPWIPCDEPYLYPRTEIQQVISCIAKKNKIYLVVNVGDIQPCNRTDDANCPYDGRYQYNTNVVYSSEGALVSRYHKKHLFYEPYYNSPKIAEYGVFDTPFGRFASIVCFDIYFNDSLNELVMKHQVSNILFPNLWFDTPPFFFSSLFHNSIAKAFNINLISANIKINNFVRGGSGIYTPSINRYVYNPLNPAPSDEYIQLQVKNLEKSKRTFPCPPIRKVPESDNPDVKTATMTNLFPVSAVMLHKYSGVVSVCKYNACCSLQYEFMSNRTSVFNINYILAITNEESVDLTNPEGVDMQSCILCPIKDFKMSCLMSLPAGIPDGEVSFRKLKMTAHNFNTSYFFPHIATFNSSTYDINTSWKHDRGEIEITEPVKKVFNAVIFGRLYPNSATATFAIFPAILLILLNLISKELLV

SEQ ID NO: 135 - protein, 554 aa, Formica exsecta
MRTRILDLLLIYSSLNFCCYGNLEDCTDGPDEDYCNENFISAPAQISTPDSPTYIAAVVEYHPEYTTDSNETLKKNSDAYVEHIKTARVHNADIIVFPEDGLTTVKFPKREEMQNWTTIIPSASFNYTPCTDSTIEVSETLKKISCAARNNTIYVVINIAEKLPCIEDVCPKDKMFYYNSNVVFDRTGKIIARYRKTNLFGEQQFNVTSKPEIVIFDTDFGVKFGTFICFDILFHEPALQLTRVHQVTDIVYPTAWFSEAPFLTAVQTQAGWSFSEDVNFLVSGYNEPASGNTGSGIYLGREGIGKAIMSKTTRDEILVFEVPKKKKVFNKDHHDHSNDYDHKVYNHKKENIHEELWRKQFSNTMADNDTIFLIHDNINVFETFSLEGNVTRNVCKNNFCCDFKVEVVTIDPSKKYRLIAFSGIRLYATVEAGVRACGIVQCLNDSVSSCGSVHDSKTVFSRIEIDTTFYDYKNILIMPSTLRSDLLPLSENWTYNEHTHDDHVHISMLLNNNTNNLTTFGIYSRYFNKNNASRIVFDIYYFIALLVSLCLSRL

SEQ ID NO: 136 - protein, 516 aa, Drosophila simulans
MAKNYWGFSLFCLALGLMLNLSQQASLFSRDFYTAGVVEFKPSNDLSDNLAGYLEIIHSQNATSTDIIVFPESTLNSAGSTTFVPNPEDQIIPCLSDPNATYYEEFLVTLSCAARNASKYIVINLTEKQKCEDVPEDTRPCASNGLNVFNTNVVFDRQGVVVSRYRKVHLYGEPKNSTFLPELSTFETDFGVTFGHFICFDILFYTPAHQLVDQGVTDFVYPAMWFSQLPFLTAVQIQLGWSYANNVNLLAAGASDPIVGNSGSGIYHGRSGSLRSVMRQESGERSIYVARVPKYRAKRRMKRDLNRQVATSSSFNIKRDYLENFTSEELKIDDGKTGNLSQNLCHGGFCCHFDLAWRSLRKPARNTSHYSYRVGIYEGWRNEKRLDVNYIRNCGLFTCSGDSIDDCGQLLPDLHRPLVTFTHLEIRVTYPQSRKFLLFPDTLLDNLLPLEPKQFEWSQKRISEESQQVRLALRKSLEVKHLLTFGIYGNYYNNECTYGVGTTEEQLECGYKNPKI

SEQ ID NO: 137 - protein, 535 aa, Trichonephila clavipes
MKRILDFQLFFTTVLSLLALELAAGPDHYRAAVAETVRFSDISCPPKEVVKRNLDIYEAAVKASAKNDVDIIVFPECGIFPCEKVNRSVLRTFVEDIPNPKRESINPCKQSDKFHNHSILSKLSCLAKKHNLYIVANTFDFKKCKPVETCDADGFDTCTSEESNCPSDGNFFYNTNIVFDRKGSLVSRYYKKHLYFERTLNKPDFQQDATFHTDFGNFTTVICFDLIFKESVEDVKKPGIINLAYPTYWYDHVPQVFFATAYQQAWAMTNKVNLLAANGHMPKTGTLGSGIYSAHKGALIYTHNPDFRTKLLISNVPRSLNKEKANSIELNEKKFYLESGAAIEVVGEEEEERNFRSDCTTTVLGEANDTMDSYRCFDSTLNDYQYFKLEGNEGTHEICSNEFCCSLSYQAHSMDEDYFLAVSGKPLTYYDTFSYGIQSCFLARCRSSDGHPCKHFLLKSRTIFQNVHIKANFTTRHVYPFALDSDVRLTNRKNWSFDGKSQITYENLNKKSSVTFFGLHGRLYDVDKILKKLLK

SEQ ID NO: 138 - protein, 509 aa, Parasteatoda tepidariorum
MTTSWFLLLSMFFVACTDIDATAYTAAAYDHARVGRLNDPPNLVVEMNLKKYIKAGKIAKSKGADIIVYPEYGIFYPGNRTQLKHFLEDVPDPKKTRTAPCDEVELYMKRPILYTLSCVAKHNAMYVVANMGDIKDCSGVSECPEDGVFHFNANIVFDRNGRLILRYYKEHLFFELGMDLAREEQDPVFTTDFGTFATYICFDILFKRMSQVAKWPQIDGIMFTSMWAENPPQGLSVAFWQGWALGNNATIVVSNIQSPSRYALGSGIFHKTHGSLGYTYNPDRVSKVVVAKVPVSHQPVLTSRPSLTAFTDTKVWDMKSEEEDVPKVCSNLLIDATDDSTEKLYCSDEKTTNYTLVKLPKSGGHVKACNNGMCCSVSYSAETIEEDYYLGVYNGTINAYDLNRYCWCEENCFLARCDAIKGRPCSTFPRKTNTVFYHVELKSNFSTPFVYPSVVSSKSRLVPMTEWEFDVGHSSDSQSSIHFHSKYGEQLLSVGFRARCFDRDPPYVR

SEQ ID NO: 139 - protein, 487 aa, Rhipicephalus sanguineus
MQGMWLLLTAVLCSSTAHAFKAAVYEHVQSKATPTETAVAENLEAYLWATGNASSEKADIIVFPEDGILYRLKDREEVAKWAEDIPDLGVMACGSQNMSLLSNLSCMAQSNSIYLVANLIDRKPCANETLTTAVTATRSSISTTNGPSDRNGPLSQVILHMLLIETFSISRCILPTTRYHKNHLFVEPLMNPADPYEFAVFKTDFGAQVGLFICFDVLFAESSLLVEKHNVTLGVMSSWWFDELPGWYSVGVQQAWSIHNGIPLLASGIQRLEMGSLGSGIYAGLRGPLNYTYSPDGKSKLLMADLSNATSLNPRYPGDELHPKQRFLNQGDVSDHAAQVLGRPKGYSRVCHGDFCCSLSYETQELNDNFVLLARNGLRNIGTYMQLGIQQCILAVCESRNNTVCKNFSTRSTTKFTELQLTAQFATNAVFPVVASDELALTPKDKWQFETTSTNASTLTLKENGKDEDILHIVLYARLYDADRFTK

SEQ ID NO: 140 - protein, 524 aa, Caerostris extrusa
MMASGFKLLCLAVALHMSSIGLCADEKYYRAAVHEIRQYNDNSSSGSELISKNMELYNQAADIASFENCDILVYAEKGLFPSKKSNSSWFIYYAEDVPSGKERANPCEQEEYSDRPILKNLSCIAQEHSLTVVATLIDIKKCIVSEDCERTTINYCVSNADDCPEDGIYRFNTQVVFNKRGTLIARYYKQHPFFEEGITTPKYAANVYYKTNYGTFVSYIGFDFFFYDSVKGVEKPWINAVTYANWWFDHTPFNYFSVSNQQAWSMNNGVAVLSSDVHEPNLGSLGSGIYIPGKGALIYSYNPDGKSKLLISNVPKSNSLSDNESLNTKFYYIDENGTGTVLNGEEPRNFKSQCSIDVLGEETSSFTDYRCHKTEVHQYTFTKLNDTSGQIDTCSNGFCCSLSYTAESMNETFYFGVSGHRLKFYDTYWFGVQACFLARCEDVDGKQCRNFLLTSDTIFHDVEITGSFETKHIYPQAMDTELRLTDRNEWKFDGQSMSYESVNQNPLLFMGLYGRLYRRDGLVK

SEQ ID NO: 141 - DNA, 1503 bp, Mus musculus
  source 1..1503: mol_type = genomic DNA
atggcttcattacattttcctcaatgggcagtgagttttgtcttctttgcccaggctgtgggttcaatggacacttttattgctgctgtgtatgaacatgctgttatactgccaaacaaaactgaaagtcctgtttccactgaagaggctttgctcctgataaacaagaacatagacattttggagagtgcaatcaagctggcagccagacagggtgcacatatcattgtgacgccagaagatggaatctatggttggatcttcaccagggagaccatttacccctacctagaggatataccagaccctgaagtgaactggattccctgtagagaccctaggaggtttggctacacaccagtacaggagagactgagctgccttgccaaggagaactctatctatattatggcaaatattggggacaagaagccatgcaatgctactgatcctcattgtcccccggatggccgttaccaatataataccaatgtggtcttcgattctaagggtaggctaacagcccgctaccataagtacaatctttttgaaccagagattcagtttgatttccccaaagattcagagctggtgacctttgacaccccgtttgggaagtttggcatcttcacttgctttgacattttctcttatgacccagctgtggtggttgtgaaggacacccaggtcgacagtgttctcttacccacggcgtggtacaacaccctgcccctgctttcagcagttccattccattcggtgtgggccagagccatgggggtcaacgtgcttgctgcaaacacccacaacaccagcatgcatatgacagggagtggaatctacagcccggaagctgtccgagtgtaccactatgacatggagacagagagtggccaactgctgctttcagagctgaggtctcggcctcgccagcacgccacccctgcagaggttaactggagcgcttatgccaggactgtgaagccgttctcatcggggcaggcagacttcccaggaaagatttattttgacgaatttagcttcaccaagcttacaggaagtgctggcaattacacagtttgccaaaaggacctgtgctgtcacctgacttacaagatgtctgaaagccgaatggacgaggtgtatgttctgggtgcctttgatggactccatacaggggaaggccagtattacctacagatatgtacattgctgaagtgtcaaaccaccaactcgagaacttgtggggaacccgtggggtcagcttttacaaagtttgaagaattctctctcagtggcacctttcggacaaaatatgttttcccacagatcgtgctaagtgggagtcaacttgccctggaaagatattatgaagtctcaagagatggacgtctgaggagtcgaggtggagcccctttgcctatcttagtgatggccctgtatggaagagtgtttgagagagaccctccgcgcttagggcagggacctgggaagctgcagtga

SEQ ID NO: 142 - DNA, 1503 bp, Mus caroli
  source 1..1503: mol_type = genomic DNA
atggcttcattacattttcctcaatgggcagtgagttttgtcctctttgcccaggctgtggggtcaatggacacttttattgctgctgtgtatgaacatgctgttatactgccaaacaaaactgaaagtcttgtttccactgaagaggctttgctcctgataaacaagaacatagacattttggagagtgcaatcaagctggcagccagacagggtgcacatatcattgtgacgccagaagacggaatctatggttggatcttcaccagggagaccatttacccctacctagaggatataccagaccctgaagtgaactggattccctgtagagaccctaggaggtttggctacacaccagtacaggagagactgagctgccttgccaaggagaactctatctatattgtggcaaatattggggacaagaagccatgcaatgctactgatcctcattgtcccccggatggccgttaccaatataataccaatgtggtcttcgattctaagggtaggctaacagcccgctaccataagtacaatctttttgaaccagagattcagtttgatttccccaaagattcagagctggtgacctttgacaccccgtttgggaagtttggcgtcttcacttgctttgacattttctcttatgacccagctgtggtggttgtgaaggacgcccaggtcgacagtgttctcttacccacggcgtggtacaacaccctgcccctgctttcagcagttccattccattcagcgtgggccagagccatgggggtcaacgtgcttgctgcaaacacccacaacaccggaatgcacatgacagggagtggaatctacagcccagaagccgtccgagtgtaccactacgacatggagacagagagtggccaactgctgctttcagagctgaggtctcggccccgccagcatgctacccctgcagaggttaactggagcgcttatgccaggactgtgaagccgttctcatcggggcaggcagacttcccaggaaagatttattttgatgaattcagcttcaccaagcttacgggaagtgctggcaattacacagtttgccaaaaagacctgtgctgtcatctgacttacaagatgtctgagagccgaatggacgaggtgtatgtcctgggtgcctttgacggactccatacagtggaaggccagtattacctacagatatgtacactgctgaagtgtcaaaccaccaactctagaacttgtggggaacccgtggggtcagcttttacaaagtttgaagaattctctctcagtggcacctttcggacaaaatatgttttcccacagattgtcctaagtgggagtcaacttgccctggaaagatattatgaggtctcaagagacggacgtctgaggagtcgaggtggagcccctttgcctatcttagtgatggccctgtatggaagagtgtttgagagagaccctccacgcttagggcagggacctgggaagctgcagtga

SEQ ID NO: 143 - DNA, 1503 bp, Mus pahari
  source 1..1503: mol_type = genomic DNA
atggtctcattacattttcctcaatgggcagtgagttttgtcctctttgcccagactgtgggttcaatggacacttttattgctgccgtatatgaacatgctgttatactgccaaacgaaacagaaactcctgttcccaccgaagaggctctgctcctgataaacaagaacatagacattttggagagcgcaatcaagctggcagccagacagggtgcacatatcattgtgacgccagaagacgggatctacggttgggtcttcaccagggagaccatttacccctacctagaggatataccagaccctgaagtgaactggattccctgtagagaccctaagaggtttggctacacaccagtacaggagagacttagctgcctcgccaaggagagctctatctatattgtggcaaatattggggacaagaagccctgcaatactactgatcctcactgtccccgggatggccgttaccagtataataccaatgtggtcttcgattctaagggtaggctaacagcccgctaccataagtacaatctttttgaaccagagattcagtttgatttccccaaagattcagagctggtgacctttgacaccccgtttgggaagtttggcatcttcacttgctttgacattttctcttatgacccagccgtggtggttgtaaaggacgcccaggttgacagtgttctcttacccacggcgtggtacaacaccctgcccctgctatcagcagctccattccattcagcgtgggccagagccatgggggtcaacgtgcttgctgcaaacacccacaacaccaggatgcacatgacagggagtggcatctacagcccggaagccgtccaagtgtaccactacgacatggagacagagagtggccaactgctgctttcagagctgaggtctcggccccgccagcatgccacccctgcagaggttgactggagcgcttatgccaggactgtgaagccgttctcatcggggcaggcagacttcccgggaatgatttattttgatgaattcagcttcaccaagcttatgggaagtgctggcaattacacagtttgccaaaaggacctgtgttgtcacctgacttataagatgtctgagagccgaatggacgaggtgtatgtcctcggtgcctttgatggactccatacagtggaaggccagtattacctacagatatgtacattgctgaagtgtcaaaccaccaacttgagaacttgtggggaacccgtggggtcagcttttacaaagtttgaagaattctctctcagtggcacctttcggacaaaatatgtcttcccacagatcgtcctaagtgggagtcaacttgccctggaaagatattatgaagtctcaagagacggtcgtctgaggagccgaggtggagccgctttgcctgtcttagtgatggccctctacggaagagtgtttgagagggaccctccacgcttagggcagggacctgggaagctgcaatga

SEQ ID NO: 144 - DNA, 1503 bp, Arvicanthis niloticus
  source 1..1503: mol_type = genomic DNA
atggtttcattatattttttacaatgtgtagtgagtcttgtcctctttgctcaggctgcgggttcaatggacacctttattgctgcggtatatgagcatgctgttatactgccaaacaaaacagaaagtcctgtttccaccgaagaggctttgctcctgatgaacaagaacatagacattctggagaatgcgatcaagctggcagccagacagggtgcacagatcattgtgaccccagaagatggaatctatggctggatcttcaccagggagactatttacccctacctagaggatataccagaccctgatgtgaactggattccctgtagagaccctaaaaggtttggctacacaccagtacaagagagactcagctgcctcgccaaggagaactctatttacatcgtggcaaatattggggacaagaagccatgcaacgctactgatcctcagtgccccagggatggccgttaccaatataataccaatgtggtcttcagttctgagggtaggctagtagcccgctaccataagtacaatctttttgaaccagagattcagtttgattttcccaaagattcagagttggtgacctttgacaccccctttgggaagtttggcgtcttcacttgctttgacattttttcttacgacccagctgtgatggttgtgaaagactttcaggttgacagtgttctcttacccacggcgtggtacaacactctgcccctgctttcagccgttccattccattcagcatgggccagaggcatgggggtcaacctgcttgctgcaaacacccacaacaccagaatgcacatgacagggagtgggatctacagtccggaggctgtcagggtttaccactacgacatggagacagagagcggccagctactgctttcagagctgaggtctcggccccgccagcaggcctcccctgcagacgttgactggagtgcctatgccaggactgtcaagccattctcatcggagcaggccgacttcccagggatgatttattttgatgaattcagcttcaccaagcttacaggaagtgctggcaattacacagtttgtcaaaaagacctgtgctgtcacctgacttacaagatgtctgagagccgaatggatgaggcttatgccctgggtgcctttgatggactccataccgtggaaggccagtattacttacagatatgtacattgctaaagtgtcaaaccactaacttgagaacttgtggggaacccgttgggtcagcttttacgaagtttgaggaattctctctcagcggcacctttgggacgaaatatgttttcccacagatcgtcttaagtgggagtcaactggccctggaaagatattatgaagtctcaagagatggacgtctgaggagccaaagtggggcctctttgcctgtcttagtgatggccctgtacggaagagtgtttgagaaagattctccacgcttaggtcagggacctgggaagttgcaatga

SEQ ID NO: 145 - DNA, 1506 bp, Homo sapiens
  source 1..1506: mol_type = genomic DNA
atgattatatcacattttccaaaatgtgtggcagtttttgccctccttgctctgagtgttggtgcactggacacttttattgctgcagtatatgagcatgcggtgatattaccaaacagaacagaaacacctgtttcaaaagaagaagctttgctcctgatgaacaagaacatagatgttttggagaaagcagttaagctggcagcgaagcagggtgcacatatcattgtgaccccagaagatggaatctatggttggatcttcaccagggagagcatttacccctatctagaggatataccagaccctggagtgaactggattccatgtagagacccctggagattcggcaacacaccagtgcaacaaagactcagctgcctggccaaggacaactctatctatgtcgtggctaatattggggacaagaagccatgcaatgctagtgactctcagtgtccccctgatggccgttaccaatacaacactgatgtggtgttcgattctcagggaaaactgttggcacgctaccataagtacaatctttttgcacctgaaattcagtttgatttccccaaggattcagaacttgtgacttttgacactccctttgggaagtttggcatttttacttgctttgacattttttctcatgacccagctgcggtggtggtggatgaagtttcaattgacagcattctctaccccacagcatggtacaacacgctgcccctcctctcggctgttcccttccattcagcatgggccaaggccatgggagtcaatctacttgctgcaaatacccacaacaccagcatgcacatgacagggagtggaatctacgccccagaagcagtcaaggtgtaccactatgacatggaaacagagagtggtcagctgttgctatcagaactgaagtctcggccccgccgtgagcccacctaccctgcagctgttgactggcatgcgtatgccagcagtgtcaagccattttcctctgaacagtcagattttctggggatgatttattttgatgagtttaccttcaccaagcttaagagaaatacaggaaattacacagcttgccagaaagatctgtgttgtcacttaacttacaagatgtctgagaagcgaacagacgagatctatgccctaggtgcttttgatggactgcacacagtagaaggccaatattacttacagatatgtgcattactgaagtgtcaaaccactgacctggaaacgtgtggagaacctgtggggtcagcttttaccaagtttgaagacttctccctcagtggcacatttggaacgcgttatgttttcccacagatcattctaagtgggagtcagcttgcccctgaaagacattatgagatttcaagagatggacgcttgaggagccgaagtggagcccctttgcctgtcttagttatggccctgtatggaagagtgtttgagaaggaccctccacgcttagggcagggatctgggaaattccagtga

SEQ ID NO: 146 - DNA, 1506 bp, Cynocephalus volans
  source 1..1506: mol_type = genomic DNA
atgattatatcacattttccaaaatgtgtggccatttctgccctctttgtcctgagtgttggtgcactggatacttttattgctgcagtgtatgagcatgctgttatcttaccaaacagaacagaaacacctgtttcaaaagaagaagctttattcctgatgaacaagaatatagatgttttggagaaagcagtcaagctggcagccaagcagggtgcacatatcattgtgacaccagaagatggaatctatggttgggtattcaccagggagagcatttacccttatctggaggatataccagactctgaagtgaactggattccatgtagagatcctcaaaggtttggctacacaccagtgcaagaaagactcagctgcctggccaaggacaactccatctatgttgtggcaaatattggggacaagaagccatgcaatgccagtgaccctcaatgtcctcccgatggtcgttaccaatacaacacggatgtggtgtttgattctgagggtaggctggtggcacgctaccataagtacaatctttttgcacctgaaattcaatttgatttccccaaaacatcagaagttgagacttttgacactccctttgggaagtttggaattttcacttgctttgacattttttctcatgacccagctgtggtggtggtggacaagtttcaagtcgacagtgttatctaccccacagcgtggtacaacacgctgccccttctctcggctgttccattccattctgcatgggccagagccatgggagtcaatctgcttgctgcaaatacccacaacaccagcatgcacatgacagggagtggtatctatgccccagaagcagtcaaggtatatcactatgacatggaaacagagagtggtcagctgatgctctcagaattgaagtctcggcctcgccgtgaacccacctatcctgcagttgttgactggggtgcatatgctgaaagtatgaagccattctcgtctgagcagtcacattttccagggctgatttattttgatgagttcaccttcaccgagcttaagagaaacacaggaaattacacagtgtgccagaaagacctgtgttgtcacttgacttacaagatgtctgagaagcgaacagatgaggtctatgcactaggtgcttttgatggactgcacacagtagaaggccaatattacttacaggtgtgcacattactgaagtgtcaaaccactgacctgagaacttgtgggcagccagtggggtcagtttttaccaagtttgaagaattctccctcagtggcacatttggaaccagttatgttttcccgcagctcattctaagtgggagtcagcttgcccctgaaagacattatgaggtttcaagagatggacgtctaaggggccgaagtggagcccctttgcctgtcttagttatggctctgtatggaagagtgtttgagaaggaccctccacgcttagggcagggacctgggaaattgcaatga

SEQ ID NO: 147 - DNA, 1506 bp, Sapajus apella
  source 1..1506: mol_type = genomic DNA
atgagtagatcatattttccaaaatgtgtggcactttctgccctccttgtcctgagtgttggtgcactggacacatttattgctgcagtatatgagcatgcggtgatattaccaaacagaacagaaacacctgtttcgaaagaagcagctttgctcctgatgaacaagaacatagatgttttggagaaagcagttaagctggcagccaagcagggtgcacatatcattgtcaccccagaagatggaatctatggttgggtcttcaccagggagagcatttacccctacctggaggatataccagaccctgaagtgaactggattccatgtagagaccctgggagattcggcaacacaccagtgcaagaaagactcagctgcctggccaaggacaactctatctatgtcgtggctaatattggggacaagaagccatgcaatgccagtgaccctcagtgtccccccgatggccgttaccaatacaacactgatgtggtgtttgattctcagggaaaattggtggcgcgctaccataagtacaatctttttgcacctgaaattcaatatgatttcccgaaggattcagaatttgtgacttttgacactccctttgggaagtttggcatttttacttgctttgacattttttcttatgacccagctgtggtggtggtggatgagtttcaagttgacagcattgtctaccccacagcatggtacaatacgctgcccctcctctcggctgttcccttccattcagcatgggccaaggccatgggagtcaatctgcttgccgcaaatacccacaacaccagcatgcacatgacagggagtggactttacgccccagacgcagtcaaggggtaccactacgacatggaaacagagagtggtcagctgatgctatcagaattgaagtctcggcctcgccgtgagcccacctaccctgtagctgttgactggcatgcttatgccagcagtgtcaagcctttctcctctgagcagtcagattttccagggatgatttattttgatgagtttaccttcactgagcttcaaagaaatacaggaaattacacagtttgccagaaagacctgtgctgtcacttaacttacaagatgtctgagaagcgaacagatgaagtctatgccctaggtgcttttgatggactgcacacggtagaaggccaatattacttacagatatgcacattactgaagtgtcaaaccaccgacctcgaaacatgtggagagcctgtgggttcagcttttaccaagtttgaagaattctccctcagtggcacattcagaacgcgttatgttttcccacagatcactctaagtgtaaatcagcttgcccctgaaagacattatgagatttcaagagatggacgcctgaggagccgaagaggagcccctttgcctgtcttagtcatggctctgtatggaagagtctttgagaaggaccctccacgcttagggcagggacctgggaagttacagtga

SEQ ID NO: 148 - DNA, 1506 bp, Panthera leo
  source 1..1506: mol_type = genomic DNA
atgattacatcatattttccaaaatatgtggcactatttgccatctgtgtcctatgtgtcggtgcactggacactttcattgctgcagtgtatgaacacgctgtcatattaccaaacagaacagagacaccagtttccaaagaagaagctttgctcctgatgaacaagaacatagatgttttggagaacgcagttaagctggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaagacaactccatctacgttgtggcaaatattggggacaagaagccatgcaatgccagcgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgttctctacccggtggcgtggtacaacacactgccccttctgtcggctgttcccttccactctgcgtgggccagagccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattctcgtctgaacagtcgaattttccggggatgatttattttgatgagctcaccttcattgagcttaagagaaacacaggaaattacacagtttgccagaaagacctgtgctgtcacttaacttacaagatgtcggagaaacgaacagacgaggtttacgcactaggtgcttttgatggactgcacacagtagaaggtcaatattacctacagatatgcacattactgaagtgtcaaatcagtgacctgcgaacttgtggggagcccgtggggtcagctttcaccaagttggaagaattctccctcagcggcacatttgcaacacgttatgttttcccacagatcattctcagtggaagtcagcttgctcctgaaaggtattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 149 - DNA, 1506 bp, Lynx canadensis
  source 1..1506: mol_type = genomic DNA
atgattacatcatattttccaaaatatgtggcgctatttgccatctgtgtcctatgtgtgggtgcattggacactttcgttgctgcagtgtatgagcacgctgtcatattgccaaacagaacagagacaccagtttccaaagaagaagctttgctcctgatgaacaagaacatagatgttttggagaacgcagttaaactggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagtgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgtcctctacccggtggcgtggtacaacacgctgccccttctgtcggctgttcccttccactctgcgtgggccagagccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgtagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgagaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 150 - DNA, 1506 bp, Panthera tigris
  source 1..1506: mol_type = genomic DNA
atgattacatcatattttccaagatatgtggcactatttgccatctgtgtcctatgtgtcagtgcactggacactttcattgcttcagtgtatgaacacgctgtcatattaccaaacagaacagagacaccagtttccagagaagaagctttgctcctgatgaacaagaacatagatgttttggagaacgcagttaagctggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagcgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgttctctacccggtggcgtggtacaacacactgccccttctgtcggctgttcccttccactctgcgtgggccagagccatgagagtcaacttgcttgctgcgaatacccacaacaccagcatgcacatgacagggagtggaatctacacaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgcgaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 151 - DNA, 1449 bp, Hippopotamus amphibius
  source 1..1449: mol_type = genomic DNA
atgattatatcatattttccaaaatatgcggccactttggccttctttgtcctgagtgccggtgctctggacactttcattgctgcagtgtatgagcacgctgttgtattaccaaacaggacagaaacacctgtgccgaaagaagcagctttgctcctgatgaacaagaatatagatgttttagagaaagcagttaaactggcagccaggcggggcgcgcatatcattgtgaccccagaagatggaatttatggctgggtcttcacaaggggcaccgtttatccctatctggaggatatcccagaccctgaagtgaactggattccatgtagagattcccagaggtttggccgcaccccagtgcaagaaaggctcagctatttggccagggacagctctatctacttcgtggcaaatattggggacaagaagccatgcaataccagtgaccctcagtgtcccccgnacggtcattaccaatacaacactgatgtggtgtttgattctgagggtaggctggtggcctgctaccacaaggacaatctttttgcacctgaaattcaatttgatttccccaaggattcagaatttgtgacttttgacacaccctttgggaagtttggcattttcacttgctttgacattttttctcatgagccagctatggtggtggtggaggagttacaggttgatagtgttctccaccccatggcctggtacaacacactgcccctgctctcggcggtccccttccattctgcgtgggcccgagccctgcgagtcaatctgctagccgccaatacccacaccaccagcatgcacgtgacaggtacttcatgccgctcctgccccaatgggcagccgtgggtgtgctatgacacggaaacagagagtggccagctgatgctctcagaattgaagttgtggcctcgaagagaacgcagttaccctgcagctgttgactggagtgcttatgccagaggtgtcaaaccattcccatctaaactgttggattttccagggatgctttattttgatgagttcaccttcaccaagcttgagagaagcacaagaaattacacagcttgtcagaaagacctgtgttgtcacnnaacttacaagatgtctgagaaacgaacccatgaggtatatgtactaggtccctttgatggactgcacacggtagaaggtcaatattacttacaggtatgcacattactgaagtgtcaaaccactgacctgagaacttgtggagagcctgtggggtcagcttttaccaaatttgaagatctctccctcaggggcacatttggaacgcgttatgttttcccacagatcattcttagtgggagtcagcttgcccctgaaagacattatgaggtttcaagagatggacgtctgcggagccaaggtggaggcccttttcctgtcttggtcctggccctgtag

SEQ ID NO: 152 - DNA, 1500 bp, Gracilinanus agilis
  source 1..1500: mol_type = genomic DNA
atggctactgcctggtttcccacttgtgtgatagttttggcgatctgtatcctcgatggcggtgcctcagacacatttaaggctgctgtatatgagcatgctgttatattgccaaataggacagaaatacctgtttctcagaaagatgccctgatgttgatgaataagaacttagatgttttggagaaggcaatcaaattggcggctaagcagggtgcagacattattgtgactcctgaagatggagtttatggctgggatttcacaagggaaaccatttacccctacttagaagatatcccagaccctcaagaaaactggattccatgtactgaagcccacagatttggccgtactccagtgcaaaaaagacttagctgcttggccagagagaattctatctacgtggtggcaaatattggagacaagaaagaatgtggtgctaatgaccccacatgcccaccagatggtcggtaccaatacaacactaatgtggtgtttgattcagatgggaaactggtggcccgctaccataagtacaatctttttgcaactgaagttcagttcaacttccctaaagattcaccatatgtgacctttgatacaccctttggaaagtttggcattttcacttgctttgacatattttcttatgaacctgctgcagctgtggtggataaattccaagtcgacagcatactgtaccccacagcctggtacaatactctgcccctcttgtctgctgttccattccattcagcctgggccagggccatgagagtcaatctcctttcagcgaacactcacaacaccaggatgcacatgacagggagtggaatctatgcaccagaggcagttaaagtttatcactatgacatggaaacaaaaagtggccaactattgctctcagaactgaagacacggccacgactttcttctgactaccctgcagtggtagattggagttcctatgccaaaagtgccaagccattcatttctgatcagccaaagttccatggaagtatctattttgatgaatttaccttcattgaacttactaaagaaacaggaaattacaccatttgccagaaagaactctgctgtcacttgagctacaagatgtctgagaaaagaactgatgaagtctatgcactaggggcttttgatggccttcacattgttgaaggacaatattatttacagatatgcacattattgaagtgccaaacaactgacttaaaaacctgtggtgagccagttggatcagctttcaccagatttgaagaattctccctcagtggcacattcaacaccagatatgtcttcccccagttagtactaagtgggactcaacttgcacatgaacaacactatgaggtatcaaatgatggtcgtctgaagagcagaagtggtgtctcattgcctgttttagtgatggcgctatatgggagagtatttgagaaggacccacatcgtttaggacagggtgatcatccttga

SEQ ID NO: 153 - DNA, 1347 bp, Puma yagouaroundi
  source 1..1347: mol_type = genomic DNA
atgaacaagaacatagatgttttggagaacgcagttaaactggcagccaggcagggcacacatatcattgttaccccagaagatggaatttatggctgggttttcacaagagagaccatttacccctatctggaagagatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagtgaccctcagtgtccccctgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgtcctctacccggtggcgtggtacaacacgctgccccttctgtcggctgttcccttccactctgcgtgggccagaaccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgacggactgcacacggtagaaggtcaatattacctacagatatgcacattactgaagtgtcaaatcagtgacctgagaacttgtggggagcccatggggtcagctttcaccaagttggaagatttctccctcagcggcacatttgcaacacgtcatgttttcccacagatcattctcagtggaagtcaacttgcgcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaataa

SEQ ID NO: 154 - DNA, 1545 bp, Microcebus murinus
  source 1..1545: mol_type = genomic DNA
atgatcacttctccgttcctggcttatgtggcaatttgggttttctgtgtcttgagaaccagctctctggacacttttattgcagctgtttacgagcatgcggtgctattgcctaatgccagcctcacaccagtatctcgtgaggaggcgctgaaatggatggatcagaatctggatattttggaaggagccatcacgtcagcagcagagcagggtgcacatattatcgtgactccagaagatggtatttatggctggcacttcagcagggaatctgtctacccgtacttggaggatatcccagaccctcaagtaaactggatcccctgtgataatcctaacagatttggccacaccccagtacaagaaagactcagctgcctggccaaggacaactctatctatgttgtggcaaatattggggacaagaagccatgcaatgccagtgaccctcagtgtccccctgatggccgttaccaatacaacactgatgtggtatttgattctcagggtaggctggtggcacgctaccataagcaaaatcttttcatggttgaaaatcaattcaatgcacccaaggagcctgagattgtgactttcaacaccacctttggaaggtttggcatttttacatgcttcgatatactcttccatgatcctgctgttaccctggtgaaagacttctacgtggataccatactcttcccaacggcttggatgaatgttttgccacatttgtcagccattgaattccactcagcgtgggctatgggcatgggggtcaatttccttgcatctaatacacattacccctcaatgaggatgacaggaagtggcatctatgcccccaattctgcaagagcatttcattatgatatgaagacaaaggagggaaaactcctcctctcacaactggattcccacccacgccaccctgtagtggtgaattggacatcttatgccagcagtgtggaagcactctcaactggaaaccaggaatttaagggcattatcttttttgatgaatacaccttcgtggagctcaaaggagttatggggaattacacagtttgtcagaaagatctctgttgtcatctgaactacaagatgtcagagaaaagatcagatgaagtttatgccctaggggcatttgatggactgcacacggtggaaggacgctattatctacagatttgtactttggtgaagtgtaaaacgactgacttaaacacttgtggtgactcagttgaaacggcttctacccggtttgaaatgttctccctcagtggcactttcggaactcagtatgtcttccctgaggtattgttgagtgagatacagcttgcacctggagaatttcaggtgtcaagtgatggacgcttgtttagcctgaagccaatatctggacctgtcttaacagtgactctgtttgggaggttgtatgagaaggaccgagcatcaaacgcttcatccaacctcatagagcaagcactaagaataatgctagcagttataatatctattgtatactcattacgttggtag

SEQ ID NO: 155 - DNA, 1257 bp, Balaenoptera musculus
  source 1..1257: mol_type = genomic DNA
atggccttcctactgaaggcgcccagcagggaatctatctacccatacttggaggatatcccagaccctcaagtgaactggatcccttgtaataatcccaacagatttggccgcaccccagtgcaagaaaggctcagctgtctggccaaggacaactccatctacatcgtggcaaatattggggacaagaagccatgcaataccagtgaccctcactgtccccctgatggtcgttaccaatacaacactgatgtggtgtttgattctgaggggaaactggtggcccgctaccataagcaaaatcttttcttgggtgaagatgagttcaatgctcccaaggaacctgaggtcgtgactttcaacaccacctttggaaagtttggcattttcacgtgctttgatattctcttccatgatcctgctgtgactctggtgaaagatttccacgtggacaccatactcttcccgacagcttggatgaatgttttgccacatttgtcagctattgaattccactcagcttgggctatgggtatgggagtcaacttccttgcatccaatctacattacccctcaaagaaaatgacaggaagtggcatctacgcacctgattctccaagagcatttcattatgatatgaagacagaggagggaaaactcctcctctcacaactggcctcccacccacaccccacagcagtagtgaattggacttcctatgccagtggtatagaagccccctcaatgggaaaccaggaatttaagggcaccatcttttttgacgagttcaccttcttggagctcaaaggagtcgcaggaaattacacagtttgtcagaaagatctctgctgtcatctaagttacaggatgtctgagaagagatcagatgaagtttatgtactaggggcatttgatggactacatactgtggaagggagctattatttacagatttgtactctgttgaagtgtaaaacaacggacttacacacttgtggtgattcagttgaaactgcttccaccaggtttgaaatgttctccctcagtggcacttttgaaacccagtatgtcttccctgaggtgctgctgagtgaaattcagcttgcacctggagaatttcaggtgtcaagtgacggacgcttgtgcagccagaagccactatctggacctatcttgacagtaactctgtttggaaggttatatgagaaggatagttcaccaaatgctttctcagaccccatggcacagatcatttaa

SEQ ID NO: 156 - DNA, 1617 bp, Rhinopithecus roxellana
  source 1..1617: mol_type = genomic DNA
atgattggatcatattttccaaaatgtgtggcagttgttgccctccttgccttgagtgttggtgcattggacacttttattgctgcagtatatgagcatacagtgatattaccaaacagaacagaaacccctgtttcaaaagaagaaggtttgctcccgatgaacaagaacatagatgttttggagaaagcagttaagctggcagcgaagcgggtattatccttttatacttgtaaaggagacttgcagtttgataaaagagtatttggaattcatgagaaacttttttgccccacttgttttgagcagggtgcacatatcattgtgaccccagaagatggaatctatggttgggtcttcaccagggagagcatttacccttatttggaggatataccagaccctggagtgaactggattccgtgtagagacccctggagatttggcaacacaccagggcaacaaagactcagctgcctggccaaggacaactctatctatgttgtggctaatactggggacaagaagccatgcaatgccagtgaccctcagtgtccccctgatggccgttaccaatacactgatgtggtgtttgattctcagggaaaactggtggcacgctaccgtaagtacaatctttttgcacctgaaattcaattcgatttctccaaggattcagaacttgtgacttttgacactccctttgggaagtttggcatttttacttgctttgacattttttctcatgacccagctgttgtggtggtggatgagtttcaagttggcagcattctctaccccacagcatggtacaacacgctgcccctcctctcggctgttcccttccattcagcatgggccaaggccatgggagtcaatctacttgctgcaaatacccacaacaccagcatgcatatgacagggagtggaatctacaccccagaagcagtcaagatgtaccactatgacatagaaacagagagtggtcagctgttgctatcagaactgaagtctcagccttgccgtgagcccacctaccctgcagctgttgactggcatgcgtatgccagcagtgtcaaaccgttttcctctgagcagtcagattttccggggatgatttattttgatgagtttaccttcactgagcttaagagaaatataggaaattacacagtttgccagaaaaatctgtgttgtcacttaacttacaagatgtctaagaagcaagcagatgaggtctatgctctaggtgcttttgatggactgcacacagtagaaggccaatattacttacagctagaaatgcttaaatatatgtgcacattactgaagtgtcaaaccactgacctggaaacgtgtggagaagctgtggggtcagcttttaccaagtttgaggacttctctctcagtggcacattcggaacgcattatgttttcccacagctcattctaagtgggagtcagcttgcccctgaaagacattatgagatttcaagagatggacacttgaggagccgaagtagagcccctttgcctgtcttagttatggccctgtatggaagagtgtttgagaaggaccctccacgcttagggcagggacccgggaagttccagtga

SEQ ID NO: 157 - DNA, 1284 bp, Felis catus
  source 1..1284: mol_type = genomic DNA
atgactgtgactccagaagatgcgctttatggatggaaattttccagagaaaccatttctctttatctgaaggatatcccggcccctcaggtgaattggattacatgtcaaaacacccagaggtttggctacaccccagtgcaacaaaggctcagctgcctggccaaggacaactccatctacgttgtggcaaatattggggacaagaagccatgcaatgccagtgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagtgttctctacccggtggcgtggtacaacacgctgccccttctgtcggctgttcccttccactctgcatgggccagagccatgagagtcaacttgcttgctgcgaatacccacaacaccagcatgcacatgacagggagtggaatctatgcaccggaagcagtcaaagtgtatcactatgacatggaaacagagagtggccagctgatgctgtccgaactgaagtcccggccgcgaagtgaacccacctatcctgcagctgttgactggagtgcttacgccagaagtgtcaagccattcttgtctgaacagtcaaattttccggggatgatttattttgatgagctcaccttcattgagcttaagagaaacacaggaaattacaccgtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgagaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagatttgaatga

SEQ ID NO: 158 - protein, 500 aa, Mus musculus
MASLHFPQWAVSFVFFAQAVGSMDTFIAAVYEHAVILPNKTESPVSTEEALLLINKNIDILESAIKLAARQGAHIIVTPEDGIYGWIFTRETIYPYLEDIPDPEVNWIPCRDPRRFGYTPVQERLSCLAKENSIYIMANIGDKKPCNATDPHCPPDGRYQYNTNVVFDSKGRLTARYHKYNLFEPEIQFDFPKDSELVTFDTPFGKFGIFTCFDIFSYDPAVVVVKDTQVDSVLLPTAWYNTLPLLSAVPFHSVWARAMGVNVLAANTHNTSMHMTGSGIYSPEAVRVYHYDMETESGQLLLSELRSRPRQHATPAEVNWSAYARTVKPFSSGQADFPGKIYFDEFSFTKLTGSAGNYTVCQKDLCCHLTYKMSESRMDEVYVLGAFDGLHTGEGQYYLQICTLLKCQTTNSRTCGEPVGSAFTKFEEFSLSGTFRTKYVFPQIVLSGSQLALERYYEVSRDGRLRSRGGAPLPILVMALYGRVFERDPPRLGQGPGKLQ

SEQ ID NO: 159 - protein, 500 aa, Mus caroli
MASLHFPQWAVSFVLFAQAVGSMDTFIAAVYEHAVILPNKTESLVSTEEALLLINKNIDILESAIKLAARQGAHIIVTPEDGIYGWIFTRETIYPYLEDIPDPEVNWIPCRDPRRFGYTPVQERLSCLAKENSIYIVANIGDKKPCNATDPHCPPDGRYQYNTNVVFDSKGRLTARYHKYNLFEPEIQFDFPKDSELVTFDTPFGKFGVFTCFDIFSYDPAVVVVKDAQVDSVLLPTAWYNTLPLLSAVPFHSAWARAMGVNVLAANTHNTGMHMTGSGIYSPEAVRVYHYDMETESGQLLLSELRSRPRQHATPAEVNWSAYARTVKPFSSGQADFPGKIYFDEFSFTKLTGSAGNYTVCQKDLCCHLTYKMSESRMDEVYVLGAFDGLHTVEGQYYLQICTLLKCQTTNSRTCGEPVGSAFTKFEEFSLSGTFRTKYVFPQIVLSGSQLALERYYEVSRDGRLRSRGGAPLPILVMALYGRVFERDPPRLGQGPGKLQ

SEQ ID NO: 160 - protein, 500 aa, Rattus rattus
MVSLYFPQCVVSFVLFAQAMGAMDTFIAAVYEHAVILPNKTESPVSNEEALLLINKNIDILENAIKLAARQGAHIIVTPEDGIYGWIFTRETIYPYLEDIPDPEVNWIPCRDPKRFGLTPVQERLSCLAKENSIYIVVNIGDKKPCNATDPQCPRDGRYQYNTNVVFDSEGRLTARYHKFNLFEPEIQFDFPKDSELVTFDTPFGKFGVFTCFDIFSYDPAVMVVKDSRVDSVLLPTAWYNTLPLLSAVPFHSAWARAMGVNVLAANTHNTRMHMTGSGIYSPDAVRVYHYDMETESGQLLLSELRSRPHQHGSPAEVDWSAYARTVKPFSSEQADFQGTIYFDEFSFTKLTGSAGNYTVCQKDLCCHLTYKMSESRMDEAYVLGAFDGLHTVEGQYYLQICTLLKCQTTNLRTCGEPVGSAFTKFEEFSLSGTFGTKYVFPQIVLSGSQLALERYYEVSRDGRLRSRGGAPLPVLVMALYGRVFERDPSRLGQGPGKVQ

SEQ ID NO: 161 - protein, 510 aa, Peromyscus californicus
MVPLYFPKCVVSFALFAQAVGALDTFIAAVYEHAVILPNKTETPVSAEEALLLMSKNIDVLEKAIKLAAEQGAHIIVTPEDGIYGWVFTRETIYPYLEDIPDPEVNWIPCRDPKRFGYTPVQERLSCLAKDNSIYVVANIGDKKPCNATDPQCPLDGRYQYNTNVVFDSEGRLTARYHKYNLFEPEIQFDFPKDSELVTFDTPFGRFGIFTCFDIFSYDPAVVTVKDFQVDSVLYPAAWYNTLPLLSAVPFHSAWARAMGVNLLAANTHNTTMHMTGSGIYSPEAVGVYHYDMETDSGQLLLSELKSRPRQSTPAAEVDWSAYARSIKPFSSEQADFPGMLYFDEFSFTKLSGSTGNVTVCQKDLCCYLTYKMSEKRMDEAYVLGAFDGLHTVEGQYYLQICTLLKCQTTNLRTCGEPVGSAFTKFEEFSLSGTFGTNYVFPQLVLSGSQLALERYYEVSRDGRLRSRGGVPLPVLVMALYGRMFEKDPPRLGQGPRKLQRNPFTGDCQD

SEQ ID NO: 162 - protein, 501 aa, Molossus molossus
MIASYFPKYGAVLALFVLSVGALDTFTAAVYEHAVILPNRTETPVSKEEALLLMNKNIDVLEKAVKLAARQGAHIIVTPEDGVYGWVFTRETIYPYLEDIPHPEVNWIPCKDPQRFGYAPVQERLSCLAKDNSIYVVANIGDKKPCNASDPQCPPDGRYQYNTDVVFDSEGRLVARYHKYNLFAPEIQFDFPKDSELVTFDTPFGKFGIFTCFDIFSHDPAVVVVTEFQVDSVLYPTAWYNTLPLLSAVPFHSAWARAMGVNLLAANTHSTRMHMTGSGIYAPEAVKVYHYDMETESGQLMLSELRSRPRREHAYPAPVDWSAHAKGVKPFSSEQSDFPGMIYFDEFTFTELKRKAGNYTVCQKDLCCHLTYRMSEKRTDEVYALGAFDGLHTVEGQYYLQICTLLKCQTTDLGTCGEPVGSAFTKFEEFALSGTFGTSYVFPQLILSGSQLAPERHYEVSRDGRLRSRSGAPLPVLVMALYGRVFEKDPPRLGQGPGKCQ

SEQ ID NO: 163 - protein, 501 aa, Homo sapiens
MIISHFPKCVAVFALLALSVGALDTFIAAVYEHAVILPNRTETPVSKEEALLLMNKNIDVLEKAVKLAAKQGAHIIVTPEDGIYGWIFTRESIYPYLEDIPDPGVNWIPCRDPWRFGNTPVQQRLSCLAKDNSIYVVANIGDKKPCNASDSQCPPDGRYQYNTDVVFDSQGKLLARYHKYNLFAPEIQFDFPKDSELVTFDTPFGKFGIFTCFDIFSHDPAAVVVDEVSIDSILYPTAWYNTLPLLSAVPFHSAWAKAMGVNLLAANTHNTSMHMTGSGIYAPEAVKVYHYDMETESGQLLLSELKSRPRREPTYPAAVDWHAYASSVKPFSSEQSDFLGMIYFDEFTFTKLKRNTGNYTACQKDLCCHLTYKMSEKRTDEIYALGAFDGLHTVEGQYYLQICALLKCQTTDLETCGEPVGSAFTKFEDFSLSGTFGTRYVFPQIILSGSQLAPERHYEISRDGRLRSRSGAPLPVLVMALYGRVFEKDPPRLGQGSGKFQ

SEQ ID NO: 164 - protein, 501 aa, Felis catus
MITSYFPKCVALLAICVLCVGALDTFIAAVYEHAVLLPNRTETPVSKEEALLLMNKNIDVLENAVQLAARQGAHIIVTPEDGIYGWVFTRETIYPYLEDIPDPEVNWIPCTDPQRFGYTPVQQRLSCLAKDNSIYVVANIGDKKPCNASDPQCPPDGRYQYNTDVVFDSEGRLVARYHKYNLFAPEIQFDFPKDSESVTFETPFGKFGIFTCFDIFSHTPAVVVVDEFQVDSVLYPVAWYNTLPLLSAVPFHSAWARAMRVNLLAANTHNTSMHMTGSGIYAPEAVKVYHYDMETESGQLMLSELKSRPRSEPTYPAAVDWSAYARSVKPFLSEQSNFPGMIYFDELTFIELKRNTGNYTVCQKDLCCHLTYKMSEKRTDEVYALGAFDGLHTVEGQYYLQICTLLKCQISDLRTCGEPVGSAFTKLEEFSLSGTFATRYVFPQIILSGSQLAPERHYEVLRDGRLRSRSRTPLPVLVLALYGRVFDKDPPRLGQGPRRFE

SEQ ID NO: 165 - protein, 503 aa, Tachyglossus aculeatus
MIFTAFPVYAVFFALWAPGARTEDSFIAAVYEHAVILPNGTQTPVTQDDALMLMDKNLDVLEKAIKQAASEGAHIIVTPEDGIYGWVFTRDTLYPYLEDIPDPQVNWIPCTDPNRFGRAPVQKRLSCLARDNSIYVVANMGDKKPCNTTDSQCPRDGRYQYNTNVVFGADGKLVARYHKYNLFEPEVQFDFPKDSQDVTFETPFGKFGIFTCFDIFSHDPAVTVVEKYQVDSVLYPTAWDNTLPLLSAAPFHSAWARSMRVNLLAANTHNTSMHMTGSGIYAPDGIRAFHYDMETESGQLLLSELKSRPRLWPSYPVAVDWSAYAKHVKPPSREYKKFGGKIYFDEFTFTDLIQAGENYTVCENDLCCHLSYKMTEKRADEVFVLGVFNGLHIVEGQYYLQVCTLLKCQTTDLRTCGEPIGSAFTKFEEFSLSGTFGTNYVFPQVILSGSQLATEQQYEVTSDGRLRNRGSVPLPILVMALYGRVYEKDPPRLGQGVRPLRPL

SEQ ID NO: 166 - protein, 483 aa, Myotis lucifugus
MITSSFQASVAVLALVTLHVSALDTFIAAVYEHAVLRPKGTETPVSRDDALLLMNKNIDILEKAIKQAAEQGAQIIVTPEDALYGWKFTRETIFPYLEDIPDPQVDWIPCQDPQRFGHTPIQTRLSCLAKNNSIYVLANIGDKKRCNSQDSTCPPNGYYQYNTNVVYDAKGQLVARYHKYHLYSEPQFDVPEKPELVTFNTTFGRFGIFTCFDILFHDPAVSLVKDFHVDTILYPTAWMNVLPLLSAIEFHSAWAMGMRVNLLAANIHHVQQNMTGSGIYAPHSPRVYHYDMETDSGRLLLSEVDSRPRTSPAYPTAVDWSAYATTIKPFPVQKNTFSGFISRDAFNFTALFENAGNLTVCHKDLCCHLSYRMLRKEENEVYVLGAFTGLHGRRRREYWQVCTMLKCKTTDLTTCGQPVETALTRFDIFSLSGTFRTEYVFPEVLLTKVHLSPGKFEVGGLEEYFYFICSLKPSKIGYGNCIT

SEQ ID NO: 167 - protein, 514 aa, Equus asinus
MISSQLLAYVAVLVFCTFQARSLETFIAAVYEHAVILPNATLTPVSREEALLLMNRNLDLLEGAIASAAKQGAHIIVTPEDGVYGWNFSRDTLYPYLEDIPDPEVNWIPCNNPHRFGHTPVQERLSCLAKDNSIYVVANFGDKKPCNASDPECPPDGRYQYNTDVVFDAQGKLVARYHKQNLFMGEDQFNAPKEPEVVTFNTTFGKFGIFTCFDILFYDPAVTLVTDFQVDTILFPTAWMNVLPHLSAIEFHSAWAMGMGVNFLASNIHYPSMKMTGSGIYAPDSPRAFHYDMKTEEGKLLLSQLDSHPHHPAVVNWTSFASGVKTLSTGNQEFNSTVFFDEFTFLELTEVTGNYTVCQKALCCHLSYKMSEKRSDEVYALGAFDGLHTVEGSYYLQVCTLLKCRTTDLHTCGDSVETASTRFDTFSLSGTFGTQHVFPEVLLSEIQLAPGEFQVSSDGRLFSLQPPSGPVLTVTLYGRLYEKDQVSDASSDLTAQALRVMLVVLIPVGFSISW

SEQ ID NO: 168 - protein, 514 aa, Egretta garzetta
MLHKKHSRSCEAAAMLPSQSLLHAAVFALTVLQALASDTFIAAVYEHAVILPDATDEPVSPDDALALMNKNMDVLEGAVKEAAQQGARIIVTPEDGIYGWRFTRETIYPYLEDIPDPAVNWIPCTDPTRFAPAPVQERLSCMARSNSIYVVANIGDKKPCNSSDAGCPRDGRYQYNTNVVFDSEGKLVARYHKYNLFMSETQFNYPKEPEAVTFETPFGKFGIFTCFDILFREPAVVLVSELQVDTVLFPTAWMNVLPFLTAVEFHSAWAMGMGVNFLAANTHYTNISMTGSGIYAPNGARTYYYNMKTEDGHLLIAELDSHPRLSPASPPAVSWSSYASSVESFSPNDHDFGGLIFHDWFTFTELTKPEGNLTVCQKDLCCHLSYKMAGKQKDEVYVLGAFDGLHVVEGQYYLQICTLLKCKNTDSNTCGQPVESAQTRFERFSLSGTFGTSYVFPEVLYSGVQLAPGEFEVLNDGRLISKKRTTKPVVTVTLFGRWYEKDHLKQDPQSPAFP

SEQ ID NO: 169 - protein, 520 aa, Bos javanicus; identical to SEQ ID NO: 115

SEQ ID NO: 170 - protein, 521 aa, Sus scrofa
MIASSFQTTLAVFALIILHVRALDTFKAAVYEHAVILPNDTKTPVSWDDALLLMNKNMDILERAIKQAAEQGSQIIVTPEDALYGWKFTRETIFPYLEDVPDPQKNWIPCQDPRRFGHTPVQVRLSCLAKNNSIYVLANIGDKKPCNSHDSKCPPNGYYQYNTNVVYDSEGKLVARYHKYHLYHELQFDVPEKPEVVTFNTPFGKFGIFTCFDILFRDPAVTLVQDFHVDTILFPAAWMNVLPLLTAIEFHSAWAMGMRVNLLAANVHHVRLNMTGSGIYAPHSPKVYHYDMETDSGKILLAEVDAHPRTSPTYPTAVNWGAYTLSIQPFPILQNSFRGFISRDEFHFTELSESAGNLTVCHKELCCHLSYRMLAKEENEVYVLGAFTGLHGRRRXEYWQVCTMLKCKSTNLTTCGQPVETALTRFELFSLSGTFGTEYVFPEVLLTKINLAPGKFEVLKDGRLVNKNGTSEPVLGVSLFGRWYIRDVPLSISGGPAPQQKMYLIISALLMIIALQNIVMV

SEQ ID NO: 171 - protein, 546 aa, Bos indicus
MLHKCSFLYKYYMNVTFTCRTQDTLAMITSSLQTSVAVFALXILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLRSLAKNNSIYVVANMGDKKPCNSHNPXCPSNGHYQYXTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAGNLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYXKDAHSISGGTSNSATAYLMIPTLLMIITLRDAVMX

SEQ ID NO: 172 - protein, 520 aa, Patella sp.; identical to SEQ ID NO: 130

SEQ ID NO: 173 - protein, 561 aa, Biomphalaria sp.
MGKEVRLLLLLLGTLSSVLANRSKFKGAVYGHAVLLPKDSLKVISRAEALDNMNKNLDVYREQVTKAKSQGAEIIVFPEDGLYGFQFTRQSIYPYLEYIPDPRTELWSPCDEPGRYNDTEVQRSLSCLAKTNSLYLVANMGDKQPCDAATDRTCPPDGRYQYNTNVVYGPDGRLLARYHKYNLFYELQFNAPELELSYFDTPFGRFGLMICFDVLFHDPGLPLILSLNISNIIFTSAWKDALPLLSALGYHSSFARGLGINMLGANIHIPDYRFHGSGIYGPDGTIISFYGSTNASKPKLLVADVNVLDDTQPNKIVSSIVQQNLKQARMNEADDKRVTKDTTKNSKKTFESLLFYDMFTFAALDKPSGHIKVNQKHVHCELRYSIPEELFGRELYAFGAFDGLHTYEGQYYFQICALVKCADARNRSTCGSVTYDAITPFVRLSISGEMQTPFVYPQIILSNGIGELSLAEPDSWTYTGKSLKTDKEFYKPVLSAALFGRDYFRDTDGQNKGDNLYKSQEDATDKEDKSEIYLGLVSGQNIFIFLLGFVCLLLLVNMAIF

SEQ ID NO: 174 - protein, 501 aa, Mya arenaria
MTVGSPGMLLTLLLVLGGRILAGLETPLADSYRAAVYEHNVSFPRHRDVVPRLEALQLMEENLGKFDEQARQAAQQKAKIIVFPEYGVYGLGWTRQKLAPFLEYIPDPAGNPWNPCDNPNGFVSAVQTLLSCIAKNNSIYVVANIGSQQPCISTDKSCPSDHVYQFNTNVVYDPRGIFIARYFKYNLDSVEMQYFDRPPVVNHTIFDTPFGRFATVCSNDILYKEPTLTLVKTMNITNLAMPAAWKEELPLMSAIEFHSAFAEGLLINVLAANLHVVDLGYYGSGLYWPTGTSINASYFYDDDPYSEGSLVVETLTPSIIPSDVWDPPQFTENKRFAPRRLVNNAEQSSLTHKIKINGDIYTAKVFKFGEAYSANVCQGSLCCDADFEGGASGDMFAFGAFEGMHSGNNSYYLQSCALIKCAGRTVESCGQPTKHTSTGIDKMSMSGNFSYPYQFPEVFTVGDHPEIVTQIWYYQGSVIIDVGLYGDLLTVAIQAIDYSRQ

SEQ ID NO: 175 - protein, 537 aa, Dissostichus mawsoni
MKHLYSRTQMLFMSLREGTMFLSVIVFSSLSVSTALGRTEPVVDSSYIAAVYEHNIILNPDPRVPLSRPAALQHMQRNLDIYEQQAARAAQQGVQILVFPEDGIQGFNFSRSSIFNYLETIPDPQQESWNPCTEPGKYNNTEVLQRLSCMARRHNLYLVANMADLQPCPLKTDPSSSCPSDGRWQFNTNVVFRSDGLLVGRYHKNNLYFEAAFDTPPQPEIITFDTPFAGRFGLIICFDILFYEPKVHLVTKGVRQLIFPTAWMNQLPLLDAIQFQHAFSLGANVTLLAANIRNDQMIMTGSGIYTPFSVKSHHAQKGDPDEGRLLVARVPVLDPLWAGQSVTDGSTAKDSGFCHKESCLDFPAPETSSSVPPSSSASSTSTFISSMMYDPFTFVLLEATEGKVNVCNGTFCCHLQYRLIPQGDSKELYALGAFGGTHTVNGRYAVQVCALVRCAGLDTSSCGQEVEEAESKMDFQLEGRFASRYVYPSVLASKFVLEQPKHFETAPDGTVTMKHSNMTGGLVTACLYGRMYHLENE

SEQ ID NO: 176 - protein, 620 aa, Priapulus caudatus
MAPIRLQCLRVGSPHRRALVVAAVLLATLQYPSATATARRTSSDDSSCTYTAAVLQYYLQTPGPGSDPSEVMRSNLLAYEEYLMQAAAQGASIIVFPETGITGFLQKDREIVLKWLQEIPEPNTEAALDGQRSSDSPDSCDSGCVATSGDDFVLESLKCMAACHKITIVANLGEVQYCQPAASSTVPTQVQSSAAAEDAVPGCPPDGRFQFNTNVVVDDNGALVVRYRKQHLYNEAAFDDPPVPEAGVFDTPFGRFTTFVCFDINFYNTSADVVLHDGVDSVAMSAAWPIALPTSSPQQTHSGWALGTGVNLLTANVRGVTDGLTFGGSGIYSGVSGNLTYTREDAPMQPELVIADVPCYRKRSWESYAHRPYSVEIYDAPVAAIARPAGGGGSGNKRRMKNKKNSRKRNEHRRRPGNEPNSENSETGDNSPRTGGEREQHTSAPPPKYFKNYVIGVLFNFTALEADIGRASVCYGDLCCHLDYAMSARGRRSDELYALAAFNDYYTAVTDQFWWQVCMVVKCRTSSYKTCGESVTRAKTKFKALTLSGDFWVEFISPSVRVDGDYLLAPLDQWSFDNDGEVMFSTKGIADPLITAEIQAFEPEKHDQSKRGVDVTCTCT

SEQ ID NO: 177 - protein, 526 aa, Ruditapes philippinarum
MDYYLWNMEAKKNVVCCFLLSIGISMITSAKVKVAVYEHVVISPDDPEALFTRQEAFQWMSKNLNVFVEQARKASEQGAKIIVFPEYGITGFDHSRNSIVPFLEDIPDSSTDWNPCMEPDRFADTLVLRNLSCTAQMYKIYIVANMGDMKTCDKMTDETCPHDGRYQFNTNVIFDNNGQLVARYHKLHMYDETPLFDPAPNAEHVYFDTPFGRFGTIVCFDIMQNEPTQVLLNKYSITNLLVTSAWNVFLPFIIPQQMYSGLAKKSGINVIASNIRNSLYQMAGSGIFGNDIQSLSDVNSNGAEGILLVSEIETEEAKIYAPNTIMSINSSGKVKQFINKTYEGTYRFGLNMSYAIMEGQSGIVTSCKDDVCCVVTYQFKSRDENETIILSVVDELLYKPGIIHMQYCAVFKCSPHTIYSCDYDDIFVINSIFADIKIQGYFDNGVTFPLVTTIDNNELTMDMDKYTFDDKNAILHSNQFENPLLAAVVYNTLHLDVIKTGASSNVAQGTTLSLSTFLLIYIYKAS

SEQ ID NO: 178 - protein, 511 aa, Paramuricea clavata
MAKQTLFFMLFVLSISSRILAKEYKVATFEHHPFFILAPEPLSREKALQMMRKNTAVYSEQARAARKQGAQIIVFPEDGIYGFFHSRETIKPYLEPIPTVSNTTSIIPCTDPKPNTEILQQLSCIARNNSIAVVANMGDIQYCSQRDPNCPKDGRYQYNTDVIFDKDGRLLAKYHKQHLYFEKAFNTPTKCEFVTFVTSFQVRFGVFTCFDMLFKDPAIDLVQTYGVQNIVVPTAWYKGFPALVSIEYHQAWSRVNCVNLVTANVYYPNRGLNFTGTGIYDCGEAKTYTYGEETIEKRLLFATLSGNREAQNDKTDMLYRWRRDIEMVKEFGMDRKIPQDIDNENRMLDDPYDSIKTFKAKIMGNVFTLTKLTSPASELSVCVKGLCCNLKYGLPEGKQFTETFAMGTFIGNNPDGFYWEVCLLLKCASSEESSCGTTVMDSGTVFQSFTMSGNFSSTALVFPMALGSGVSLFSTDKVKFANKSISGKALQKPLLTASLLGRVYSKDKPHN

SEQ ID NO: 179 - protein, 559 aa, Nilaparvata lugens
MGDIQYSIFKSEIHNHNNAVERKKMISTVRSFQRLFLLFLVACTLFATSIIVNGYLNSGRDKYVAAVVEYAPLEWDGFEKPKDLFLRNANRFVTIIERAAKENADIIVFPECGLTTLNIPVKREGITEYTTFIPNPVEKRTPCTDNDTGLAEHLRVLSCAAKNNKMYVAVNMDEKITCESDMQGCPKDHLIFFNTNIVFDRNGTIIAKYRKFNLFKEPEFNSTIFPIYAVFKTDFGATFGMFICFDILFAQPAEVIVSKYKVTDILYSTAWFSELPFLTAVQTQAGYSYSMNVNLLASGYNRPDLYSGGSGIYAGEDGPVVTTMPAFRTTRLLVGELTIYGTGRKRPVCEYADSCPLSKGKVIDIHFNDTHDKEGMLFLKDDLSKYSTEFIEETTSYEHAMIQHLGDDKETNLEYSRNGFNCSITVTWFRDDTSSNNKGPVYRLVAHSGIRSFAGVRSAYIEVCALVACADRTIESCGHRPSFENVSSRTQVKNVNIHTESLNITTPFVSTLDSNIYPLMGNELSFIESIDKFRGYIQKTYLFLEKPKDDLITFGVYKL

SEQ ID NO: 180 - protein, 512 aa, Ischnura sp.
MGLLHGILFVLLIPLCAAHRKTYRAAVVEYHPLGDRHDNASHILNLNVAEYETLIEEAKRKGADIVVFPEYGLTQLAASSSRNLAIQFAQSVPTPPAPGTPSSAAQVPCLLPNSSMAIRRLSCSARENQIYVVVNLLEIAPCSITDHACPEDGFFIYNTNVAFDRNGGVVSKYHKYNLFKEPSVNTPKHSMYSTFDTDFGVTFGMFICFDILFEDPGYTLVKRMNVNNMIYSTAWISELPFLTATQTQQSWSLGLGVNLLASGFNNPMGGNGGSGIYAGGSGRLNLLMPSTAGTTLLLADVPVVSSNRLRGDSLSEPELSLDLQTGNQATSGHMFVLHDDLSNYKSSEVLKNGLNELNMCQNNLCCNFKADVQLSSNGSQAPKYRFVITDSIIKYARTYPAHVQVCGLIYCKGDDVSKCVYFPDLPETLDNVSFRSLGISGNFSSPRMVLPSTLTTSLEPLKISSGNLMEKDEAITLEYNSDTRGLLTFALYGRIFQQNMIRNEEIMDNDIY

SEQ ID NO: 181 - protein, 533 aa, Argiope bruennichi
MKLPGIQFLCFFVMLSMGSAQMRSRRPYYRAAVHEHKQYDDKTSSGREIINKNLRKYEVAARLAARNYVDLIVFPEKGLFPMKMDNPTWFLDYAENIPRGDEMTNPCYQEKFSSSPILTNLSCIARKYSLFVVATLIDVKDCKVRRHCENRKNKNSCVSDGSDCPDNGHFNFNTLVVFDRMGTLIVRYYKRHPFIPLEKGISTPQYPESRYFDTEFGSFATDIGFDFLINDSFIDIAKRPETTGVTYGNWWFDHTPFFYFSVPNQQAWCLTNKVTVLSSDVHSPNLASLGSGIYIPGKGAVIYSYNPDGKSKLLISNIPTSFFYDFQSTLPPLNKRFFYINDDDSISELNGEEPRNFKDKCGENVLGMNPSSLTDYRCKQSEVHQYTFVKLNKTEDYINVCSNNFCCSLEYQAESMDETFYFAVSGNRLKFYDIYWFGVQSCFLSRCEPVDSKPCRNFLLKSNTKFNSVKITGSFDTNHIYPHVLDSELRLTDRDEWKFDGTSQLSYQNLKGKNLLHADLYGRLYREDGLVNN

SEQ ID NO: 182 - protein, 573 aa, Patella sp.
MMYKIMLLRIVATYTCLSLVEGYRPSYRAAVYEHALFQPTHFRTVSRNQALRDMKKNLDIYAEQTRIASSQNVDIIVFPEDGIYGLNFNRDTIKPYLEDIPDPTEITWNPCNQSEYTNGVEVQITLSCMARNNGIYIVANMGDKKVCDADSICPDDGQRQYNTDVVFDPQGNLVARYHKQHLYFEAQFDTPSTVERVTFDTPFGKFGIITCFDILFKSPAVDLVEDLGVKNVIFPTAWMDSLPLLSAIQFHSAFAKLFKVNLLAANLHFPLFNFHGSGIYSPRRTEAYYYNDGLFSPGKLLIADVKSSPTHYRRHQVVPDTHGFRGGNSLGASFVHGQDTAEVNTQSRRNGGHYNTASRNGIISHQLADQASGVHLNNDLGPLTHRSIRNRIHNHAHLHRVNRHMVENGWATIMADNDNYQYLPLTEPKAKVKICQNEVCCYLDYQKQNTSNDELYALGVFDGLHQIEGLSWYTQVCVLLKCEFTDIASCGQPTKIAAGHFEYLKLWGTFTTEYVFPEVLVSDDGKLRLEHEHWKYLHNRIEFVNRGKERPLLSATLYGRHYDRDNSTVSNSV

SEQ ID NO: 183 - protein, 568 aa, Lonchura striata
MLYHDPAVVMASRFQINTILFLTAWVNTLPLLSAAQFYLVQALGMGVNFLSANTHNSTLDMTGSGIYAPDSPRAYYYNTEMDNGHLLVTELSSHPHLSSDYPVAVNWTLSAIFPCSTCGQPVETVQTKFEMFSLSGIFGISYIFPEVLYSGVQLAPGEFKGAHIIVTPEDGIYGWRFTRESIYPYLENIPDPVVNWIPCTDPSRFGPAPVQERLSCMARNNSIYVVANIGDKKPCDSSDPSCPRDGRYQYNTDVVFDAQGKLVARYHKYNLFLGEDQFNYPKEPEAVTFETPFGKFGIFTCFDILFYEPAVVLVSKMQVDTVLFPTAWMNVLPFLTAIEFHSAWAMGMRVNVLAANTHNTSMEMTGSGIYAPAGARTYSYNKKTEDGHLLIAELDAHPRLSPVSPPAVSWNSYALSVERFSQNDHEFTGIIFEDLFTFTELTKPGENLTVCQKNLCCHLSYKMAEKRDDEVYVLGAFDGLHVIEGQYYLQICTLLKCPSTNLSTCGQPVETAQTKFEMFSLSGTFGTSYVFPEVLYSGVQLAPGEFKVMGHPCDLSDISGQKPTTRAT

SEQ ID NO: 184 - DNA, 642 bp, Corynebacterium glutamicum
  source 1..642: mol_type = genomic DNA
gtggctgctagcgcttcaggcaagagtaaaacaagtgccggggcaaaccgtcgtcgcaatcgaccaagcccccgacagcgtctcctcgatagcgcaaccaaccttttcaccacagaaggtattcgcgtcatcggtattgatcgtatcctccgtgaagctgacgtggcgaaggcgagcctctattcccttttcggatcgaaggacgccttggttattgcatacctggagaacctcgatcagctgtggcgtgaagcgtggcgtgagcgcaccgtcggtatgaaggatccggaagataaaatcatcgcgttctttgatcagtgcattgaggaagaaccagaaaaagatttccgcggctcgcactttcagaatgcggctagtgagtaccctcgccccgaaactgatagcgaaaagggcattgttgcagcagtgttagagcaccgcgagtggtgtcataagactctgactgatttgctcactgagaagaacggctacccaggcaccacccaggcgaatcagctgttggtgttccttgatggtggacttgctggatctcgattggtccacaacatcagtcctcttgagacggctcgcgatttggctcggcagttgttgtcggctccacctgcggactactcaatttag

SEQ ID NO: 185 - protein, 213 aa, Corynebacterium glutamicum
MAASASGKSKTSAGANRRRNRPSPRQRLLDSATNLFTTEGIRVIGIDRILREADVAKASLYSLFGSKDALVIAYLENLDQLWREAWRERTVGMKDPEDKIIAFFDQCIEEEPEKDFRGSHFQNAASEYPRPETDSEKGIVAAVLEHREWCHKTLTDLLTEKNGYPGTTQANQLLVFLDGGLAGSRLVHNISPLETARDLARQLLSAPPADYSI

SEQ ID NO: 186 - DNA, 669 bp, Corynebacterium glutamicum
  source 1..669: mol_type = genomic DNA
atggcaggagcagtgggacgcccccggagatcagctccgcgacgggcaggcaagaatcctcgcgaggagattcttgacgcctctgctgagcttttcacccgtcaaggcttcgcaacaacctccacgcatcaaatcgctgatgccgtgggaatccgccaagcctcgctgtattatcacttcccgtccaagacggaaatcttcctcaccctgctgaaatctactgtcgagccgtccactgtgctcgccgaagacttaagcaccctggacgccggacctgagatgcgcctctgggcaatcgttgcctccgaagtgcgtctgctgctgtccaccaagtggaacgtcggtcgcctgtaccaactccccatcgttggttctgaagagttcgccgagtaccacagccagcgcgaagccctcaccaacgtcttccgcgacctcgccaccgaaatcgtcggtgacgacccccgcgcagaactccccttccacatcaccatgtcggtgatcgaaatgcgtcgcaacgacggcaagattccaagcccgctttccgcagacagcctcccggagaccgcaattatgcttgccgacgcctccctcgccgtcctcggcgcgccgctgcccgccgaccgggtcgaaaaaacgcttgaactaatcaagcaggctgacgcgaaataa

SEQ ID NO: 187 - protein, 222 aa, Corynebacterium glutamicum
MAGAVGRPRRSAPRRAGKNPREEILDASAELFTRQGFATTSTHQIADAVGIRQASLYYHFPSKTEIFLTLLKSTVEPSTVLAEDLSTLDAGPEMRLWAIVASEVRLLLSTKWNVGRLYQLPIVGSEEFAEYHSQREALTNVFRDLATEIVGDDPRAELPFHITMSVIEMRRNDGKIPSPLSADSLPETAIMLADASLAVLGAPLPADRVEKTLELIKQADAK

SEQ ID NO: 188 - DNA, 1371 bp, Corynebacterium glutamicum
  source 1..1371: mol_type = genomic DNA
ttagaacaacgccccagcgaaggtcagctcgacagcttgggctgcagcttccaccgcgtccagcagtgctaccaattcgtcatcagtggtggtcagcggcggcgcgatgtggaatcggttgccggagatcatcggccacacgccgcgttccttgaattctgcagcaccggcagccatggcagtggcgtctgcattgaactccactgcccagaagaatccgatgccccgcacgtcagcgatcgctacgttttcttccgctagttcacgaaggcgaggttcgatcagttcagcgccaagtcgagctacgcgtggaatgatctctccttccgcgtaaatctccagcgctgccttggcgggtgctactgcaagtgggtgtccggagtaggtgagtccgccggagtatgcctctgatccgaaggtatcgcggattgattgggtcatcacgatgccaccgagtggggcgtaacctgcgttaacacccttggcgaaggtgatcatgtctggctggaaatcgtcgccagcatgctcgtaagcaaacagttttccggtgcgtccgaatccgaccatgacttcgtcggcgatgaagaggatgccgtgcttgttgcaaagttcgcgcacgccatttaagtaacctgctggtggcaggatgattcctgatgatcccaccactggctccaggacgatcgctgcgatcatgccagcaccttcaaacgcgatgacatcttccaagtgcttgagtgcgcgttcgcattcttcttcttgggtggtggcaaagaatgaggagtggtgcaggaatggtgcccagaagtggtagatatctgggtcggtggtggggttgcccaggcggcggtgttcgccggtgagcatcatcgcggatccggttgcgccgtggtagctgcggtatgcggacagaattttgttgcgtccggtgtgcaggcgagccatgcggatggagtgctcgatggcgtcggcgccgccgttggtgaaaaacacgtgggagaattcgccacgggccatcgacacgatctttgcagcaacatcagagcgcacatcattgccgaaggccgggttgatgttggtcaaccgggctgcttggcgctggatcgcctcaactaatcgagggttgttgtggcctaagtttgccgagacaagttgggaacccatgtcgatgaaggcgttgccgtcgaagtcgtacagcgtggaaccttcggcagctgcccatactttggggctgattttgtcctgcgcggaccaggagtggaaaacgtgcgcgcgatcgttgtcgaatgcccgtttttcctcttcttttgcttgcacagatccgaattcgatgaattcaccgtcaaagttggtgtaacccttcaatgccaa

SEQ ID NO: 189 - protein, 456 aa, Corynebacterium glutamicum
MALKGYTNFDGEFIEFGSVQAKEEEKRAFDNDRAHVFHSWSAQDKISPKVWAAAEGSTLYDFDGNAFIDMGSQLVSANLGHNNPRLVEAIQRQAARLTNINPAFGNDVRSDVAAKIVSMARGEFSHVFFTNGGADAIEHSIRMARLHTGRNKILSAYRSYHGATGSAMMLTGEHRRLGNPTTDPDIYHFWAPFLHHSSFFATTQEEECERALKHLEDVIAFEGAGMIAAIVLEPVVGSSGIILPPAGYLNGVRELCNKHGILFIADEVMVGFGRTGKLFAYEHAGDDFQPDMITFAKGVNAGYAPLGGIVMTQSIRDTFGSEAYSGGLTYSGHPLAVAPAKAALEIYAEGEIIPRVARLGAELIEPRLRELAEENVAIADVRGIGFFWAVEFNADATAMAAGAAEFKERGVWPMISGNRFHIAPPLTTTDDELVALLDAVEAAAQAVELTFAGALF

SEQ ID NO: 190 - DNA, 492 bp, Corynebacterium glutamicum
  source 1..492: mol_type = genomic DNA
atgacttcagcaatcaccaccgcaactgatcttcgctccgtactgcgaaacgtaccaaccccaattagcttcattgcaacccacaccgatcagcctctgggcatgatcgttggttcattcgtcagcattagcgccgaaccaccattggtaggcatcttcttgcagaagagctcttcttcatggccagctatcgagcaggcattagttaccggccaagagctaggcatttctatcctcggcggggcacacgcagaccatgtgcgtaagctttctggcccatccgaccagcgctttgaaaaccttgggtgggcatccaccgaaaacggtgcgattcaccttgaaggcgctgatgcacaactaaccacgaaacttcatgatctccaggaaatcggcgatcacttctttgcagttctagaagttattgacgcttccgctgaccaagacttcagctcagcgctggtgtaccaccgctcacaggtgtcctcgctgtag

SEQ ID NO: 191 - protein, 163 aa, Corynebacterium glutamicum
MTSAITTATDLRSVLRNVPTPISFIATHTDQPLGMIVGSFVSISAEPPLVGIFLQKSSSSWPAIEQALVTGQELGISILGGAHADHVRKLSGPSDQRFENLGWASTENGAIHLEGADAQLTTKLHDLQEIGDHFFAVLEVIDASADQDFSSALVYHRSQVSSL

SEQ ID NO: 192 - DNA, 1146 bp, Corynebacterium glutamicum
  source 1..1146: mol_type = genomic DNA
atgacattaactttccattggttcctatccacttcaggcgattcccgcggcatcatcggcggcggtcacggtgcagaaaaatccggcacctcccgcgaattgagccacagctacctcaagcagttggcgctagctgccgagaccaacggttttgaatctgtcctgacaccaacgggcacgtggtgcgaagatgcgtggattactgacgcttctttgattgaggcgacaaaacgcttgaagttcctcgttgcgcttcgccctgggcagattggacctacgctgtctgctcaaatggcttctactttccagcgtctgtctggcaaccgtttgctgatcaatgtggtcaccggtggggaagatgcggagcagcgtgcgtttggtgatttcttgaacaaggaggagcgctacgcccgtaccggagaattcttggatatcgtgagccgcttgtggcgaggcgaaaccgtcacgcaccacggtgaacacctgcaggtggagcaagctagccttgcgcatccgccagagattattccggagattctttttggtggatcgtcgccagctgcaggtgaggtggctgcacgttatgcggacacctatctcacgtggggtgaaactcccgatcaggtggcgcagaaaatcaactggatcaacgagctagcagcacagcgcggccgggaactgcgccatggaatccgcttccatgtgatcacccgcgatacgtctgaagaagcatgggtggtggcagagaagttgattagcggggtcactccagaacaggtcgctaaggctcaagccgggtttgcaacgtctaagtcggaggggcagcgccggatggctgagctgcacagcaagggtcgtgcctttactagtggctcaactgctcgtgatctggaggtgtatcccaatgtgtgggcaggcgtcggtttgcttcgcggaggtgcaggaacagcccttgtgggctcgcatgaagaggtcgccgatcgcatcgaagaatacgcagcactcggcttggatcagtttgtactgtcgggttatccaaacttggaggaggccttccacttcggtgagggtgtgattccggagctgctgcgccgcggtgtggatatcaaaaatcaagaatcacgagttttggaacctgttgggtaa

SEQ ID NO: 193 - protein, 381 aa, Corynebacterium glutamicum
MTLTFHWFLSTSGDSRGIIGGGHGAEKSGTSRELSHSYLKQLALAAETNGFESVLTPTGTWCEDAWITDASLIEATKRLKFLVALRPGQIGPTLSAQMASTFQRLSGNRLLINVVTGGEDAEQRAFGDFLNKEERYARTGEFLDIVSRLWRGETVTHHGEHLQVEQASLAHPPEIIPEILFGGSSPAAGEVAARYADTYLTWGETPDQVAQKINWINELAAQRGRELRHGIRFHVITRDTSEEAWVVAEKLISGVTPEQVAKAQAGFATSKSEGQRRMAELHSKGRAFTSGSTARDLEVYPNVWAGVGLLRGGAGTALVGSHEEVADRIEEYAALGLDQFVLSGYPNLEEAFHFGEGVIPELLRRGVDIKNQESRVLEPVG

SEQ ID NO: 194 - DNA, 1179 bp, Corynebacterium glutamicum
  source 1..1179: mol_type = genomic DNA
atgagtgaaaacaatcccactaccttgcactggttcctacccacctatggcgattctcgcggaatcacagccggcgggcatggcttcggcttccactccggaagccggacagcagacctcgattacctctcccaaattgccctggccgctgaacgaaacggttttgaatccgtcctgactcccactggattgtggtgcgaagatgcgtggatcaccaccgcagcgctgctgtctaggacatcaaaactgaaattcctcgttgctattcgaccaggccaagttagccccaccatcatcgcgcagcagggtgctgccttccagaaattctcaaataaccgcctgctcatcaacgtcgtggtgggtggcgaagaccatgaacagcgcgctttcgctgattattcttccaaagaggagcgctaccacaaggctgatgaaaccttagagatcatcgatcacctatggaacagcgcagaacctctaaatttccagggtgaattcctcagtgtggaaaacgcggtattgaaggaacagcccgaggtttccccaccgatttactttggcggatcctcacaactcggcatcgaaatcgcagcccaacattccgatgtttatctcacctggggtgaacctgcggaaaaggtagaggagaagcttgcccgggtgcgcgccgaagcagataagcgaaaccgcgaactagactatggcatccgcctgcatgtcattgctcgaccaactgaggatgaagcctggtcagtggctcaaaatcttcttgaccaacttgatcaggaagaggttgcccgcattcaggaagggcttgcgcgttctcaatcggaaggtcagcgtcgcatgacggaacttcatggacaaggggcagcattcacagcaggagcagatgctcgctcccttgaaattgcaccgaatctctgggcaggtgttgggctagtccgcggtggcgccggcacagcgttggtgggttcctatgagcaagtcgcgcaagcaattttgcgataccgcgatattggtctgagccacttcattttctccggctatccacatttggaggaaacctatcacgtgggcgaaggagtggtacctgagctcctcaaattgggtgttccggtgaacaaccatgaagaacaacgcaacgacgtggtagcgactccgtttatttccagatag

SEQ ID NO: 195 - protein, 392 aa, Corynebacterium glutamicum
MSENNPTTLHWFLPTYGDSRGITAGGHGFGFHSGSRTADLDYLSQIALAAERNGFESVLTPTGLWCEDAWITTAALLSRTSKLKFLVAIRPGQVSPTIIAQQGAAFQKFSNNRLLINVVVGGEDHEQRAFADYSSKEERYHKADETLEIIDHLWNSAEPLNFQGEFLSVENAVLKEQPEVSPPIYFGGSSQLGIEIAAQHSDVYLTWGEPAEKVEEKLARVRAEADKRNRELDYGIRLHVIARPTEDEAWSVAQNLLDQLDQEEVARIQEGLARSQSEGQRRMTELHGQGAAFTAGADARSLEIAPNLWAGVGLVRGGAGTALVGSYEQVAQAILRYRDIGLSHFIFSGYPHLEETYHVGEGVVPELLKLGVPVNNHEEQRNDVVATPFISR

SEQ ID NO: 196 - DNA, 1068 bp, Corynebacterium glutamicum
  source 1..1068: mol_type = genomic DNA
atgcttgcacagcgaacactccccattcacatcaccgccccccacctacccgtcgcgcgcgtatttcatcaaattcgcgccacagacgccgatcgcacctctctgcaacgcgatcttgaactctcccaagctggcatcacgcggcatgtgtcagcgcttattgatgcaggtctcgtggaggaaacccgagtggattccggggcgcgctcggggcgaccgcgcacaaaattaggcatcgacggccgccatctcaccgcctggggagtgcacattggcctgcgcagcacggattttgcggtgtgcgatttagccggccgagtgattaggtatgagcgcgtggaccatgaagtttcacactccacgccgtcggaaacgctgaattttgtcgcacataggttacaaacattgagcgccggcttgcccgagccccgcaatgtgggcgtggcattatctgcccacttaagcgccaacggaaccgtcacttccgaagattatggctggtcagaggtggaaattggggcacacctccccttccccgccaccatcggatcaggtgttgcggcgatggccggttcggaaattatcaacgcgccactgacccaatccacgcagtccacgctgtatttctacgcccgcgaaatggtctcccacgcctggattttcaacggcgctgtccaccgccccaacagcggccgcacgccgacggcgttcggaaatacaaataccttaaaagatgcttttcgacgtggactcacaccaacaactttctccgatttagtccaactctcccacaccaacccgcttgcacgacagatcctcaacgagcgcgcccacaaacttgccgacgccgtaaccaccgccgttgatgttgtcgaccccgaagccgtcgtcttcgccggcgaagccttcaccctggatccggaaactcttcgcattgtggtgacccagctccgagcaaacaccggcagccaactgagaatccaacgcgcagacgcctacattctccgcaccgcggccatccaggtggcgctgcatccgatccgtcaagatccgttggcatttgtgtaa

SEQ ID NO: 197 - protein, 355 aa, Corynebacterium glutamicum
MLAQRTLPIHITAPHLPVARVFHQIRATDADRTSLQRDLELSQAGITRHVSALIDAGLVEETRVDSGARSGRPRTKLGIDGRHLTAWGVHIGLRSTDFAVCDLAGRVIRYERVDHEVSHSTPSETLNFVAHRLQTLSAGLPEPRNVGVALSAHLSANGTVTSEDYGWSEVEIGAHLPFPATIGSGVAAMAGSEIINAPLTQSTQSTLYFYAREMVSHAWIFNGAVHRPNSGRTPTAFGNTNTLKDAFRRGLTPTTFSDLVQLSHTNPLARQILNERAHKLADAVTTAVDVVDPEAVVFAGEAFTLDPETLRIVVTQLRANTGSQLRIQRADAYILRTAAIQVALHPIRQDPLAFV

SEQ ID NO: 198 - DNA, 1065 bp, Corynebacterium glutamicum
  source 1..1065: mol_type = genomic DNA
gtgcgttatattgctttagcgaaagtaacttccatgcatcaacgttcgatgagcttcactttcattcgtacttttttcgtactttttggcatcacgttgttagtgtcatgcgttccagagcctcctgactcctacactaaagaatccactgtgctgcggtatcaggtctctgatttcaatctaaacttcgtggaattagcagttgcgcttgggtatttgaacaacattgagctccaagtagtcggatctgtacaaggcggcgttgagtccattgaatcgctcaaaaaggatgacattgacttcgcggcagtccccttcattggccttgttgcaggagagatagccaccggtgcgcccatcaaagcagtggccgcaagttacggaatttcccacgattcttcttctgcacttctagtccttaaagacagtgagatacacgaagtgcacgatctcattggcaaaacagttggcataaacaccctcggtgctctgggatctgcgatggttgagcgtcatctattcgacgccggtctcaccgaacctgagatcgtgagcgtcactcaacgtgcattacccggtgagtacttagaacaacgcctctaccaggggcaagttgatgcaatttgggtcaccgatagcgctaaacaccaagcgcttgaaactggagattttcggatcttggcagaggattcagaccttgtgcaggaactcaacactggctgcatggtggtgtcgcaaaaactcatcgacgagcaccccgcagtggttggagaattagtggatggagtagctcaggcaatcgagtttgaacgatcccactcccctgaagaagtgcgcgaagtttatttcaactacctcgaagcccatggtcagagtgatagaatatccagctttagatattgggagcattcgggcatcgcaacccgaggtggagtgctcagtgatagggagttcagcatgtggtcccactggattgaccgccaatacgacgtccccgatatcaatccagcaagtatttacaccaaccaattcaacccataccgaaaagtaaacccctcgccataa

SEQ ID NO: 199 - protein, 354 aa, Corynebacterium glutamicum
MRYIALAKVTSMHQRSMSFTFIRTFFVLFGITLLVSCVPEPPDSYTKESTVLRYQVSDFNLNFVELAVALGYLNNIELQVVGSVQGGVESIESLKKDDIDFAAVPFIGLVAGEIATGAPIKAVAASYGISHDSSSALLVLKDSEIHEVHDLIGKTVGINTLGALGSAMVERHLFDAGLTEPEIVSVTQRALPGEYLEQRLYQGQVDAIWVTDSAKHQALETGDFRILAEDSDLVQELNTGCMVVSQKLIDEHPAVVGELVDGVAQAIEFERSHSPEEVREVYFNYLEAHGQSDRISSFRYWEHSGIATRGGVLSDREFSMWSHWIDRQYDVPDINPASIYTNQFNPYRKVNPSP

SEQ ID NO: 200 - DNA, 732 bp, Corynebacterium glutamicum
  source 1..732: mol_type = genomic DNA
ctaggcaggtgtggtgatttcgagccactcaaggagttgagcggtgtaggaggcaaaggaggggtgggtgcggcgatcgccggggatatctacagtcaaactgtgtgtgatggcgccgtcgtcaagaagaaggacgtggtcggccagggcgatggcctcggaaacatcatgggtgaccagcagaactcccaagtttcgggtgttcacggttttgagcagcaggtcttgggctgtcagtcttgtcagagcatcgagggcgccgaagggttcgtcgagaagcaaaagctctggctcggagatgagcgctcgcgcaagggagacgcgctgggcttggccgccggagagcgtgaggggccaggcggcgtcggagtcgggaagtttgacttctgcaagcagtgccgaggcgcgttcttgtgcttctgaccaggaaatatcagtgcggttgaggccaaaggtcacattatcgagcaccgttttccacggcaacaggcgaggctcttggaaggcaacggccgggtttccggaaatttctacagagccggaatgctctttagataggcccgccaacacgcgcaggatggtggattttcctgaacctgagcgtccgatcagcgcggttacttcgccgcagttgatggtgaggtcgattccttggaggacttctttagttccgtatgatttgcgcaatccacggacagtggctgcgggtttgagtgacaatgtggcagtcat

SEQ ID NO: 201 - protein, 243 aa, Corynebacterium glutamicum
MTATLSLKPAATVRGLRKSYGTKEVLQGIDLTINCGEVTALIGRSGSGKSTILRVLAGLSKEHSGSVEISGNPAVAFQEPRLLPWKTVLDNVTFGLNRTDISWSEAQERASALLAEVKLPDSDAAWPLTLSGGQAQRVSLARALISEPELLLLDEPFGALDALTRLTAQDLLLKTVNTRNLGVLLVTHDVSEAIALADHVLLLDDGAITHSLTVDIPGDRRTHPSFASYTAQLLEWLEITTPA

SEQ ID NO: 202 - DNA, 771 bp, Corynebacterium glutamicum
  source 1..771: mol_type = genomic DNA
ttatgcgtttcggtagcggaaggtgtgacgttcccaagctctgatcagcgcttcagacagcagaccgaggctggcgtagacaatgaggccgaaaataacgaggtcggtgcggtaaaaatcgcgcgcattgttgatgaggaagccgagtccttcgcgggcgttgacctgttccgccacgatcagtgagagccaggcggccgcacttgcttggcgcaggccgacaaaaagttgcggcgcggcagaaggaatgatgatggtccgcaacctctggaaaaatccgaagcccatcacgtggccggcttccagaagctttggatcaatttgcctgaacccgctggcggtgttgaggtacagcggatacagcacgccgaggctaataatcagtactttcggcagctcaccgataccgaaccagatgataaacagcggcacaagacccaggtgaggcagcgcgcgggcagcttgaatgagcggatcaacggcggtgtctgcaaatctggacatgcctgtcaatacacctagggaaatgcctaggacagcaccgagggcgaagcctagaaggacgcgttggcttgagacgaggagggcgtcgagaagcgtgccatttgtggcgacctcccagctggcggccaagatcgtggttggtgccggcagaatccgctccgggatggcgcccaggctcgagccgagctgccagaaaacaagcaccgcaagcggtgaatagatgcgcgcgggcagcgcgattttggggcgagtcaaggttgtagtcat

SEQ ID NO: 203 - protein, 256 aa, Corynebacterium glutamicum
MTTTLTRPKIALPARIYSPLAVLVFWQLGSSLGAIPERILPAPTTILAASWEVATNGTLLDALLVSSQRVLLGFALGAVLGISLGVLTGMSRFADTAVDPLIQAARALPHLGLVPLFIIWFGIGELPKVLIISLGVLYPLYLNTASGFRQIDPKLLEAGHVMGFGFFQRLRTIIIPSAAPQLFVGLRQASAAAWLSLIVAEQVNAREGLGFLINNARDFYRTDLVIFGLIVYASLGLLSEALIRAWERHTFRYRNA

SEQ ID NO: 204 - DNA, 810 bp, Corynebacterium glutamicum
  source 1..810: mol_type = genomic DNA
ttaaccgatcgtcataacgatgaccgtggtgattccgacgatgaccacaaatgctctaaaaacactggggcgaagtcggcgggcgtaccgtccgccgatgtatccacctaaagctgagccaagcgcgattaaggcaacggtcggccaggagatcaactcaggggagatgattataaaaacactggctgcgatgagattaactgccgccactgtgaggtttttgatggcgttgagttcctgcatctgcagcgttgatgtgatgccaagcaatgcgatgagaagaatgccttgagctgcgctgaagtaccctccatacatgcccagcaacagggcaccacatactgagacgatgaaggtggttcggctcgggaaaggcaattgcctaaacccagcagagatgccacctgaagtatgagcgccaacatgcttcttaattgatggacctgcgataaccaacagcgtgccgaatccaatcagccagggaattactgctgtaaaaacatctgcggagaaatgcagcaagagcgaggcgccggtgatccctccgaggattgatgctggcagcagaaatctgatggtttttacatgggcgtgtagttcacgtctataagcaaccacacccgaaatacttccaggaacgatgccgatggtgttggcgatggtggcagtcaacggtggaaggcccaggaacacgagaataggaaacgacactaatgttcctgctccgacaattacattgatgcttcctgcgaggaaacctgcaccaaagatcgctcctaattcaacaatttgtaaccactcgattgacat

SEQ ID NO: 205 - protein, 269 aa, Corynebacterium glutamicum
MSIEWLQIVELGAIFGAGFLAGSINVIVGAGTLVSFPILVFLGLPPLTATIANTIGIVPGSISGVVAYRRELHAHVKTIRFLLPASILGGITGASLLLHFSADVFTAVIPWLIGFGTLLVIAGPSIKKHVGAHTSGGISAGFRQLPFPSRTTFIVSVCGALLLGMYGGYFSAAQGILLIALLGITSTLQMQELNAIKNLTVAAVNLIAASVFIIISPELISWPTVALIALGSALGGYIGGRYARRLRPSVFRAFVVIVGITTVIVMTIG

SEQ ID NO: 206 - DNA, 1455 bp, Corynebacterium glutamicum
  source 1..1455: mol_type = genomic DNA
atggtgtggggcatggaacacacttcagcattgacgctcatagactcggttttggaccctgacagcttcatttcttggaatgaaactccccaatatgacaacctcaatcaaggctatgcagagaccttggagcgggctcgaagcaaggccaaatgcgatgaatcggtaattactggagaaggcaccgtggagggcattccggtagccgttattttgtccgatttttccttcctcggcggttctttgggcacggtcgcgtcggtgcgcatcatgaaggcgattcaccgcgccacagagctgaaactcccactgctggtctcccctgcttccggtggtgcgcgcatgcaggaagacaatcgagcttttgtcatgatggtgtccataaccgcggctgtgcagcgtcaccgcgaggcgcatttgccgttcctggtgtatttgcgcaatcccacgatgggtggcgccatggcctcgtggggttcatctgggcatctcacttttgcggaacccggcgcgcagataggtttcctgggtcctcgcgtggtggagttaaccactgggcatgcgcttccagacggtgtgcagcaggcggagaatttggtgaaaactggtgtgattgatggaattgtgtcgccactccaattgcgtgcagcggtggcaaaaaccctcaaggttattcagccggtagaggcaacggatcgtttttctccaacaactcctggcgtggcacttccggtgatggaggcgattgcgcgttctcgtgacccgcagaggcctggaatcggggagattatggaaacgttgggggcagacgtcgtcaagctttctggtgcgcgtgctggcgcattgagcccggctgtgcgcgttgccctggcgcgcatcgggggccggcccgtggtgctgattgggcaggatcgccgcttcacgcttgggccgcaggagctgcgttttgcgcgtcgtggcatttcgctggcgcgcgagctaaacctgccgatcgtgtccatcatcgacacctccggcgccgaattgtcgcaggcggctgaggagctcggcatcgcaagctcgattgcgcgcaccttgtccaagcttatcgacgctcccctccccaccgtttcggtcattattggtcagggcgttggcggtggcgcgctggccatgctgcccgccgatctggtctacgcggccgaaaacgcgtggctgtccgcattgccaccagagggcgcctcggccatcctcttccgcgacaccaaccacgccgcggaaatcatagagcgacaaggcgtgcaggcgcacgcacttttaagccaagggcttatcgacgggatcgtcgccgaaaccgagcactttgttgaagaaattctcggcacaatcagcaacgccctctccgaattggataacaatccggagagggcgggacgcgacagtcgcttcacacgatttgagcgtttagcgcagtaa

SEQ ID NO: 207 - protein, 484 aa, Corynebacterium glutamicum
MVWGMEHTSALTLIDSVLDPDSFISWNETPQYDNLNQGYAETLERARSKAKCDESVITGEGTVEGIPVAVILSDFSFLGGSLGTVASVRIMKAIHRATELKLPLLVSPASGGARMQEDNRAFVMMVSITAAVQRHREAHLPFLVYLRNPTMGGAMASWGSSGHLTFAEPGAQIGFLGPRVVELTTGHALPDGVQQAENLVKTGVIDGIVSPLQLRAAVAKTLKVIQPVEATDRFSPTTPGVALPVMEAIARSRDPQRPGIGEIMETLGADVVKLSGARAGALSPAVRVALARIGGRPVVLIGQDRRFTLGPQELRFARRGISLARELNLPIVSIIDTSGAELSQAAEELGIASSIARTLSKLIDAPLPTVSVIIGQGVGGGALAMLPADLVYAAENAWLSALPPEGASAILFRDTNHAAEIIERQGVQAHALLSQGLIDGIVAETEHFVEEILGTISNALSELDNNPERAGRDSRFTRFERLAQ

SEQ ID NO: 208 - DNA, 216 bp, Corynebacterium glutamicum
  source 1..216: mol_type = genomic DNA
ttagttctttgcctccaattcgaggagttcatcgccgccggtgacgtcggagaaatcatcaaaagaaaccttagctacgatgccaggtcccggtgccatgattggtgcctcgagtttgatggtttctaccgttcccagttgcatgccggtttccacgggatcgccttcatcgacaaaatagtggacgattccagcaaaaggtgcgtagatcttcat

SEQ ID NO: 209 - protein, 71 aa, Corynebacterium glutamicum
MKIYAPFAGIVHYFVDEGDPVETGMQLGTVETIKLEAPIMAPGPGIVAKVSFDDFSDVTGGDELLELEAKN

SEQ ID NO: 210 - DNA, 1464 bp, Corynebacterium glutamicum
  source 1..1464: mol_type = genomic DNA
atggccacggccgagaacacaacacaggagaatcggaaaatcctgttcaacgcatttgatatgaactgcgttgcgcatcagtccccaggactgtggacacacccgaaggataaggcgcgagactacaacactcttgattactgggtgcaccttgccaagactttggagaagggccttttcgacggccttttcatcgcagatgtgcttggaacttacgatgtttatggttctagtaatgaagcggcgttgagcagtggtgcgcaggtgcctgtcaatgatccgatccttcttgtttctgcgatggcctatgccacaaagaacctcgggtttggcattactgcaggtactgcctatgagcacccgtatccttttgcgcggcgtctggccacacttgatcacctgactaatgggcgtgtggggtggaatgtggttactggctatcttccctctgctgctcaaaacatgggtgacaccgatcagctgccacatgatgagcgctatgacaaagcagatgaatacctggaagtgatctacaagcttctcgagggctcctgggaagacgatgctgttcaaaacaatacggagacgagtgtctttacggactcctccaaagtgcacgccattaatcatcatggcaagtactttgatgtgccgggcattgccatcactgagccgagtgtgcagcgtacgccggtgatctaccaggcgggtgcatcgccgcgcggattgaaattcgctggtgagaatgcagaagcagtgtttatcaattccagcaccgtggaggcaatcaccaagactgtcgcaaaaattcgcgctgctgcggtcgctgcgggacgtgatccacatgcggtgaagatctttgcgatgcaaaccatcatcactggtgaaacagaagcagatgcgcaggcaaagctggaggaatacagtcgctatatcgatcctgtcggtggtctgaccttgatgtctggatggaccggcgcggatctgtcgcagtatgacctggatgaaccgatcaccaatattgagtcaaacgctattcagtccactgcagccaccattagcaacggcaccggtgaaggtgcgtggacggtacgcaaactgggtgaggcaaccggcatcggcggcttcggaccagtgcttgtgggatctggcgctaacgttgccgcggaacttgcacgcatccaggatctcagcgatgttgatggtttcaaccttgcttatgccatcaccccaggaacttttgaagatgtcgtggactttgtggtgcctgagctgcaaaaacttagccgctacaagacggaatacgcgccgggttccttgcgcaacaaattgctcggtaaaggtgatcgcctggacgatacccaccgcggcgcaagctaccgcctaggcgctcggaactccaccgccactattgatctcagttccatatccgcccaactagtttcccagggagcccactcatga

SEQ ID NO: 211 - protein, 487 aa, Corynebacterium glutamicum
MATAENTTQENRKILFNAFDMNCVAHQSPGLWTHPKDKARDYNTLDYWVHLAKTLEKGLFDGLFIADVLGTYDVYGSSNEAALSSGAQVPVNDPILLVSAMAYATKNLGFGITAGTAYEHPYPFARRLATLDHLTNGRVGWNVVTGYLPSAAQNMGDTDQLPHDERYDKADEYLEVIYKLLEGSWEDDAVQNNTETSVFTDSSKVHAINHHGKYFDVPGIAITEPSVQRTPVIYQAGASPRGLKFAGENAEAVFINSSTVEAITKTVAKIRAAAVAAGRDPHAVKIFAMQTIITGETEADAQAKLEEYSRYIDPVGGLTLMSGWTGADLSQYDLDEPITNIESNAIQSTAATISNGTGEGAWTVRKLGEATGIGGFGPVLVGSGANVAAELARIQDLSDVDGFNLAYAITPGTFEDVVDFVVPELQKLSRYKTEYAPGSLRNKLLGKGDRLDDTHRGASYRLGARNSTATIDLSSISAQLVSQGAHS

SEQ ID NO: 212 - DNA, 1083 bp, Corynebacterium glutamicum
  source 1..1083: mol_type = genomic DNA
atgatctcaccgcaaacaatcatcgacaatcttgctccagtcctcgcggagatcgcagcaacggctgcgcagcgcgaacaggatcgagaattcagccgtgatttggcaaagcagcttagtgccggtgggttcactaaactccgcattccagttgaatttggcggtttgggttttagccttccagaagcatttgaggtgctggtggcagcggccgctgcagattcaaatatcgcgcagggattgcggcctcacttcttggcagtggagagcttattgatcgctccttattcagagcaccgcaccaagtggctgcgaaaaatcgctgagaaaggcgtggtcattggcaatgcgttgactgaagtgggaaacaagccaggtgagctgaaaaccaagatccgaaaggaaggcgagtcttacgttctcagcgacaccaagttctattccacgggcagtctttacgccgactggattcaagtacatgcgaaagatgaggaagatcaagatgttttcgcctttgttgatcgcgacgcatcaggcgttgttttggtggattactgggacgaatttgggcaacaactatttgcctccggaaccagcttctttgaaaaagtcgtggtagatccactggacattgtcacccgtgattacaccgcacccagcgctttccaggcgctggcgcagtcacatcatttgtctacgttgactggtatttcgcaggcaatcacccgtgacattgttacctatgtacaaaaccgcacccgtattttcagccacggaagtggtgaccttccacgctttgatccgcaggtgcaacaggtggtgggtgaggtgaaagccaagtcgtatgcagtcgagaaaatctttcagggtttcgcacaagaactggatcttgtcgtcgataaggcaaaagccggcactgctacggaggtcgatctggccgctgtcgacctgagcgcctaccaagcccagttagcggtggcacctttggtgctgagccaggccacccaggcctttgaggttggcgcagatgccttaaacggcggccacgtggctgcccaatacacgatcggatccttgtcatga

SEQ ID NO: 213 - protein, 360 aa, Corynebacterium glutamicum
MISPQTIIDNLAPVLAEIAATAAQREQDREFSRDLAKQLSAGGFTKLRIPVEFGGLGFSLPEAFEVLVAAAAADSNIAQGLRPHFLAVESLLIAPYSEHRTKWLRKIAEKGVVIGNALTEVGNKPGELKTKIRKEGESYVLSDTKFYSTGSLYADWIQVHAKDEEDQDVFAFVDRDASGVVLVDYWDEFGQQLFASGTSFFEKVVVDPLDIVTRDYTAPSAFQALAQSHHLSTLTGISQAITRDIVTYVQNRTRIFSHGSGDLPRFDPQVQQVVGEVKAKSYAVEKIFQGFAQELDLVVDKAKAGTATEVDLAAVDLSAYQAQLAVAPLVLSQATQAFEVGADALNGGHVAAQYTIGSLS

SEQ ID NO: 214 - DNA, 1200 bp, Corynebacterium glutamicum
  source 1..1200: mol_type = genomic DNA
atgagccaagaaattttgagccattttgcacccgcattagagcgcattcgaagcggcgccgtcgagcgcgaacagcagcgcgccttgccagtggaagagattaaagagctggtagagctaggttttactgggcttcgagtgcccgaagaactaggcggtgcgggcgcttccctggaaagcgtagttgagttactgatcgagatcgcgggcgccgattccaatatcgcccaagccctgcgcggacattttgccttcgtggaactactcctggaagcgccggagagcgaattccgcacccattggctgcgcgaagtcgccaccggaagacttgtgggcaacgccgaaagtgagaaacgcggcgtttacggcgatccgcagaccttcatcgatgaggtggagactgaaaacggaccgattttcgtgctcaacggcactaagttttataccaccggcacctattttgcggactacacctggaccaccgcgctgctgcgcaacctcaacggccaagaaactttggtcagtttgccggtcgatctgcacgcgccgggcgtggatgttgttgatgattggagcgggtttgggcaaaagctcaccgcctctggaacgaccacgtttaaagacctggaggtggatccgcggtggatcattccacgcactgatgcgcccacgctggtgtggacgtatctgcagctgagcctgctgaccgtgctggttggcagtgccgcagcagctgtcgatgaggtggttgcccgcgcccaatcctccaccagaaatgcgtggaaccctggcgtcgagcgccgcagcgatccggccgcaaccatagcgatcggcgacgcacgcagccgagtcaccgttattcgtggagcgcttctcgacgccacccgccacgtttccaacgccgccacgatcgtaacccccgaagccttcaacgaggcggacgctattgttgcagcgctctggcccatcgtctccggacaagctttggtggtgacatccaacgttttcgatgcggtgggtgcatctgcagtgcttggtgagcattccattgatcgccactggcgcaatgtgcgtaccgtgtcctcaaacaacccggtgttcctggccaagaatgcagtgggggagtatgccctcaacggcactcctgtgggtaccaacattggtaaagcactgagccgtccggtgagcctaagcagctag

SEQ ID NO: 215 - protein, 399 aa, Corynebacterium glutamicum
MSQEILSHFAPALERIRSGAVEREQQRALPVEEIKELVELGFTGLRVPEELGGAGASLESVVELLIEIAGADSNIAQALRGHFAFVELLLEAPESEFRTHWLREVATGRLVGNAESEKRGVYGDPQTFIDEVETENGPIFVLNGTKFYTTGTYFADYTWTTALLRNLNGQETLVSLPVDLHAPGVDVVDDWSGFGQKLTASGTTTFKDLEVDPRWIIPRTDAPTLVWTYLQLSLLTVLVGSAAAAVDEVVARAQSSTRNAWNPGVERRSDPAATIAIGDARSRVTVIRGALLDATRHVSNAATIVTPEAFNEADAIVAALWPIVSGQALVVTSNVFDAVGASAVLGEHSIDRHWRNVRTVSSNNPVFLAKNAVGEYALNGTPVGTNIGKALSRPVSLSS

SEQ ID NO: 216 - DNA, 1296 bp, Corynebacterium glutamicum
  source 1..1296: mol_type = genomic DNA
gtgtctgagaaaagtccaggagtgatggctagcggagcggagctgattcgtgccgccgacattcaaacggcgcaggcacgaatttcctccgtcattgcaccaactccattgcagtattgccctcgtctttctgaggaaaccggagcggaaatctaccttaagcgtgaggatctgcaggatgttcgttcctacaagatccgcggtgcgctgaactctggagcgcagctcacccaagagcagcgcgatgcaggtatcgttgccgcatctgcaggtaaccatgcccagggcgtggcctatgtgtgcaagtccttgggcgttcagggacgcatctatgttcctgtgcagactccaaagcaaaagcgtgaccgcatcatggttcacggcggagagtttgtctccttggtggtcactggcaataacttcgacgaagcatcggctgcagcgcatgaagatgcagagcgcaccggcgcaacgctgatcgagcctttcgatgctcgcaacaccgtcatcggtcagggcaccgtggctgctgagatcttgtcgcagctgacttccatgggcaagagtgcagatcacgtgatggttccagtcggcggtggcggacttcttgcaggtgtggtcagctacatggctgatatggcacctcgcactgcgatcgttggtatcgaaccagcgggagcagcatccatgcaggctgcattgcacaatggtggaccaatcactttggagactgttgatccctttgtggacggcgcagcagtcaaacgtgtcggagatctcaactacaccatcgtggagaagaaccagggtcgcgtgcacatgatgagcgcgaccgagggcgctgtgtgtactgagatgctcgatctttaccaaaacgaaggcatcatcgcggagcctgctggcgcgctgtctatcgctgggttgaaggaaatgtcctttgcacctggttctgtcgtggtgtgcatcatctctggtggcaacaacgatgtgctgcgttatgcggaaatcgctgagcgctccttggtgcaccgcggtttgaagcactacttcttggtgaacttcccgcaaaagcctggtcagttgcgtcacttcctggaagatatcctgggaccggatgatgacatcacgctgtttgagtacctcaagcgcaacaaccgtgagaccggtactgcgttggtgggtattcacttgagtgaagcatcaggattggattctttgctggaacgtatggaggaatcggcaattgattcccgtcgcctcgagccgggcacccctgagtacgaatacttgacctaa

SEQ ID NO: 217 - protein, 431 aa, Corynebacterium glutamicum
MSEKSPGVMASGAELIRAADIQTAQARISSVIAPTPLQYCPRLSEETGAEIYLKREDLQDVRSYKIRGALNSGAQLTQEQRDAGIVAASAGNHAQGVAYVCKSLGVQGRIYVPVQTPKQKRDRIMVHGGEFVSLVVTGNNFDEASAAAHEDAERTGATLIEPFDARNTVIGQGTVAAEILSQLTSMGKSADHVMVPVGGGGLLAGVVSYMADMAPRTAIVGIEPAGAASMQAALHNGGPITLETVDPFVDGAAVKRVGDLNYTIVEKNQGRVHMMSATEGAVCTEMLDLYQNEGIIAEPAGALSIAGLKEMSFAPGSVVVCIISGGNNDVLRYAEIAERSLVHRGLKHYFLVNFPQKPGQLRHFLEDILGPDDDITLFEYLKRNNRETGTALVGIHLSEASGLDSLLERMEESAIDSRRLEPGTPEYEYLT

SEQ ID NO: 218 - DNA, 1536 bp, Escherichia coli
  source 1..1536: mol_type = genomic DNA
atggctacatcagtacagacaggtaaagctaagcagctcacattacttggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctggggccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttatctgcactccggtgcccccgttgctatcgaaatggattcgaagaccttcttccctgacttctctaaagtgggcaccctggtagtatttgttgccttcattttgagttatatgggcgtagaagcatccgcaacccacgtcaatgaaatgagcaacccagggcgcgactatccgttggctatgttactgctgatggtggcggcaatctgcttaagctctgttggtggtttgtctattgcgatggtcattccgggtaatgaaatcaacctctccgcaggggtaatgcaaacctttaccgttctgatgtcccatgtggcaccagaaattgagtggacggttcgcgtgatctccgcactgctgttgctgggtgttctggcggaaatcgcctcctggattgttggtccttctcgcgggatgtatgtaacagcgcagaaaaacctgctgccagcggcattcgctaaaatgaacaaaaatggcgtaccggtaacgctggtcatttcgcagctggtgattacgtctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcaaataccggcgtcactctggagccaatcaacagtcagaacgcaccaaaaggtcacttcttcctgcacccgcgtgcacgttcaccacactatattgtgatgaatgacaagaaacactaa

SEQ ID NO: 219 - protein, 511 aa, Escherichia coli
MATSVQTGKAKQLTLLGFFAITASMVMAVYEYPTFATSGFSLVFFLLLGGILWFIPVGLCAAEMATVDGWEEGGVFAWVSNTLGPRWGFAAISFGYLQIAIGFIPMLYFVLGALSYILKWPALNEDPITKTIAALIILWALALTQFGGTKYTARIAKVGFFAGILLPAFILIALAAIYLHSGAPVAIEMDSKTFFPDFSKVGTLVVFVAFILSYMGVEASATHVNEMSNPGRDYPLAMLLLMVAAICLSSVGGLSIAMVIPGNEINLSAGVMQTFTVLMSHVAPEIEWTVRVISALLLLGVLAEIASWIVGPSRGMYVTAQKNLLPAAFAKMNKNGVPVTLVISQLVITSIALIILTNTGGGNNMSFLIALALTVVIYLCAYFMLFIGYIVLVLKHPDLKRTFNIPGGKGVKLVVAIVGLLTSIMAFIVSFLPPDNIQGDSTDMYVELLVVSFLVVLALPFILYAVHDRKGKANTGVTLEPINSQNAPKGHFFLHPRARSPHYIVMNDKKH

SEQ ID NO: 220 - DNA, 2847 bp, Corynebacterium glutamicum
  source 1..2847: mol_type = genomic DNA
ttattgtgccatgttgttttgctccaagatgcttaagaccacctctgcaaggtgcgttgcttgttttccggtgccttgttcgatctgggtgcggcaggagaaaccgtcagcaatcacatgtccttctgcttttctgaccttggggaacagctctcgttcaccaagtgcgaaggacatttcagcgtggtctttttcaaagccccagtttccggcaagcccgcaacaaccagtggcaatttgttcatcttttacacccaaagcttcaagaacgagtgccgattgttgtgggtcgcctagcgaacgctcgtggcagtgcacctgggtaagcgcagttgattctgttagctggaggcttccagactcgactagctcggtgatctttggtgcgatgacctcagcgaatggtttggtcagtgctgcaaggcgtgccagatcagggttatcggagagttctgttgcctcatcttgaagcatgacggtgcacgaaggttccaaaccaacgactgttagaccttggtccaggtagggtttcatcactttcgccgtttgttctaggactttctttgtcatgctcaattggccggtggaatgccaggtgagtccacagcagacgaagccatctgggatgaccacgttgtaaccgagggcttcaagagttttgatcgctgcgtgagctggtccggtgtcgaggttggtgttgaaggaatcgggccacagcaccaccgtttcacctgagtttttcttcggcttgtacttgcgcagcgagcggtgggcgaaggaaatcaacgggcgatcagcgagcccgccgaccttgcgcaccactggggcggtgagtgctgactgcatggtggcgtcgataagcgtaggaagaagcggtattttatgggcaacgtgtcccagaagcggcagccagcccatgacgtaatgggccatggggcgaagtcggccggcgtagtgtttgtccaggaattcggctttgtaggtggacatgtcgacgttgactggacattccgatgcgcatgctttgcaggaaaggcacaggtcaagggcttcattgacttcttcgctgcggtagccgtcggcgatggattcaccgcggaacatctcagagagcaagcgggcgcggcctctggtggaatgtacttcgtcgccggtgatttggaaggacgggcacatcgcgtcggattctgagcggcatgcggatacacccacgcagcgattcaccgcgttgatcatggaacctttgtctttagagaatttgtgtacgggcgtgatgtcgagggcgcgctggcccgggtccatgcgaagtccttgcatgacaggatctgcccagaccaacactcccggattgaagatgcgctggggatcgaaaatcagcttgaattcttcgaagagtgcacgcatttctgctgaatacatgcggtcaaggaaggatgagcgggcgcgaccgtctccatgctcgcccgagaggctgccaccataagacgccaccaaggtggaggcttcattcatgaacgcctcgaatttcttcaggccttccttggtagagaaatcaaaactgatgcgaacgtggacgcagccttctccaaagtgtccaaatggaataccctggtaatcgaacttatccatcagcgcataaagatcgcggagataatcagctaaattctctggaggcaccgccgagtcttcccaattcggccacgcttccccaccatcagctaagcgcgtgacaatgcccgccgaggattcacggatgcgccacaattcccgcatttcagaaggctcagacacgaccacgtaatcaatggtgtcaacggcggttgcgacttcctcggcggcttgtaccgcggcctgcagggtgtcacttcctgtctcgcagtacaaccatccgccggcttcaatgccgatgcggtttcctggaagattctgcccagcttctgattgtccctgtttactgcgcagcgcagcgaggagatctccgcccatgccttcaatggttgctaccccaggcagtcgcaatttggcggctgctcgggcggcgtcaaaaaccgtgtcgaaagccaggacagcaagcgctttcactttgggtgttggaaccaacttcaccgtcaaccgagtaatgattccaatggttccctcggtgcccgccattgctttggccatgtcgtgggcaagataatgcaaaccgtagcccgacacttggcgagggaaacgacccagttctttactaataaggtcctgattcttggacgctaagtcggtgagcttctgattgatctcagcatcatcgcagccatcttttgtcacagtgacttctcggccatcgctgagcatgagcgtgacatccacgagattttccgcagctgtaccgaatgcaaccgagtgtgaaccacacgcattgttggcaaccatgccaccgatcgtgcaccgggaatgcgtggaaggatccgggccgtaagttaatccgaattctgcggctgcatcgcgcaaggcatcacacaccacaccgggttccacaactgcagtttgtgcaactggatcaatatctaaaatgcggttgaaatagcgtgacgtatcgatgatcaaaccttcaccgatcgcatttccagcaaccgagcttcctccaccgcgcccaacaacagaccaccctcgtgccaccgcaacagcaatggcatcacgaatttgttccacattttctggttcagctacagctgcagggacgcgtctaaaaattcctgcatcagaagaatatgctgcacgggttgtcatatccgtgcgcagcttcgcgcgagaatgctggctaaatacttctgggtcgagtctgttgaatttgatggtgtgagtcat

SEQ ID NO: 221 - protein, 948 aa, Corynebacterium glutamicum
MTHTIKFNRLDPEVFSQHSRAKLRTDMTTRAAYSSDAGIFRRVPAAVAEPENVEQIRDAIAVAVARGWSVVGRGGGSSVAGNAIGEGLIIDTSRYFNRILDIDPVAQTAVVEPGVVCDALRDAAAEFGLTYGPDPSTHSRCTIGGMVANNACGSHSVAFGTAAENLVDVTLMLSDGREVTVTKDGCDDAEINQKLTDLASKNQDLISKELGRFPRQVSGYGLHYLAHDMAKAMAGTEGTIGIITRLTVKLVPTPKVKALAVLAFDTVFDAARAAAKLRLPGVATIEGMGGDLLAALRSKQGQSEAGQNLPGNRIGIEAGGWLYCETGSDTLQAAVQAAEEVATAVDTIDYVVVSEPSEMRELWRIRESSAGIVTRLADGGEAWPNWEDSAVPPENLADYLRDLYALMDKFDYQGIPFGHFGEGCVHVRISFDFSTKEGLKKFEAFMNEASTLVASYGGSLSGEHGDGRARSSFLDRMYSAEMRALFEEFKLIFDPQRIFNPGVLVWADPVMQGLRMDPGQRALDITPVHKFSKDKGSMINAVNRCVGVSACRSESDAMCPSFQITGDEVHSTRGRARLLSEMFRGESIADGYRSEEVNEALDLCLSCKACASECPVNVDMSTYKAEFLDKHYAGRLRPMAHYVMGWLPLLGHVAHKIPLLPTLIDATMQSALTAPVVRKVGGLADRPLISFAHRSLRKYKPKKNSGETVVLWPDSFNTNLDTGPAHAAIKTLEALGYNVVIPDGFVCCGLTWHSTGQLSMTKKVLEQTAKVMKPYLDQGLTVVGLEPSCTVMLQDEATELSDNPDLARLAALTKPFAEVIAPKITELVESGSLQLTESTALTQVHCHERSLGDPQQSALVLEALGVKDEQIATGCCGLAGNWGFEKDHAEMSFALGERELFPKVRKAEGHVIADGFSCRTQIEQGTGKQATHLAEVVLSILEQNNMAQ

SEQ ID NO: 222 - DNA, 1881 bp, Corynebacterium glutamicum
  source 1..1881: mol_type = genomic DNA
gtgaatgtggcagcttctcaacagcccactcccgccacggttgcaagccgtggtcgatccgccgcccctgagcggatgacaggtgcaaaggcaattgttcgatcgctcgaggagcttaacgccgacatcgtgttcggtattcctggtggtgcggtgctaccggtgtatgacccgctctattcctccacaaaggtgcgccacgtcttggtgcgccacgagcagggcgcaggccacgcagcaaccggctacgcgcaggttactggacgcgttggcgtctgcattgcaacctctggcccaggagcaaccaacttggttaccccaatcgctgatgcaaacttggactccgttcccatggttgccatcaccggccaggtcggaagtggcctgctgggtaccgacgctttccaggaagccgatatccgcggcatcaccatgccagtgaccaagcacaacttcatggtcaccaaccctaacgacattccacaggcattggctgaggcattccacctcgcgattactggtcgccctggccctgttctggtggatattcctaaggatgtccagaacgctgaattggatttcgtctggccaccaaagatcgacctgccaggctaccgcccagtttcaacaccacatgctcgccagatcgagcaggcagtcaagctgatcggtgaggccaagaagcccgtcctttacgttggtggtggcgtaatcaaggctgacgcacacgaagagcttcgtgcgttcgctgagtacaccggcatcccagttgtcaccaccttgatggctttgggtactttcccagagtctcacgagctgcacatgggtatgccaggcatgcatggcactgtgtccgctgttggtgcactgcagcgcagcgacctgctgattgctatcggctcccgctttgatgaccgcgtcaccggtgacgttgacaccttcgcgcctgacgccaagatcattcacgccgacattgatcctgccgaaatcggcaagatcaagcaggttgaggttccaatcgtgggcgatgcccgcgaagttcttgctcgtctgctggaaaccaccaaggcaagcaaggcagagaccgaggacatctccgagtgggttgactacctcaagggcctcaaggcacgtttcccgcgtggctacgacgagcagccaggcgatctgctggcaccacagtttgtcattgaaaccctgtccaaggaagttggccccgacgcaatttactgcgccggcgttggccagcaccaaatgtgggcagctcagttcgttgactttgaaaagccacgcacctggctcaactccggtggactgggcaccatgggctacgcagttcctgcggcccttggagcaaaggctggcgcacctgacaaggaagtctgggctatcgacggcgacggctgtttccagatgaccaaccaggaactcaccaccgccgcagttgaaggtttccccattaagatcgcactaatcaacaacggaaacctgggcatggttcgccaatggcagaccctattctatgaaggacggtactcaaatactaaacttcgtaaccagggcgagtacatgcccgactttgttaccctttctgagggacttggctgtgttgccatccgcgtcaccaaagcggaggaagtactgccagccatccaaaaggctcgagagatcaacgaccgcccagtagtcatcgacttcatcgtcggtgaagacgcacaggtatggccaatggtgtctgctggatcatccaactccgatatccagtacgcactcggattgcgcccattctttgatggtgatgaatctgcagcagaagatcctgccgacattcacgaagccgtcagcgacattgatgccgccgttgaatcgaccgaggcataa

SEQ ID NO: 223 - protein, 626 aa, Corynebacterium glutamicum
MNVAASQQPTPATVASRGRSAAPERMTGAKAIVRSLEELNADIVFGIPGGAVLPVYDPLYSSTKVRHVLVRHEQGAGHAATGYAQVTGRVGVCIATSGPGATNLVTPIADANLDSVPMVAITGQVGSGLLGTDAFQEADIRGITMPVTKHNFMVTNPNDIPQALAEAFHLAITGRPGPVLVDIPKDVQNAELDFVWPPKIDLPGYRPVSTPHARQIEQAVKLIGEAKKPVLYVGGGVIKADAHEELRAFAEYTGIPVVTTLMALGTFPESHELHMGMPGMHGTVSAVGALQRSDLLIAIGSRFDDRVTGDVDTFAPDAKIIHADIDPAEIGKIKQVEVPIVGDAREVLARLLETTKASKAETEDISEWVDYLKGLKARFPRGYDEQPGDLLAPQFVIETLSKEVGPDAIYCAGVGQHQMWAAQFVDFEKPRTWLNSGGLGTMGYAVPAALGAKAGAPDKEVWAIDGDGCFQMTNQELTTAAVEGFPIKIALINNGNLGMVRQWQTLFYEGRYSNTKLRNQGEYMPDFVTLSEGLGCVAIRVTKAEEVLPAIQKAREINDRPVVIDFIVGEDAQVWPMVSAGSSNSDIQYALGLRPFFDGDESAAEDPADIHEAVSDIDAAVESTEA

SEQ ID NO: 224 - DNA, 519 bp, Corynebacterium glutamicum
  source 1..519: mol_type = genomic DNA
atggctaattctgacgtcacccgccacatcctgtccgtactcgttcaggacgtagacggaatcatttcccgcgtatcaggtatgttcacccgacgcgcattcaacctcgtgtccctcgtgtctgcaaagaccgaaacacacggcatcaaccgcatcacggttgttgtcgacgccgacgagctcaacattgagcagatcaccaagcagctcaacaagctgatccccgtgctcaaagtcgtgcgacttgatgaagagaccactatcgcccgcgcaatcatgctggttaaggtctctgcggacagcaccaaccgtccgcagatcgtcgacgccgcgaacatcttccgcgcccgagtcgtcgacgtggctccagactctgtggttattgaatccacaggcaccccaggcaagctccgcgcactgcttgacgtgatggaaccattcggaatccgcgaactgatccaatccggacagattgcactcaaccgcggtccgaagaccatggctccggccaagatctaa

SEQ ID NO: 225 - protein, 172 aa, Corynebacterium glutamicum
MANSDVTRHILSVLVQDVDGIISRVSGMFTRRAFNLVSLVSAKTETHGINRITVVVDADELNIEQITKQLNKLIPVLKVVRLDEETTIARAIMLVKVSADSTNRPQIVDAANIFRARVVDVAPDSVVIESTGTPGKLRALLDVMEPFGIRELIQSGQIALNRGPKTMAPAKI

SEQ ID NO: 226 - DNA, 1017 bp, Corynebacterium glutamicum
  source 1..1017: mol_type = genomic DNA
atggctattgaactgctttatgatgctgacgctgacctctccttgatccagggccgtaaggttgccatcgttggctacggctcccagggccacgcacactcccagaacctccgcgattctggcgttgaggttgtcattggtctgcgcgagggctccaagtccgcagagaaggcaaaggaagcaggcttcgaggtcaagaccaccgctgaggctgcagcttgggctgacgtcatcatgctcctggctccagacacctcccaggcagaaatcttcaccaacgacatcgagccaaacctgaacgcaggcgacgcactgctgttcggccacggcctgaacattcacttcgacctgatcaagccagctgacgacatcatcgttggcatggttgcgccaaagggcccaggccacttggttcgccgtcagttcgttgatggcaagggtgttccttgcctcatcgcagtcgaccaggacccaaccggaaccgcacaggctctgaccctgtcctacgcagcagcaatcggtggcgcacgcgcaggcgttatcccaaccaccttcgaagctgagaccgtcaccgacctcttcggcgagcaggctgttctctgcggtggcaccgaggaactggtcaaggttggcttcgaggttctcaccgaagctggctacgagccagagatggcatacttcgaggttcttcacgagctcaagctcatcgttgacctcatgttcgaaggtggcatcagcaacatgaactactctgtttctgacaccgctgagttcggtggctacctctccggcccacgcgtcatcgatgcagacaccaagtcccgcatgaaggacatcctgaccgatatccaggacggcaccttcaccaagcgcctcatcgcaaacgttgagaacggcaacaccgagcttgagggccttcgtgcttcctacaacaaccacccaatcgaggagaccggcgctaagctccgcgacctcatgagctgggtcaaggttgacgctcgcgcagaaaccgcttaa

SEQ ID NO: 227 - protein, 338 aa, Corynebacterium glutamicum
MAIELLYDADADLSLIQGRKVAIVGYGSQGHAHSQNLRDSGVEVVIGLREGSKSAEKAKEAGFEVKTTAEAAAWADVIMLLAPDTSQAEIFTNDIEPNLNAGDALLFGHGLNIHFDLIKPADDIIVGMVAPKGPGHLVRRQFVDGKGVPCLIAVDQDPTGTAQALTLSYAAAIGGARAGVIPTTFEAETVTDLFGEQAVLCGGTEELVKVGFEVLTEAGYEPEMAYFEVLHELKLIVDLMFEGGISNMNYSVSDTAEFGGYLSGPRVIDADTKSRMKDILTDIQDGTFTKRLIANVENGNTELEGLRASYNNHPIEETGAKLRDLMSWVKVDARAETA

SEQ ID NO: 228 - DNA, 1842 bp, Corynebacterium glutamicum
  source 1..1842: mol_type = genomic DNA
ttagtcgacctgacggactgcacccttatcagcggaggtagccatctttgcgtatgcgcgcagtgccttggtgacaacgcggttacggttgactggctgccatggcttctcggaggcgttcatagcgtcgcggcggcgctggagttcctcgtcggagacctgaacttcgagcttgcggttgtgaacgtcgatggagacgatgtcgccgttttcgatcagaccaatgactccgccgtgtgctgcttctggggagacgtggccgatggacagtcctgaggaacctccggagaaacggccgtcggtgatcagtgcacacttcttgcccaggccggatcccttgaggaatgcggttgggtgaagcatttcctgcatgcctggtccacctgatgggccttcgtagcggacgaccagaacttcgccagcttggatggtcttggtcaggatgacagagactgcctcttcctggctttcgacaactcgtgctggtccggtgaagttccacagctcttcttcgatacctgcggacttgatcactgcgccgtcaggggagatgttgccgcgaagaacaaccaggccgccgtcggcggtgtaggcgtgttcaacgtcgcggatgcagcccttggcagcgtcggtgtcgagttcgtcccagcggttctcggtggagaatgcttcggtggtgcggatgccacctggggctgcgtggaagagttcggttgctacttcggtggtcttgccagagcggatatcccagtcatccaaccaaccttcaaggtcgttggagtgaacggagtggacgtccttgttcagcaggccaccgcggttgagctcgccgagcagtgctggaatgccaccggcgcggtggacgtcttccatgtggtagtcggagtttggtgcaaccttggacaggcaggggacgtttttggacagttcgtcgatgtctgcgaggtcgaagtcaacttcgccttcctgggcagctgcgaggatgtggaggatggtgttggtggatccacccatggccatatccagtgccattgcgttttcgaatgccttcttggtggcaatgccacgtggcagaacggattcgtcttcttcaccgtagtagcggcggcacagttcaacgacggtttcgccggccttttcaaacagtgcgcgacgtgctgcgtgggttgccagagtggagccgttgcccgggagagaaagtcccagagcttcggtgaggcagttcatggagttcgcggtgaacataccggagcaggagccacaggttgggcatgcggatgcttcaacggctgcaaggcctgcgtcgtcgactgcatcgcttgcggatgcggagatcgcggtgatgaggtcggttggtgcgtgtgcaacgccgtcaacgacgacagccttgccagcttccattgggccaccggaaacgaagaccactgggatgttcaggcgcattgctgcgttgagcatgcctggggtgatcttgtcacagttggagatacacaccatggcgtcggcggtgtgtgcgttgaccatgtattcgacggagtcggcgatgatttcacgggatggcagggagtacagcatgccgccgtgtcccatggcgatgccgtcatcgacggcgatggtgttgaattcctttggaacgccaccggctttgcgcactgcatctgccacaatatcgccgacgttcttaaggtgaacgtgtccgggcacgaactgggtgtaggagtttacgatggcaacaattggcttgccgaactcattttccttggtgccggtggcacgccaaagggcgcgagcgccagctgcattgcgaccgacggtggtgacttttgaacgaagtgggatcat

SEQ ID NO: 229 - protein, 613 aa, Corynebacterium glutamicum
MIPLRSKVTTVGRNAAGARALWRATGTKENEFGKPIVAIVNSYTQFVPGHVHLKNVGDIVADAVRKAGGVPKEFNTIAVDDGIAMGHGGMLYSLPSREIIADSVEYMVNAHTADAMVCISNCDKITPGMLNAAMRLNIPVVFVSGGPMEAGKAVVVDGVAHAPTDLITAISASASDAVDDAGLAAVEASACPTCGSCSGMFTANSMNCLTEALGLSLPGNGSTLATHAARRALFEKAGETVVELCRRYYGEEDESVLPRGIATKKAFENAMALDMAMGGSTNTILHILAAAQEGEVDFDLADIDELSKNVPCLSKVAPNSDYHMEDVHRAGGIPALLGELNRGGLLNKDVHSVHSNDLEGWLDDWDIRSGKTTEVATELFHAAPGGIRTTEAFSTENRWDELDTDAAKGCIRDVEHAYTADGGLVVLRGNISPDGAVIKSAGIEEELWNFTGPARVVESQEEAVSVILTKTIQAGEVLVVRYEGPSGGPGMQEMLHPTAFLKGSGLGKKCALITDGRFSGGSSGLSIGHVSPEAAHGGVIGLIENGDIVSIDVHNRKLEVQVSDEELQRRRDAMNASEKPWQPVNRNRVVTKALRAYAKMATSADKGAVRQVD

SEQ ID NO: 230 - DNA, 3474 bp, Corynebacterium glutamicum
  source 1..3474: mol_type = genomic DNA
gtgactgctatcacccttggcggtctcttgttgaaaggaataattactctagtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctttcggcgatggcgcggtatatgtcgaacgtgctgtgattaaccctcagcatattgaagtgcagatccttggcgatcacactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccgtggaattcttggtcgatgaaaagggcaaccacgtcttcatcgaaatgaacccacgtatccaggttgagcacaccgtgactgaagaagtcaccgaggtggacctggtgaaggcgcagatgcgcttggctgctggtgcaaccttgaaggaattgggtctgacccaagataagatcaagacccacggtgcagcactgcagtgccgcatcaccacggaagatccaaacaacggcttccgcccagataccggaactatcaccgcgtaccgctcaccaggcggagctggcgttcgtcttgacggtgcagctcagctcggtggcgaaatcaccgcacactttgactccatgctggtgaaaatgacctgccgtggttccgactttgaaactgctgttgctcgtgcacagcgcgcgttggctgagttcaccgtgtctggtgttgcaaccaacattggtttcttgcgtgcgttgctgcgggaagaggacttcacttccaagcgcatcgccaccggattcattgccgatcacccgcacctccttcaggctccacctgctgatgatgagcagggacgcatcctggattacttggcagatgtcaccgtgaacaagcctcatggtgtgcgtccaaaggatgttgcagctcctatcgataagctgcctaacatcaaggatctgccactgccacgcggttcccgtgaccgcctgaagcagcttggcccagccgcgtttgctcgtgatctccgtgagcaggacgcactggcagttactgataccaccttccgcgatgcacaccagtctttgcttgcgacccgagtccgctcattcgcactgaagcctgcggcagaggccgtcgcaaagctgactcctgagcttttgtccgtggaggcctggggcggcgcgacctacgatgtggcgatgcgtttcctctttgaggatccgtgggacaggctcgacgagctgcgcgaggcgatgccgaatgtaaacattcagatgctgcttcgcggccgcaacaccgtgggatacaccccgtacccagactccgtctgccgcgcgtttgttaaggaagctgccagctccggcgtggacatcttccgcatcttcgacgcgcttaacgacgtctcccagatgcgtccagcaatcgacgcagtcctggagaccaacaccgcggtagccgaggtggctatggcttattctggtgatctctctgatccaaatgaaaagctctacaccctggattactacctaaagatggcagaggagatcgtcaagtctggcgctcacatcttggccattaaggatatggctggtctgcttcgcccagctgcggtaaccaagctggtcaccgcactgcgccgtgaattcgatctgccagtgcacgtgcacacccacgacactgcgggtggccagctggcaacctactttgctgcagctcaagctggtgcagatgctgttgacggtgcttccgcaccactgtctggcaccacctcccagccatccctgtctgccattgttgctgcattcgcgcacacccgtcgcgataccggtttgagcctcgaggctgtttctgacctcgagccgtactgggaagcagtgcgcggactgtacctgccatttgagtctggaaccccaggcccaaccggtcgcgtctaccgccacgaaatcccaggcggacagttgtccaacctgcgtgcacaggccaccgcactgggccttgcggatcgtttcgaactcatcgaagacaactacgcagccgttaatgagatgctgggacgcccaaccaaggtcaccccatcctccaaggttgttggcgacctcgcactccacctcgttggtgcgggtgtggatccagcagactttgctgccgatccacaaaagtacgacatcccagactctgtcatcgcgttcctgcgcggcgagcttggtaaccctccaggtggctggccagagccactgcgcacccgcgcactggaaggccgctccgaaggcaaggcacctctgacggaagttcctgaggaagagcaggcgcacctcgacgctgatgattccaaggaacgtcgcaatagcctcaaccgcctgctgttcccgaagccaaccgaagagttcctcgagcaccgtcgccgcttcggcaacacctctgcgctggatgatcgtgaattcttctacggcctggtcgaaggccgcgagactttgatccgcctgccagatgtgcgcaccccactgcttgttcgcctggatgcgatctctgagccagacgataagggtatgcgcaatgttgtggccaacgtcaacggccagatccgcccaatgcgtgtgcgtgaccgctccgttgagtctgtcaccgcaaccgcagaaaaggcagattcctccaacaagggccatgttgctgcaccattcgctggtgttgtcaccgtgactgttgctgaaggtgatgaggtcaaggctggagatgcagtcgcaatcatcgaggctatgaagatggaagcaacaatcactgcttctgttgacggcaaaatcgatcgcgttgtggttcctgctgcaacgaaggtggaaggtggcgacttgatcgtcgtcgtttcctaa

SEQ ID NO: 231 - protein, 1157 aa, Corynebacterium glutamicum
MTAITLGGLLLKGIITLVSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS

SEQ ID NO: 232 - DNA, 1233 bp, Corynebacterium glutamicum
  source 1..1233: mol_type = genomic DNA
ttaacgcagcggatcgaacggcttgatgatcgggttggtgttgcccgtagccatcagctccgccaaatacttaccggtggcagggcccagcaccacaccccacatgccgtgaccaccggcaacgtaaatgttctccgccttggtctgcccaatcaacggacgcccatccggagtgaccgggcgggaacccacccactcatcctgacgatcgttgaaatcaatacctctcataaccttcttcgcctgcgaaacaatcgcatccacccggccctgctggaacggctcatccggaccgcggaactccatggtgcctgcaatgcggaaacggccctcatacggcgtgcaggccatgcggtggtggggaaggtacacagaatgcttggcaggaatatccgttgccacagagaaggaatagccacgaccagcctgaacaagagttttcacaccgtattcacgcgttagacccggcagccaggcacccgttgccacaaccaccttgtccgcttcttcacggctaccatccgccaaaatgaccgcgggacgatcacccttcgccacatgcacaacttctgccccggcgcggatcacgccaccacgcttcaccacagcatccgccaatgactgcacgtatggacccggctcgatgaaacgctggccttccaaacggtaagccacctgaatttgctcattcagcatcggcgccaactcttgtggattctccagtcgagacatctccactttctggccgtgcctgctcacgccatcaatttccttacggaaacccgccgattggcgctcttcctcaaaaccaataacaaatggaccttcatgggtgaggccttccacgccaccgatcgacagttcatcaaaagcttcgagcgcgaccttatcgatctccgtgaggtccgccatcgtggaatcccacttgcgttgaaaagcctgcgccataaattgcgccaagaaaagccacagtttgggatccacacgaagtggcatatgcatcggcgacaccggattgaacagctctttcggaccatacgtccacagccccggctccgacaacggaatagttttcgccggcgctaaccaaccagcattaccccacgaagaacctgcagcgacaccatcccgatcaaggacgctcacctcgaacccgtgctcctgtaaatgccaggcggtggcaagacccactatgccggctccaacaacaatgacttttccagacgaactcat

SEQ ID NO: 233 - protein, 410 aa, Corynebacterium glutamicum
MSSSGKVIVVGAGIVGLATAWHLQEHGFEVSVLDRDGVAAGSSWGNAGWLAPAKTIPLSEPGLWTYGPKELFNPVSPMHMPLRVDPKLWLFLAQFMAQAFQRKWDSTMADLTEIDKVALEAFDELSIGGVEGLTHEGPFVIGFEEERQSAGFRKEIDGVSRHGQKVEMSRLENPQELAPMLNEQIQVAYRLEGQRFIEPGPYVQSLADAVVKRGGVIRAGAEVVHVAKGDRPAVILADGSREEADKVVVATGAWLPGLTREYGVKTLVQAGRGYSFSVATDIPAKHSVYLPHHRMACTPYEGRFRIAGTMEFRGPDEPFQQGRVDAIVSQAKKVMRGIDFNDRQDEWVGSRPVTPDGRPLIGQTKAENIYVAGGHGMWGVVLGPATGKYLAELMATGNTNPIIKPFDPLR

SEQ ID NO: 234 - DNA, 972 bp, Corynebacterium glutamicum
  source 1..972: mol_type = genomic DNA
ctagatcttgcgcatcctcacccgttccaccaagtgatcgctaccttttttcagtaccaacgacgcgcgaactcgggtgggaagaatattctccaccaagttgggcaggttgatcgattgccacagttctcgagcgacggcgatggactctggatcagccatgtcggcgtaatgggagaagtgggcaccggggcgacggaatgcagtgtcgcggagtttgaggaagcggtcgatgtaccatttttcgatatcttcggtgcgggcatctacgtagacgctgaagtcgaaaaggtcactgaccatcaatgttgggccagtttggaggacgtttaagccttcgacgatcaaaatgtcgggttggcggactgtggtgaattcgcctggaactcggtcgtacgcggtgtgggagtagacaggtgcgttgacttcgagttttccggattttacgtcggtgacaaagcggaggagtgcacgttggtcgtagctttcggggaatccttttcgggacattaatccgcggcggattagttccgcgccgggatagaggaatccgtcggtggtgacgaggtccacgcgggggtgggaattccagcgctgaagcagaacttggaggagtcgggcggtggttgatttaccgacggcgacggatcccgcgacaccaatgacaaacggcacagagatagagggggaagttccgaggaaggtttcggtggctgcagtaagttgctgtcgggccgctacctggaggtgaatcagacgggacagcggaaggtagacttctgccacttcagcgaggtcaatgttttctccgatgcctcgaagttcaatgacttctttttgggtcagcacctgaggcattgagtttctcagctcgcgccattgtgcgcggtcgaaatcaaggtaggggctgaaatctggtgtgcgtggggaaggtttcacaccagttgtgcttgcagcgttttgctctgccat

SEQ ID NO: 235 - protein, 323 aa, Corynebacterium glutamicum
MAEQNAASTTGVKPSPRTPDFSPYLDFDRAQWRELRNSMPQVLTQKEVIELRGIGENIDLAEVAEVYLPLSRLIHLQVAARQQLTAATETFLGTSPSISVPFVIGVAGSVAVGKSTTARLLQVLLQRWNSHPRVDLVTTDGFLYPGAELIRRGLMSRKGFPESYDQRALLRFVTDVKSGKLEVNAPVYSHTAYDRVPGEFTTVRQPDILIVEGLNVLQTGPTLMVSDLFDFSVYVDARTEDIEKWYIDRFLKLRDTAFRRPGAHFSHYADMADPESIAVARELWQSINLPNLVENILPTRVRASLVLKKGSDHLVERVRMRKI

SEQ ID NO: 236 - DNA, 1263 bp, Corynebacterium glutamicum
  source 1..1263: mol_type = genomic DNA
ctagacttcgcgatacgccagtgccgcgtcccaaatttgcgcagcaacctcgattttgctgccgtgctccacatcgactaccccaccgtgagcatccaaaatccagccctcattgtgcttttgcccaaacactttgcccatgcccacctcattacacatgaggaggtcgcagcccttcttctgcagtttcttgcgcgcatactccaaggcggtggtgtgctcgtctccagtttccgcagcaaaacccacgatgacaggattactgtccagctctcctgcttcacgacgcttcaccgtggtagccaaaatatccgggttttccaccaacgagatggtgctcaatgcgtcttcatcagaatctgagcccttcttcaacttcgatgtcgcctgcgatgcgggcgtgaaatcagctaccgctgccgccatgacgatgaaatcagattggccagctcgttcctggactgcatcaaacatgtcttgtgtggacaccaccggcacgatctctgcgcctgccggagtgggcagctccgcagcatttcccgccacgatgctgacatgagcacctcgctgtgctgcgatttcacccaacgcaaaaccttgacggcccgaggaactattgccaataaagcgcacaggatcaatatgctcatgcgtgccaccagcagtgatcagcactttcttgcccgccaaatcctgaggcaacctcgccccggcgtgcaccgcattggctaaatcaacaatctgctctggatccggcagccggccagggcctgtatctttaccggtgagtcgaccatgcgcaggctcaatcacggtaatcccccgctgcctcagcgttgccacattggctacggtagccggattaaaccacatctccgtatgcatggccggcgcaataaccacggggcacgttgccaccagcagcgtcgccgccaacagatcgtcaccgcgacctgccaccacacgcgccatcaaatcggctgtcgccggcgcaatcacgatcaaatcagcctcctggccaactttcacatgctgcaccgaatccaccgcatcaaacaccgttgtagacaccggattgccagacaacgcttcaaacgtcgccttccccacaaacttcaacgcggattccgtaggaaccacccgcacattatcgcccgcttctttaaacgcgcgcacgatgtgacacgccttgtacgctgcgattccgccggcaacaccaacaaccacgttgcggggtgcggaggggcttgaaacatgagtcat

SEQ ID NO: 237 - protein, 420 aa, Corynebacterium glutamicum
MTHVSSPSAPRNVVVGVAGGIAAYKACHIVRAFKEAGDNVRVVPTESALKFVGKATFEALSGNPVSTTVFDAVDSVQHVKVGQEADLIVIAPATADLMARVVAGRGDDLLAATLLVATCPVVIAPAMHTEMWFNPATVANVATLRQRGITVIEPAHGRLTGKDTGPGRLPDPEQIVDLANAVHAGARLPQDLAGKKVLITAGGTHEHIDPVRFIGNSSSGRQGFALGEIAAQRGAHVSIVAGNAAELPTPAGAEIVPVVSTQDMFDAVQERAGQSDFIVMAAAVADFTPASQATSKLKKGSDSDEDALSTISLVENPDILATTVKRREAGELDSNPVIVGFAAETGDEHTTALEYARKKLQKKGCDLLMCNEVGMGKVFGQKHNEGWILDAHGGVVDVEHGSKIEVAAQIWDAALAYREV

SEQ ID NO: 238 - DNA, 483 bp, Corynebacterium glutamicum
  source 1..483: mol_type = genomic DNA
atgaaagctgtttgcccaggatcattcgaccccatcaccctcggccacctcgacatcgtgacccgcgccgccgcgcaatttagcgaagtcaccatcttggtcaccgccaaccccaacaaaaactcaggactgttcaccgtcgcagaacgcatggatctcatccgcgaatccaccgcacacctcgacaacgtaaaagtagacacctgggcctcactgctcgtggattacaccaccgaacatggcatcggcgccctagtcaaaggcctgcgaagctccctcgactacgaatacgagctccccatggcacagatgaacaggcgactcacgggcgtagacaccttcttcctcctcaccgacgagaaatacggctacgtcagctccacactgtgcaaagaagttgcccgcttcggtggcgatgtctccggcctcctacctgaggtagtggcaaaagccgtgacagaaaaatacagcaaccagcactag

SEQ ID NO: 239 - protein, 160 aa, Corynebacterium glutamicum
MKAVCPGSFDPITLGHLDIVTRAAAQFSEVTILVTANPNKNSGLFTVAERMDLIRESTAHLDNVKVDTWASLLVDYTTEHGIGALVKGLRSSLDYEYELPMAQMNRRLTGVDTFFLLTDEKYGYVSSTLCKEVARFGGDVSGLLPEVVAKAVTEKYSNQH

SEQ ID NO: 240 - DNA, 603 bp, Corynebacterium glutamicum
  source 1..603: mol_type = genomic DNA
atgttgcgcattggactaacaggagggatcggcagcggtaaatctaccgttgccgatcttttgtcatctgaaggatttctcatcgtcgacgcggaccaagttgcccgcgatatcgtcgaacccggacaaccggcattagcagagctagctgaagcttttggccaagacatcttaaaacccgacggcactctagaccgcgcgggattagcagccaaagcatttgtcagcgaagaacaaacagcgctgctcaatgccattacccaccctcgtatcgccgaagagtcagctcgtcgattcaacgaagccgaagatcaaggcgccaaagttgcggtttatgacatgcctttgcttgtagaaaaaggccttgaccgcaagatggaccttgtcgtcgtagttgatgttgacgtagaggaacgcgtccgcagacttgtggaaaaacgtggcctcacagaggacgacgtgcggcgtcgaatcgcttctcaagtgcccgacgacgtcagacttaaagccgctgacatcgttgtggacaataacggcacgctagaggaccttcatgctgaagcaagcaagctgattgctgagattcttagtcgcgtgaattag

SEQ ID NO: 241 - protein, 200 aa, Corynebacterium glutamicum
MLRIGLTGGIGSGKSTVADLLSSEGFLIVDADQVARDIVEPGQPALAELAEAFGQDILKPDGTLDRAGLAAKAFVSEEQTALLNAITHPRIAEESARRFNEAEDQGAKVAVYDMPLLVEKGLDRKMDLVVVVDVDVEERVRRLVEKRGLTEDDVRRRIASQVPDDVRLKAADIVVDNNGTLEDLHAEASKLIAEILSRVN

SEQ ID NO: 242 - DNA, 1054 bp, Escherichia coli
  source 1..1054: mol_type = genomic DNA
agtgaacatatatatcgggtggcttttcaaattaatccctttgattatgggcttaatttgtatcgcgttaggtggctttgtgctggaaagttcagggcaaagcgagtatttcgtcgcgggtcatgtgctgatttctctggcggccatatgcctggcattattcactaccgcatttattatcatttcgcagctcacgcgcggcgttaatacgttttacaatacattgttccccattattggctatgcggggtcaattatcaccatgatatggggttgggcactgttagcaggcaatgatgtgatggcagacgagtttgtcgccggccatgttattttcggcgttggtatgattgccgcctgtgtatcgacggtggcagcgtcatccggtcactttctgctcattcccaaaaatgcagcggggagcaagagcgacggaacaccggtacaggcttattcttcattaatcggtaactgcctcattgccgttcccgttttactcaccctgctcggtttcatttggtctattacgctgttacgtagtgctgacataactccgcattatgtcgcgggtcacgtattgcttgggttaaccgcaatctgtgcctgtctaattggccttgttgccacaattgtccatcaaacacgtaatacgttttcaactaaagaacactggctgtggtgttattgggttatttttctcggctcaatcacggtactgcaggggatatacgtcttagtcagttccgatgcaagcgcccgactggctcccggcattattcttatttgcctcggaatgatctgttacagcatattctcaaaagtctggctactggcactggtatggagacgtacctgttcgttagccaacagaataccgatgattcccgtcttcacctgcctgttttgccttttcctggcatcgtttcttgcggaaatggcgcagaccgacatgggatattttattccttcgcgagttctggtcggtttgggagcggtatgctttacgttgttctcaatcgtttcaatattagaagcgggttctgctaaaaaataa

SEQ ID NO: 243 - protein, 350 aa, Escherichia coli
MNIYIGWLFKLIPLIMGLICIALGGFVLESSGQSEYFVAGHVLISLAAICLALFTTAFIIISQLTRGVNTFYNTLFPIIGYAGSIITMIWGWALLAGNDVMADEFVAGHVIFGVGMIAACVSTVAASSGHFLLIPKNAAGSKSDGTPVQAYSSLIGNCLIAVPVLLTLLGFIWSITLLRSADITPHYVAGHVLLGLTAICACLIGLVATIVHQTRNTFSTKEHWLWCYWVIFLGSITVLQGIYVLVSSDASARLAPGIILICLGMICYSIFSKVWLLALVWRRTCSLANRIPMIPVFTCLFCLFLASFLAEMAQTDMGYFIPSRVLVGLGAVCFTLFSIVSILEAGSAKK

SEQ ID NO: 244 - DNA, 815 bp, Corynebacterium glutamicum
  source 1..815: mol_type = genomic DNA
ttaaaaggactccgcttcgcctgggaaggtacccgcgtggatatcggcgatgtaggcctgcgcggcgtcgtgcaaggaatcgcccaaggtggcgtactcgcggacgaagcgtggcttcttgccgcggttgaggccgaaggcatcctgccacaccaaaacctgcccatctgtgccattgccggcaccgattccgatagtggtgatggaaagatcctcggtaacctcgcgcgctgcctctgctggaaccatctccaacacaaccgcaaacgcacccgcctgctccaacgcgcgggcatcggcgatgagctttccagaactcgcgccacgaccctgaaccacgtggccgcccaaggaatgctcggactgcggggtgtacccgatgtggccgacaaccggaattccagcatcaacaatgcgtcgaatcgtctgcgcgatctccacgccaccctcgatcttcaccgcagccgcacccgtttcacgcatgacccggatcgcggactccaccgcctgatttgggccacctcataggtaccaaacggcagatcaaccaccacaagcgcacgcttcgtagcgatcgtcaccgccttggccagcacaatcatctcatccaaggtgatcgacaaggtggtatcgcgacccagcacaacgttggcagcggaatcaccaacaaggagcatatcgacgccagcctcatcaaaaatgcgcgccgaaagcgcatcatagctggtgagaaccgaaactttctggccgtttactttagcttcgcggaaatgacgggtgcggattttctttgcatcaatgcctgacatgggcat

SEQ ID NO: 245 - protein, 271 aa, Corynebacterium glutamicum
MPMSGIDAKKIRTRHFREAKVNGQKVSVLTSYDALSARIFDEAGVDMLLVGDSAANVVLGRDTTLSITLDEMIVLAKAVTIATKRALVVVDLPFGTYEVSPNQAVESAIRVMRETGAAAVKIEGGVEIAQTIRRIVDAGIPVVGHIGYTPQSEHSLGGHVVQGRGASSGKLIADARALEQAGAFAVVLEMVPAEAAREVTEDLSITTIGIGAGNGTDGQVLVWQDAFGLNRGKKPRFVREYATLGDSLHDAAQAYIADIHAGTFPGEAESF

SEQ ID NO: 246 - DNA, 840 bp, Corynebacterium glutamicum
  source 1..840: mol_type = genomic DNA
ctagagctcgatattgtcgatcaaccgcaccggccccacgaaaatcgcgccgaccaccaacgctggttgggtgagcaggccgtcgatttctaatggttcgagggtggctggatcgacaatttccaggtgatccaagcgcacgccgtcggcgctggccaaggtgtcgcgcgcaccttggatatctagcgcttcaccagctgcttttcgacgctgcaacccactcaacacctgcggcagcaccagagcttgcgctcgctgatccgcagaaagacgttgattgcggctggattcggctaagccatcggcgccacgaataatcggaacgggacgaatctccacgggaatgtctagatcggcaaccaatcgccgaatcaccgcaacctgctgagcatctttttgtccaaaatatgcacgatcagggcgcaccaaattgaacagcttcgccaccacggtagccacaccatcgaaatggccaggcctgctggcaccctccaattttgttccgatggaaccggtgcgcgcccacactagtggcaagccaccggggtacatttcctccacatcgggtgcgaacacaatatccacacctgcctcttcaagcagtgctaaatcggcgtcgagttggcggggatagttgcggtaatcatcgcaatcaccgagtgcttcaaactgcaggggattgacaaaaatactggctacaacagtgtcgttttcagcgcgtgctgctttaaccaacgaggcgtgtccgctgtgtagcgcacccatggtggggacgagcccgacggatttgtggtggaggagggcgtcgataagcgcctgctttgtggttgctacctgcat

SEQ ID NO: 247 - protein, 279 aa, Corynebacterium glutamicum
MQVATTKQALIDALLHHKSVGLVPTMGALHSGHASLVKAARAENDTVVASIFVNPLQFEALGDCDDYRNYPRQLDADLALLEEAGVDIVFAPDVEEMYPGGLPLVWARTGSIGTKLEGASRPGHFDGVATVVAKLFNLVRPDRAYFGQKDAQQVAVIRRLVADLDIPVEIRPVPIIRGADGLAESSRNQRLSADQRAQALVLPQVLSGLQRRKAAGEALDIQGARDTLASADGVRLDHLEIVDPATLEPLEIDGLLTQPALVVGAIFVGPVRLIDNIEL

SEQ ID NO: 248 - DNA, 411 bp, Corynebacterium glutamicum
  source 1..411: mol_type = genomic DNA
ctaaatgcttctcgacgtcaaaagcccggatccaggtagtgcttccgcaagatcgttgccgagcgcaacgatgcggttgtcggcgtccacgtgcacaatctttggctcatacgccttggcttccgcatcagttgcctgaaggtagctcatgatgatcacaagatcgccaggattaataaggtgtgcagcggcaccattgatgcaaatattgcccgttccggcgtcgcccacaatgacataagtttccagacgagcgccgttggtgatgtctacgatggcaactttttcgccttcgatcaatccggcggcgtgaaccaggtcggcgtcgatggttacagagccaacataatctagatcagcttgagtgacagtggctcggtgaatcttacttccgaggatggtgcgcagcat

SEQ ID NO: 249 - protein, 136 aa, Corynebacterium glutamicum
MLRTILGSKIHRATVTQADLDYVGSVTIDADLVHAAGLIEGEKVAIVDITNGARLETYVIVGDAGTGNICINGAAAHLINPGDLVIIMSYLQATDAEAKAYEPKIVHVDADNRIVALGNDLAEALPGSGLLTSRSI

SEQ ID NO: 250 - DNA, 903 bp, Corynebacterium glutamicum
  source 1..903: mol_type = genomic DNA
atgaaaattgcgatcgttggcgctggtgcagttggtggatatttcggagcgttgttacaagaatctggtgcagatatcacgatggttgcacgtggacgaacattagaagccttgaagtctaaaggactccacatcaacgatgcaagaggcgaacgctacgtaccaattcctgcagttgcgagcgtgcaagaactaaaagatgcagatgtagtgatgattgctactaaagcattatcgcggtctttagatctcgctgaacttttgggtgggatacctgcgaattcggtggtcgcgattactcagaattcgattgaatctgctgatctagcagcgaagagtatcggtgctgatcgtgtgtggcctggtgtggttcgtgggttctttgttcatgaggggccagcctcagtgtcatacaagggaggcccactgtcctacacgtttggtgattctggtgaactttctaggcaattcgcaagcactcttgaacaggccggtattgacggagttctgcatcccgatattttggtggatgtgtgggagaaagccatgttcgtagaggttttcggcgggttgggggctttcgtcgaaaagcaattaggtaccttgcgtacgcattttagggcttccctggaagccttgatggaagaggtggctgaggtggctcgcgcggcaggtgttgcgttgccgagcgatgcggtggagcgcaccatgaattttgcggatcggatgcctgagaattcgacgagttcgatgcagcgtgatttggccgcgggagtggctagtgagcttgaggctcagacaggtgcaattgtgcgggcagcgcacaaagtgggtgtgaaaactccgcttcatgaccttatttatgctggtcttaagctgaaagaagaggaaaattcactttag

SEQ ID NO: 251 - protein, 300 aa, Corynebacterium glutamicum
MKIAIVGAGAVGGYFGALLQESGADITMVARGRTLEALKSKGLHINDARGERYVPIPAVASVQELKDADVVMIATKALSRSLDLAELLGGIPANSVVAITQNSIESADLAAKSIGADRVWPGVVRGFFVHEGPASVSYKGGPLSYTFGDSGELSRQFASTLEQAGIDGVLHPDILVDVWEKAMFVEVFGGLGAFVEKQLGTLRTHFRASLEALMEEVAEVARAAGVALPSDAVERTMNFADRMPENSTSSMQRDLAAGVASELEAQTGAIVRAAHKVGVKTPLHDLIYAGLKLKEEENSL

SEQ ID NO: 252 - DNA, 1290 bp, Corynebacterium glutamicum
  source 1..1290: mol_type = genomic DNA
atggaaacgactcaaaccagcacgattgcgtcgaaagactctcgtagtgcctggcgcaagacagacaccatgtggatgctgggcctttacggcacggcaatcggcgcgggcgtgctgttcctgccaatcaatgccggtgttggcggtatgatcccgctgatcatcatggctatccttgcgttcccgatgacattttttgctcaccgcggcctgactcgcttcgtactgtctggtaaaaacccaggcgaagacatcaccgaggttgtagaagaacactttggtattggcgcaggtaaactgattaccctgctctacttcttcgctatctacccgatcctgctggtttatagcgtggcaatcaccaatacagttgaaagcttcatgtctcaccagctgggtatgacaccaccgccgcgtgcgattctgtcgctgatcctgatcgtgggtatgatgaccatcgttcgcttcggtgagcagatgatcgttaaagcgatgagtattctggtattcccgtttgttggcgtactgatgctgctggctctgtacctgatcccgcagtggaacggcgcagcactggaaacgctgtctctggacactgcatcggcaaccggaaacggtctgtggatgaccctgtggctggcaattccggtaatggtgttctcgttcaaccactctccgatcatctcttctttcgccgttgcgaagcgtgaagagtacggcgatatggcagaacagaaatgctcgaagatccttgcattcgcacacatcatgatggtgctgaccgtaatgttcttcgtcttcagctgcgtactgagcctgactccggcagacctggctgcggctaaagagcagaacatctcgattctgtcttacctggctaaccactttaacgcaccggttatcgcgtggatggcaccgattatcgcgattatcgctatcaccaaatccttcctcggccactacctgggcgcacgtgaaggcttcaacggtatggtggttaaatctctgcgtggtaaaggtaagtctatcgaaatcaacaagctgaaccgtatcactgcgctgttcatgctggtaacgacctggattgttgccaccctgaacccgagcatcctgggtatgattgaaaccctgggcggcccaatcatcgcgatgatcctgttcctgatgccgatgtacgcaattcagaaagtaccggcaatgcgtaagtacagcggtcacatcagcaacgtattcgttgtcgtgatgggtctgattgcaatctccgcaatcttctactctctgttcagctaa

SEQ ID NO: 253 - protein, 429 aa, Corynebacterium glutamicum
METTQTSTIASKDSRSAWRKTDTMWMLGLYGTAIGAGVLFLPINAGVGGMIPLIIMAILAFPMTFFAHRGLTRFVLSGKNPGEDITEVVEEHFGIGAGKLITLLYFFAIYPILLVYSVAITNTVESFMSHQLGMTPPPRAILSLILIVGMMTIVRFGEQMIVKAMSILVFPFVGVLMLLALYLIPQWNGAALETLSLDTASATGNGLWMTLWLAIPVMVFSFNHSPIISSFAVAKREEYGDMAEQKCSKILAFAHIMMVLTVMFFVFSCVLSLTPADLAAAKEQNISILSYLANHFNAPVIAWMAPIIAIIAITKSFLGHYLGAREGFNGMVIKSLRGKGKSIEINKLNRITALFMLVTTWIVATLNPSILGMIETLGGPIIAMILFLMPMYAIQKVPAMRKYSGHISNVFVVVMGLIAISAIFYSLFS

SEQ ID NO: 254 - DNA, 1281 bp, Corynebacterium glutamicum
  source 1..1281: mol_type = genomic DNA
atgagttcagtttcgctgcaggattttgatgcagagcgaattggtttgttccacgaggacattaagcgcaagtttgatgagctcaagtcaaaaaatctgaagctggatcttactcgcggtaagccttcgtcggagcagttggatttcgctgatgagttgttggcgttgcctggtaagggtgatttcaaggctgcggatggtactgatgtccgtaactatggcgggctggatggcatcgttgatattcgccagatttgggcggatttgctgggtgttcctgtggagcaggtcttggcgggggatgcttcgagcttgaacatcatgtttgatgtgatcagctggtcgtacattttcggtaacaatgattcggttcagccttggtcgaaggaagaaaccgttaagtggatttgccctgttccgggctatgatcgccatttctccatcacggagcgtttcggctttgagatgatttctgtgccaatgaatgaagacggccctgatatggatgctgttgaggaattggtgaagaatccgcaggttaagggcatgtgggttgttccggtgttttctaacccgactggtttcacggtgacagaagacgtcgcaaagcgtctaagcgcaatggaaaccgcagctccggacttccgcgttgtgtgggataatgcctacgccgttcatacgctgaccgatgaattccctgaggttatcgatatcgtcgggcttggtgaggccgctggcaacccgaaccgtttctgggcgttcacttctacttcgaagatcactctcgcgggtgcgggcgtgtcgttcttcctcacctctgcggagaaccgcaagtggtacaccggccatgcgggtatccgtggcattggccctaacaaggtcaatcagttggctcatgcgcgttactttggcgatgctgagggagtgcgcgcggtgatgcgtaagcatgctgcgtcgttggctccgaagttcaacaaggttctggagattctggattctcgccttgctgagtacggtgtcgcgcagtggactgtccctgcgggcggttacttcatttcccttgatgtggttcctggtacggcgtctcgcgtggctgagttggctaaggaagccggcatcgcgttgacgggtgcgggttcttcttacccgctgcgtcaggatccggagaacaaaaatctccgtttggcaccgtcgctgcctccagttgaggaacttgaggttgccatggatggcgtggctacctgtgtgctgttggcagcagcggagcattacgctaactaa

SEQ ID NO: 255 - protein, 429 aa, Corynebacterium glutamicum; identical to SEQ ID NO: 253

SEQ ID NO: 256 - DNA, 1503 bp, Corynebacterium glutamicum
  source 1..1503: mol_type = genomic DNA
ttaggcttcctcaagcttcagggtcttctgggtgcgtgcccactgctgctcaaacagtgctggctcggaagcaagcttcttgccgtaggaagggatcatgtccttcagcttgtcgccccactcgatcatgcggtcaccgaagcaacgctcaagcagctcgatcattgcggaaggtgcgatggatgctccaggggaagcaccgagcaatccggcgatggtgccttcggagttgttgatcaaggtggtgccgaattccagggaaccgaacttagggaatcctgcaggcttaataacctgaacacgctgtccggcaacgatggtctcccaatcgccgttttgtgcctctggcatgtactcgcgaagagcatccatacgcttgtcctggtccttgagaacttcagtgacaaggtacttggtcagatcaaattcctgagcagcaacgccaaggtaggaaggaatgttgtctgggcggatggacttgaacaggtccaggtaggagccttccttcaagaacttaggggtccagccaccgtaaggtccaaagagcagacccttttcaccctcgataacgcgggtgtcaaggtgaggaacagacattggaggagcgccaacagatgccttgccatataccttggctgcgtgctgctcgatcagttcctcgttggtgcaacgaagccacaggccggatactgggaatccagcgaagcccttgacctgtgggatgcctgcgctgcgaagcagatccagtgcgtatccgcctgcgccgacgaacacgaagtttgccttgatggtcttggtgtcgccagtgtgtacgttcttgacggtcacgatccactttgcgccatcagccttgatgctcttgacttcgtggccatagcggatttcagtgccttcaacttcagctgcatccaggtactgcttggtctgagcaccgtagttgatgtcggtgccttcatcgatccaagagattgctactggatcagagaagtcacggccctttgccatcaaaggcagcttctcggtgaaggtagcttcatcgtcagcgtaggtcatgccctggaagagtgggtgatccttcaaagcttcgtagcgagccttgatgtatgcaacctgatctgcgccctggccgaaagatacgtgaggaacagggttgatgaattccttaggatcagacagcactccctcttcaacgaggtgagaccagaactgacgggaaacctggaacttctcgttgattcctacagccttggcaatttcaaccttgcccttaacctctggggtgtagttcagctcgcatagagcagagtggccggttcctgcattgttccacggggaggacgactcttgtgccggtccatccaaacgctcgaagacgatctgagtccagcttggctccagctgacgcagcattgcacccagcgtggagctcatgataccggcaccaatgagaactacatctgcctcatcggtaatcctcggtgcgttcttcggggaatctgacat

SEQ ID NO: 257 - protein, 500 aa, Corynebacterium glutamicum
MSDSPKNAPRITDEADVVLIGAGIMSSTLGAMLRQLEPSWTQIVFERLDGPAQESSSPWNNAGTGHSALCELNYTPEVKGKVEIAKAVGINEKFQVSRQFWSHLVEEGVLSDPKEFINPVPHVSFGQGADQVAYIKARYEALKDHPLFQGMTYADDEATFTEKLPLMAKGRDFSDPVAISWIDEGTDINYGAQTKQYLDAAEVEGTEIRYGHEVKSIKADGAKWIVTVKNVHTGDTKTIKANFVFVGAGGYALDLLRSAGIPQVKGFAGFPVSGLWLRCTNEELIEQHAAKVYGKASVGAPPMSVPHLDTRVIEGEKGLLFGPYGGWTPKFLKEGSYLDLFKSIRPDNIPSYLGVAAQEFDLTKYLVTEVLKDQDKRMDALREYMPEAQNGDWETIVAGQRVQVIKPAGFPKFGSLEFGTTLINNSEGTIAGLLGASPGASIAPSAMIELLERCFGDRMIEWGDKLKDMIPSYGKKLASEPALFEQQWARTQKTLKLEEA

SEQ ID NO: 258 - DNA, 1302 bp, Corynebacterium glutamicum
  source 1..1302: mol_type = genomic DNA
atggggtccggcaagacgatctccgttgcggggccatcagtaattttggtgtacgccattattggtttcatgcttttcttcgtcatgcgtgccatgggagagctgctgctcgccaatttgaattacaaatctttgcgcgatgcggtctctgatattttgggtcctggcgcaggttttgtcaccggctggacatattggttctgctggattgccacaggcatggcggacatcgtggcgatcactggatacacccaatactggtggcctgagatcccattgtggcttccaggtgtgctcaccattgcgttgctgtttgccctgaacttggctgcggtacgactgttcggtgagatggagttttggttcgccatcatcaaaatcgtggctatcgtgtccttgatcgtcgtgggacttttcatggtggtcacagcctttgaatcacctaatggcaccaccgcgcagttcaacaacctcattgagcatggcggatttttccccaacggcatcaccggtttcttggctggtttccagatcgctatctttgcgttcgtcgggattgaacttgccggcactgcagctgcagagactgagaatcccaccaagacgcttcctcgggcaatcaactccattcccatccgcatcgtggtgttctatgttttggcgttggctgtcatcatgatggtcaccccatgggatcaggtccgtgctgacaacagcccattcgtgcagatgttcgcgctggcaggaatcccagcggcggcaggcatcattaactttgtggtcatcacttctgcagcgtcgtctgccaacagtggtattttctccacctcccgcatgttgtatggattgtctttggaaggcgcagctccgaaacggtggagccggttgtccaagaacttggtgccagccaggggattgactttttctgtgatttgcctcattccagcggtgggtttgctgtacgctggcggcactgtcatcgaggcattcacactgatcaccacggtttcttcggtgttgttcatggtggtgtggtcctacattttggtggcttatatcgtctaccgccgcaacagcccggaattacacaaaaagtcgattttcaaaatgcctggcggcgtggtcatggcagttgtggtgttggtgttcttcgcagcgatgttggtggtgctgtccctggagccggatacccgtgcagcgctcatcgcgacgccagtgtggttcatcattttgggtatcggttggttgtccatcggtggagctaagggcgctaagcatcgcagccaaataacctcccactaa

SEQ ID NO: 259 - protein, 433 aa, Corynebacterium glutamicum
MGSGKTISVAGPSVILVYAIIGFMLFFVMRAMGELLLANLNYKSLRDAVSDILGPGAGFVTGWTYWFCWIATGMADIVAITGYTQYWWPEIPLWLPGVLTIALLFALNLAAVRLFGEMEFWFAIIKIVAIVSLIVVGLFMVVTAFESPNGTTAQFNNLIEHGGFFPNGITGFLAGFQIAIFAFVGIELAGTAAAETENPTKTLPRAINSIPIRIVVFYVLALAVIMMVTPWDQVRADNSPFVQMFALAGIPAAAGIINFVVITSAASSANSGIFSTSRMLYGLSLEGAAPKRWSRLSKNLVPARGLTFSVICLIPAVGLLYAGGTVIEAFTLITTVSSVLFMVVWSYILVAYIVYRRNSPELHKKSIFKMPGGVVMAVVVLVFFAAMLVVLSLEPDTRAALIATPVWFIILGIGWLSIGGAKGAKHRSQITSH

SEQ ID NO: 260 - DNA, 987 bp, Corynebacterium glutamicum
  source 1..987: mol_type = genomic DNA
ttagagcaagtcgcgcactgcctcgcgctcggcctgcaattcctgagcattcgcgtcgatgcgggcgcgctggaaatcggaaatctccaggccttcaacgatttcccactcaccgttgcgggatacggttggcagaccgacaaaaatgccctcaggaatgccgtatgcaccggtggaaggaattgccgcagaggaccacgcctcggtgccctgtacccaatcgcgcatgtgatcaatcgcagaggatgctgcagaagctgcagaagactttccacggacctcaatgatttcagcgccacggttagccacgcgaggaatgaactcctccacataccaatcgtgatcaaccaggtcagtgaccttttctccaccaacggttgcgtaggtgatgtctgggaactgggttgcggagtgatttccccagaccacaatgttgttaaattccgcagatccacggccaagcttggtggccagctgggagatcgcacggttgtgatcaaggcgcatcattgcgttgaagcgggatgctggaacatctggggccgcagctgaagcaatcaacgcgttggtgttcgctgggtttccaacaactaggacacgaatgtcatctgcggcgttgtcattgatagctttaccttgaggtccgaaaatcttgccgttgttagccagcaaatctgcgcgctcttcgccttttccgcgaggcttcgcaccgaccaaaaacgccgcattagcgccgtcgaatgcctcattggcatccgcggtgatggtgatgtttcgcaggagggggaaggcagaatccagaagttccatagccacaccctctgccccgccaagagcctgagggatctccagaagtttcagttctacaggggtgtcggtgccgaatacttcaccgttggcgatgcgccacaacagtgaataagagatttgaccagctgcgccggtgacggtgaccttcttggtggagacgttctgcggggaattcat

SEQ ID NO: 261 - protein, 328 aa, Corynebacterium glutamicum
MNSPQNVSTKKVTVTGAAGQISYSLLWRIANGEVFGTDTPVELKLLEIPQALGGAEGVAMELLDSAFPLLRNITITADANEAFDGANAAFLVGAKPRGKGEERADLLANNGKIFGPQGKAINDNAADDIRVLVVGNPANTNALIASAAAPDVPASRFNAMMRLDHNRAISQLATKLGRGSAEFNNIVVWGNHSATQFPDITYATVGGEKVTDLVDHDWYVEEFIPRVANRGAEIIEVRGKSSAASAASSAIDHMRDWVQGTEAWSSAAIPSTGAYGIPEGIFVGLPTVSRNGEWEIVEGLEISDFQRARIDANAQELQAEREAVRDLL

SEQ ID NO: 262 - DNA, 3898 bp, Choroflexus aurantiacus
  source 1..3898: mol_type = genomic DNA
aacatcatggtatactatacctatcgataattcttcaactaattgcataacagaacagcgatggcgacgggggagtccatgagcggaacaggacgactggcaggaaagattgcgttaattaccggtggcgccggcaatatcggcagtgaattgacacgtcgctttctcgcagagggagcgacggtcattattagtggacggaatcgggcgaagttgaccgcactggccgaacggatgcaggcagaggcaggagtgccggcaaagcgcatcgatctcgaagtcatggatgggagtgatccggtcgcggtacgtgccggtatcgaagcgattgtggcccgtcacggccagatcgacattctggtcaacaatgcaggaagtgccggtgcccagcgtcgtctggccgagattccactcactgaagctgaattaggccctggcgccgaagagacgcttcatgccagcatcgccaatttacttggtatgggatggcatctgatgcgtattgcggcacctcatatgccggtaggaagtgcggtcatcaatgtctcgaccatcttttcacgggctgagtactacgggcggattccgtatgtcacccctaaagctgctcttaatgctctatctcaacttgctgcgcgtgagttaggtgcacgtggcatccgcgttaatacgatctttcccggcccgattgaaagtgatcgcatccgtacagtgttccagcgtatggatcagctcaaggggcggcccgaaggcgacacagcgcaccattttttgaacaccatgcgattgtgtcgtgccaacgaccagggcgcgcttgaacgtcggttcccctccgtcggtgatgtggcagacgccgctgtctttctggccagtgccgaatccgccgctctctccggtgagacgattgaggttacgcacggaatggagttgccggcctgcagtgagaccagcctgctggcccgtactgatctgcgcacgattgatgccagtggccgcacgacgctcatctgcgccggcgaccagattgaagaggtgatggcgctcaccggtatgttgcgtacctgtgggagtgaagtgatcatcggcttccgttcggctgcggcgctggcccagttcgagcaggcagtcaatgagagtcggcggctggccggcgcagactttacgcctcccattgccttgccactcgatccacgcgatccggcaacaattgacgctgtcttcgattggggggccggcgagaataccggcgggattcatgcagcggtgattctgcctgctaccagtcacgaaccggcaccgtgcgtgattgaggttgatgatgagcgggtgctgaattttctggccgatgaaatcaccgggacaattgtgattgccagtcgcctggcccgttactggcagtcgcaacggcttacccccggcgcacgtgcgcgtgggccgcgtgtcatttttctctcgaacggtgccgatcaaaatgggaatgtttacggacgcattcaaagtgccgctatcggtcagctcattcgtgtgtggcgtcacgaggctgaacttgactatcagcgtgccagcgccgccggtgatcatgtgctgccgccggtatgggccaatcagattgtgcgcttcgctaaccgcagccttgaagggttagaatttgcctgtgcctggacagctcaattgctccatagtcaacgccatatcaatgagattaccctcaacatccctgccaacattagcgccaccaccggcgcacgcagtgcatcggtcggatgggcggaaagcctgatcgggttgcatttggggaaagttgccttgattaccggtggcagcgccggtattggtgggcagatcgggcgcctcctggctttgagtggcgcgcgcgtgatgctggcagcccgtgatcggcataagctcgaacagatgcaggcgatgatccaatctgagctggctgaggtggggtataccgatgtcgaagatcgcgtccacattgcaccgggctgcgatgtgagtagcgaagcgcagcttgcggatcttgttgaacgtaccctgtcagcttttggcaccgtcgattatctgatcaacaacgccgggatcgccggtgtcgaagagatggttatcgatatgccagttgagggatggcgccataccctcttcgccaatctgatcagcaactactcgttgatgcgcaaactggcgccgttgatgaaaaaacagggtagcggttacatccttaacgtctcatcatactttggcggtgaaaaagatgcggccattccctaccccaaccgtgccgattacgccgtctcgaaggctggtcagcgggcaatggccgaagtctttgcgcgcttccttggcccggagatacagatcaatgccattgcgccgggtccggtcgaaggtgatcgcttgcgcggtaccggtgaacgtcccggcctctttgcccgtcgggcgcggctgattttggagaacaagcggctgaatgagcttcacgctgctcttatcgcggctgcgcgcaccgatgagcgatctatgcacgaactggttgaactgctcttacccaatgatgtggccgcactagagcagaatcccgcagcacctaccgcgttgcgtgaactggcacgacgttttcgcagcgaaggcgatccggcggcatcatcaagcagtgcgctgctgaaccgttcaattgccgctaaattgctggctcgtttgcataatggtggctatgtgttgcctgccgacatctttgcaaacctgccaaacccgcccgatcccttcttcacccgagcccagattgatcgcgaggctcgcaaggttcgtgacggcatcatggggatgctctacctgcaacggatgccgactgagtttgatgtcgcaatggccaccgtctattaccttgccgaccgcaatgtcagtggtgagacattccacccatcaggtggtttgcgttacgaacgcacccctaccggtggcgaactcttcggcttgccctcaccggaacggctggcggagctggtcggaagcacggtctatctgataggtgaacatctgactgaacaccttaacctgcttgcccgtgcgtacctcgaacgttacggggcacgtcaggtagtgatgattgttgagacagaaaccggggcagagacaatgcgtcgcttgctccacgatcacgtcgaggctggtcggctgatgactattgtggccggtgatcagatcgaagccgctatcgaccaggctatcactcgctacggtcgcccagggccggtcgtctgtacccccttccggccactgccgacggtaccactggtcgggcgtaaagacagtgactggagcacagtgttgagtgaggctgaatttgccgagttgtgcgaacaccagctcacccaccatttccgggtagcgcgcaagattgccctgagtgatggtgccagtctcgcgctggtcactcccgaaactacggctacctcaactaccgagcaatttgctctggctaacttcatcaaaacgacccttcacgcttttacggctacgattggtgtcgagagcgaaagaactgctcagcgcattctgatcaatcaagtcgatctgacccggcgtgcgcgtgccgaagagccgcgtgatccgcacgagcgtcaacaagaactggaacgttttatcgaggcagtcttgctggtcactgcaccactcccgcctgaagccgatacccgttacgccgggcggattcatcgcggacgggcgattaccgtgtaaattctacgccacaggaaccactaccaaaccagcatagtaagagaacgatagagacgttgcaatgcgacgtctctatcatatttccggccccccctagacaaacccccacgtcttcgtgtagactagaaacaggaggctgtatgcacgtccaacaaga

SEQ ID NO: 263 - protein, 1220 aa, Choroflexus aurantiacus
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 264 - DNA, 1266 bp, Escherichia coli
  source 1..1266: mol_type = genomic DNA
atgagcaacaatgaattccatcagcgtcgtctttctgccactccgcgcggggttggcgtgatgtgtaacttcttcgcccagtcggctgaaaacgccacgctgaaggatgttgagggcaacgagtacatcgatttcgccgcaggcattgcggtgctgaataccggacatcgccaccctgatctggtcgcggcggtggagcagcaactgcaacagtttacccacaccgcgtatcagattgtgccgtatgaaagctacgtcaccctggcggagaaaatcaacgcccttgccccggtgagcgggcaggccaaaaccgcgttcttcaccaccggtgcggaagcggtggaaaacgcggtgaaaattgctcgcgcccataccggacgccctggcgtgattgcgtttagcggcggctttcacggtcgtacgtatatgaccatggcgctgaccggaaaagttgcgccgtacaaaatcggcttcggcccgttccctggttcggtgtatcacgtaccttatccgtcagatttacacggcatttcaacacaggactccctcgacgccatcgaacgcttgtttaaatcagacatcgaagcgaagcaggtggcggcgattattttcgaaccggtgcagggcgagggcggtttcaacgttgcgccaaaagagctggttgccgctattcgccgcctgtgcgacgagcacggtattgtgatgattgctgatgaagtgcaaagcggctttgcgcgtaccggtaagctgtttgccatggatcattacgccgataagccggatttaatgacgatggcgaaaagcctcgcgggcgggatgccgctttcgggcgtggtcggtaacgcgaatattatggacgcacccgcgccgggcgggcttggcggcacctacgccggtaacccgctggcggtggctgccgcgcacgcggtgctcaacattatcgacaaagaatcactctgcgaacgcgcgaatcaactgggccagcgtctcaaaaacacgttgattgatgccaaagaaagcgttccggccattgctgcggtacgcggcctggggtcgatgattgcggtagagtttaacgatccgcaaacgggcgagccgtcagcggcgattgcacagaaaatccagcaacgcgcgctggcgcaggggctgctcctgctgacctgtggcgcatacggcaacgtgattcgcttcctgtatccgctgaccatcccggatgcgcaattcgatgcggcaatgaaaattttgcaggatgcgctgagcgattaa

SEQ ID NO: 265 - protein, 421 aa, Escherichia coli
MSNNEFHQRRLSATPRGVGVMCNFFAQSAENATLKDVEGNEYIDFAAGIAVLNTGHRHPDLVAAVEQQLQQFTHTAYQIVPYESYVTLAEKINALAPVSGQAKTAFFTTGAEAVENAVKIARAHTGRPGVIAFSGGFHGRTYMTMALTGKVAPYKIGFGPFPGSVYHVPYPSDLHGISTQDSLDAIERLFKSDIEAKQVAAIIFEPVQGEGGFNVAPKELVAAIRRLCDEHGIVMIADEVQSGFARTGKLFAMDHYADKPDLMTMAKSLAGGMPLSGVVGNANIMDAPAPGGLGGTYAGNPLAVAAAHAVLNIIDKESLCERANQLGQRLKNTLIDAKESVPAIAAVRGLGSMIAVEFNDPQTGEPSAAIAQKIQQRALAQGLLLLTCGAYGNVIRFLYPLTIPDAQFDAAMKILQDALSD

SEQ ID NO: 266 - DNA, 1503 bp, Rattus norvegicus
  source 1..1503: mol_type = genomic DNA
atggccttcttgttgactacccgacggctggtctgcagttcccagaaaaacctccacctcttcacacctggatccagatacatcagccaagctgctgccaaagttgactttgagtttgattatgatggaccactcatgaagacagaagtcccgggccctagatctcaggagctaatgaaacagctgaacacaatccagaatgcagaggccgtgcactttttctgcaactacgaagagagccgaggcaactacctcgtggacgtggatggcaaccgcatgttggacctgtattctcagatctcctctgtacccatcggttacaaccatccggctctggcgaaactcgttcaacagcctcaaaacgcgagcactttcatcaacagacctgccctgggcatcctgcctccagagaactttgtggacaagctccgggagtccttgatgtcggtggcgcccaaaggcatgtgtcagctcatcacgatggcctgcgggtcctgctccaatgagaatgcattcaagaccatcttcatgtggtaccggagtaaagaacgaggtcagagaggtttctccaaagaggagctggagacttgcatggttaaccagagtcctggatgcccagactacagcatcctctccttcatgggtgctttccacgggaggaccatgggttgcttagcgaccacacactccaaagcaattcacaagattgacatcccttcctttgactggcccattgctccattcccacggctgaaatatcccctggaggagtttgtgacggacaatcagcaagaggaggcccgctgtctagaagaggtggaggatctaattgtgaaatatcggaaaaagaagagaacagtggctgggatcatcgtggagcccatccagtccgaaggtggagacaaccacgcatcagatgacttcttccggaagctgagagacatagccaggaagcatggctgtgccttcttggtggacgaggttcagactggaggaggctgtacaggcaagttctgggcccatgaacactggggcttggatgacccagccgacgtgatgtcgttcagcaagaagatgatgactgggggcttcttccacaaggaggagtttcgaccaagtgctccttaccggatcttcaacacctggctgggggacccatccaagaacttgctgctggctgaggtcatcaacatcatcaagcgggaagacctgctcaacaacgtggcccatgccgggaagaccctactgaccgggctgctggacctccaggcccagtacccccagttcgtcagccgggtgaggggacgaggcaccttctgttccttcgacactcccgacaaagccatacggaataaactcatcctaattgccaggaacaaaggtgtggtactggggggctgcggtgacaaatccatacgtttccgtcccacgctggtcttcagggatcaccatgcccacttgttcctcaacattttcagtggtatcttagcagacttcaagtaa

SEQ ID NO: 267 - protein, 500 aa, Rattus norvegicus
MAFLLTTRRLVCSSQKNLHLFTPGSRYISQAAAKVDFEFDYDGPLMKTEVPGPRSQELMKQLNTIQNAEAVHFFCNYEESRGNYLVDVDGNRMLDLYSQISSVPIGYNHPALAKLVQQPQNASTFINRPALGILPPENFVDKLRESLMSVAPKGMCQLITMACGSCSNENAFKTIFMWYRSKERGQRGFSKEELETCMVNQSPGCPDYSILSFMGAFHGRTMGCLATTHSKAIHKIDIPSFDWPIAPFPRLKYPLEEFVTDNQQEEARCLEEVEDLIVKYRKKKRTVAGIIVEPIQSEGGDNHASDDFFRKLRDIARKHGCAFLVDEVQTGGGCTGKFWAHEHWGLDDPADVMSFSKKMMTGGFFHKEEFRPSAPYRIFNTWLGDPSKNLLLAEVINIIKREDLLNNVAHAGKTLLTGLLDLQAQYPQFVSRVRGRGTFCSFDTPDKAIRNKLILIARNKGVVLGGCGDKSIRFRPTLVFRDHHAHLFLNIFSGILADFK

SEQ ID NO: 268 - DNA, 1368 bp, Bacillus smithii
  source 1..1368: mol_type = genomic DNA
atgaaaacgatggatgtgattcaaacaaaaaaagaagatcaacagtatgtcttgcattcatggtccaaacaaaagaatattcatccgaaagtcatcacgaaagctgaaggcatttttttctgggatgatgaaaaccgaaaatactatgatatgtgttcacaactcgtttacttgaatgttggacatcagcatcctaaattgttggaagaatttaagcatatggatgaaattcctatcgctgcaccttcatttgcgacaccatcaaaatcagaactcgctcgcaaaattattgaagttgcgccggacaacatggcgaaagtattttttgcaaacggtggagcagattcaaatgatcatgctattaaaatcgcaaggatggtcacaggacgttataaaattttttcaaggtatcgttcgtaccacggtgccacattcggcgccggcaatttaaccggtgaaaatcgtcgatttgctgttgaacctgggatgccgggttttattaaatttgatacaccttatttgtaccgggagtctattaattttgaaaatgaagaagcggcagcaaagttttaccttgatcggttgcacaatcaaatgctatatgaaggaccggaatccattgctgctgtatttcttgaacccattccgggcagcaatggtgttctcattccgccaaaaggatatcttgaaggggtgcgtgaattatgtgaccgctacggaattcttatggtttgtgatgaagtgatgtcgggatttggaagaacaggaaaaatgttcgcggtcgatcattttaacttccaaccagatatgatgacgtttgctaaaggggtaacaagtggttatagcccgcttggcggtgtgattgtcagccgaaaagtggcggagtttttcgatgaccatgtattaatgacaggtttaacatacagcggacataccgtttcaacccaaatcgggtgtgcaacgcttgatatttataaagaagaaggtctcattgaaaatgcttctgaagtaggtgaagtgctaggagcaagattggaaaaactaagagcgtttaactcagttggtgatgttcgtcatatcggtttattcgcagccgttgaacttgttagagacaaaaagacaaaggaaccgattatatcgtatgggatggattatggaaaagatgaagcgggattaatgaaaacattcattcatttattgaatgaaaagggattttatacgtactcgcatgaatccagtgtgatcattgcacctccattaatcattacagaacaacagataaatgaggcgatggatcttttcgaagaagcattgaccaagtttgaaaaggagcatttattctctgctggaacattggtaactaaataa

SEQ ID NO: 269 - protein, 455 aa, Bacillus smithii
MKTMDVIQTKKEDQQYVLHSWSKQKNIHPKVITKAEGIFFWDDENRKYYDMCSQLVYLNVGHQHPKLLEEFKHMDEIPIAAPSFATPSKSELARKIIEVAPDNMAKVFFANGGADSNDHAIKIARMVTGRYKIFSRYRSYHGATFGAGNLTGENRRFAVEPGMPGFIKFDTPYLYRESINFENEEAAAKFYLDRLHNQMLYEGPESIAAVFLEPIPGSNGVLIPPKGYLEGVRELCDRYGILMVCDEVMSGFGRTGKMFAVDHFNFQPDMMTFAKGVTSGYSPLGGVIVSRKVAEFFDDHVLMTGLTYSGHTVSTQIGCATLDIYKEEGLIENASEVGEVLGARLEKLRAFNSVGDVRHIGLFAAVELVRDKKTKEPIISYGMDYGKDEAGLMKTFIHLLNEKGFYTYSHESSVIIAPPLIITEQQINEAMDLFEEALTKFEKEHLFSAGTLVTK

(19) United States
(12) Patent Application Publication
(10) Pub. No.: US 2026/0015634 A1
(43) Pub. Date: Jan. 15, 2026

(54) METHOD TO PRODUCE ORGANOSULFUR COMPOUNDS USING GENETICALLY MODIFIED MICROORGANISMS

(71) Applicant: Chemical Evolution Ltd., Calgary (CA)

(72) Inventors: Alejandra ENRIQUEZ, Calgary (CA); Trevor RANDALL, Calgary (CA); Dustin LILLICO, Calgary (CA); Markus WEISSENBERGER, Calgary (CA)

(21) Appl. No.: 19/020,904

(22) Filed: Jan. 14, 2025

(30) Foreign Application Priority Data
Jul. 12, 2024 (CA) 3248304

(51) Int. Cl. C12P 11/00; C12N 1/20; C12N 9/02; C12N 9/80; C12R 1/15

(52) U.S. Cl. CPC C12P 11/00; C12N 1/20; C12N 9/0069; C12N 9/0073; C12N 9/80; C12R 2001/15; C12Y 113/11019; C12Y 114/13008; C12Y 305/01092

(57) ABSTRACT

A method of producing an organosulfur compound from a prokaryotic cell wherein said method comprises the steps of:
a. providing a live prokaryotic cell capable of expressing at least one gene for the production of said organosulfur compound;
b. exposing said live prokaryotic cell to a culture media with a pH of between 4 and 11 containing a carbon source and a sulfur source thereby creating an incubation mixture;
c. incubating said live prokaryotic cell in said incubation mixture under aerobic or anaerobic conditions at a temperature ranging from 0° C. to 60° C. for a period of time sufficient for the expression of said at least one gene for the production of said organosulfur compound;
d. recovering said organosulfur compound from the bacterial cells and/or spent media; and
e. optionally, re-exposing said live prokaryotic cell to an unused media or spent media for the continuous production of said organosulfur compound of interest.

### REFERENCE TO AN ELECTRONIC SEQUENCE LISTING

The contents of the electronic sequence listing (2025-08-04_SequenceListing_ST26_23156-198US1.xml; Size: 446,439 bytes; and Date of Creation: Jul. 29, 2025) is herein incorporated by reference in its entirety.

### FIELD OF THE INVENTION

The present invention relates to a method to produce an organosulfur compound through the fermentation of a sugar source and a sulfur source by genetically manipulated microorganisms containing naturally present or added metabolic pathways.

### BACKGROUND OF THE INVENTION

The importance of organosulfur compounds in industry is quite vast, especially in the fields of nutrition and supplementation, cosmetics, and pharmaceuticals. As examples, cysteamine, in its bitartrate form, is an approved drug for the treatment of cystinosis while as part of cosmetic creams, it is used as a skin-lightening agent for people with hyperpigmentation disorders. On the other hand, taurine has been shown to be beneficial to human and animal health and development. For this reason, taurine is commonly found supplemented into animal feed for livestock, as a stimulant in energy drinks, sold directly as a supplement, and in baby formula.

Currently, most of these materials are synthesized chemically, biologically, or through the extraction from protein hydrolysates. As an example, most of the taurine on the market is chemically synthesized from either ethylene oxide or monoethanolamine (MEA). Manufacturing taurine using ethylene oxide, although the most common, comes with several drawbacks including its toxicity, volatility, and explosive potential, in addition to the severe conditions of temperature and pressure under which the reaction takes place. On the other hand, the industrial production of taurine from MEA is a two-step batch process in which the first step is the reaction of MEA with sulfuric acid to produce the ester 2-aminoethyl hydrogen sulfate (AES) and then second involving a subsequent reaction of AES with a sulfite reagent. This general manufacturing process has limitations which includes the yield of the intermediate esters involved within the above-mentioned reactions and the need for high temperatures and corrosive acids to achieve production. Additionally, the reliance of chemical pathways on non-renewable sources, and associated environmental pollution have emerged as critics that are incentivizing the implementation of newer and greener technologies for compound production.

To overcome these limitations, the present invention utilizes a prokaryotic biological system for the production of taurine through specific genetic modifications that incorporate pathways exclusively associated with eukaryotes. Taurine production by eukaryotes is facilitated by very distinct sets of metabolic pathways which includes the conversion of methionine and cysteine via cysteine sulfinic acid decarboxylase (CSAD) coupled with the oxidation of hypotaurine to generate taurine as the final step in the pathway.

U.S. Pat. No. 11,220,691 B2 describes an approach to produce or increase hypotaurine or taurine production in unicellular organisms. More particularly, this document relates to genetic modification of unicellular organisms that include bacteria, algal, microalgal, diatoms, yeast, or fungi. This document relates to methods to increase taurine levels in the cells by binding taurine or decreasing taurine degradation. According to this document, the invention can be used in organisms that contain native or heterologous (transgenic) taurine biosynthetic pathways or cells that have taurine by enrichment. This patent also relates to methods to increase taurine levels in the cells and to use the said cells or extracts or purifications from the cells that contain the invention to produce plant growth enhancers, food, animal feed, aquafeed, food or drink supplements, animal-feed supplements, dietary supplements, health supplements or taurine.

PCT patent application number WO2023/146544 describes methods for the fermentative production of taurine-containing products from unicellular organisms. More particularly, the invention relates to genetic modifications of the taurine and/or substrate biosynthetic pathways in unicellular organisms that include bacteria, algae, microalgae, diatoms, yeast, or fungi. The invention also relates to fermentation and processing methods for the production of various taurine-containing products. The invention also relates to the use of the cells, fermentation broth or extracts that contain taurine to produce products for use in food, feed, beverages, dietary and health supplements, cosmetics, personal care, pharmaceuticals, or agricultural production.

PCT patent application number WO217/083351 A1 discloses non-naturally occurring microorganisms that produce taurine and/or taurine precursors, e.g., hypotaurine, sulfoacetaldehyde, or cysteate, utilizing exogenously added enzyme activities. Methods of producing taurine and/or taurine precursors in microbial cultures, and feed and nutritional supplement compositions that include taurine and/or taurine precursors produced in the microbial cultures, such as taurine- and/or taurine precursor-containing biomass, are also provided.

Korean patent KR 2010/0130564A discloses a recombinant mutant microorganism having taurine generating ability and a method for producing taurine using the same, and more particularly, to a recombinant mutant microorganism having taurine generating ability and a method for producing taurine using the same.

Chinese patent CN102965317 discloses a method for screening taurine strains and a method for producing taurine through fermenting the strains, which belong to the technical field of bioengineering. According to the strains, soil is firstly subjected to boiling water bath and then is diluted and coated, endospore strains are obtained through staining and microscopy, the screened strains are then subjected to shake-flask culture one by one through adopting primary fermentation, a taurine standard substance is added into fermentation liquor to be used as an internal standard, a high performance liquid chromatography is adopted for qualitative analytical screening, the screened strains are subjected to morphological, physiological and biochemical and molecular biological identification, and finally, the Bacillus subtilis of one high-yield taurine is obtained. The invention further discloses a method for producing taurine through adopting the strains. The method is adopted to ferment 72 hours, and the taurine yield is 1 gram/liter.

PCT patent application number WO2024125795 A1 discloses a method for isolating taurine from a fermentation broth following the microbial fermentation of a production strain, having the following isolation steps in the given order: (i) isolating the fermentation supernatant by separating the biomass from the fermentation broth, (ii) precipitating taurine from the fermentation supernatant by adding a water-miscible solvent 1 (e.g. ethanol), (iii) isolating and dissolving the taurine precipitate in a solvent 2 (water), (iv) crystallizing the dissolved taurine, and (v) isolating and optionally drying the crystallized taurine, wherein the isolated taurine is obtained with a degree of purity of >80% (w/w). In this manner, a simple method is provided for isolating fermentatively prepared taurine with a high degree of purity.

In J. Agric. Food Chem. 2018, 66, 51, 13454-13463, Joo et al. reported, for the first time in bacteria, the production of taurine in metabolically engineered Corynebacterium glutamicum. The taurine-producing strain was developed by introducing CS, CDO1, and CSAD genes. Interestingly, while the control strain could not produce taurine, the engineered strains successfully produced taurine via the newly introduced metabolic pathway.

U.S. Pat. No. 11,326,171 B2 provides non-naturally occurring microorganisms that produce taurine and/or taurine precursors, e.g., hypotaurine, sulfoacetaldehyde, or cysteate, utilizing exogenously added enzyme activities. This patent relates to methods of producing taurine and/or taurine precursors in microbial cultures, and feed and nutritional supplement compositions that include taurine and/or taurine precursors produced in the microbial cultures, such as taurine- and/or taurine precursor-containing biomass, are also provided.

In light of the state of the art, there still exists a need to develop a large scale, environmentally conscious process for the production of organosulfur compounds (such as taurine), which is a cost-effective alternative to current chemical and biological processes and that relies on commodity chemicals with world-wide availability. In contrast to existing chemical methods, the process described herein is operated at mild conditions of temperature, reducing energy consumption and preventing product degradation and by-product generation. This is the first report to the inventor's knowledge of a strain utilizing the ADO pathway for taurine production at scale.

### SUMMARY OF THE INVENTION

The embodiments of the present invention are generally related to novel methods for the production of organosulfur compounds that utilize genetically engineered microorganisms.

According to a first embodiment of the present invention, there is provided a method of producing an organosulfur compound from a prokaryotic cell wherein said prokaryotic cell comprises a vanin (vnn) polynucleotide sequence selected from the group consisting of: vanin-1 (vnn1), vanin-2 (vnn2), and vanin-3 (vnn3).

According to another aspect of the present invention, there is provided a method of producing an organosulfur compound from a prokaryotic cell wherein said method comprises the steps of:
a) exposing said prokaryotic cell to a culture media with a pH of between 4 and 11 containing a carbon source and a sulfur source thereby creating an incubation mixture;
b) incubating said prokaryotic cell in said incubation mixture under aerobic or anaerobic conditions at a temperature ranging from 0° C. to 60° C. for a period of time sufficient for the expression of said genes for the production of said organosulfur compound;
c) recovering said organosulfur compound from the bacterial cells and/or spent media;
d) optionally, re-exposing said live prokaryotic cell to unused or spent media for the continuous production of said organosulfur compound.
   and wherein said prokaryotic cell comprises at least one of the following polynucleotide sequences:
i. a vanin (vnn) polynucleotide sequence selected from the group consisting of:
   vanin-1 (vnn1), wherein said vnn1 polynucleotide sequence has at least 70% sequence coverage to SEQ 3 or SEQ 98, and at least 70% sequence identity to SEQ 3 or SEQ 98;
   vanin-2 (vnn2), wherein said vnn2 polynucleotide sequence has at least 70% sequence coverage to SEQ 100, and at least 70% sequence identity to SEQ 100; and
   vanin-3 (vnn3), wherein said vnn3 polynucleotide sequence has at least 70% sequence coverage to SEQ 141, and at least 70% sequence identity to SEQ 141;
ii. a cysteamine dioxygenase (ado) polynucleotide sequence which has at least 70% sequence coverage to SEQ 1, and at least 70% sequence identity to SEQ 1; and
iii. a flavin-containing monooxygenase 1 (fmo1) polynucleotide sequence which has at least 70% sequence coverage to SEQ 5 or SEQ 99, and at least 70% of sequence identity to SEQ 5 or SEQ 99.

According to a preferred embodiment of the present invention, there is provided a method of growing a prokaryotic cell, wherein the method comprises the steps of:
providing a live prokaryotic cell capable of expressing genes for the production of an organosulfur compound of interest;
exposing said live prokaryotic cell to a culture media with a pH of between 4 and 11 thereby creating an incubation mixture;
incubating said live prokaryotic cell in said incubation mixture under aerobic and/or anaerobic conditions at a temperature ranging from 0° C. to 60° C. for a period of time ranging from 3 hours to 15 days allowing for the growth of said live prokaryotic cell;

According to a preferred embodiment of the present invention, there is provided a method of growing a prokaryotic cell wherein said method comprises the steps of:
exposing said live prokaryotic cell to a culture media with a pH of between 4 and 11, thereby creating an incubation mixture;
incubating said live prokaryotic cell in said incubation mixture under aerobic and/or anaerobic conditions at a temperature ranging from 0° C. to 60° C. for a period of time ranging from 3 hours to 15 days allowing for the growth of said live prokaryotic cell;
   and wherein said prokaryotic cell comprises at least one of the following polynucleotide sequences:
i. a vanin (vnn) polynucleotide sequence selected from the group consisting of:
   vanin-1 (vnn1), wherein said vnn1 polynucleotide sequence has at least 70% sequence coverage to SEQ 3 or SEQ 98, and at least 70% sequence identity to SEQ 3 or SEQ 98;
   vanin-2 (vnn2), wherein said vnn2 polynucleotide sequence has at least 70% sequence coverage to SEQ 100, and at least 70% sequence identity to SEQ 100; and
   vanin-3 (vnn3), wherein said vnn3 polynucleotide sequence has at least 70% sequence coverage to SEQ 141, and at least 70% sequence identity to SEQ 141;
ii. a cysteamine dioxygenase (ado) polynucleotide sequence which has at least 70% sequence coverage to SEQ 1, and at least 70% sequence identity to SEQ 1; and
iii. a flavin-containing monooxygenase 1 (fmo1) polynucleotide sequence which has at least 70% sequence coverage to SEQ 5 or SEQ 99, and at least 70% of sequence identity to SEQ 5 or SEQ 99.

According to a preferred embodiment of the present invention said live prokaryotic cell can be re-exposed to an unused media or a spent media for the continuous production of said organosulfur compound.

According to a preferred embodiment of the present invention, the live prokaryotic cell capable of expressing genes for the production of an organosulfur compound is a bacterial cell. Preferably, said bacterial cell is selected from the group consisting of the genera: Brevibacterium, Bacillus, Corynebacterium, Escherichia, Lactococcus, Pseudomonas, Rhodococcus, and Serratia. More preferably, the bacterial cell belongs to the genus Corynebacterium. Preferably, the bacterial cell is Corynebacterium glutamicum.

According to a preferred embodiment of the present invention, the carbon source is selected from the group comprising of: long-chain saccharides, short chain saccharides, monosaccharides, sugar alcohols, organic acids and their corresponding salts and/or combinations thereof. According to a more preferred embodiment of the present invention, the carbon source is selected from the group comprising of: glucose, mannose, maltose, lactose, galactose, fructose, sucrose, molasses, ribose, arabinose, xylose, gluconic acid and gluconate salts, arabitol, mannitol, sorbitol, maltitol, xylitol, erythritol, lactitol, acetic acid, acetate salts, starch and starch hydrolysates, corn steep liquor (CSL), whey or whey permeate, bagasse hydrolysate, glycerol, fumaric acid and its salts, lactic acid and its salts, ethanol, methanol, and/or combinations thereof.

According to a preferred embodiment of the present invention, said sulfur source is selected from the group consisting of: inorganic compounds comprising sulfur in any oxidation state; organic compounds comprising sulfur in any oxidation state; and their corresponding salts. Some non-limiting examples include elemental sulfur; sulfides; thiols (i.e., cysteamine, ethanethiol, propanethiol, cysteine, N-acetyl-cysteine); sulfate, sulfite, and thiosulfate salts (i.e., ammonium, magnesium, potassium); and/or combinations thereof.

According to a preferred embodiment of the present invention, said organosulfur compound of interest is selected from the group consisting of: cysteamine; hypotaurine; and taurine. According to an even more preferred embodiment of the present invention, said organosulfur compound of interest is taurine.

It is known to those skilled in the art that a plethora of different techniques can be utilized for the extraction and purification of the desired compound from the cells or the mixture in which the cells are. These methods include, but are not limited to, centrifugation, filtration, dialysis, crystallization, ion exchange, chromatography, electrodialysis, solvent extraction, evaporation and nanofiltration.

According to a preferred embodiment of the present invention, there is provided a method of growing a modified prokaryotic organism, wherein the method comprises the steps of:
providing a live prokaryotic cell capable of expressing genes for the production of an organosulfur compound;
exposing said live prokaryotic cell to a culture media with a pH of between 4 and 11 thereby creating an incubation mixture comprising a first cell mass;
growing said incubation mixture comprising a first cell mass under aerobic and/or anaerobic conditions at a temperature ranging from 0° C. to 60° C. for a period of time necessary so that it creates a second cell mass of the organism greater than the first cell mass;

According to a preferred embodiment of the present invention, there is provided a method of growing a modified prokaryotic organism, wherein the method comprises the steps of:
providing a live prokaryotic cell capable of expressing genes for the production of an organosulfur compound of interest;
exposing said live prokaryotic cell to a culture media with a pH of between 4 and 11 thereby creating an incubation mixture comprising a first cell mass;
growing said incubation mixture comprising a first cell mass under aerobic and/or anaerobic conditions at a temperature ranging from 0° C. to 60° C. for a period of time necessary so that it creates a second cell mass of the organism greater than the first cell mass;
   and wherein said prokaryotic organism comprises at least one of the following polynucleotide sequences:
i. a vanin (vnn) polynucleotide sequence selected from the group consisting of:
   vanin-1 (vnn1), wherein said vnn1 polynucleotide sequence has at least 70% sequence coverage to SEQ 3 or SEQ 98, and at least 70% sequence identity to SEQ 3 or SEQ 98;
   vanin-2 (vnn2), wherein said vnn2 polynucleotide sequence has at least 70% sequence coverage to SEQ 100, and at least 70% sequence identity to SEQ 100; and
   vanin-3 (vnn3), wherein said vnn3 polynucleotide sequence has at least 70% sequence coverage to SEQ 141, and at least 70% sequence identity to SEQ 141;
ii. a cysteamine dioxygenase (ado) polynucleotide sequence which has at least 70% sequence coverage to SEQ 1, and at least 70% sequence identity to SEQ 1; and
iii. a flavin-containing monooxygenase 1 (fmo1) polynucleotide sequence which has at least 70% sequence coverage to SEQ 5 or SEQ 99, and at least 70% of sequence identity to SEQ 5 or SEQ 99.

According to another aspect of the present invention, there is provided a method to produce an organosulfur compound from a prokaryotic cell wherein said prokaryotic cell comprises at least one of the following polynucleotide sequences:
a vanin (vnn) polynucleotide sequence selected from the group consisting of:
   i. a vanin 1 (vnn1) polynucleotide sequence, wherein said vnn1 polynucleotide sequence is selected from the group consisting of: SEQ 3; SEQ 45, SEQ 46, SEQ, 47, SEQ 48, SEQ 49, SEQ 50, SEQ 51, SEQ 52, SEQ 53, SEQ 54, and SEQ 98;
   ii. vanin 2 (vnn2) polynucleotide sequence, wherein said vnn2 polynucleotide sequence is selected from the group consisting of: SEQ 100; SEQ 101; SEQ 102; SEQ 103; SEQ 104; SEQ 105; SEQ 106; SEQ 107; SEQ 108; SEQ 109; SEQ 110; SEQ 111; SEQ 112; and SEQ 113; and
   iii. vanin 3 (vnn3) polynucleotide sequence, wherein said vnn3 polynucleotide sequence is selected from the group consisting of: SEQ 141; SEQ 142; SEQ 143; SEQ 144; SEQ 145; SEQ 146; SEQ 147; SEQ 148; SEQ 149; SEQ 150; SEQ 151; SEQ 152; SEQ 153; SEQ 154; SEQ 155; SEQ 156; and SEQ 157;
a cysteamine dioxygenase (ado) polynucleotide sequence selected from the group consisting of: SEQ 1; SEQ 24; SEQ 25; SEQ 26; SEQ 27; SEQ 28; and SEQ 29; and
a flavin-containing monooxygenase 1 (fmo1) polynucleotide sequence selected from the group consisting of: SEQ 5; SEQ 71; SEQ 72; SEQ 73; SEQ 74; SEQ 75; SEQ 76; SEQ 77; SEQ 78; SEQ 79; SEQ 80; SEQ 81; and SEQ 99.

According to another aspect of the present invention, there is provided a method to produce an organosulfur compound from a prokaryotic cell wherein said prokaryotic cell, upon transcription and translation under the control of a native or synthetic promoter and Ribosomal binding site (RBS):
said vanin (vnn) polynucleotide sequence provides a vanin (VNN) polypeptide sequence selected from the group consisting of:
   i. a vanin-1 (VNN1) polypeptide sequence, wherein said VNN1 polypeptide sequence has at least 70% sequence coverage to SEQ 4, and at least 25% sequence identity to SEQ 4;
   ii. a vanin-2 (VNN2) polypeptide sequence, wherein said VNN2 polypeptide sequence has at least 70% sequence coverage to SEQ 114, and at least 25% sequence identity to SEQ 114;—and
   iii. a vanin-3 (VNN3) polypeptide sequence, wherein said VNN3 polypeptide sequence has at least 70% sequence coverage to SEQ 158, and at least 25% sequence identity to SEQ 158; and
said cysteamine dioxygenase (ado) polynucleotide sequences provides a cysteamine dioxygenase (ADO) polypeptide sequence which has at least 70% sequence coverage to SEQ 2, and at least 25% sequence identity to SEQ 2; and
said flavin-containing monooxygenase 1 (fmo1) polynucleotide sequence provides a flavin-containing monooxygenase 1 (FMO1) polypeptide sequence which has at least 70% sequence coverage to SEQ 6, and at least 50% of sequence identity to SEQ 6.

According to another aspect of the present invention, there is provided a method to produce an organosulfur compound from a prokaryotic cell wherein said prokaryotic cell comprises at least one of the following:
a vanin-1 (VNN1) polypeptide sequence selected from the group consisting of: SEQ 4; SEQ 55, SEQ 56, SEQ, 57, SEQ 58, SEQ 59, SEQ 60, SEQ 61, SEQ 62, SEQ 63, SEQ 64, SEQ 65, SEQ, 66, SEQ 67, SEQ 68, SEQ 69, and SEQ 70;
a vanin-2 (VNN2) polypeptide sequence selected from the group consisting of: SEQ 114; SEQ 115; SEQ 116; SEQ 117; SEQ 118; SEQ 119; SEQ 120; SEQ 121; SEQ 122; SEQ 123; SEQ 124; SEQ 125; SEQ 126; SEQ 127; SEQ 128; SEQ 129; SEQ 130; SEQ 131; SEQ 132; SEQ 133; SEQ 134; SEQ 135; SEQ 136; SEQ 137; SEQ 138; SEQ 139; and SEQ 140;
a vanin-3 (VNN3) polypeptide sequence selected from the group consisting of SEQ 158; SEQ 159; SEQ 160; SEQ 161; SEQ 162; SEQ 163; SEQ 164; SEQ 165; SEQ 166; SEQ 167; SEQ 168; SEQ 169; SEQ 170; SEQ 171; SEQ 172; SEQ 173; SEQ 174; SEQ 175; SEQ 176; SEQ 177; SEQ 178; SEQ 179: SEQ 180; SEQ 181; SEQ 182; and SEQ 183;
a cysteamine dioxygenase (ADO) polypeptide sequence selected from the group consisting of: SEQ 2; SEQ 30; SEQ 31; SEQ 32; SEQ 33; SEQ 34; SEQ 35; SEQ 36; SEQ 37; SEQ 38; SEQ 39; SEQ 40; SEQ 41; SEQ 42; SEQ 43; and SEQ 44; and
a flavin-containing monooxygenase 1 (FMO1) polypeptide sequence selected from the group consisting of SEQ 6; SEQ 82; SEQ 83; SEQ 84; SEQ 85; SEQ 86; SEQ 87; SEQ 88; SEQ 89; SEQ 90; SEQ 91; SEQ 92; SEQ 93; SEQ 94; SEQ 95; SEQ 96; and SEQ 97.

According to a preferred embodiment of the present invention, there is provided a method to produce an organosulfur compound from a prokaryotic cell wherein said prokaryotic cell further comprises at least one gene to promote the production of an organosulfur compound. Preferably, said at least one gene to promote the production of an organosulfur compound is selected from group consisting of: mcbR (SEQ 184), amtr (SEQ 186), xsc-like (SEQ 188), ssuI (SEQ 190), ssuD1 (SEQ 192), ssuD2 (SEQ 194), ssuR (SEQ 196), accA (SEQ 206), accB (SEQ 208), seuA (SEQ 210), seuB (SEQ 212), seuC (SEQ 214), ilvA (SEQ 216), gldc (SEQ 220), ilvB (SEQ 222), ilvN (SEQ 224), ilvC (SEQ 226), ilvD (SEQ 228), pyc (SEQ 230), dadA-like (SEQ 232), coaA (SEQ 234), coaBC (SEQ 236), coaD (SEQ 238), coaE (SEQ 240), panB (SEQ 244), panC (SEQ 246), panD (SEQ 248), panE (SEQ 250), aspB (SEQ 254), mqo (SEQ 256), mdh (SEQ 260), mcr (SEQ 262), puuE (SEQ 264), abat (SEQ 266), and pydD (SEQ 268).

According to a preferred embodiment of the present invention, there is provided a method to produce an organosulfur compound from a prokaryotic cell wherein upon transcription and translation under the control of a native or synthetic promoter and Ribosomal binding site (RBS), said at least one gene to promote the production of an organosulfur compound generate a polypeptide sequence selected from the group consisting of: SEQ 185, SEQ 187, SEQ 189, SEQ 191, SEQ 193, SEQ 195, SEQ 197, SEQ 207, SEQ 209, SEQ 211, SEQ 213, SEQ 215, SEQ 217, SEQ 221, SEQ 223, SEQ 225, SEQ 227, SEQ 229, SEQ 231, SEQ 233, SEQ 235, SEQ 237, SEQ 239, SEQ 241, SEQ 245, SEQ 247, SEQ 249, SEQ 251, SEQ 255, SEQ 257, SEQ 261, SEQ 263, SEQ 265, SEQ 267, and SEQ 269.

According to another embodiment of the present invention, there is provided a method to produce an organosulfur compound from a prokaryotic cell wherein said prokaryotic cell further comprises at least one gene related to cellular transportation of said organosulfur compound. Preferably said gene related to cellular transportation of said organosulfur compound is selected from the group consisting of: ssuA (SEQ 198), ssuB (SEQ 200), ssuC (SEQ 202), tauE (SEQ 204), gadC (SEQ 218), yhiM (SEQ 242), sdaC (SEQ 252), and cycA (SEQ 258).

According to a preferred embodiment of the present invention, there is provided a method to produce an organosulfur compound from a prokaryotic cell wherein upon transcription and translation under the control of a native or synthetic promoter and Ribosomal binding site (RBS), said at least one gene related to cellular transportation of said organosulfur compound generate a polypeptide sequence selected from the group consisting of: SEQ 199, SEQ 201, SEQ 203, SEQ 205, SEQ 219, SEQ 243, SEQ, SEQ 253, and SEQ 259.

According to a preferred embodiment of the present invention, there is provided a method to produce an organosulfur compound from a prokaryotic cell further comprising a promoter and Ribosomal Binding Site (RBS) sequence which drives gene expression, wherein the genetic sequence for the promoter/RBS is selected from the group consisting of: SEQ 7; SEQ 8; SEQ 9: SEQ 10; SEQ 11; SEQ 12; SEQ 13; SEQ 14; SEQ 15; SEQ 16; SEQ 17; SEQ 18; SEQ 19; SEQ 20; SEQ 21; SEQ 22; and SEQ 23.

### BRIEF DESCRIPTION OF THE ACCOMPANYING FIGURE

The invention may be more completely understood in consideration of the following description of various embodiments of the invention in connection with the accompanying figure, in which:

FIG. 1 is a schematic representation of various known pathways to make an organosulfur compound.

FIG. 2 is a graphical representation of the results of a growth medium testing for taurine-producing bacteria according to a preferred embodiment of the present invention; and

FIG. 3 is a graphical representation of the growth of the strain variants between the two different growth media according to a preferred embodiment of the present invention.

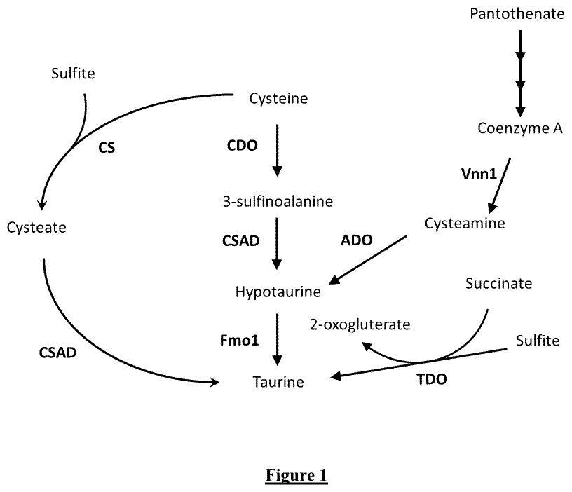

### DETAILED DESCRIPTION OF THE INVENTION

According to a first aspect of the present invention, there is provided a method of producing an organosulfur compound from a prokaryotic cell wherein said method comprises the steps of:
exposing said live prokaryotic cell to a culture media with a pH of between 4 and 11 containing a carbon source and a sulfur source thereby creating an incubation mixture;
incubating said live prokaryotic cell in said incubation mixture under aerobic or anaerobic conditions at a temperature ranging from 0° C. to 60° C. for a period of time sufficient for the expression of said genes for the production of said organosulfur compound;
recovering said organosulfur compound from the prokaryotic cells and/or spent media;
optionally, re-exposing said live prokaryotic cell to unused or spent media for the continuous production of said organosulfur compound.
   and wherein said prokaryotic cell comprises at least one of the following polynucleotide sequences:
i. a vanin (vnn) polynucleotide sequence selected from the group consisting of vanin-1 (vnn1), vanin-2 (vnn2), and vanin-3 (vnn3);
ii. a cysteamine dioxygenase (ado) polynucleotide sequence; and
iii. a flavin-containing monooxygenase 1 (fmo1) polynucleotide sequence

According to a preferred embodiment of the present invention, the live prokaryotic cell capable of expressing genes for the production of said organosulfur compound is a genetically modified prokaryotic cell which comprises a polynucleotide sequence of vanin, wherein said polynucleotide sequence of vanin is selected from the group consisting of: vanin-1 (vnn1); vanin-2 (vnn2); and vanin-3 (vnn3).

In some embodiments, vnn-1 genes can be acquired from eukaryotic organisms such as Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit what organisms these genes can be acquired from. In a preferred embodiment of the present invention, the vanin-1 (vnn-1) polynucleotide sequence SEQ 3 isolated from the eukaryotic species Sus scrofa (pig) is utilized in the process described herein under the transcriptional control of a native or artificial promoter and a ribosomal binding site. However, in other embodiments of the present invention, polynucleotide sequences that are homologous and/or substantially similar to SEQ 3 may also be used. In another embodiment of the present invention, polynucleotide sequences that are homologous and/or substantially similar to SEQ 98 may also be used. Polynucleotide sequences for vanin-1 in these embodiments will have at least 70% sequence coverage, or more preferentially greater than 75%, 80%, 85%, 90%, 95%, 97%, or 98% sequence coverage, or most preferentially greater than 99% sequence coverage to SEQ 3 or SEQ 98, and the polynucleotide sequence of vanin-1 has at least 70% sequence identity, or more preferentially 80%, 85%, 90%, 95%, 97%, or 98% sequence identity, or most preferentially greater than 99% sequence identity to SEQ 3 or SEQ 98. These polynucleotide sequences may include, but by no means are limited to, the following sequences: SEQ 45, SEQ 46, SEQ 47, SEQ 48, SEQ 49, SEQ 50, SEQ 51, SEQ 52, SEQ 53, and SEQ 54.

According to a preferred embodiment of the present invention, the vanin-1 (VNN1) polypeptide SEQ 4 from the eukaryotic species Sus scrofa (pig), is utilized to produce an organosulfur compound by the cell, whereby SEQ 4 is produced from the transcription and translation of the vanin-1 polynucleotide SEQ 3 or SEQ 98. However, in other embodiments of the invention, a polypeptide sequence that is homologous and/or substantially similar to SEQ 4 may also be used in the present invention to produce an organosulfur compound. Polypeptide sequences for vanin-1 in such embodiments will, preferably, have at least 70% sequence coverage, or more preferentially greater than 80%, 90%, 95%, 98%, or most preferentially greater than 99% sequence coverage of SEQ 4, and a sequence identity of at least 25% to SEQ 4, or more preferentially greater than 30%, 35%, 40%, 45%, 50%, 55%, 60%, 65%, 70% 75%, 80%, 85%, 90%, 95%, 97%, or most preferentially greater than 99% sequence identity to SEQ 4. These polypeptide sequences may include, but are by no means limited to, the following sequences: SEQ 55, SEQ 56, SEQ, 57, SEQ 58, SEQ 59, SEQ 60, SEQ 61, SEQ 62, SEQ 63, SEQ 64, SEQ 65, SEQ, 66, SEQ 67, SEQ 68, SEQ 69, and SEQ 70.

According to a preferred embodiment of the present invention, vnn-2 genes can be acquired from eukaryotic organisms such as Bos taurus, Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit the list of organisms from which these genes can be acquired from. In a preferred embodiment of the present invention, the vanin-2 (vnn2) polynucleotide sequence (SEQ 100) isolated from the eukaryotic species Bos taurus (cattle), can be utilized within this invention in place of vanin-1 (vnn1) (such as SEQ 3 or SEQ 98). Said vnn2 polynucleotide sequence is utilized in the process described herein under the transcriptional control of a native or artificial promoter and a ribosomal binding site. However, in other embodiments of the present invention, a polynucleotide sequence that is homologous and/or substantially similar to SEQ 100 may also be used. In another embodiment of the present invention, polynucleotide sequences for vanin-2 in these embodiments have at least 70% sequence coverage, or more preferentially greater than 80%, 85%, 90%, 95%, 96%, or 97% sequence coverage, or most preferentially greater than 99% sequence coverage of SEQ 100, and the polynucleotide sequence of vanin-2 has at least 70% sequence identity, or more preferentially 80%, 85%, 90%, 95%, or 96% sequence identity, or most preferentially greater than 99% sequence identity to SEQ 100. These polynucleotide sequences may include, but by no means are limited to, the following sequences: SEQ 101; SEQ 102; SEQ 103; SEQ 104; SEQ 105; SEQ 106; SEQ 107; SEQ 108; SEQ 109; SEQ 110; SEQ 111; SEQ 112; and SEQ 113.

According to a preferred embodiment of the present invention, the vanin-2 (VNN2) polypeptide sequence SEQ 114 isolated from the eukaryotic species Sus scrofa (pig) can be utilized within this invention, whereby SEQ 114 is produced from the transcription and translation of the vanin-2 polynucleotide SEQ 100. However, in other embodiments of the invention, polypeptide sequences that are homologous and/or substantially similar to SEQ 114 may also be used. Polypeptide sequences for vanin-2 in these embodiments will, preferably, have at least 70% sequence coverage, or more preferentially greater than 75%, 80%, 90%, 95%, or most preferentially greater than 99% sequence coverage of SEQ 114, and a sequence identity of at least 25% to SEQ 114, or more preferentially greater than 30%, 35%, 40%, 45%, 50%, 55%, 60%, 65%, 70% 75%, 80%, 85%, 90%, 95%, 98%, or most preferentially greater than 99% sequence identity to SEQ 114. These polypeptide sequences may include, but are by no means limited to, the following sequences: SEQ 115; SEQ 116; SEQ 117; SEQ 118; SEQ 119; SEQ 120; SEQ 121; SEQ 122; SEQ 123; SEQ 124; SEQ 125; SEQ 126; SEQ 127; SEQ 128; SEQ 129; SEQ 130; SEQ 131; SEQ 132; SEQ 133; SEQ 134; SEQ 135; SEQ 136; SEQ 137; SEQ 138; SEQ 139; and SEQ 140.

According to a preferred embodiment of the present invention, vnn-3 genes can be acquired from eukaryotic organisms such as Bos taurus, Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit what organisms these genes can be acquired from. In a preferred embodiment of the present invention, the vanin-3 (vnn-3) polynucleotide sequence SEQ 141 isolated from the eukaryotic species Mus musculus (house mouse) can be utilized within this invention in place of vanin-1 (vnn-1) SEQ 3 or SEQ 98. Said vnn3 polynucleotide sequence is utilized in the process described herein under the transcriptional control of a native or artificial promoter and a ribosomal binding site. However, in other embodiments of the invention, polynucleotide sequences that are homologous and/or substantially similar to SEQ 141 may also be used. In another embodiment of the present invention, polynucleotide sequences for vanin-3 in these embodiments will have at least 70% sequence coverage, or more preferentially greater than 75%, 80%, 85%, 90%, 95%, 96%, or 97% sequence coverage, or most preferentially greater than 99% sequence coverage of SEQ 141, and the polynucleotide sequence of vanin-3 has at least 70% sequence identity, or more preferentially 75%, 80%, 90%, 95%, or 97% sequence identity, or most preferentially greater than 99% sequence identity to SEQ 141. These polynucleotide sequences may include, but by no means are limited to, the following sequences: SEQ 142; SEQ 143; SEQ 144; SEQ 145; SEQ 146; SEQ 147; SEQ 148; SEQ 149; SEQ 150; SEQ 151; SEQ 152; SEQ 153; SEQ 154; SEQ 155; SEQ 156; and SEQ 157.

According to a preferred embodiment of the present invention, the vanin-3 (VNN3) polypeptide SEQ 158 isolated from the eukaryotic species Mus musculus (house mouse) can be utilized within this invention, whereby SEQ 158 is produced from the transcription and translation of the vanin-3 polynucleotide SEQ 141. However, in other embodiments of the invention, polypeptide sequences that are homologous and/or substantially similar to SEQ 158 may also be used. Polypeptide sequences for vanin-3 in these embodiments will, preferably, have at least 70% sequence coverage, or more preferentially greater than 75%, 80%, 85%, 90%, 95%, 96%, 97%, 98%, or most preferentially greater than 99% sequence coverage of SEQ 158, and a sequence identity of at least 25% to SEQ 158, or more preferentially greater than 30%, 35%, 40%, 45%, 50%, 55%, 60%, 65%, 70% 75%, 80%, 85%, 90%, 95%, 98%, or most preferentially greater than 99% sequence identity to SEQ 158. These polypeptide sequences may include, but are by no means limited to, the following sequences: SEQ 159; SEQ 160; SEQ 161; SEQ 162; SEQ 163; SEQ 164; SEQ 165; SEQ 166; SEQ 167; SEQ 168; SEQ 169; SEQ 170; SEQ 171; SEQ 172; SEQ 173; SEQ 174; SEQ 175; SEQ 176; SEQ 177; SEQ 178; SEQ 179; SEQ 180; SEQ 181; SEQ 182; and SEQ 183.

According to a preferred embodiment of the present invention, the live prokaryotic cell capable of expressing genes for the production of said organosulfur compound is a genetically modified prokaryotic cell which comprises a polynucleotide sequence of cysteamine dioxygenase (ado) and a polynucleotide sequence of vanin, wherein said polynucleotide sequence of vanin is selected from the group consisting of: vanin-1 (vnn1); vanin-2 (vnn2); and vanin-3 (vnn3).

In some embodiments, the ado genes can be acquired from eukaryotic organisms such as Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit which organisms these genes can be acquired from. In a preferred embodiment of the present invention, the cysteamine dioxygenase polynucleotide (ado) sequence SEQ 1 isolated from the eukaryotic species Sus scrofa (pig) is utilized in the process described herein under the transcriptional control of a native or artificial promoter and a ribosomal binding site. However, in other embodiments of the invention, polynucleotide sequences that are homologous and/or substantially similar to SEQ 1 may also be used. In another embodiment of the present invention, polynucleotide sequences for cysteamine dioxygenase in these embodiments will, preferably, have at least 70% sequence coverage, or more preferably greater than 80%, 90%, 95%, 98%, or most preferentially greater than 99% sequence coverage to SEQ 1, and sequence identities of at least 70%, or more preferentially greater than 80%, 90%, 95%, 97% sequence identity, and most preferentially 99% sequence identity to SEQ 1. These polynucleotide sequences may include, but by no means limited to, the following sequences: SEQ 24, SEQ 25, SEQ 26, SEQ 27, SEQ 28, and SEQ 29.

According to a preferred embodiment of the present invention, the cysteamine dioxygenase polypeptide (ADO) sequence SEQ 2 isolated from the eukaryotic species Sus scrofa (pig), is utilized according to a preferred embodiment of this invention, whereby SEQ 2 is produced from the transcription and translation of the cysteamine dioxygenase polynucleotide SEQ 1. However, according to other preferred embodiments of the invention, polypeptide sequences that are homologous and/or substantially similar to SEQ 2 may also be used in the present invention to produce an organosulfur compound. Polypeptide sequences for cysteamine dioxygenase in these embodiments will, preferably, have at least 70% sequence coverage, or more preferentially greater than 80%, 90%, 95%, 98%, or most preferentially greater than 99% sequence coverage to SEQ 2, and a sequence identity of, preferably, at least 25% to SEQ 2, or more preferentially greater than 30%, 35%, 40%, 45%, 50%, 55%, 60%, 65%, 70% 75%, 80%, 85%, 90%, 95%, 97%, or most preferentially greater than 99% sequence identity to SEQ 2. These polypeptide sequences may include, but are not limited to, the following sequences: SEQ 30, SEQ 31, SEQ 32, SEQ 33, SEQ 34, SEQ 35, SEQ 36, SEQ 37, SEQ 38, SEQ 39, SEQ 40, SEQ 41, SEQ 42, SEQ 43, and SEQ 44.

According to a preferred embodiment of the present invention, the live prokaryotic cell capable of expressing genes for the production of said organosulfur compound is a genetically modified prokaryotic cell which is selected from the group consisting of: a polynucleotide sequence of cysteamine dioxygenase (ado); a polynucleotide sequence of vanin; and a polynucleotide sequence of Flavin-Containing Monooxygenase 1 (fmo1).

In some embodiments, the fmo1 gene is acquired from eukaryotic organisms such as Sus scrofa, Capra hircus, Microtus fortis, Panthera pardus, Homo sapiens, Varanus komodoensis, Apodemus sylvaticus, Eublepharis macularius, Alca torda, Chordeiles acutipennis, Grantiella picta, Caloenas nicobarica, Regulus satrapa, and Lutra lutra, although the listed organisms are only given as examples and are in no way meant to limit what organisms these genes can be acquired from. In a preferred embodiment of the present invention, the fmo1 gene is sourced from Sus scrofa (pig). In a preferred embodiment of the present invention, the flavin-containing monooxygenase 1 (fmo1) polynucleotide sequence SEQ 5 isolated from the eukaryotic species Sus scrofa (pig), is utilized in the process described herein under the transcriptional control of a native or artificial promoter and a ribosomal binding site. However, in other embodiments of the invention, polynucleotide sequences that are homologous and/or substantially similar to SEQ 5 may also be used in the present invention to produce an organosulfur compound. In another embodiment of the present invention, polynucleotide sequences that are homologous and/or substantially similar to SEQ 99 may also be used in a preferred embodiment of the present invention. Polynucleotide sequences for flavin-containing monooxygenase 1 in these embodiments will, preferably, have at least 70% sequence coverage, or more preferentially greater than 75%, 80%, 85%, 90%, 95%, 97%, 98%, or most preferentially greater than 99% sequence coverage to SEQ 5 or SEQ 99, and a sequence identity of at least 70%, or more preferentially greater than 75%, 80%, 85%, 90%, 95%, 97%, or most preferentially greater than 99% sequence identity to SEQ 5 or SEQ 99. These polynucleotide sequences may include, but are by no means limited to, the following sequences: SEQ 71, SEQ 72, SEQ 73, SEQ 74, SEQ 75, SEQ 76, SEQ 77, SEQ 78, SEQ 79, SEQ 80, and SEQ 81.

According to a preferred embodiment of the present invention, the flavin-containing monooxygenase 1 (FMO1) polypeptide sequence SEQ 6 from the eukaryotic species Sus scrofa (pig) is utilized within this invention, whereby SEQ 6 is produced from the transcription and translation of the flavin-containing monooxygenase 1 polynucleotide SEQ 5 or SEQ 99. However, in other embodiments of the invention, polypeptide sequences that are homologous and substantially similar to SEQ 6 may also be used in the present invention to produce an organosulfur compound. Polypeptide sequences for flavin-containing monooxygenase 1 in these embodiments will, preferably, have at least 70% sequence coverage, or more preferentially greater than 75%, 80%, 85%, 90%, 95%, 97%, 98%, or most preferentially greater than 99% sequence coverage of SEQ 6, and a sequence identity of at least 50%, or more preferably greater than 55%, 60%, 65%, 70%, 75%, 80%, 85%, 90%, 95%, 97%, 98% sequence identity, or most preferentially greater than 99% sequence identity to SEQ 6. These polypeptide sequences may include, but are by no means limited to, the following sequences: SEQ 82, SEQ 83, SEQ 84, SEQ 85, SEQ 86, SEQ 86, SEQ 87, SEQ 88, SEQ 89, SEQ 90, SEQ 91, SEQ 92, SEQ 93, SEQ 94, SEQ 95, SEQ 96, and SEQ 97.

According to a preferred embodiment of the present invention, the prokaryotic cell further comprises a promoter and Ribosomal Binding Site (RBS) sequence which drives gene expression, wherein the genetic sequence for the promoter/RBS comprises at least one of the following: SEQ 7; SEQ 8; SEQ 9: SEQ 10; SEQ 11; SEQ 12; SEQ 13; SEQ 14; SEQ 15; SEQ 16; SEQ 17; SEQ 18; SEQ 19; SEQ 20; SEQ 21; SEQ 22; and SEQ 23.

According to a preferred embodiment of the present invention, the cell has been genetically modified to add, remove, or inactivate at least one gene whose function is to alter positively the synthesis of an organosulfur compound. These modifications could be achieved through several mechanisms, including but not limited to: the deletion of the offending gene product from the genome; the modification of the promoter sequence to reduce or prevent gene expression; the degradation of the messenger RNA produced by the polynucleotide sequence, which is used to produce the final polypeptide sequence; the introduction of sequences or genes that directly regulate gene/protein production, or are used to produce antisense RNA products to prevent and downregulate the production of a polypeptide through a process called antisense transcription; or a combination of several methods. Some ways not limited to those listed to upregulate or modify gene activity or transcription/translation can be brought about by expressing multiple copies of the same gene in the genome under the original promoter, replacing the promoter with another naturally occurring or synthetic promoter, making key mutations in the gene to artificially increase protein activity, and/or replacing the gene with another copy of the gene of interest from an alternate genetic source, or a combination of the above methods to upregulate or modify gene activity or transcription/translation.

According to a preferred embodiment of the present invention, the at least one gene to promote the production of an organosulfur compound is selected from the group consisting of: transcriptional repressors controlling sulfur assimilation; transcriptional repressors controlling nitrogen assimilation; taurine dehydrogenases and FAD-dependent oxidoreductases; oxidoreductases; sulfitonases; sulfur compound transporter proteins; taurine dioxygenases; oxidoreductases; FMNH₂-dependent sulfonatases; NAD(P)H-dependent FMN reductases; reductases that reduce taurine precursors to produce substrates for other metabolic pathways in the cell; taurine dioxygenases; sulfoacetaldehyde acetyltransferases; PLP dependent aminotransferases; branched-chain-amino-acid aminotransferases; taurine: 2-oxoglutarate aminotransferases; taurine-pyruvate aminotransferases; aminotransferase family proteins; regulatory proteins for organosulfur compound degradation; genes that divert precursor molecules away from taurine such as carboxylases or synthase/synthesis proteins; carboxyl transferases; malonyl-CoA reductases; aminotransferase family proteins; FMNH₂-dependent monooxygenases; taurine-pyruvate aminotransferases; hydroxymethyltransferases; synthetases; synthases; reductases; decarboxylases; dehydratase proteins; and kinases or any combination thereof.

According to a preferred embodiment of the present invention, the prokaryotic cell comprises the addition, deletion, repression, or modification of one or more genes that encode protein(s) which are responsible for the production of an organosulfur compound. Such modifications can include at least one of the following polynucleotide sequences selected from the list: mcbR (SEQ 184), amtr (SEQ 186), xsc-like (SEQ 188), ssuI (SEQ 190), ssuD1 (SEQ 192), ssuD2 (SEQ 194), ssuR (SEQ 196), accA (SEQ 206), accB (SEQ 208), seuA (SEQ 210), seuB (SEQ 212), seuC (SEQ 214), ilvA (SEQ 216), gldc (SEQ 220), ilvB (SEQ 222), ilvN (SEQ 224), ilvC (SEQ 226), ilvD (SEQ 228), pyc (SEQ 230), dadA-like (SEQ 232), coaA (SEQ 234), coaBC (SEQ 236), coaD (SEQ 238), coaE (SEQ 240), panB (SEQ 244), panC (SEQ 246), panD (SEQ 248), panE (SEQ 250), aspB (SEQ 254), mqo (SEQ 256), mdh (SEQ 260), mcr (SEQ 262), puuE (SEQ 264), abat (SEQ 266), and pydD (SEQ 268). These polynucleotide sequences upon transcription and translation may provide the following polypeptide sequences: MCBR (SEQ 185), AMTR (SEQ 187), XSC-like (SEQ 189), SSUI (SEQ 191), SSUD1 (SEQ 193), SSUD2 (SEQ 195), SSUR (SEQ 197), ACCA (SEQ 207), ACCB (SEQ 209), SEUA (SEQ 211), SEUB (SEQ 213), SEUC (SEQ 215), ILVA (SEQ 217), GLDC (SEQ 221), ILVB (SEQ 223), ILVN (SEQ 225), ILVC (SEQ 227), ILVD (SEQ 229), PYC (SEQ 231), DADA-like (SEQ 233), COAA (SEQ 235), COABC (SEQ 237), COAD (SEQ 239), COAE (SEQ 241), PANB (SEQ 245), PANC (SEQ 247), PAND (SEQ 249), PANE (SEQ 251), ASPB (SEQ 255), MQO (SEQ 257), MDH (SEQ 261), MCR (SEQ 263), PUUE (SEQ 265), ABAT (SEQ 267), and PYDD (SEQ 269).

According to a preferred embodiment of the present invention, the prokaryotic cell comprises the addition, deletion, repression, or modification of one or more genes that encode protein(s) which are responsible for the cellular transportation of said organosulfur compound. Organosulfur import/export proteins that may be used in the present invention include but are not limited to sulfur compound transporter proteins, glutamatein/γ-aminobutyrate (GABA) electrogenic-like antiporters such as amino acid transporters; polyamine transporters; and/or amino acid permease proteins.

According to a preferred embodiment of the present invention, the prokaryotic cell is modified through the addition, alteration, and/or deletion of genes related to the cellular transportation of organosulfur compounds. Such modifications can include at least one of the polynucleotide sequences from the list: ssuA (SEQ 198), ssuB (SEQ 200), ssuC (SEQ 202), tauE (SEQ 204), gadC (SEQ 218), yhiM (SEQ 242), sdaC (SEQ 252), and cycA (SEQ 258). These polynucleotide sequences upon transcription and translation may provide the following respective polypeptide sequences: SSUA (SEQ 199), SSUB (SEQ 201), SSUC (SEQ 203), TAUE (SEQ 205), GADC (SEQ 219), YHIM (SEQ 243), SDAC (SEQ 253), and CYCA (SEQ 259). The sequences and related genes/proteins provided herein associated with the cellular transportation are by way of example and are in no way limiting and/or comprehensive.

According to a preferred embodiment of the present invention, the live prokaryotic cell capable of expressing genes for the production of said organosulfur compound is a bacterial cell. Preferably, said bacterial cell is selected from the group consisting of the genera: Brevibacterium, Bacillus, Corynebacterium, Escherichia, Lactococcus, Pseudomonas, Rhodococcus, and Serratia. More preferably, the bacterial cell belongs to the genus Corynebacterium. Preferably, the bacterial cell is Corynebacterium glutamicum.

According to a preferred embodiment of the present invention, the carbon source is selected from the group consisting of: long-chain saccharides, short chain saccharides, monosaccharides, sugar alcohols, organic acids and their corresponding salts and/or combinations thereof. According to a more preferred embodiment of the present invention, the carbon source is selected from the group consisting of: glucose, mannose, maltose, lactose, galactose, fructose, sucrose, molasses, ribose, arabinose, xylose, gluconic acid and gluconate salts, arabitol, mannitol, sorbitol, maltitol, xylitol, erythritol, lactitol, acetic acid, acetate salts, starch and starch hydrolysates, corn steep liquor (CSL), whey or whey permeate, bagasse hydrolysate, glycerol, fumaric acid and its salts, lactic acid and its salts, ethanol, methanol, and/or combinations thereof.

According to a preferred embodiment of the present invention, said sulfur source is selected from the group consisting of: inorganic compounds comprising sulfur in any oxidation state; organic compounds comprising sulfur in any oxidation state; and their corresponding salts. Some non-limiting examples include elemental sulfur; sulfides; thiols (i.e., cysteamine, ethanethiol, propanethiol, cysteine, N-acetyl-cysteine); sulfate, sulfite, and thiosulfate salts (i.e., ammonium, magnesium, potassium); and/or combinations thereof.

According to a preferred embodiment of the present invention, said organosulfur compound is selected from the group comprising cysteamine, hypotaurine and taurine. According to an even more preferred embodiment of the present invention, said organosulfur compound of interest is taurine.

According to a preferred embodiment of the present invention, the pH of said culture media is maintained between 4 and 11. Preferably, the pH of said culture media is maintained between 5 and 9. More preferably, the pH of said culture media is maintained between 6 and 8.

According to a preferred embodiment of the present invention, the temperature of incubation is maintained between 0 and 60° C. Preferably, the temperature of incubation is maintained between 2° and 50° C. More preferably, the temperature of incubation is maintained between 25 and 40° C.

In some embodiments of the present invention, one or more “growth-promoting agents” may be added to the incubation mixture to promote the growth and/or the expression of the genes necessary for the production of said organosulfur compound. These “growth-promoting agents” may be selected from the group consisting of: nitrogen-based growth-promoting agents, phosphorus-based growth promoting, and other growth promoting agents, and combinations thereof.

In a preferred embodiment of the present invention, the nitrogen-based growth-promoting agents are selected from the group consisting of: ammonia, inorganic and organic ammonium salts, nitrite and nitrate salts, urea, amines and amino acids, peptides, extracts from animal and plant-based industries (i.e., meat and/or yeast extract, soybean meal, corn meal, corn flour, soybean flour, gelatin, collagen, etc.), and combinations thereof.

In a preferred embodiment of the present invention, the phosphorus-based growth-promoting agents are selected from the group consisting of: phosphate salts including, but not limited to, phosphate salts of Groups I and II, hydrogen phosphate salts of Groups I and II, dihydrogen phosphate salts of Groups I and II, and combinations thereof.

In another preferred embodiment of the present invention, the other growth promoting agents are selected from the group consisting of: vitamins, inorganic salts of transition and non-transition metals, heavy metal-containing salts, and/or combinations thereof. Some further examples include but are not limited to Vitamin B2, Vitamin B1, Vitamin B5, Vitamin B7, sodium chloride, iron sulfate, magnesium sulfate, manganese sulfate, calcium carbonate, sodium hydroxide, etc.

According to a preferred embodiment of the present invention, oxygen may be added to the incubation mixture. The incorporation of oxygen may be done through vigorous mixing or through a controlled flow of air or another oxygen-enriched gas. In certain embodiments of the present invention, a certain concentration of dissolved oxygen may be targeted for increased production of the organosulfur compound. Preferably, that oxygen concentration of the incubation mixture exceeds 5%. More preferably the oxygen concentration of the incubation mixture exceeds 10%.

In some embodiments of the present invention, other components may be added to the incubation mixture to avoid potential undesired effects. Non-limiting examples of these components include antifoamers to avoid foam formation and overflowing, and antimicrobials to avoid contamination from other non-desirable organisms that may compete with the live prokaryotic cell for resources.

It is known to those skilled in the art that various techniques can be utilized for the extraction and purification of the desired compound from the cells or the mixture in which the cells are. These methods include, but are not limited to, centrifugation, filtration, dialysis, crystallization, ion exchange, electrodialysis, chromatography, solvent extraction, evaporation and nanofiltration.

In some embodiments of the present invention, the ado and vnn1 genes can be acquired from eukaryotic organisms such as Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit what organisms these genes can be acquired from. In a preferred embodiment of this invention, the ADO and vnn1 genes are obtained from the organism Sus scrofa.

In another preferred embodiment of the present invention, the fmo1 gene is acquired from eukaryotic organisms such as Sus scrofa, Capra hircus, Microtus fortis, Panthera pardus, Homo sapiens, Varanus komodoensis, Apodemus sylvaticus, Eublepharis macularius, Alca torda, Chordeiles acutipennis, Grantiella picta, Caloenas nicobarica, Regulus satrapa, and Lutra lutra, although the listed organisms are only given as examples and are in no way meant to limit what organisms these genes can be acquired from. In a preferred embodiment of the present invention, the fmo1 gene is sourced from Sus scrofa, and this gene encodes a protein that directly oxidizes hypotaurine to taurine.

According to a preferred embodiment of the present invention, there is provided a method of growing a prokaryotic organism, wherein the method comprises the steps of:
providing a live prokaryotic cell capable of expressing genes for the production of an organosulfur compound;
exposing said live prokaryotic cell to a culture media with a pH of between 4 and 11 containing a sugar source and a sulfur source; thereby creating an incubation mixture;
incubating said live prokaryotic cell in said incubation mixture under aerobic and/or anaerobic conditions at a temperature ranging from 0° C. to 60° C. for a period of time ranging from 3 hours to 15 days allowing for the growth of said live prokaryotic cell;

According to a preferred embodiment of the present invention, there is provided a method of growing a prokaryotic organism capable of expressing genes for the production of an organosulfur compound, wherein the method comprises the steps of:
providing a live prokaryotic cell capable of expressing genes for the production of an organosulfur compound;
exposing said live prokaryotic cell to a culture media with a pH of between 4 and 11 thereby creating an incubation mixture comprising a first cell mass; and
growing said incubation mixture comprising a first cell mass under aerobic and/or anaerobic conditions at a temperature ranging from 0° C. to 60° C. for a period of time necessary to create a second cell mass of the prokaryotic organism greater than the first cell mass.

According to a preferred embodiment of the present invention, the live prokaryotic strain capable of expressing genes for the production of an organosulfur compound is a bacterial cell. Preferably, said bacterial cell is selected from the group consisting of the genera: Brevibacterium, Bacillus, Corynebacterium, Escherichia, Lactococcus, Pseudomonas, Rhodococcus, and Serratia. More preferably, the cell belongs to the genus Corynebacterium. Preferably, the bacterial cell is Corynebacterium glutamicum.

According to a preferred aspect of the present invention, the period of time necessary to create a second cell mass of the prokaryotic organism greater than the first cell mass ranges from 3 hours to 15 days.

Within the context of this disclosure, it is understood that the person skilled in the art knows that a polynucleotide is defined as the collection of individual nucleotides in any organization or size that relates to the DNA sequence. The term expression within this invention refers to the generation of a polypeptide sequence which is produced based on its polynucleotide sequence or gene. The term gene references a DNA sequence that encodes for a specific polypeptide sequence. A gene can include both sequences between coding regions (introns) and the encoding sequence itself (exon). Genetic modification or related statements herein refer to the alteration of the genetic code of an organism which includes the insertion or deletion of DNA sequences within an organism. Within the context of this disclosure, it is understood that the person skilled in the art knows that a genetic modification can include insertion and maintenance of an expression vector into the organism, or the direct modification of the organism's genome by directly adding or deleting genes through processes like, but not limited to, 2 step allelic exchange or CRISPR cloning. Within the context of this disclosure, it is understood that the person skilled in the art knows that the term enzyme within the present invention defines a polypeptide sequence, specifically in the form of a protein, that can modify a biological molecule or take part within its generation through direct or indirect interactions. The process by which an enzyme influences the modification and/or production of a biological molecule and/or product is termed enzymatic activity.

The following examples are intended purely to illustrate the invention and should not be considered as limiting its scope.

### Experiment #1—Growth Media Testing for Taurine-Producing Bacteria

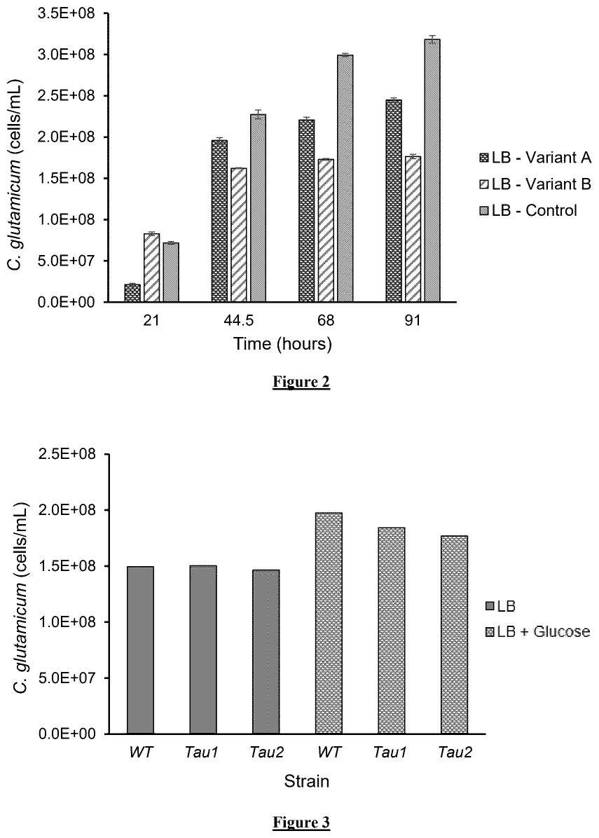

A seed culture of Corynebacterium glutamicum comprising polynucleotide sequences of vnn1, ado and fmo1 was established by inoculating a LB medium and variations of said medium. These LB media variants were evaluated to determine optimal growth of the organism. Growth medium LB-Variant A contained yeast extract (5 g/L), sodium chloride (10 g/L) and soybean flour (10 g/L). Growth medium LB Variant B contained yeast extract (5 g/L), sodium chloride (10 g/L) and gelatin (10 g/L). Growth medium LB-Control contained yeast extract (5 g/L), sodium chloride (10 g/L) and peptone (10 g/L). All growth media variants were adjusted with NaOH to pH 7.0. Cultures were grown at 30° C. for 4 days, with samples taken daily to measure optical density at 600 nm. Results are shown in FIG. 2. Although cell growth in variations of the LB media was lower compared to the control, Corynebacterium glutamicum was still successfully cultivated in these variant media.

### Experiment #2—Seed Culture Preparations of Taurine-Producing Bacteria

To determine the growth rate of the genetically modified organisms in comparison to their wild-type counterpart, seed cultures of variants of Corynebacterium glutamicum cultures were prepared using strains comprising polynucleotide sequences of vnn1, ado and fmo1 with different promoters (Tau 1 and Tau 2) as well as a non-modified wild-type Corynebacterium glutamicum (WT). Cultures were prepared by inoculating said strains into LB broth and LB broth with 0.5% w/w glucose. Media was adjusted with NaOH to pH 7.0. All cultures were grown at 30° C. for 90 hours, after which time optical density at 600 nm was measured to compare growth of the strain variants between the two different growth media. Results are shown in FIG. 3. The incorporation of the polynucleotide sequences of vnn1, ado and fmo1 in Tau 1 and Tau 2 did not have any inhibitory effects on the growth of the cells in comparison to the wild-type strain.

### Experiment #3—Fermentation Culture Preparations of Taurine-Producing Bacteria

A seed culture of variants of Corynebacterium glutamicum cultures was prepared using the strains from Example 2 in LB broth with 0.5% w/w glucose as described in experiment #2. Fermentation media was inoculated with 1/20 volume of the seed culture. The fermentation media contained yeast extract (2 g/L), glucose (40 g/L), calcium carbonate (10 g/L), ammonium sulfate (15 g/L), dibasic potassium phosphate (1 g/L), monobasic potassium phosphate (1 g/L), sodium chloride (2 g/L), anhydrous magnesium sulfate (1 g/L), calcium chloride (80 mg/L), iron (III) chloride hexahydrate (3 mg/L), zinc sulfate heptahydrate (0.9 mg/L), copper (II) sulfate pentahydrate (0.2 mg/L), manganese sulfate (0.4 mg/L), sodium molybdate dihydrate (0.1 mg/L), boric acid (0.3 mg/L), thiamine hydrochloride (0.2 mg/L), biotin (0.2 mg/L), adjusted with KOH to pH 7.0. Cultures were grown for up to 96 hours, during which time samples were collected, the cells separated, and taurine was quantified via HPLC in both the supernatant and cell pellet.

While the foregoing invention has been described in some detail for purposes of clarity and understanding, it will be appreciated by the person skilled in the art, once they have been made familiar with this disclosure that various changes in form and detail can be made without departing from the true scope of the invention in the appended claims.

1. A method of producing an organosulfur compound from a prokaryotic cell wherein said prokaryotic cell comprises a vanin (vnn) polynucleotide sequence selected from the group consisting of: vanin-1 (vnn1), vanin-2 (vnn2), and vanin-3 (vnn3).

2. A method of producing an organosulfur compound from a prokaryotic cell wherein said method comprises the steps of:
providing a live prokaryotic cell capable of expressing at least one gene for the production of said organosulfur compound;
exposing said live prokaryotic cell to a culture media with a pH of between 4 and 11 containing a carbon source and a sulfur source thereby creating an incubation mixture;
incubating said live prokaryotic cell in said incubation mixture under aerobic or anaerobic conditions at a temperature ranging from 0° C. to 60° C. for a period of time sufficient for the expression of said at least one gene for the production of said organosulfur compound;
recovering said organosulfur compound from the bacterial cells and/or spent media; and
optionally, re-exposing said live prokaryotic cell to an unused media or spent media for the continuous production of said organosulfur compound;
wherein said prokaryotic cell comprises at least one of the following polynucleotide sequences:
i. a vanin (vnn) polynucleotide sequence selected from the group consisting of:
vanin-1 (vnn1), wherein said vnn1 polynucleotide sequence has at least 70% sequence coverage to SEQ 3 or SEQ 98, and at least 70% sequence identity to SEQ 3 or SEQ 98;
vanin-2 (vnn2), wherein said vnn2 polynucleotide sequence has at least 70% sequence coverage to SEQ 100, and at least 70% sequence identity to SEQ 100; and
vanin-3 (vnn3), wherein said vnn3 polynucleotide sequence has at least 70% sequence coverage to SEQ 141, and at least 70% sequence identity to SEQ 141;
ii. a cysteamine dioxygenase (ado) polynucleotide sequence which has at least 70% sequence coverage to SEQ 1, and at least 70% sequence identity to SEQ 1; and
iii. a flavin-containing monooxygenase 1 (fmo1) polynucleotide sequence which has at least 70% sequence coverage to SEQ 5 or SEQ 99, and at least 70% of sequence identity to SEQ 5 or SEQ 99.

3. The method according to claim 1, wherein said prokaryotic cell comprises at least one of the following polynucleotide sequences:
a vanin (vnn) polynucleotide sequence selected from the group consisting of:
i. a vanin 1 (vnn1) polynucleotide sequence, wherein said vnn1 polynucleotide sequence is selected from the group consisting of: SEQ 3; SEQ 45, SEQ 46, SEQ, 47, SEQ 48, SEQ 49, SEQ 50, SEQ 51, SEQ 52, SEQ 53, SEQ 54, and SEQ 98;
ii. vanin 2 (vnn2) polynucleotide sequence, wherein said vnn2 polynucleotide sequence is selected from the group consisting of: SEQ 100; SEQ 101; SEQ 102; SEQ 103; SEQ 104; SEQ 105; SEQ 106; SEQ 107; SEQ 108; SEQ 109; SEQ 110; SEQ 111; SEQ 112; and SEQ 113; and
iii. vanin 3 (vnn3) polynucleotide sequence, wherein said vnn3 polynucleotide sequence is selected from the group consisting of: SEQ 141; SEQ 142; SEQ 143; SEQ 144; SEQ 145; SEQ 146; SEQ 147; SEQ 148; SEQ 149; SEQ 150; SEQ 151; SEQ 152; SEQ 153; SEQ 154; SEQ 155; SEQ 156; and SEQ 157;
a cysteamine dioxygenase (ado) polynucleotide sequence selected from the group consisting of: SEQ 1; SEQ 24; SEQ 25; SEQ 26; SEQ 27; SEQ 28; and SEQ 29; and
a flavin-containing monooxygenase 1 (fmo1) polynucleotide sequence selected from the group consisting of: SEQ 5; SEQ 71; SEQ 72; SEQ 73; SEQ 74; SEQ 75; SEQ 76; SEQ 77; SEQ 78; SEQ 79; SEQ 80; SEQ 81; and SEQ 99.

4. The method according to claim 3 wherein, upon transcription and translation under the control of a native or synthetic promoter and Ribosomal binding site (RBS):
said vanin (vnn) polynucleotide sequence provides a vanin (VNN) polypeptide sequence selected from the group consisting of:
i. a vanin-1 (VNN1) polypeptide sequence, wherein said VNN1 polypeptide sequence has at least 70% sequence coverage to SEQ 4, and at least 25% sequence identity to SEQ 4;
ii. a vanin-2 (VNN2) polypeptide sequence, wherein said VNN2 polypeptide sequence has at least 70% sequence coverage to SEQ 114, and at least 25% sequence identity to SEQ 114;—and
iii. a vanin-3 (VNN3) polypeptide sequence, wherein said VNN3 polypeptide sequence has at least 70% sequence coverage to SEQ 158, and at least 25% sequence identity to SEQ 158; and
said cysteamine dioxygenase (ado) polynucleotide sequences provides a cysteamine dioxygenase (ADO) polypeptide sequence which has at least 70% sequence coverage to SEQ 2, and at least 25% sequence identity to SEQ 2; and
said flavin-containing monooxygenase 1 (fmo1) polynucleotide sequence provides a flavin-containing monooxygenase 1 (FMO1) polypeptide sequence which has at least 70% sequence coverage to SEQ 6, and at least 50% of sequence identity to SEQ 6.

5. The method according to claim 4 wherein:
said vanin-1 (VNN1) polypeptide sequence is selected from the group consisting of: SEQ 4; SEQ 55, SEQ 56, SEQ, 57, SEQ 58, SEQ 59, SEQ 60, SEQ 61, SEQ 62, SEQ 63, SEQ 64, SEQ 65, SEQ, 66, SEQ 67, SEQ 68, SEQ 69, and SEQ 70;
said vanin-2 (VNN2) polypeptide sequence is selected from the group consisting of: SEQ 114; SEQ 115; SEQ 116; SEQ 117; SEQ 118; SEQ 119; SEQ 120; SEQ 121; SEQ 122; SEQ 123; SEQ 124; SEQ 125; SEQ 126; SEQ 127; SEQ 128; SEQ 129; SEQ 130; SEQ 131; SEQ 132; SEQ 133; SEQ 134; SEQ 135; SEQ 136; SEQ 137; SEQ 138; SEQ 139; and SEQ 140;
said vanin-3 (VNN3) polypeptide sequence is selected from the group consisting of SEQ 158; SEQ 159; SEQ 160; SEQ 161; SEQ 162; SEQ 163; SEQ 164; SEQ 165; SEQ 166; SEQ 167; SEQ 168; SEQ 169; SEQ 170; SEQ 171; SEQ 172; SEQ 173; SEQ 174; SEQ 175; SEQ 176; SEQ 177; SEQ 178; SEQ 179: SEQ 180; SEQ 181; SEQ 182; and SEQ 183;
said cysteamine dioxygenase (ADO) polypeptide sequence is selected from the group consisting of: SEQ 2; SEQ 30; SEQ 31; SEQ 32; SEQ 33; SEQ 34; SEQ 35; SEQ 36; SEQ 37; SEQ 38; SEQ 39; SEQ 40; SEQ 41; SEQ 42; SEQ 43; and SEQ 44; and
said flavin-containing monooxygenase 1 (FMO1) polypeptide sequence is selected from the group consisting of SEQ 6; SEQ 82; SEQ 83; SEQ 84; SEQ 85; SEQ 86; SEQ 87; SEQ 88; SEQ 89; SEQ 90; SEQ 91; SEQ 92; SEQ 93; SEQ 94; SEQ 95; SEQ 96; and SEQ 97.

6. The method according to claim 1, wherein said prokaryotic cell further comprises at least one of the following:
an addition, deletion and/or alteration of at least one gene to promote the production of an organosulfur compound; and
an addition, deletion and/or alteration of at least one gene related to the cellular transportation of an organosulfur compound.

7. The method according to claim 6 wherein said at least one gene to promote the production of an organosulfur compound is selected from list comprised of: mcbR (SEQ 184), amtr (SEQ 186), xsc-like (SEQ 188), ssuI (SEQ 190), ssuD1 (SEQ 192), ssuD2 (SEQ 194), ssuR (SEQ 196), accA (SEQ 206), accB (SEQ 208), seuA (SEQ 210), seuB (SEQ 212), seuC (SEQ 214), ilvA (SEQ 216), gldc (SEQ 220), ilvB (SEQ 222), ilvN (SEQ 224), ilvC (SEQ 226), ilvD (SEQ 228), pyc (SEQ 230), dadA-like (SEQ 232), coaA (SEQ 234), coaBC (SEQ 236), coal (SEQ 238), coaE (SEQ 240), panB (SEQ 244), panC (SEQ 246), panD (SEQ 248), panE (SEQ 250), aspB (SEQ 254), mqo (SEQ 256), mdh (SEQ 260), mcr (SEQ 262), puuE (SEQ 264), abat (SEQ 266), and pydD (SEQ 268).

8. The method according to claim 7 wherein, upon transcription and translation under the control of a native or synthetic promoter and Ribosomal binding site (RBS), said at least one gene to promote the production of an organosulfur compound provides a polypeptide sequence selected from the group consisting of: SEQ 185, SEQ 187, SEQ 189, SEQ 191, SEQ 193, SEQ 195, SEQ 197, SEQ 207, SEQ 209, SEQ 211, SEQ 213, SEQ 215, SEQ 217, SEQ 221, SEQ 223, SEQ 225, SEQ 227, SEQ 229, SEQ 231, SEQ 233, SEQ 235, SEQ 237, SEQ 239, SEQ 241, SEQ 245, SEQ 247, SEQ 249, SEQ 251, SEQ 255, SEQ 257, SEQ 261, SEQ 263, SEQ 265, SEQ 267, and SEQ 269.

9. The method according to claim 6 wherein said at least one gene related to the cellular transportation of an organosulfur compound is selected from list comprised of: ssuA (SEQ 198), ssuB (SEQ 200), ssuC (SEQ 202), tauE (SEQ 204), gadC (SEQ 218), yhiM (SEQ 242), sdaC (SEQ 252), and cycA (SEQ 258).

10. The method according to claim 9 wherein, upon transcription and translation under the control of a native or synthetic promoter and Ribosomal binding site (RBS), said at least one gene involved in the transport of an organosulfur compound generates a polypeptide sequence selected from the group consisting: SEQ 199, SEQ 201, SEQ 203, SEQ 205, SEQ 219, SEQ 243, SEQ 253, and SEQ 259.

11. The method according to claim 1, whether said prokaryotic cells further comprises a promoter and Ribosomal Binding Site (RBS) sequence which drives gene expression, wherein the genetic sequence for the promoter/RBS comprises at least one of the following: SEQ 7; SEQ 8; SEQ 9: SEQ 10; SEQ 11; SEQ 12; SEQ 13; SEQ 14; SEQ 15; SEQ 16; SEQ 17; SEQ 18; SEQ 19; SEQ 20; SEQ 21; SEQ 22; and SEQ 23.

12. The method according to claim 1, where the live prokaryotic cell is a bacterial cell.

13. The method according to claim 1, where the live prokaryotic cell is selected from the group consisting of the genera: Brevibacterium, Bacillus, Corynebacterium, Escherichia, Lactococcus, Pseudomonas, Rhodococcus, and Serratia.

14. The method according to claim 1, where the live prokaryotic cell is Corynebacterium glutamicum.

15. The method according to claim 1, where the carbon source is selected from the group consisting of: hydrolysates and other complex sugar-based mixtures, long-chain saccharides, short chain saccharides, monosaccharides, sugar alcohols, organic acids and their corresponding salts and/or combinations thereof.

16. The method according to claim 1, where the sulfur source is selected from the group consisting of: inorganic compounds comprising sulfur in any oxidation state; organic compounds comprising sulfur in any oxidation state; and their corresponding salts.

17. The method according to claim 1, where said organosulfur compound is selected from the group consisting of: cysteamine, hypotaurine, and taurine.

18. The method according to claim 1, where said organosulfur compound is taurine.

19. A method of growing a genetically modified prokaryotic cell, wherein the method comprises the steps of:
a. providing a live genetically modified prokaryotic cell capable of expressing at least one gene for the production of an organosulfur compound;
b. exposing said live genetically modified prokaryotic cell to a culture media with a pH of between 4 and 11; thereby creating an incubation mixture; and
c. incubating said live prokaryotic cell in said incubation mixture under aerobic and/or anaerobic conditions at a temperature ranging from 0° C. to 60° C. for a period of time ranging from 3 hours to 15 days allowing for the growth of said live genetically modified prokaryotic cell.
